(19) United States
(12) Patent Application Publication
(10) Pub. No.: US 2024/0092789 A1
(43) Pub. Date: Mar. 21, 2024

(54) COMPOUNDS FOR ELECTRONIC DEVICES

(71) Applicant: Merck Patent GmbH, Darmstadt (DE)

(72) Inventors: Elvira MONTENEGRO, Weinheim (DE); Teresa MUJICA-FERNAUD, Tenerife (ES); Rachel TUFFIN, Chandlers Ford (GB); Frank VOGES, Bad Duerkheim (DE); Amir Hossain PARHAM, Frankfurt am Main (DE); Alexander Christian COMELY, Barcelona (ES); Antoni MORAGAS SOLA, Barcelona (ES)

(21) Appl. No.: 17/766,289

(22) PCT Filed: Sep. 16, 2020
(86) PCT No.: PCT/EP2020/075790

§ 371 Date: Apr. 4, 2022

(30) Foreign Application Priority Data
Oct. 11, 2019 (EP) 19202698.7

(51) Int. Cl. C07D 487/10; C07D 209/70; C07D 307/94; C07D 333/78; H10K 50/11; H10K 50/15; H10K 85/60

(52) U.S. Cl. CPC C07D 487/10; C07D 209/70; C07D 307/94; C07D 333/78; H10K 50/11; H10K 50/15; H10K 85/6572; H10K 85/6574; H10K 85/6576

(57) ABSTRACT

The present application relates to a compound of the formula (I), to the use thereof in electronic devices, to processes for preparing the compound, and electronic devices comprising the compound.

The present application relates to spirobifluorene derivatives in which one or more of the benzene rings have been exchanged for a heteroaryl ring. The compounds are suitable for use in electronic devices.

Electronic devices in the context of this application are understood to mean what are called organic electronic devices, which contain organic semiconductor materials as functional materials. More particularly, these are understood to mean OLEDs (organic electroluminescent devices). The term OLEDs is understood to mean electronic devices which have one or more layers comprising organic compounds and emit light on application of electrical voltage. The construction and general principle of function of OLEDs are known to those skilled in the art.

In electronic devices, especially OLEDs, there is great interest in an improvement in the performance data. In these aspects, it has not yet been possible to find any entirely satisfactory solution.

A great influence on the performance data of electronic devices is possessed by emission layers and layers having a hole-transporting function. Novel compounds are also being sought for use in these layers, especially hole-transporting compounds and compounds that can serve as hole-transporting matrix material, especially for phosphorescent emitters, in an emitting layer. For this purpose, there is a search especially for compounds that have a high glass transition temperature, high stability, and high conductivity for holes. A high stability of the compound is a prerequisite for achieving a long lifetime of the electronic device.

In the prior art, triarylamine compounds such as spirobifluoreneamines and fluoreneamines in particular are known as hole transport materials and hole-transporting matrix materials for electronic devices.

However, there is still a need for alternative compounds suitable for use in electronic devices, especially for compounds having one or more of the abovementioned advantageous properties. There is still a need for improvement in the performance data achieved when the compounds are used in electronic devices, especially in respect of lifetime, operating voltage and efficiency of the devices.

It has now been found that particular spirobifluorene derivatives in which one or more benzene rings have been exchanged for heteroaryl rings are of excellent suitability for use in electronic devices. They are especially suitable for use in OLEDs, and even more particularly therein for use as hole transport materials and for use as hole-transporting matrix materials, especially for phosphorescent emitters. The compounds found lead to high lifetime, high efficiency and low operating voltage of the devices. Further preferably, the compounds found have a high glass transition temperature, high stability and high conductivity for holes.

The present application thus provides compounds of the following formula (I):

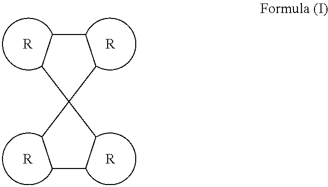

where the units R are the same or different at each instance and are selected from units of the formulae (R-1) to (R-3)

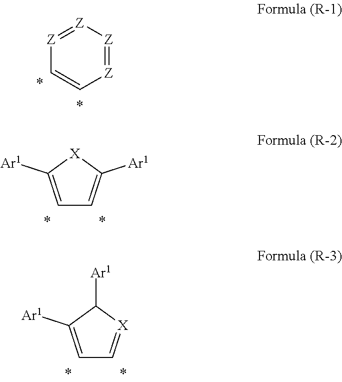

and where:
the units of the formulae (R-1) to (R-3) are each bonded to the remainder of the formula (I) via the positions identified by *;
at least one unit R in formula (I) is selected from units of the formulae (R-2) and (R-3);
X is the same or different at each instance and is selected from O, S and NAr⁰;
Z is the same or different at each instance and is selected from N and CR¹;
Ar⁰ is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar¹ is the same or different at each instance and is selected from H, D, F, Cl, Br, I, C(═O)R², CN, Si(R²)₃, P(═O)(R²)₂, OR², S(═O)R², S(═O)₂R², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by one or more R² radicals; and where one or more CH₂ groups in the alkyl, alkoxy, alkenyl and alkynyl groups mentioned may be replaced by —R²C═CR²—, —C≡C—, Si(R²)₂, C═O, C═NR², —C(═O)O—, —C(═O)NR²—, NR², P(═O)(R²), —O—, —S—, SO or SO₂;
R¹ is the same or different at each instance and is selected from H, D, F, Cl, Br, I, C(═O)R⁵, CN, Si(R⁵)₃, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form an aliphatic or heteroaliphatic ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN; and
at least one A group conforming to a formula (A) is bonded to at least one unit R, either directly to the ring of the unit R or to an Ar⁰ or Ar¹ group:

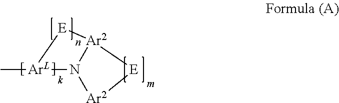

where:
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar² is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals;
E is the same or different at each instance and is a single bond or a divalent group selected from C(R⁴)₂, Si(R⁴)₂, N(R⁴), O, and S;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
k is 0 or 1, where, in the case that k=0, the Ar^{L} group is absent and the nitrogen atom of the group of the formula (A) constitutes the attachment position, and at the same time n=0; and
m is 0 or 1, where, in the case that m=0, the E group in question is absent and the Ar² groups are not bonded to one another;
n is 0 or 1, where, in the case that n=0, the E group in question is absent and the Ar^{L} and Ar² groups are not bonded to one another.

When an A group is bonded to a unit R, this means that no R¹ or R² group is bonded at the position in question, such that this position is free for the bond to the A group.

The definitions which follow are applicable to the chemical groups that are used in the present application. They are applicable unless any more specific definitions are given.

An aryl group in the context of this invention is understood to mean either a single aromatic cycle, i.e. benzene, or a fused aromatic polycycle, for example naphthalene, phenanthrene or anthracene. A fused aromatic polycycle in the context of the present application consists of two or more single aromatic cycles fused to one another. Fusion between cycles is understood here to mean that the cycles share at least one edge with one another. An aryl group in the context of this invention contains 6 to 40 aromatic ring atoms. In addition, an aryl group does not contain any heteroatom as aromatic ring atoms, but only carbon atoms.

A heteroaryl group in the context of this invention is understood to mean either a single heteroaromatic cycle, for example pyridine, pyrimidine or thiophene, or a fused heteroaromatic polycycle, for example quinoline or carbazole. A fused heteroaromatic polycycle in the context of the present application consists of two or more single aromatic or heteroaromatic cycles that are fused to one another, where at least one of the aromatic and heteroaromatic cycles is a heteroaromatic cycle. Fusion between cycles is understood here to mean that the cycles share at least one edge with one another. A heteroaryl group in the context of this invention contains 5 to 40 aromatic ring atoms of which at least one is a heteroatom. The heteroatoms of the heteroaryl group are preferably selected from N, O and S.

An aryl or heteroaryl group, each of which may be substituted by the abovementioned radicals, is especially understood to mean groups derived from benzene, naphthalene, anthracene, phenanthrene, pyrene, dihydropyrene, chrysene, perylene, triphenylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, benzimidazolo[1,2-a]benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the context of this invention is a system which does not necessarily contain solely aryl groups, but which may additionally contain one or more non-aromatic rings fused to at least one aryl group. These non-aromatic rings contain exclusively carbon atoms as ring atoms. Examples of groups covered by this definition are tetrahydronaphthalene, fluorene and spirobifluorene. In addition, the term “aromatic ring system” includes systems that consist of two or more aromatic ring systems joined to one another via single bonds, for example biphenyl, terphenyl, 7-phenyl-2-fluorenyl, quaterphenyl and 3,5-diphenyl-1-phenyl. An aromatic ring system in the context of this invention contains 6 to 40 carbon atoms and no heteroatoms in the ring system. The definition of “aromatic ring system” does not include heteroaryl groups.

A heteroaromatic ring system conforms to the abovementioned definition of an aromatic ring system, except that it must contain at least one heteroatom as ring atom. As is the case for the aromatic ring system, the heteroaromatic ring system need not contain exclusively aryl groups and heteroaryl groups, but may additionally contain one or more non-aromatic rings fused to at least one aryl or heteroaryl group. The nonaromatic rings may contain exclusively carbon atoms as ring atoms, or they may additionally contain one or more heteroatoms, where the heteroatoms are preferably selected from N, O and S. One example of such a heteroaromatic ring system is benzopyranyl. In addition, the term “heteroaromatic ring system” is understood to mean systems that consist of two or more aromatic or heteroaromatic ring systems that are bonded to one another via single bonds, for example 4,6-diphenyl-2-triazinyl. A heteroaromatic ring system in the context of this invention contains 5 to 40 ring atoms selected from carbon and heteroatoms, where at least one of the ring atoms is a heteroatom. The heteroatoms of the heteroaromatic ring system are preferably selected from N, O and S.

The terms “heteroaromatic ring system” and “aromatic ring system” as defined in the present application thus differ from one another in that an aromatic ring system cannot have a heteroatom as ring atom, whereas a heteroaromatic ring system must have at least one heteroatom as ring atom. This heteroatom may be present as a ring atom of a non-aromatic heterocyclic ring or as a ring atom of an aromatic heterocyclic ring.

In accordance with the above definitions, any aryl group is covered by the term “aromatic ring system”, and any heteroaryl group is covered by the term “heteroaromatic ring system”.

An aromatic ring system having 6 to 40 aromatic ring atoms or a heteroaromatic ring system having 5 to 40 aromatic ring atoms is especially understood to mean groups derived from the groups mentioned above under aryl groups and heteroaryl groups, and from biphenyl, terphenyl, quaterphenyl, fluorene, spirobifluorene, dihydrophenanthrene, dihydropyrene, tetrahydropyrene, indenofluorene, truxene, isotruxene, spirotruxene, spiroisotruxene, indenocarbazole, or from combinations of these groups.

In the context of the present invention, a straight-chain alkyl group having 1 to 20 carbon atoms and a branched or cyclic alkyl group having 3 to 20 carbon atoms and an alkenyl or alkynyl group having 2 to 40 carbon atoms in which individual hydrogen atoms or CH₂ groups may also be substituted by the groups mentioned above in the definition of the radicals are preferably understood to mean the methyl, ethyl, n-propyl, i-propyl, n-butyl, i-butyl, s-butyl, t-butyl, 2-methylbutyl, n-pentyl, s-pentyl, cyclopentyl, neopentyl, n-hexyl, cyclohexyl, neohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl radicals.

An alkoxy or thioalkyl group having 1 to 20 carbon atoms in which individual hydrogen atoms or CH₂ groups may also be replaced by the groups mentioned above in the definition of the radicals is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

The wording that two or more radicals together may form a ring, in the context of the present application, shall be understood to mean, inter alia, that the two radicals are joined to one another by a chemical bond. In addition, however, the abovementioned wording shall also be understood to mean that, if one of the two radicals is hydrogen, the second radical binds to the position to which the hydrogen atom was bonded, forming a ring.

In a preferred embodiment of the invention, exactly one or exactly two unit(s) R in formula (I) are selected from units of the formulae (R-2) and (R-3), and the other units R conform to the formula (R-1). More preferably, exactly one unit R in formula (I) is selected from units of the formulae (R-2) and (R-3) and the remaining three units R conform to the formula (R-1).

In a preferred embodiment of the invention, exactly one or exactly two unit(s) R in formula (I) conform to the formula (R-2), and the other units R conform to the formula (R-1). More preferably, exactly one unit R in formula (I) conforms to the formula (R-2), and the remaining three units R conform to the formula (R-1).

X is preferably the same or different at each instance and is selected from O and S; more preferably, X is S.

Preferably not more than three Z groups, more preferably not more than two Z groups, even more preferably not more than one Z group, most preferably no Z group, in the unit of the formula (R-1) is N. The remaining groups are correspondingly CR¹. It is additionally preferable that no adjacent Z groups in one ring are N. It is still further preferred that not more than 3 Z groups in any formula (I) are N; more preferably, not more than 2 Z groups in any formula (I) are N; even more preferably, not more than one Z group in any formula (I) is N; most preferably, no Z group is N.

Ar⁰ is preferably the same or different at each instance and is selected from aromatic ring systems which have 6 to 40 aromatic ring atoms and are each substituted by R² radicals. More preferably, Ar⁰ is the same or different at each instance and are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Very particular preference is given to phenyl, biphenyl, terphenyl, quaterphenyl, naphthyl, fluorenyl, especially 9,9′-dimethylfluorenyl and 9,9′-diphenylfluorenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Most preferably, Ar⁰ is phenyl substituted by R² radicals, where R² is preferably H.

When an A group is bonded to an Ar¹ group, the Ar¹ group in question is preferably selected from aromatic ring systems which have 6 to 40 aromatic ring atoms and are substituted by R² radicals, and heteroaromatic ring systems which have 5 to 40 aromatic ring atoms and are substituted by R² radicals; more preferably from aromatic ring systems which have 6 to 40 aromatic ring atoms and are substituted by R² radicals; even more preferably from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R²; even more preferably from phenyl, biphenyl, terphenyl, quaterphenyl, naphthyl, fluorenyl, especially 9,9′-dimethylfluorenyl and 9,9′-diphenylfluorenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R²; most preferably from phenyl substituted by R² radicals, where R² is preferably H.

Ar¹ is preferably the same or different at each instance and is selected from H, D, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms, where the alkyl groups, alkoxy groups, aromatic ring systems and heteroaromatic ring systems are each substituted by one or more R² radicals. Ar¹ is more preferably the same or different at each instance and is selected from aromatic ring systems which have 6 to 40 aromatic ring atoms and are substituted by R² radicals. Even more preferably, Ar¹ is the same or different at each instance and are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Even greater preference is given to phenyl, biphenyl, terphenyl, quaterphenyl, naphthyl, fluorenyl, especially 9,9′-dimethylfluorenyl and 9,9′-diphenylfluorenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R² radicals. Most preferably, Ar¹ is phenyl substituted by R² radicals, where R² is preferably H.

R¹ is preferably the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═0, C═NR⁵, —NR⁵—, —O—, —S—, —C(═O)O— or —C(═O)NR⁵—. More preferably, R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R¹ is H.

Preferred R¹ groups are shown in the following table:

| | |
|---|---|
| 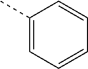 | R¹-1 |
| 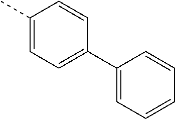 | R¹-2 |
| 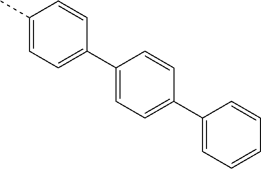 | R¹-3 |
| 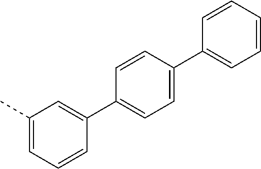 | R¹-4 |
| 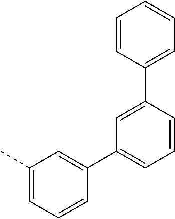 | R¹-5 |
| 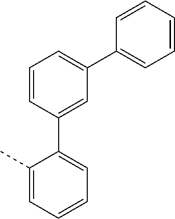 | R¹-6 |
| 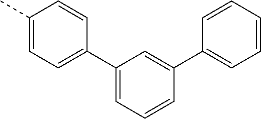 | R¹-7 |
| 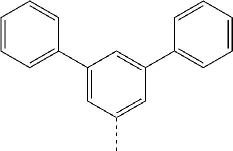 | R¹-8 |
| 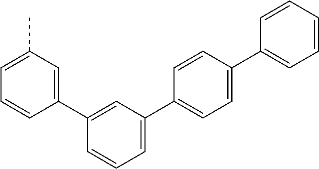 | R¹-9 |
| 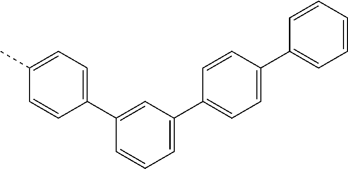 | R¹-10 |
| 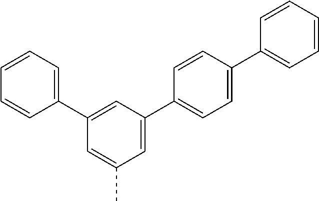 | R¹-11 |
| 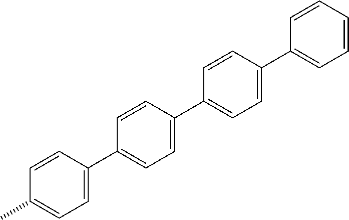 | R¹-12 |
| 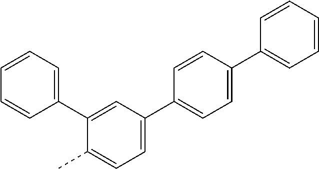 | R¹-13 |
| 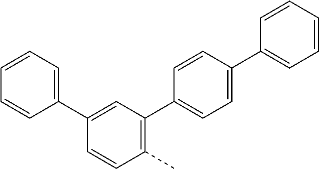 | R¹-14 |
| 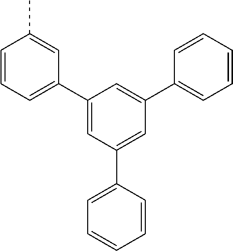 | R¹-15 |
| 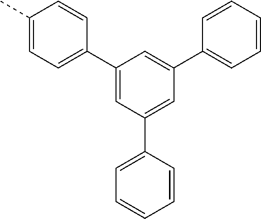 | R¹-16 |
| 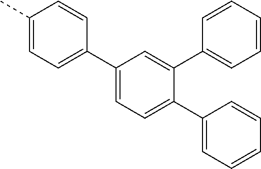 | R¹-17 |
| 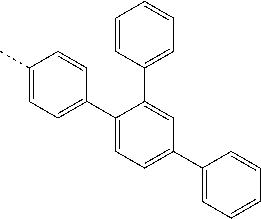 | R¹-18 |
| 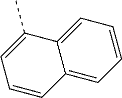 | R¹-19 |
| 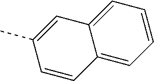 | R¹-20 |
| 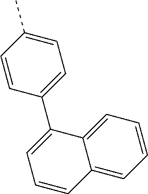 | R¹-21 |
| 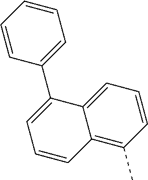 | R¹-22 |
| 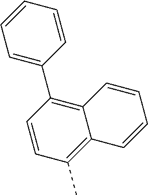 | R¹-23 |
| 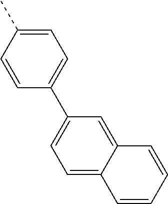 | R¹-24 |
| 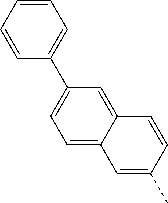 | R¹-25 |
| 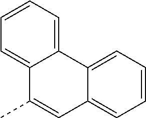 | R¹-26 |
| 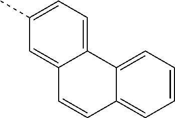 | R¹-27 |
| 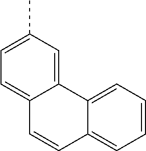 | R¹-28 |
| 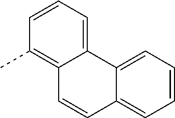 | R¹-29 |
| 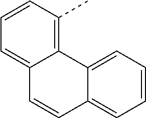 | R¹-30 |
| 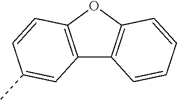 | R¹-31 |
| 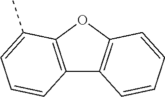 | R¹-32 |
| 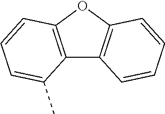 | R¹-33 |
| 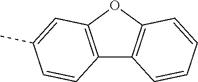 | R¹-34 |
| 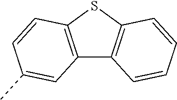 | R¹-35 |
| 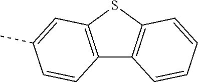 | R¹-36 |
| 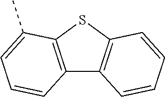 | R¹-37 |
| 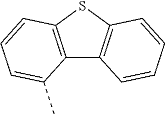 | R¹-38 |
| 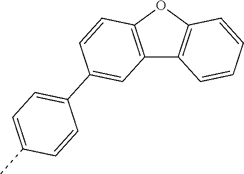 | R¹-39 |
| 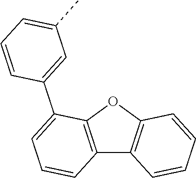 | R¹-40 |
| 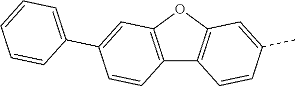 | R¹-41 |
| 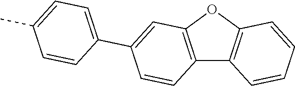 | R¹-42 |
| 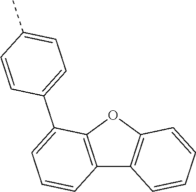 | R¹-43 |
| 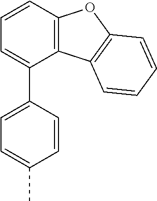 | R¹-44 |
| 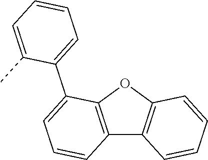 | R¹-45 |
| 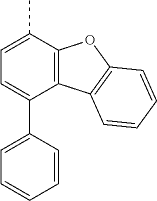 | R¹-46 |
| 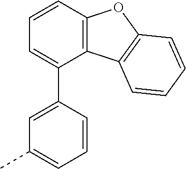 | R¹-47 |
| 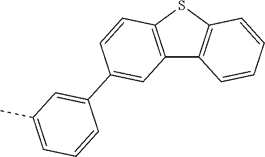 | R¹-48 |
| 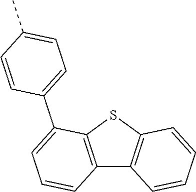 | R¹-49 |
| 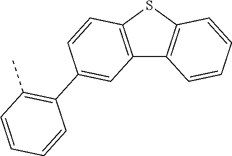 | R¹-50 |
| 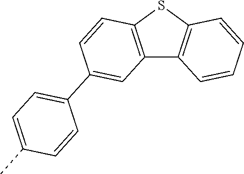 | R¹-51 |
| 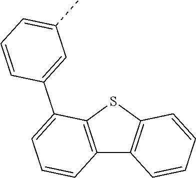 | R¹-52 |
| 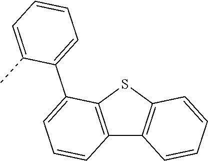 | R¹-53 |
| 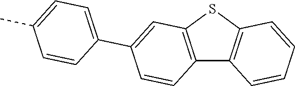 | R¹-54 |
| 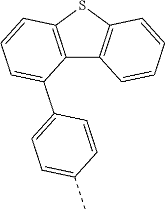 | R¹-55 |
| 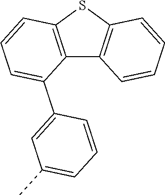 | R¹-56 |
| 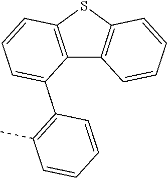 | R¹-57 |
| 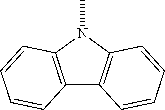 | R¹-58 |
| 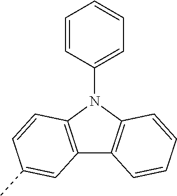 | R¹-59 |
| 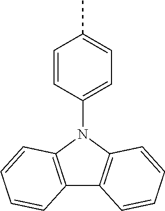 | R¹-60 |
| 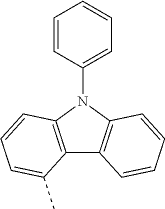 | R¹-61 |
| 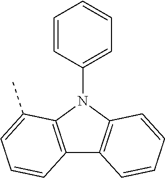 | R¹-62 |
| 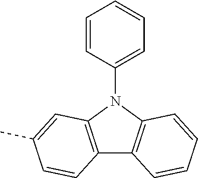 | R¹-63 |
| 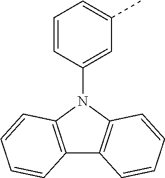 | R¹-64 |
| 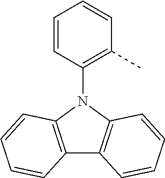 | R¹-65 |
| 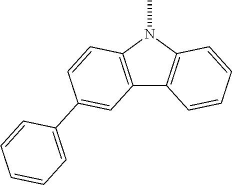 | R¹-66 |
| 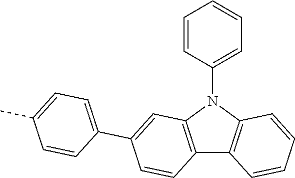 | R¹-67 |
| 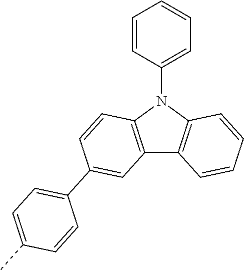 | R¹-68 |
| 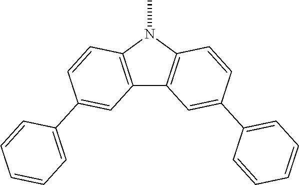 | R¹-69 |
| 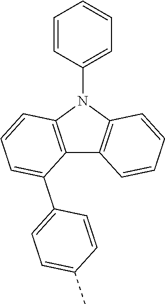 | R¹-70 |
| 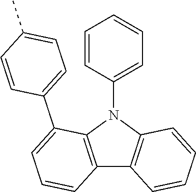 | R¹-71 |
| 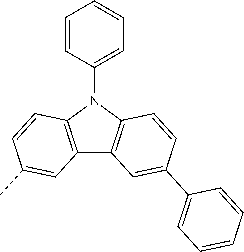 | R¹-72 |
| 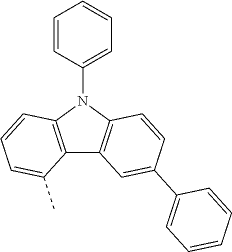 | R¹-73 |
| 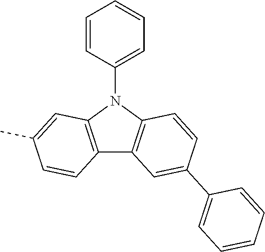 | R¹-74 |
| 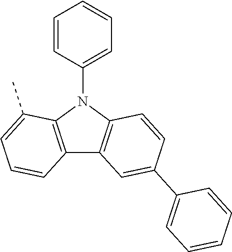 | R¹-75 |
| 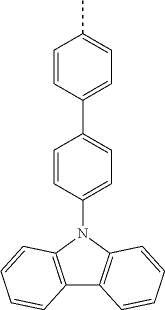 | R¹-76 |
| 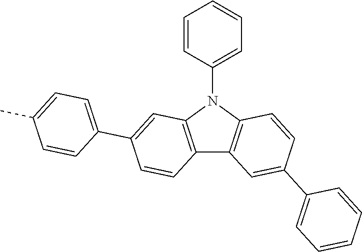 | R¹-77 |
| 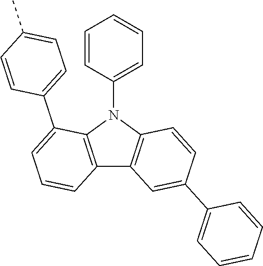 | R¹-78 |
| 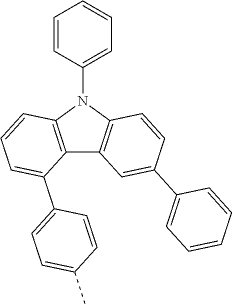 | R¹-79 |
| 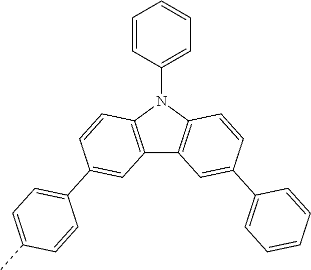 | R¹-80 |
| 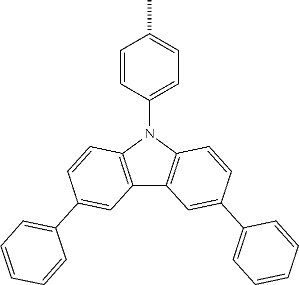 | R¹-81 |
| 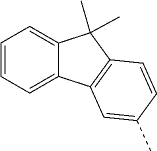 | R¹-82 |
| 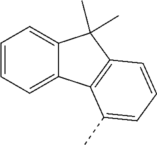 | R¹-83 |
| 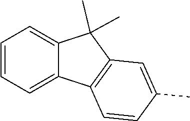 | R¹-84 |
| 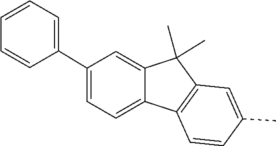 | R¹-85 |
| 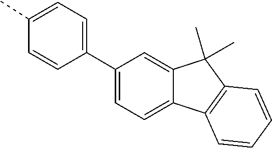 | R¹-86 |
| 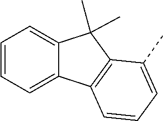 | R¹-87 |
| 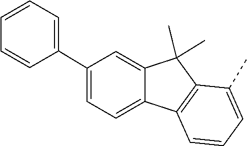 | R¹-88 |
| 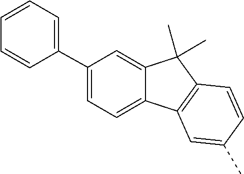 | R¹-89 |
| 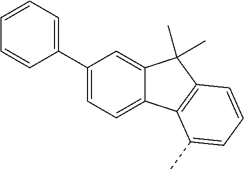 | R¹-90 |
| 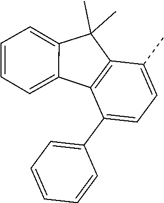 | R¹-91 |
| 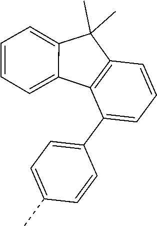 | R¹-92 |
| 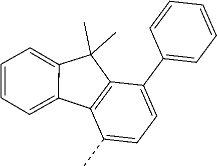 | R¹-93 |
| 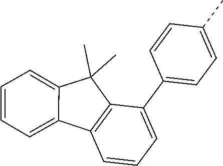 | R¹-94 |
| 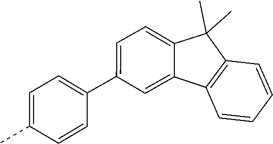 | R¹-95 |
| 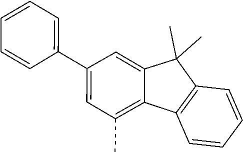 | R¹-96 |
| 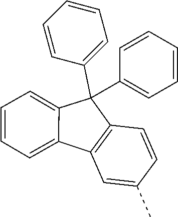 | R¹-97 |
| 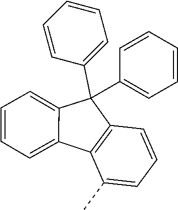 | R¹-98 |
| 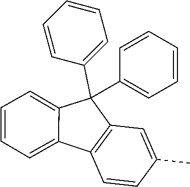 | R¹-99 |
| 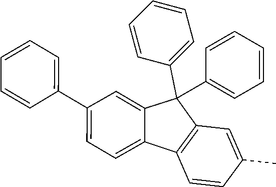 | R¹-100 |
| 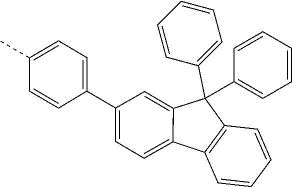 | R¹-101 |
| 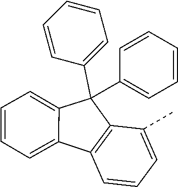 | R¹-102 |
| 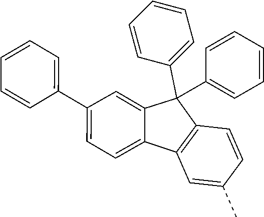 | R¹-103 |
| 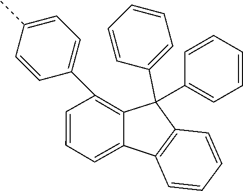 | R¹-104 |
| 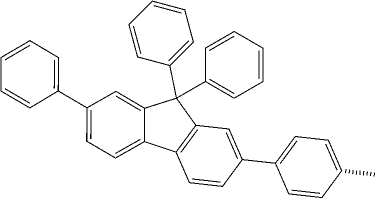 | R¹-105 |
| 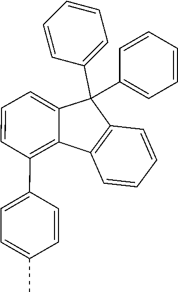 | R¹-106 |
| 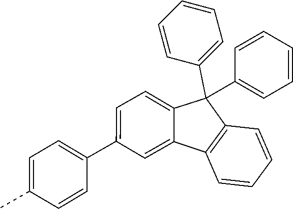 | R¹-107 |
| 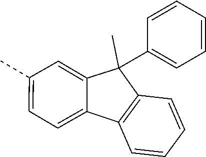 | R¹-108 |
| 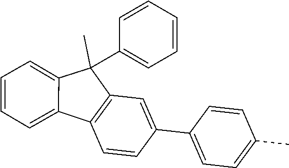 | R¹-109 |
| 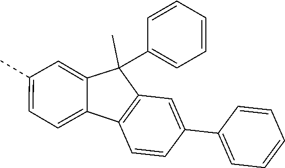 | R¹-110 |
| 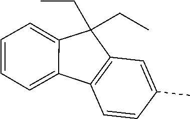 | R¹-111 |
| 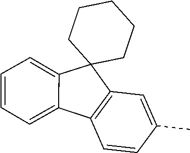 | R¹-112 |
| 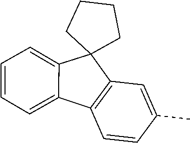 | R¹-113 |
| 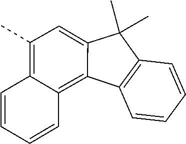 | R¹-114 |
| 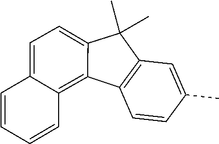 | R¹-115 |
| 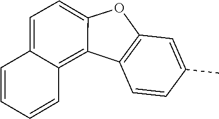 | R¹-116 |
| 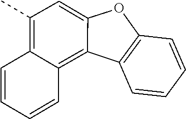 | R¹-117 |
| 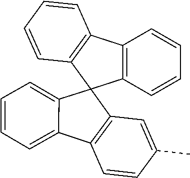 | R¹-118 |
| 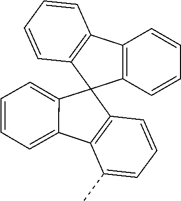 | R¹-119 |
| 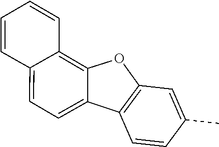 | R¹-120 |
| 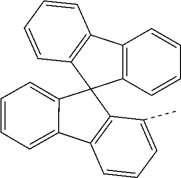 | R¹-121 |
| 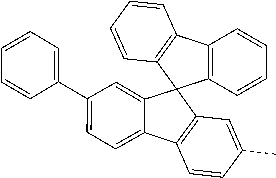 | R¹-122 |
| 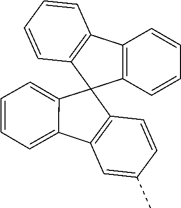 | R¹-123 |
| 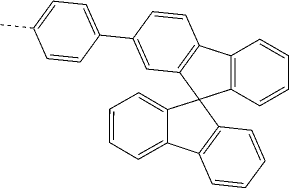 | R¹-124 |
| 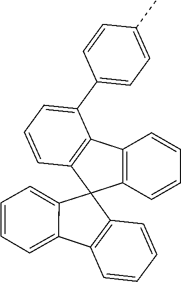 | R¹-125 |
| 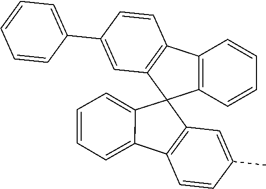 | R¹-126 |
| 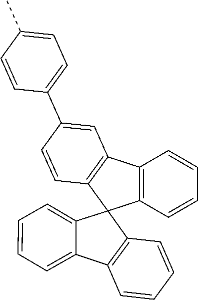 | R¹-127 |
| 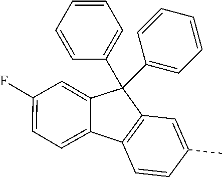 | R¹-128 |
| 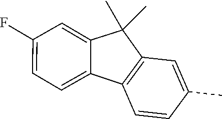 | R¹-129 |
| 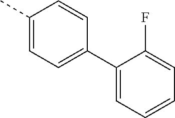 | R¹-130 |
| 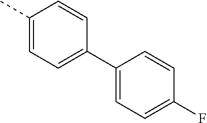 | R¹-131 |
| 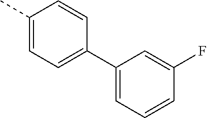 | R¹-132 |
| 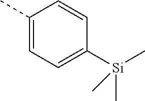 | R¹-133 |
| 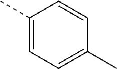 | R¹-134 |
| 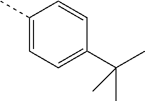 | R¹-135 |
| 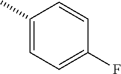 | R¹-136 |
| 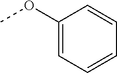 | R¹-137 |
| 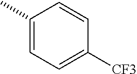 | R¹-138 |
| 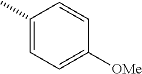 | R¹-139 |
| 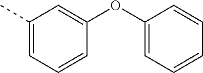 | R¹-140 |
| 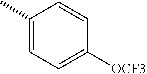 | R¹-141 |
| 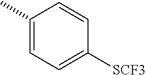 | R¹-142 |
| —F | R¹-143 |
| —Cl | R¹-144 |
| —Br | R¹-145 |
| —I | R¹-146 |
| —H | R¹-147 |
| —CH₃ | R¹-148 |
| 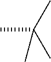 | R¹-149 |
| —CH₂CH₃ | R¹-150 |
| 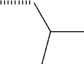 | R¹-151 |
| —CF₃ | R¹-152 |
| —CF₂CF₃ | R¹-153 |
| —OCF₃ | R¹-154 |
| —SCF₃ | R¹-155 |
| —SF₅ | R¹-156 |
| —OCF₂CF₃ | R¹-157 |
| —SCF₂CF₃ | R¹-158 |
| 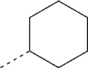 | R¹-159 |
| 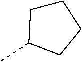 | R¹-160 |
| 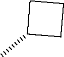 | R¹-161 |
| 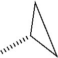 | R¹-162 |
| —CN | R¹-163 |
| —SCN | R¹-164 |
| —OCH₂CH₃ | R¹-165 |
| —OCH₃ | R¹-166 |
| —SCH₃ | R¹-167 |
| —Si(CH₃)₃ | R¹-168 |
| —Si(CH₃)₂t-Bu | R¹-169 |
| —Si(iPr)₃ | R¹-170 |
| —Si(CH₃)₂Ph | R¹-171 |
| Si—(Ph)₃ | R¹-172 |
| C—(Ph)₃ | R¹-173 |
| —D | R¹-174 |
| —CD₃ | R¹-175 |
| —CD₂—CD₃ | R¹-176 |
| —C(CD₃)₃ | R¹-177 |
| —CD₂—(CD₃)₂ | R¹-178 |
| —OCD₃ | R¹-179 |
| —SCD₃ | R¹-180 |
| —Si(CD₃)₃ | R¹-181 |
| 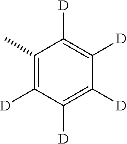 | R¹-182 |
| 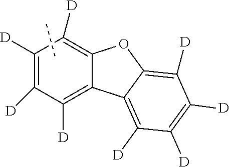 | R¹-183 |
| 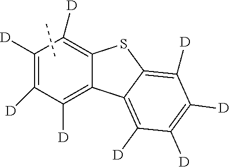 | R¹-184 |
| 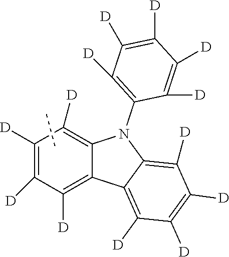 | R¹-185 |
| —CD₂(CH₃)₂ | R¹-186 |
| —CD₂(CH₃)₃ | R¹-187 |

Particular preference is given here to the R¹-1, R¹-2, R¹-143, R¹-148, R¹-149 and R¹-177 groups.

R² is preferably the same or different at each instance and is selected from H, D, F, CN, Si(R⁵)₃, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, —R⁵C═CR⁵—, Si(R⁵)₂, C═O, C═NR⁵, —NR⁵—, —O—, —S—, —C(═O)O— or —C(═O)NR⁵—. More preferably, R² is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R² is H.

Preferably, only one or two A groups are present in formula (I); more preferably, only one A group is present in formula (I).

When two A groups are present in formula (I), they are preferably bonded to two different units R.

Ar^{L} is preferably the same or different at each instance and is selected from aromatic ring systems which have 6 to 20 aromatic ring atoms and are substituted by R² radicals, and heteroaromatic ring systems which have 5 to 20 aromatic ring atoms and are substituted by R² radicals. Particularly preferred Ar^{L} groups are the same or different at each instance and are selected from divalent groups derived from benzene, biphenyl, terphenyl, naphthalene, fluorene, indenofluorene, indenocarbazole, spirobifluorene, dibenzofuran, dibenzothiophene, and carbazole, each of which are substituted by R² radicals. Even more preferably, Ar^{L} is a divalent group derived from benzene, biphenyl or naphthalene, each of which is substituted by one or more R² radicals, where the R² radicals in this case are preferably H.

Preferably, k is 0.

Preferred —(Ar^{L})ₖ— groups in the case that k=1 conform to the following formulae:

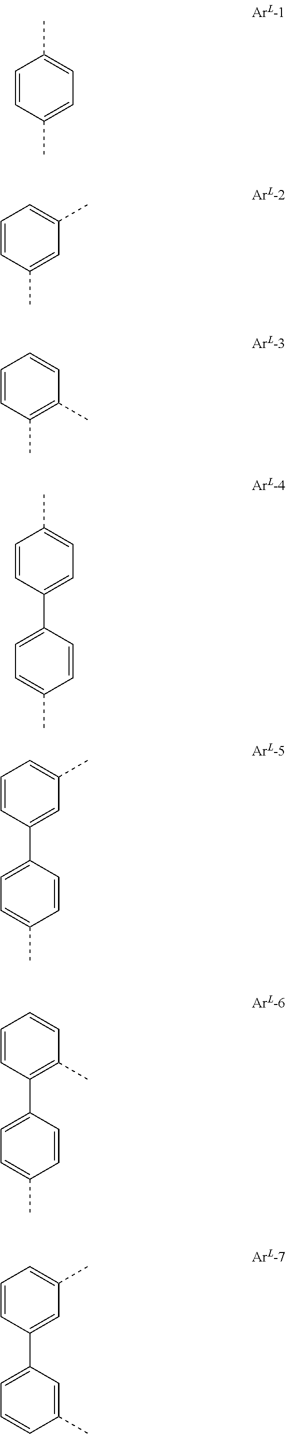

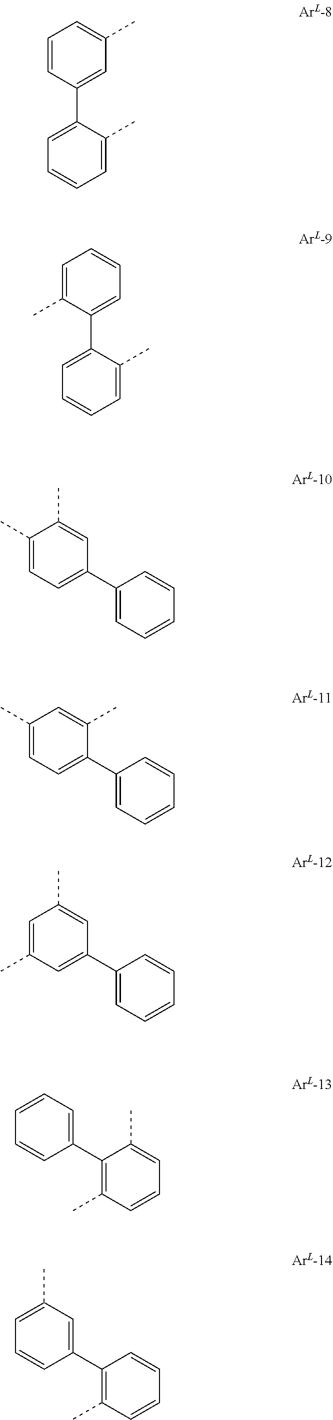

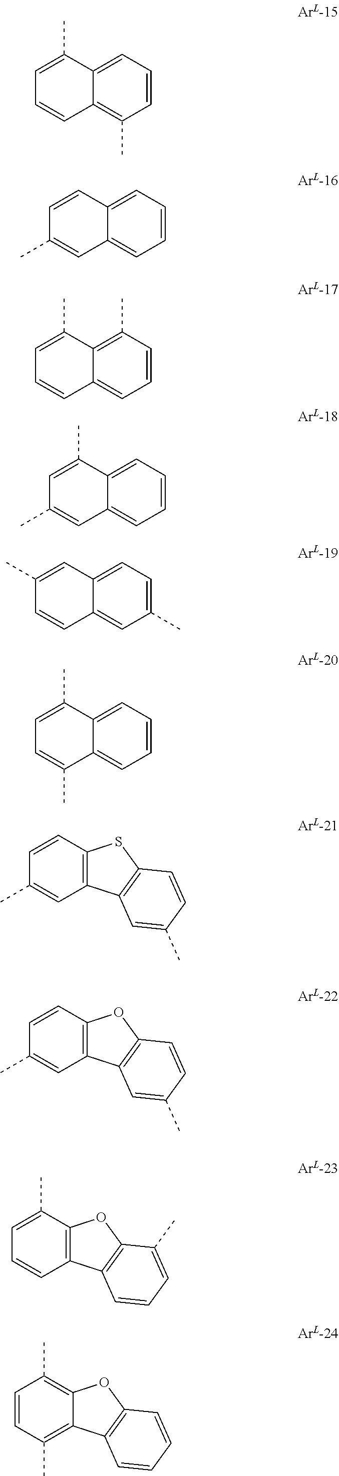

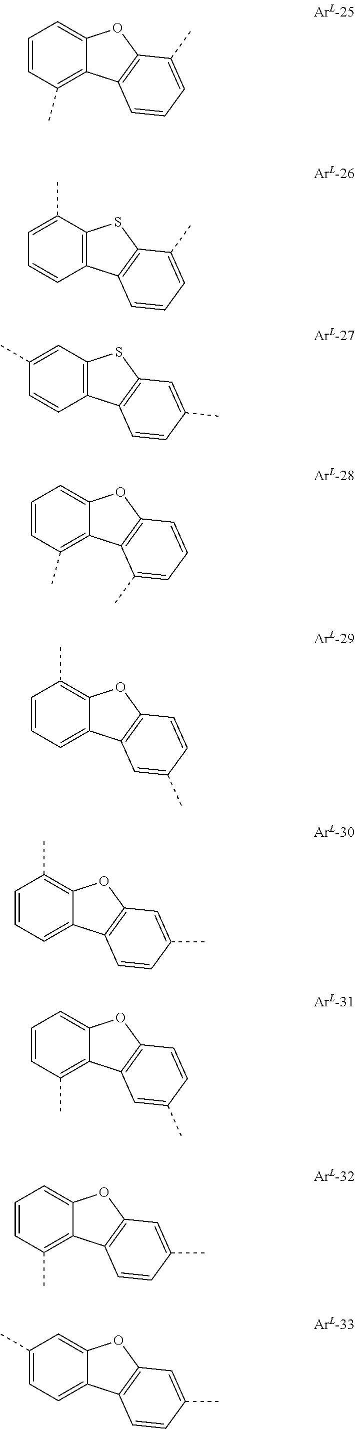

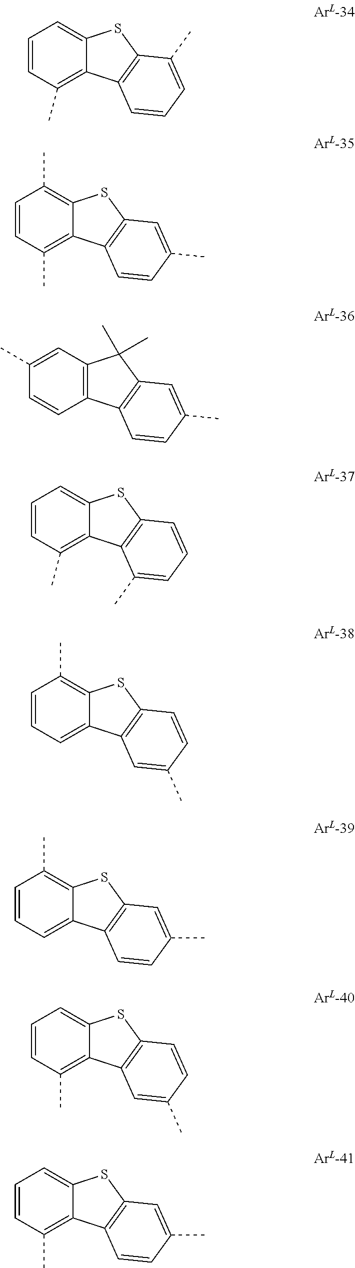

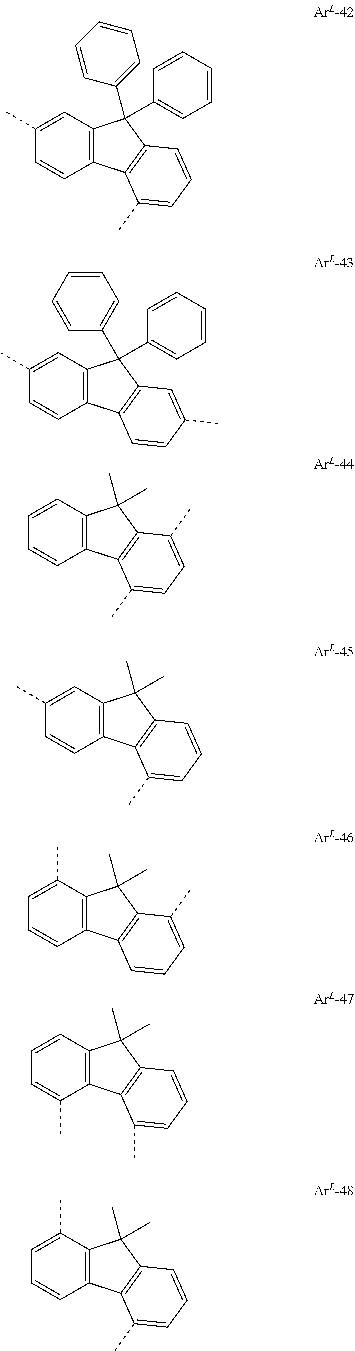

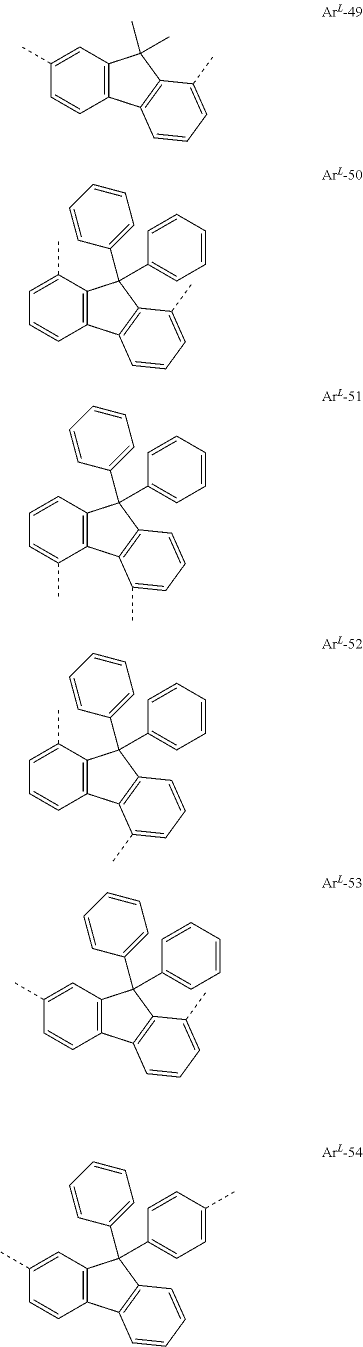

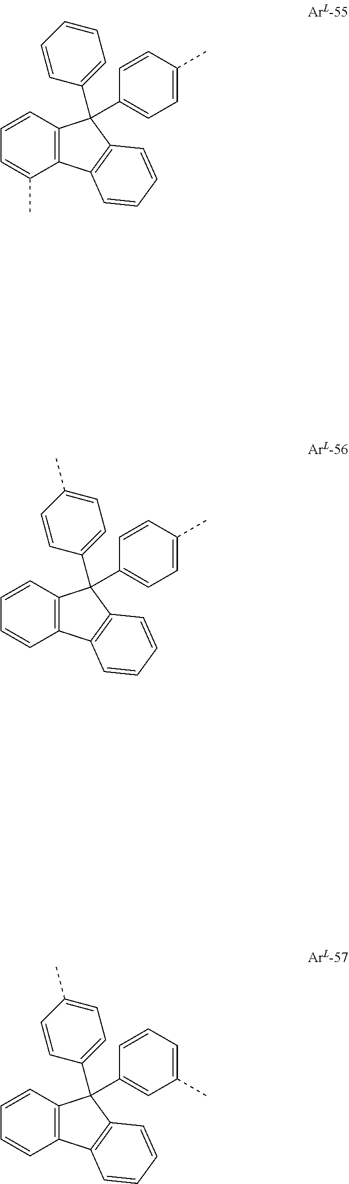

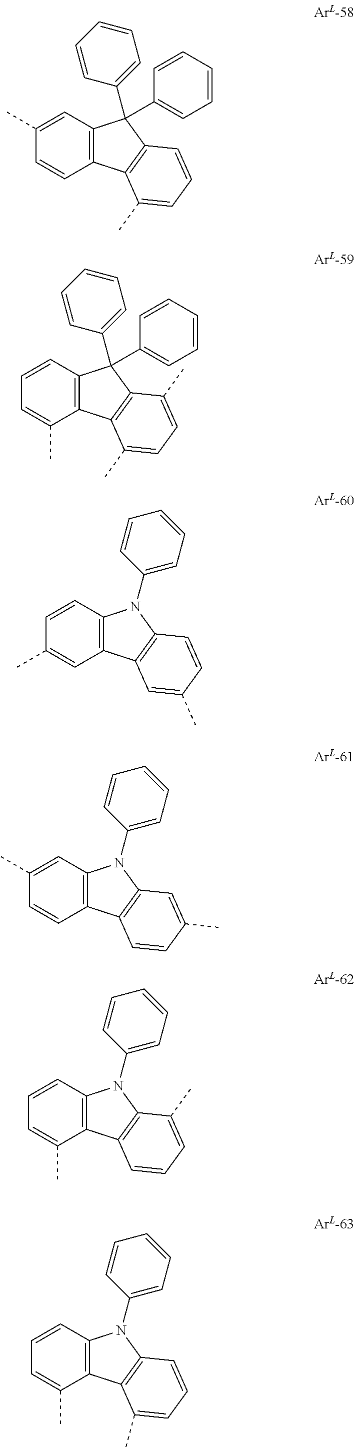

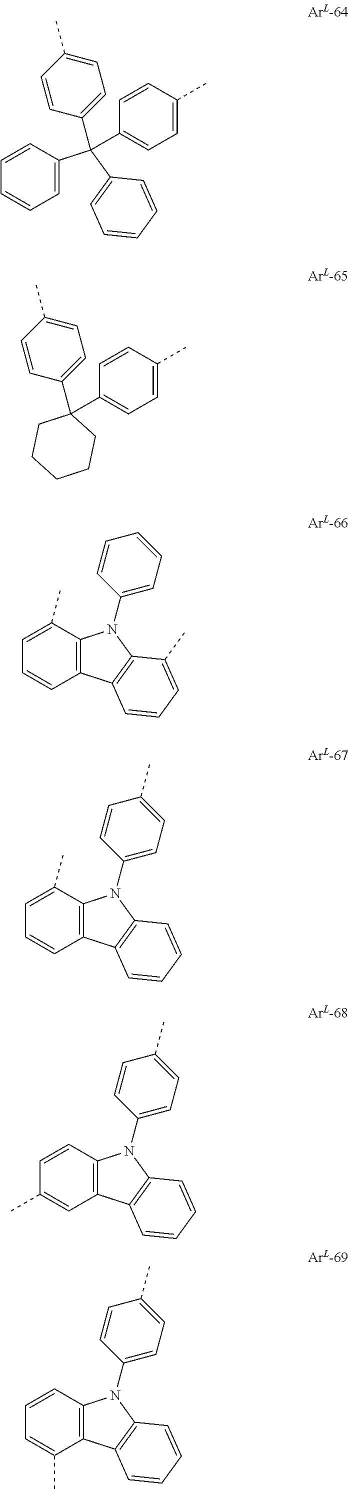

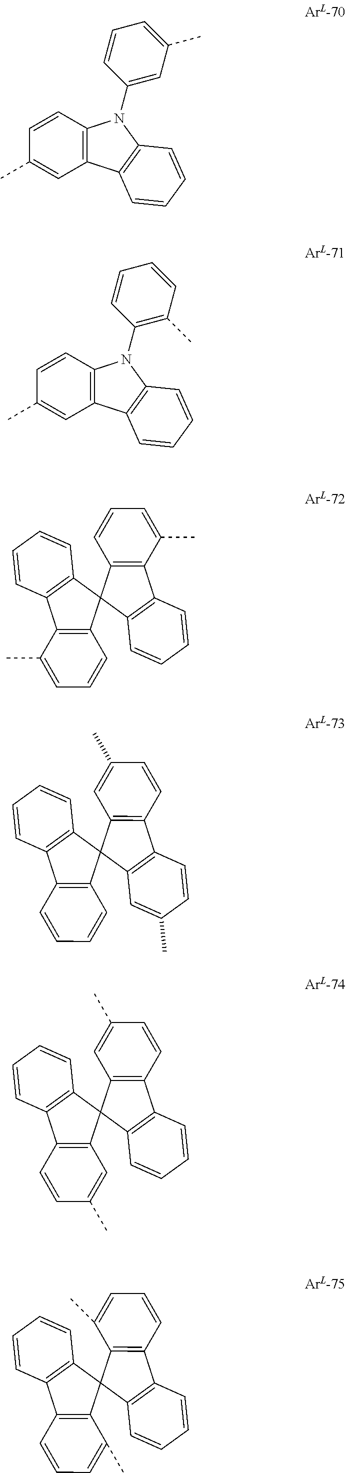

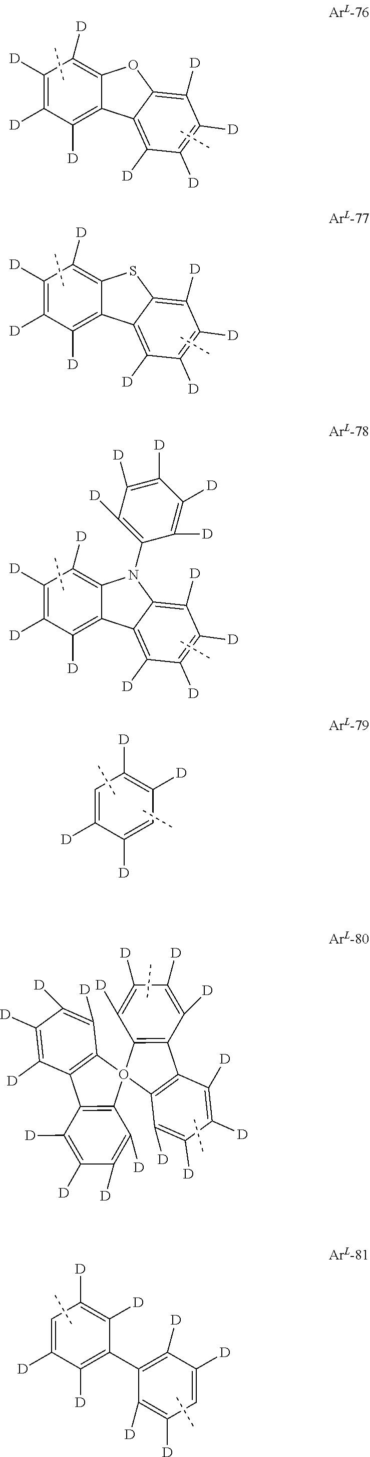

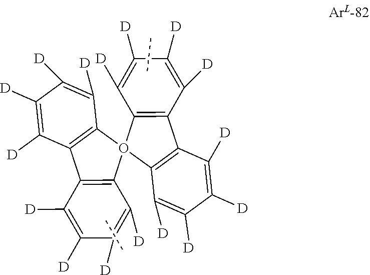

where the dotted lines represent the bonds to the rest of the formula (I), and where the groups at the positions shown as unsubstituted are each substituted by R² radicals, where the R² radicals in these positions are preferably H. Among the abovementioned formulae, particular preference is given to the formulae (Ar^{L}-1), (Ar^{L}-2), (Ar^{L}-3), (Ar^{L}-4), (Ar^{L}-15), (Ar^{L}-20), (Ar^{L}-25), (Ar^{L}-36).

Preferably, Ar² is the same or different at each instance and is selected from monovalent groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where the monovalent groups are each substituted by one or more R³ radicals. Alternatively, the Ar² groups are the same or different at each instance and may preferably be selected from combinations of groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, carbazole, benzofuran, benzothiophene, indole, quinoline, pyridine, pyrimidine, pyrazine, pyridazine and triazine, where the groups are each substituted by one or more R³ radicals.

In a preferred embodiment, the Ar² groups are fully or partly deuterated.

Particularly preferred Ar² groups are the same or different at each instance and are selected from phenyl, biphenyl, terphenyl, quaterphenyl, naphthyl, fluorenyl, especially 9,9′-dimethylfluorenyl and 9,9′-diphenylfluorenyl, benzofluorenyl, spirobifluorenyl, indenofluorenyl, indenocarbazolyl, dibenzofuranyl, dibenzothiophenyl, carbazolyl, benzofuranyl, benzothiophenyl, benzofused dibenzofuranyl, benzofused dibenzothiophenyl, naphthyl-substituted phenyl, fluorenyl-substituted phenyl, spirobifluorenyl-substituted phenyl, dibenzofuranyl-substituted phenyl, dibenzothiophenyl-substituted phenyl, carbazolyl-substituted phenyl, pyridyl-substituted phenyl, pyrimidyl-substituted phenyl, and triazinyl-substituted phenyl, where the groups mentioned are each substituted by R³ radicals.

Particularly preferred Ar² groups are the same or different and are selected from the following formulae:

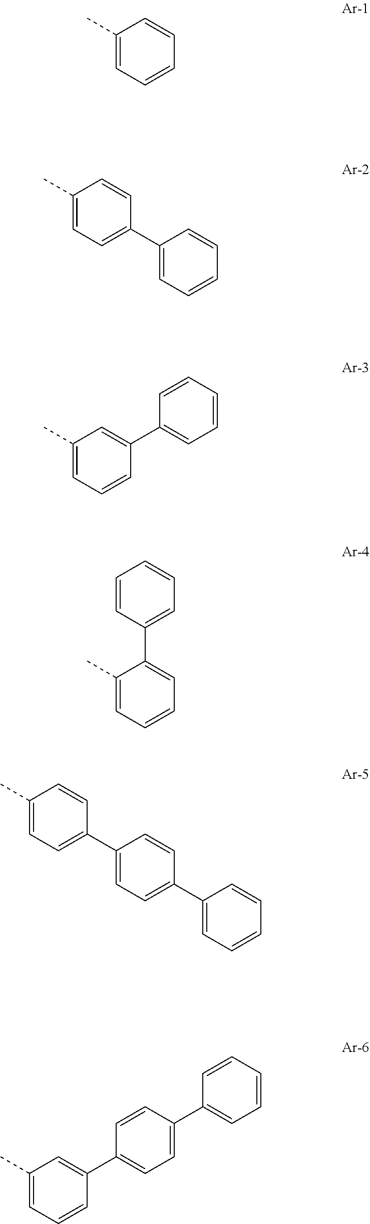

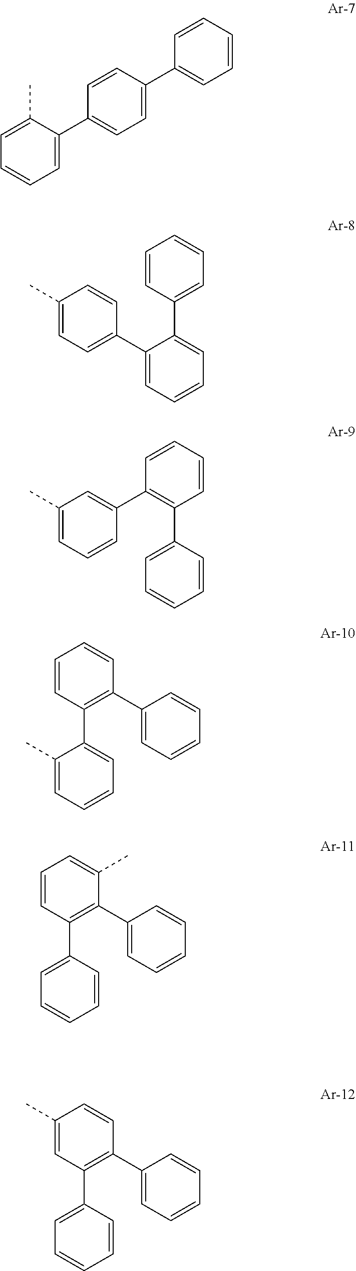

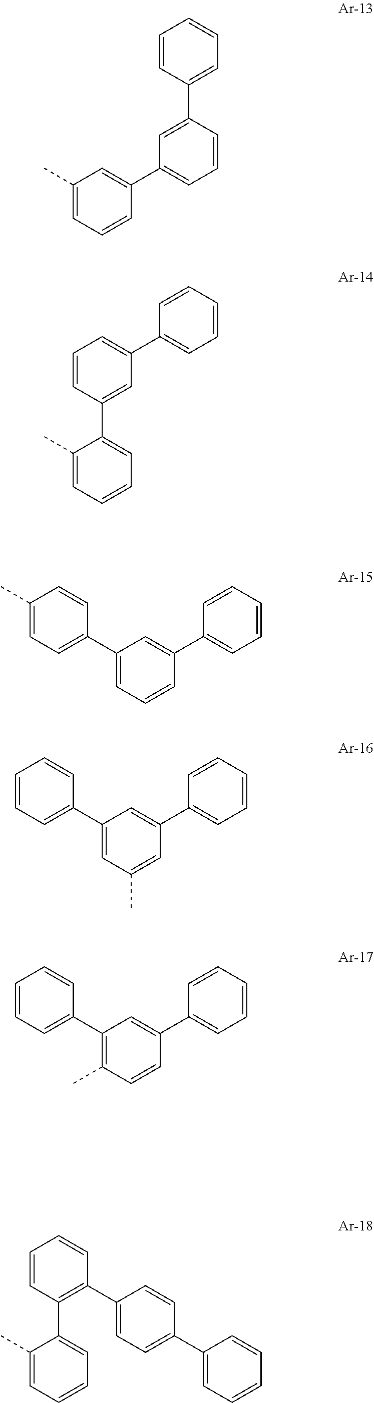

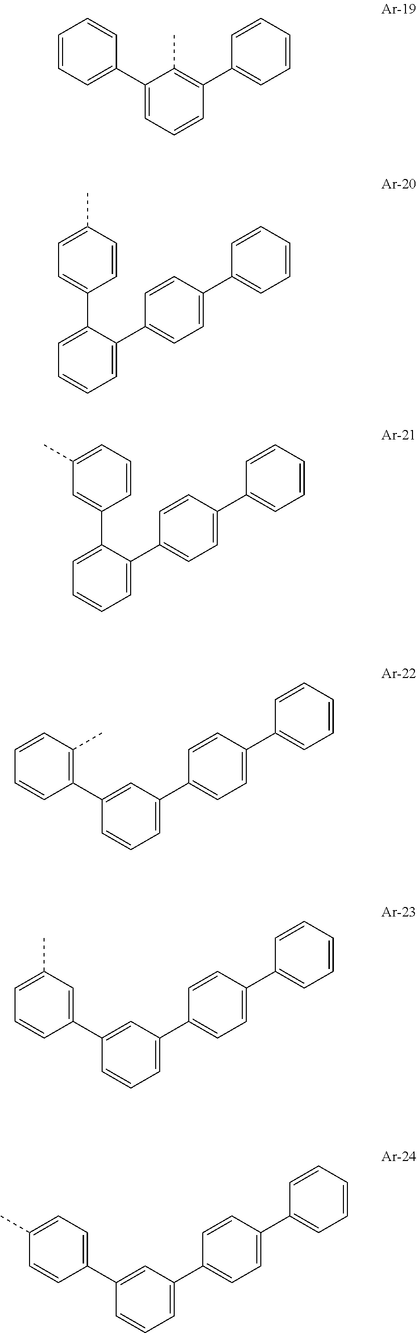

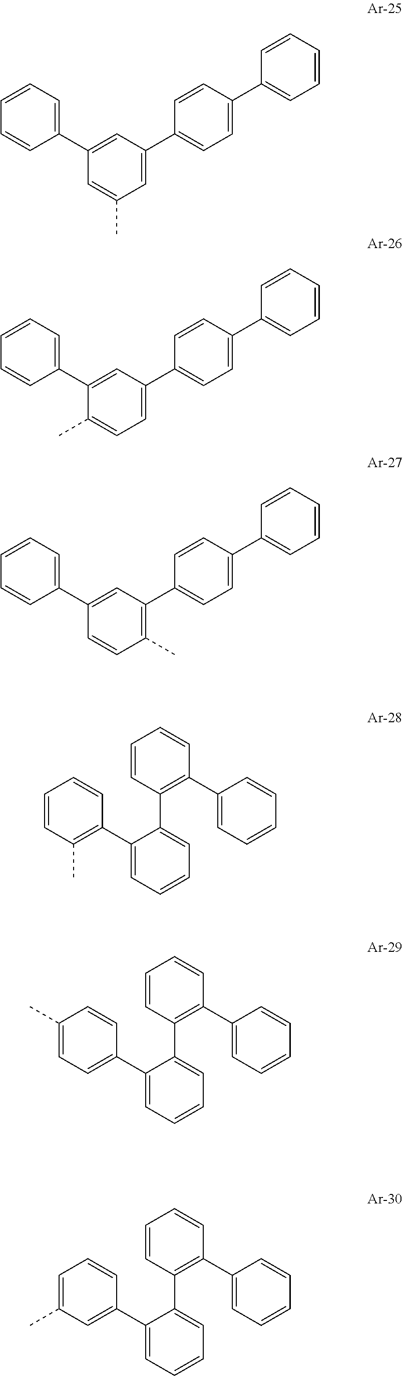

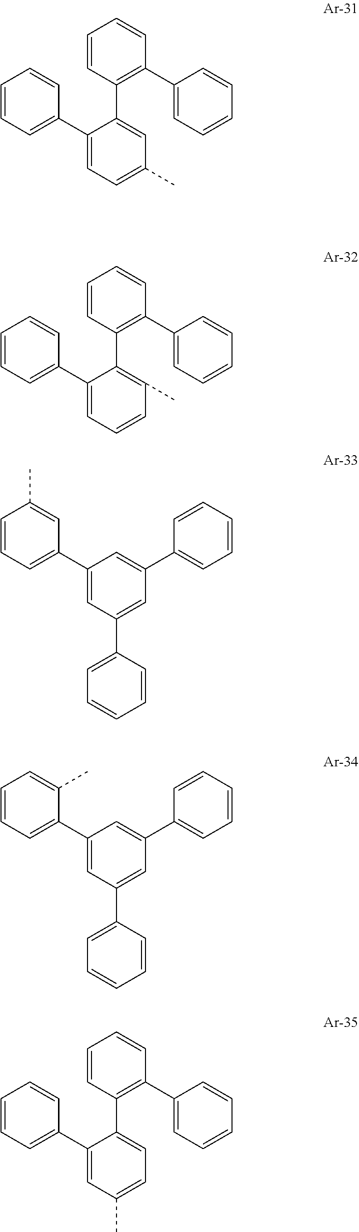

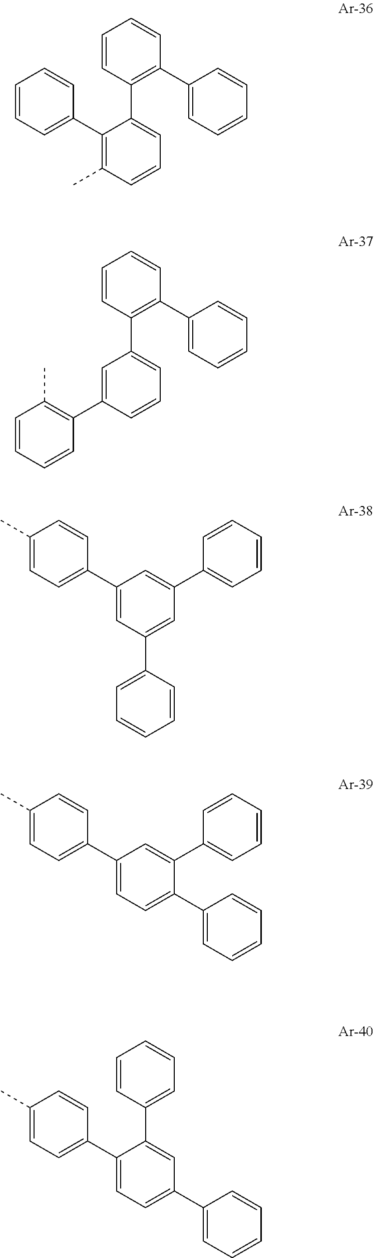

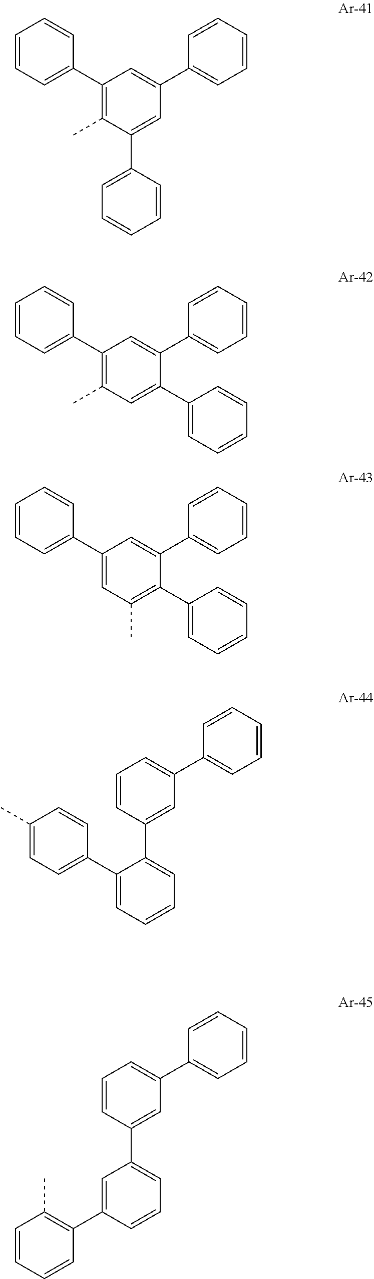

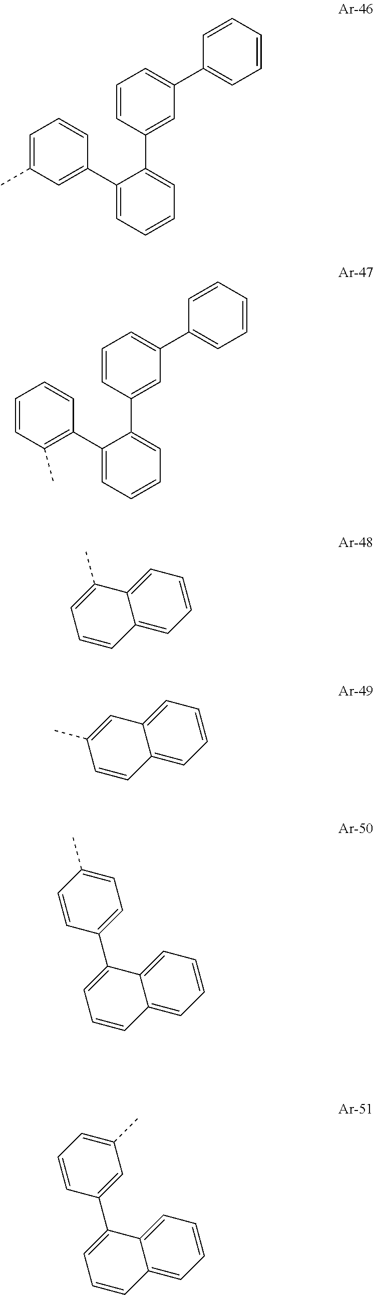

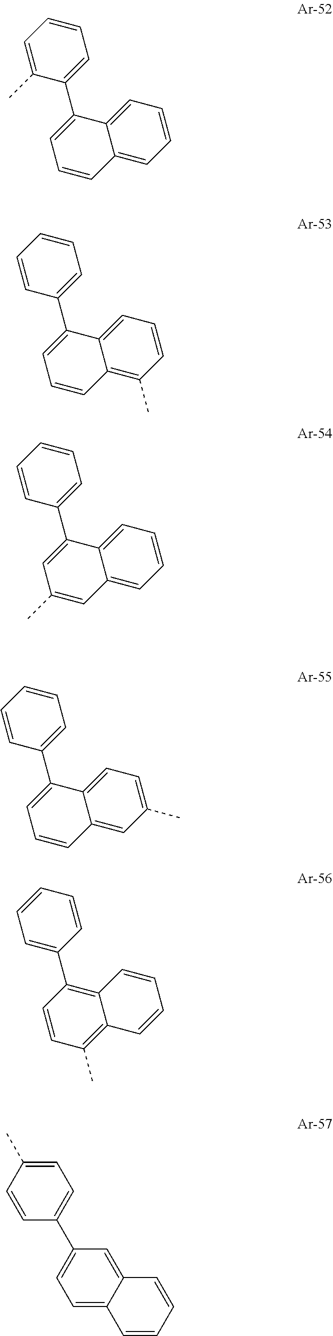

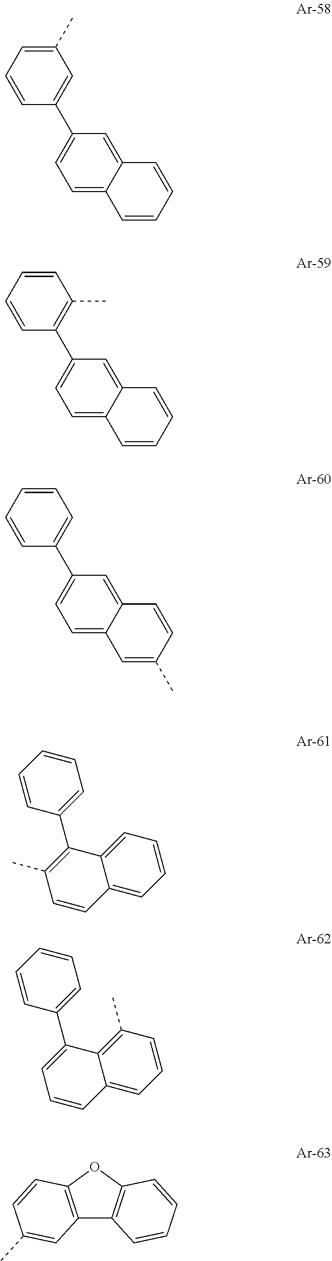

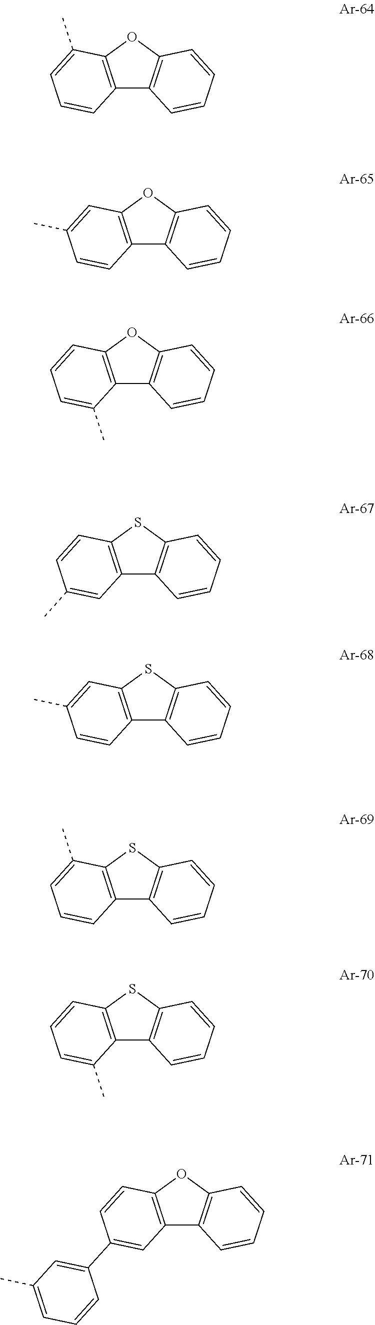

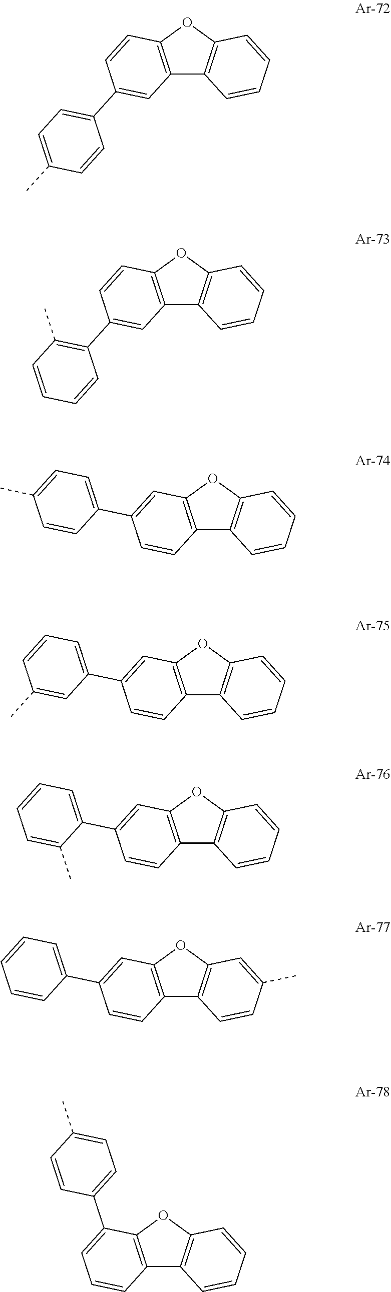

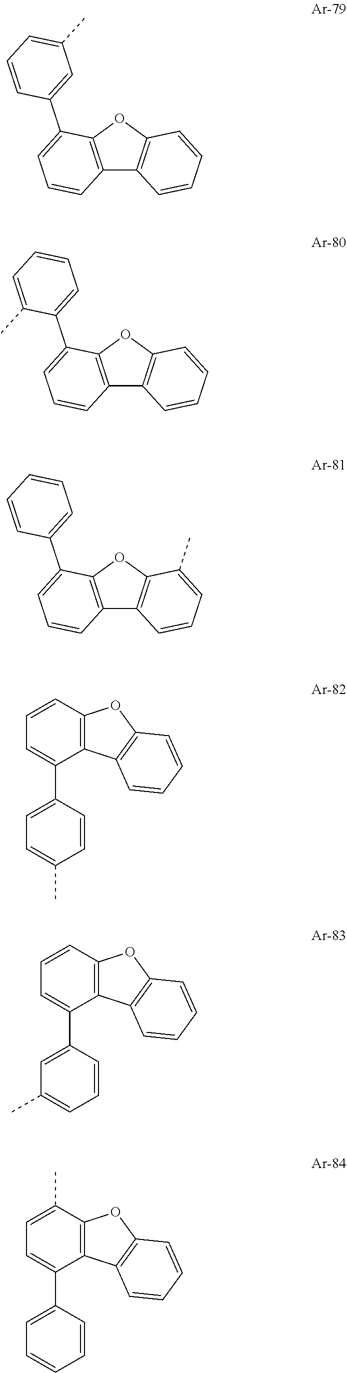

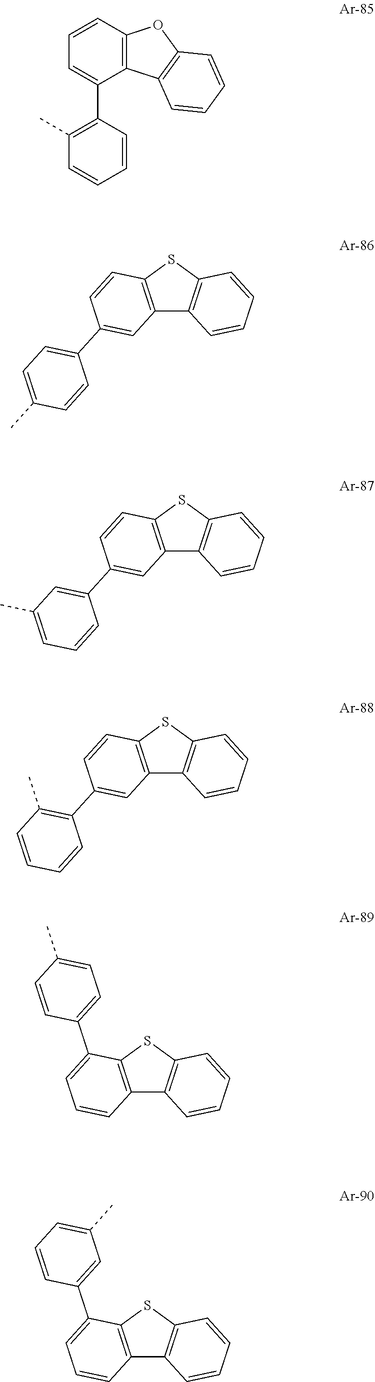

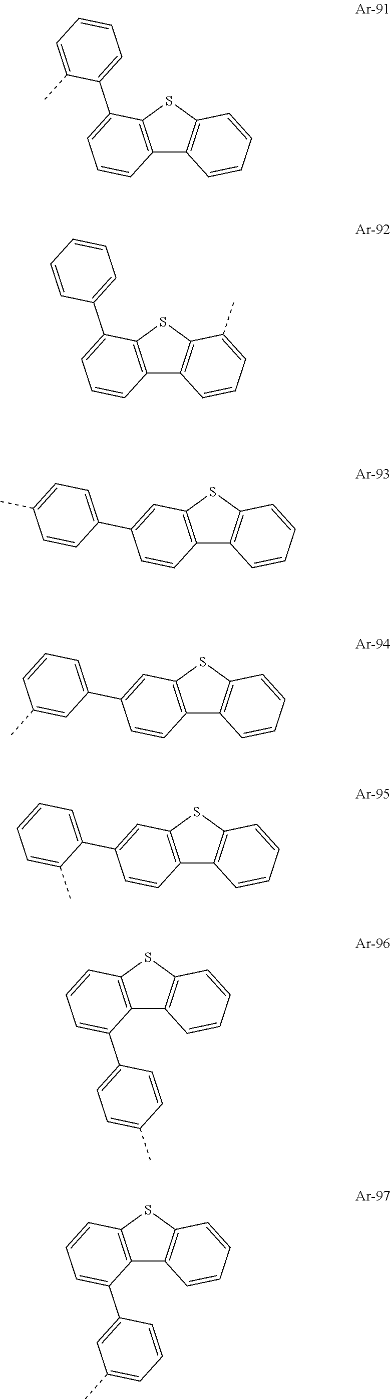

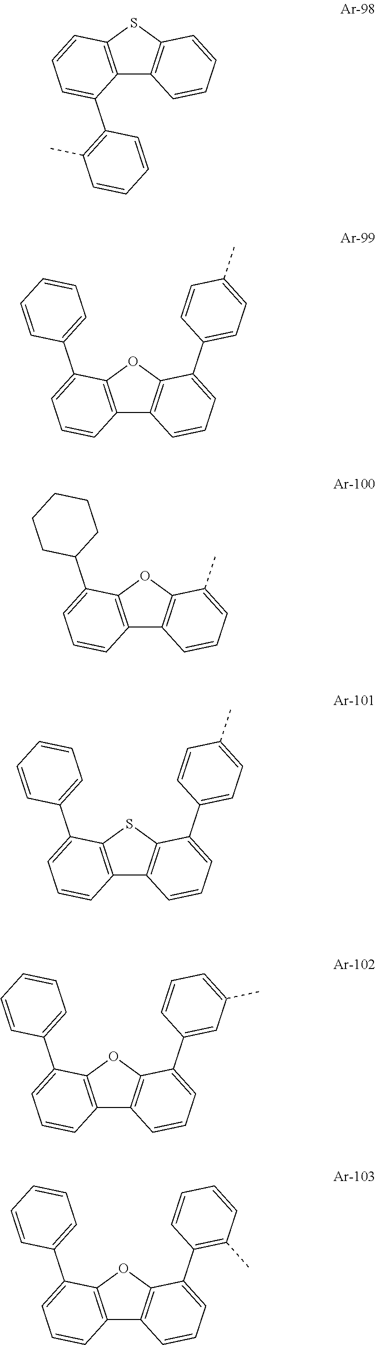

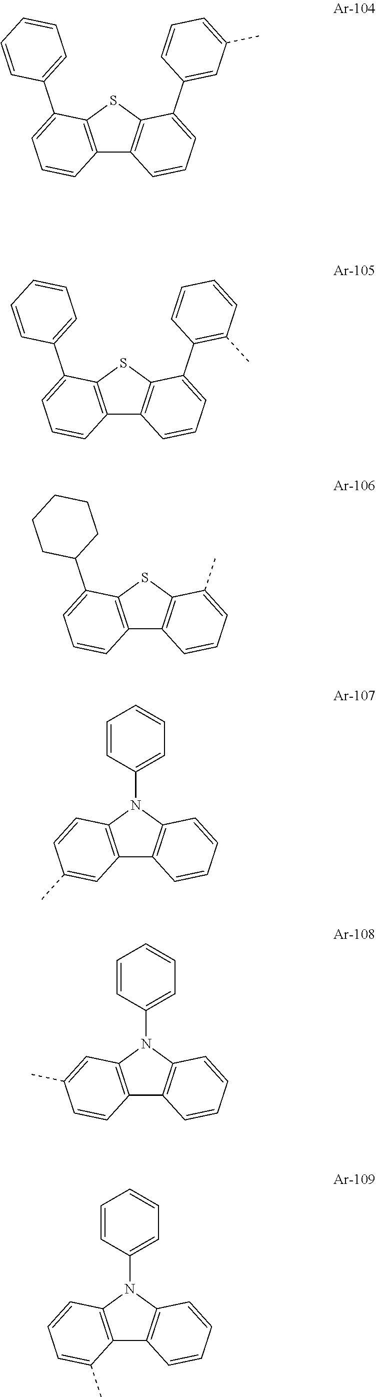

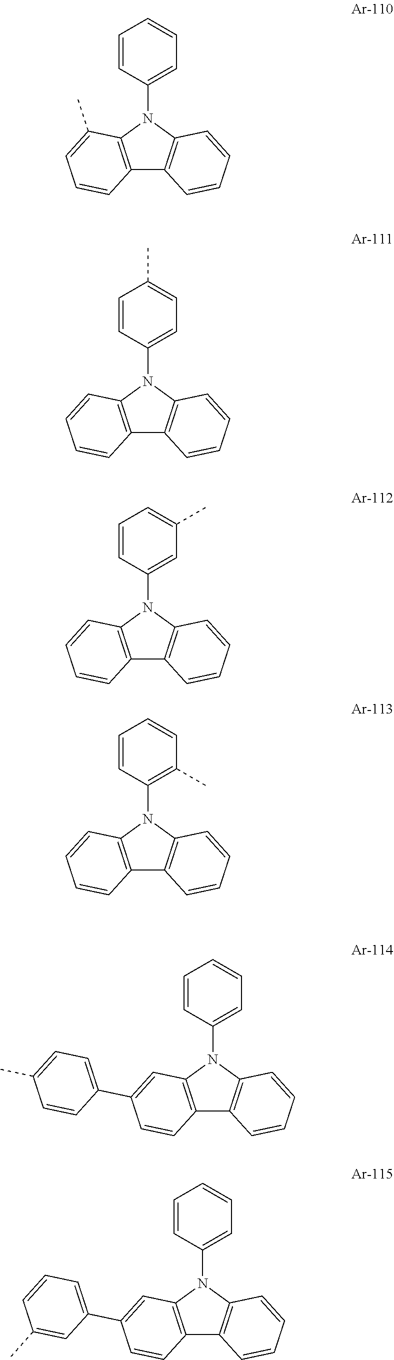

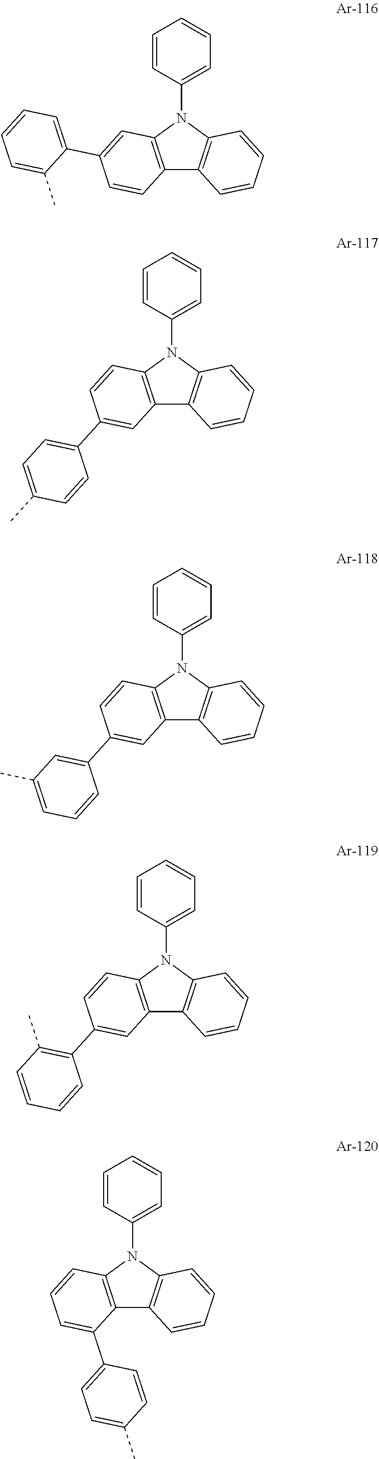

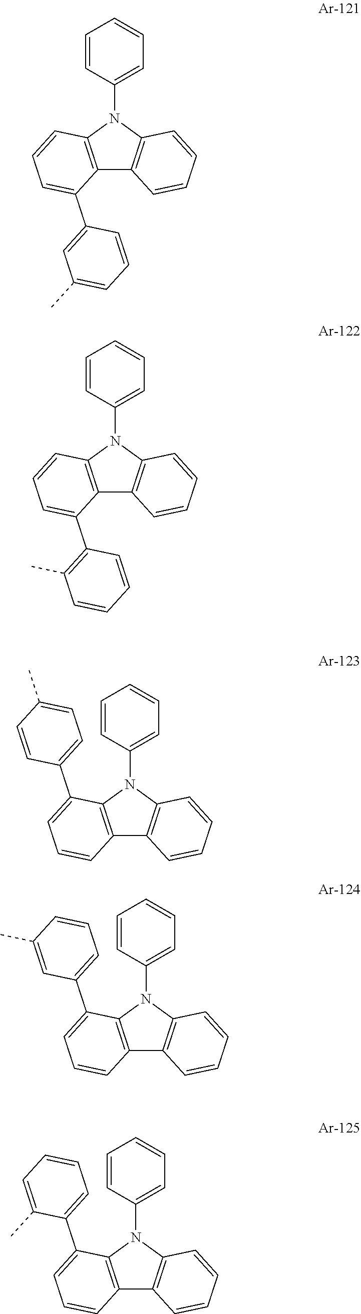

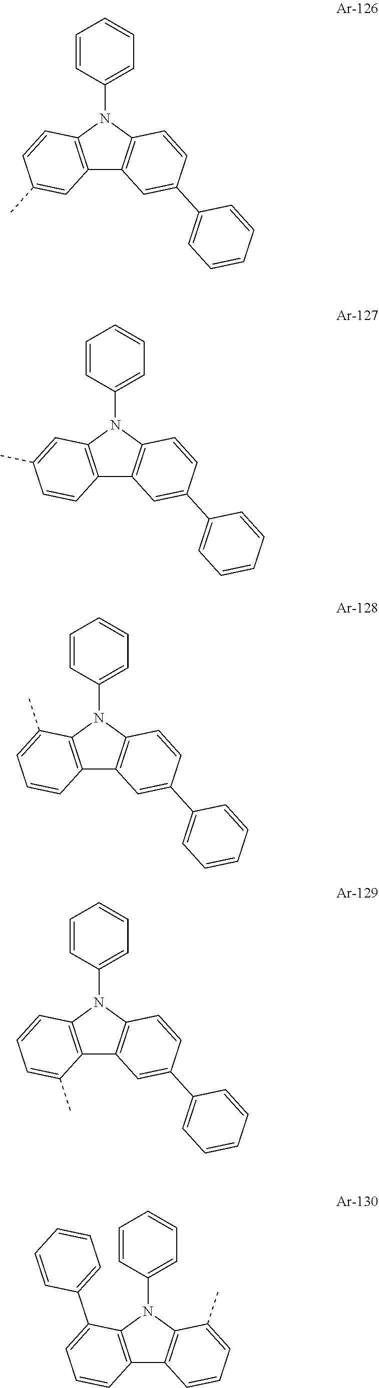

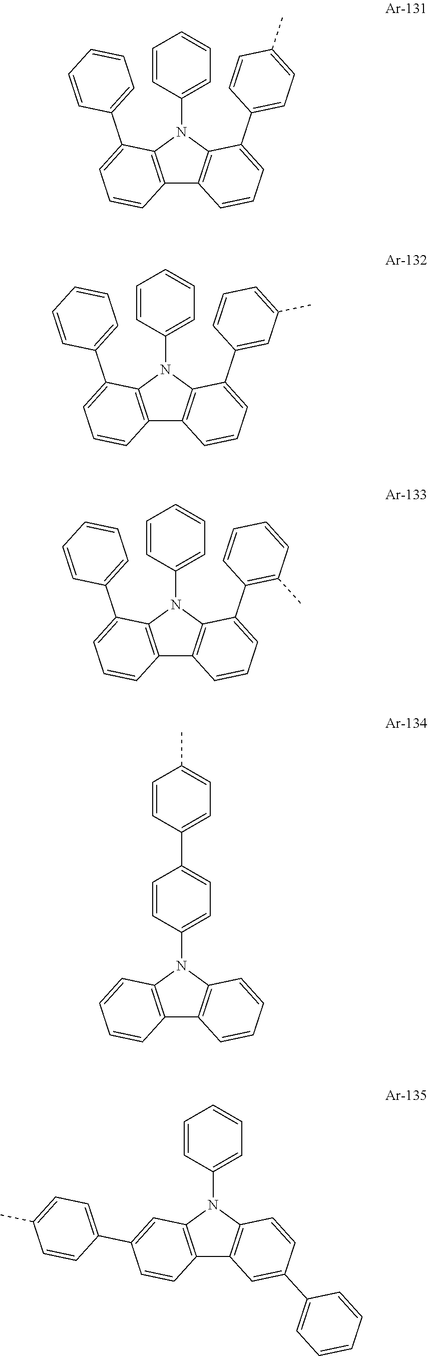

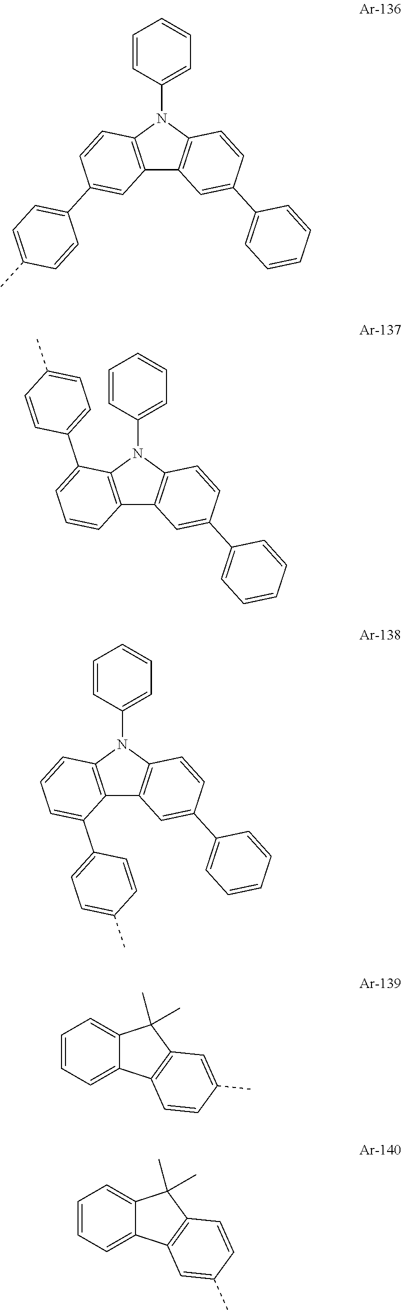

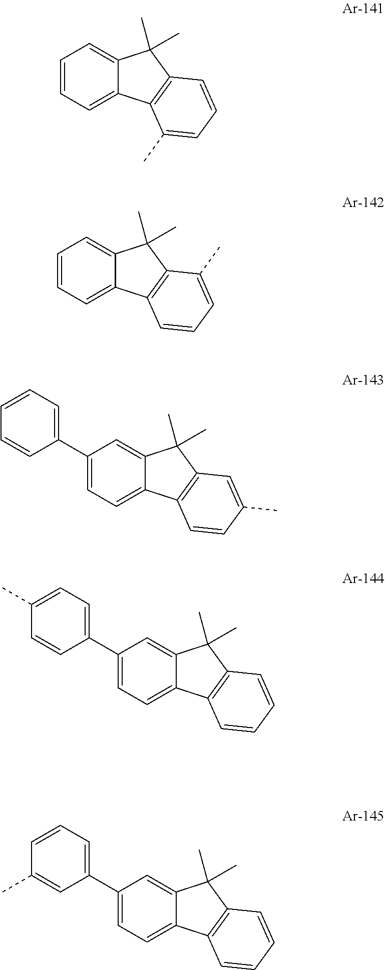

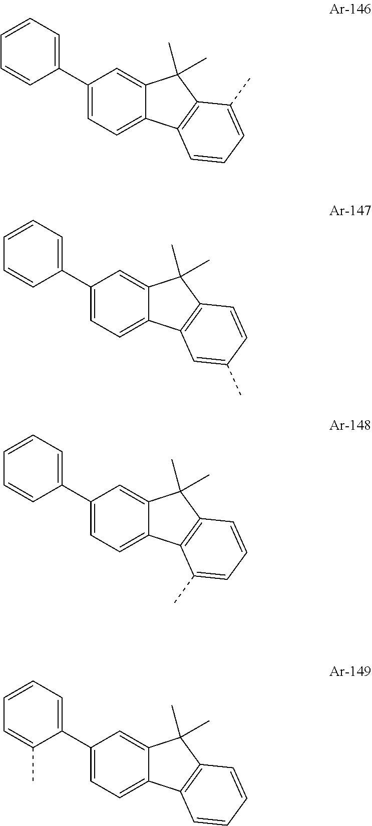

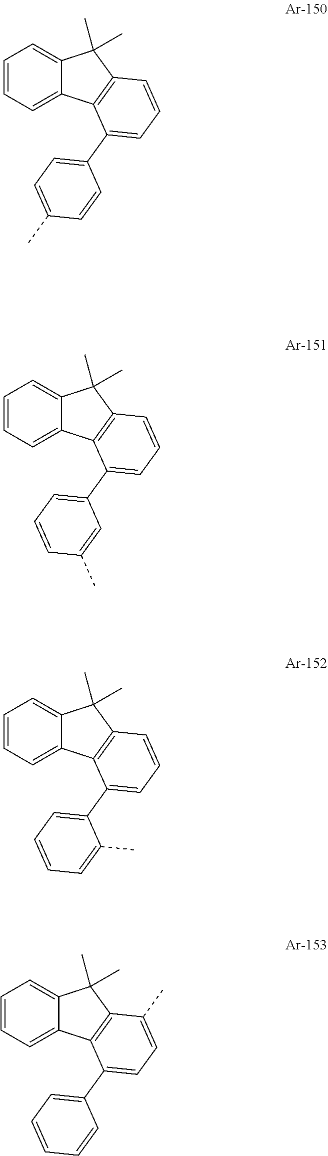

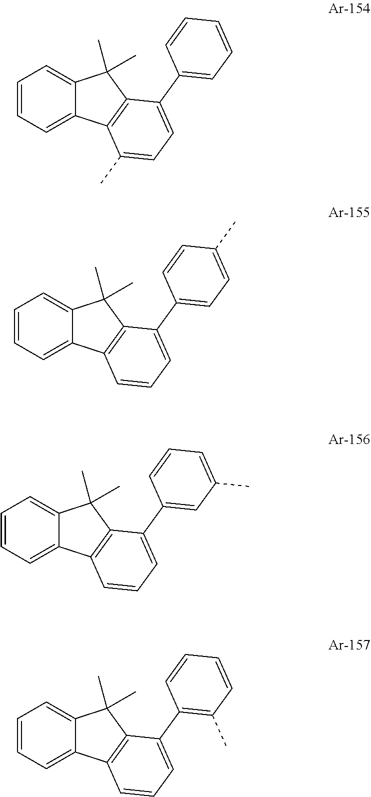

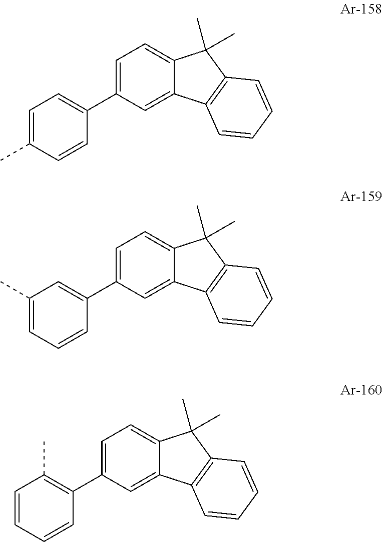

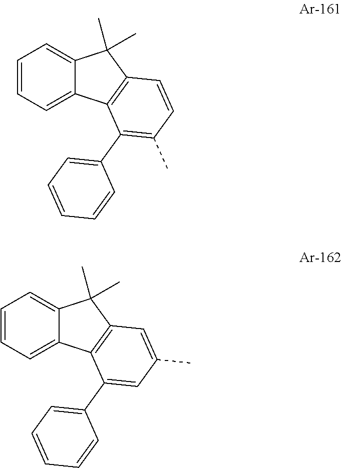

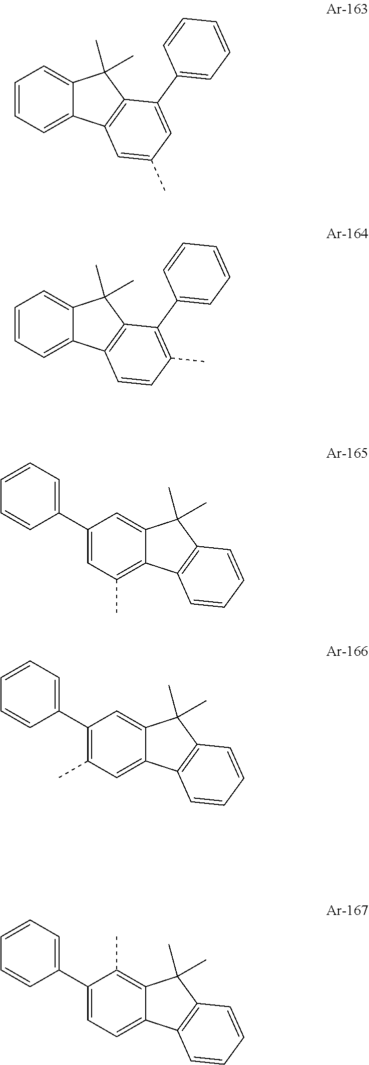

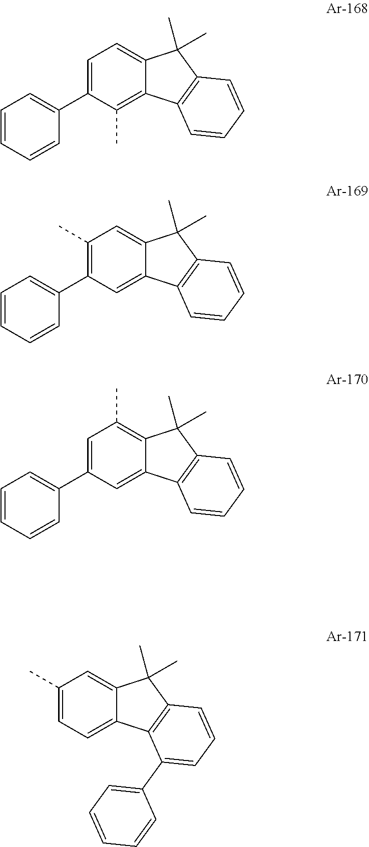

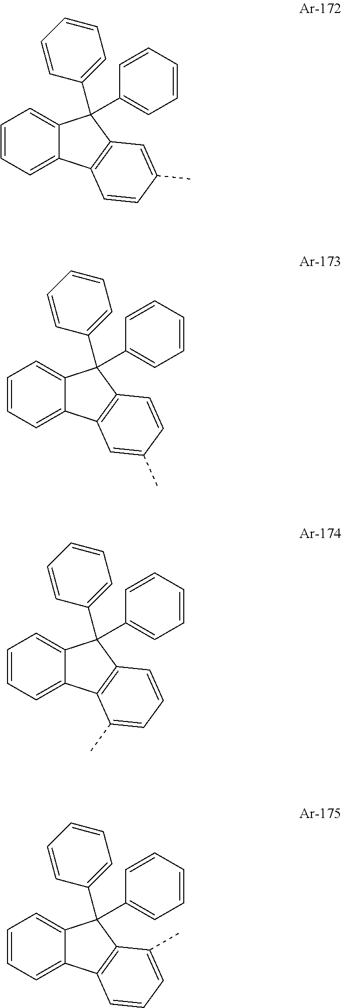

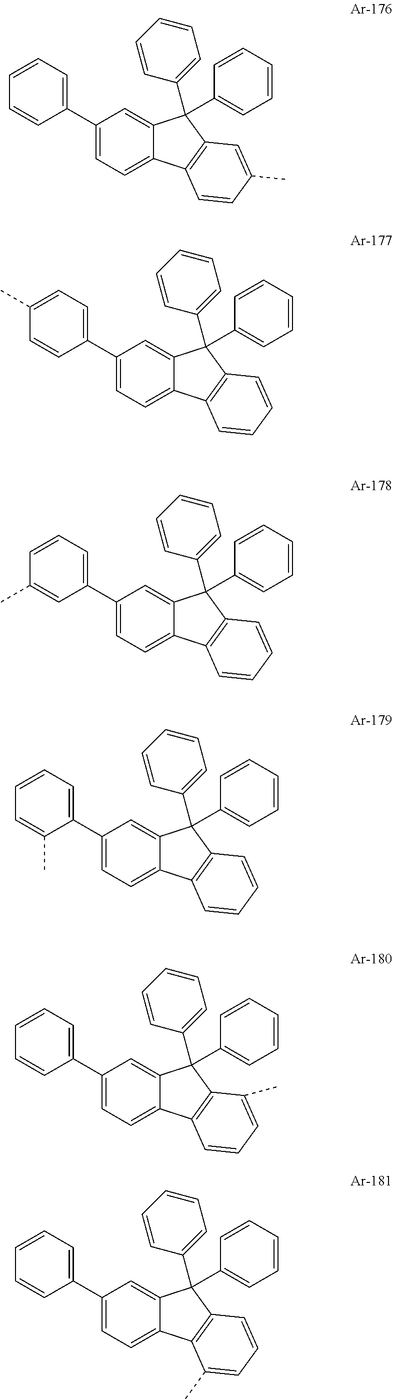

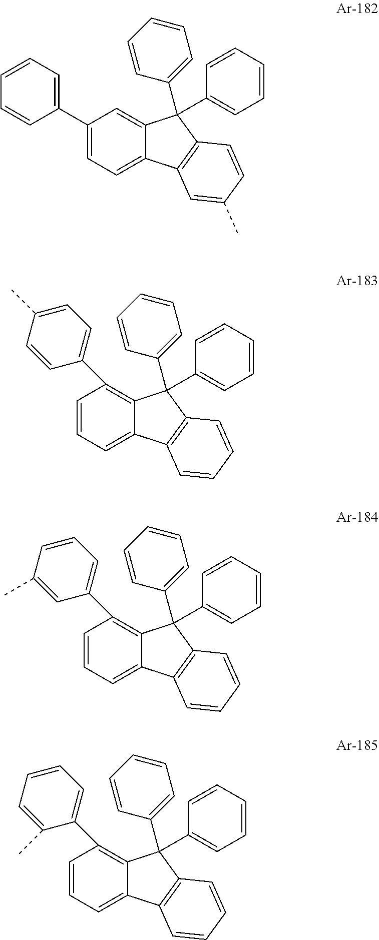

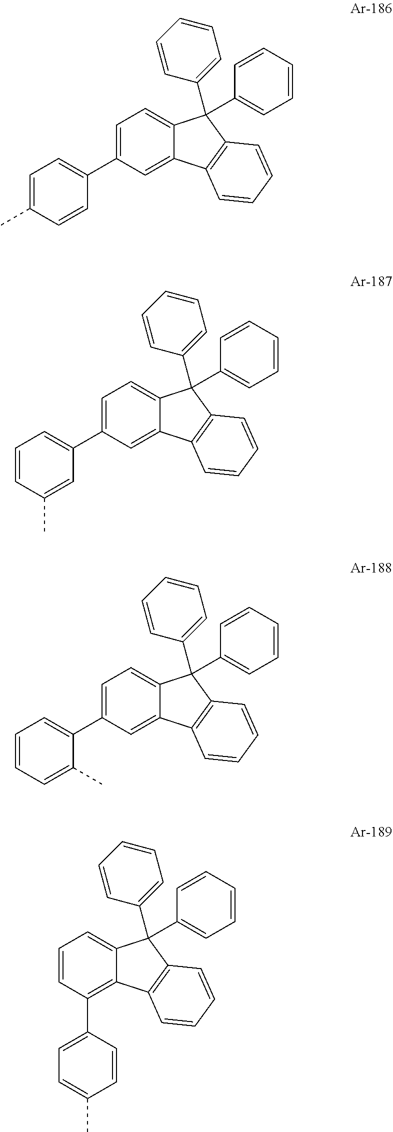

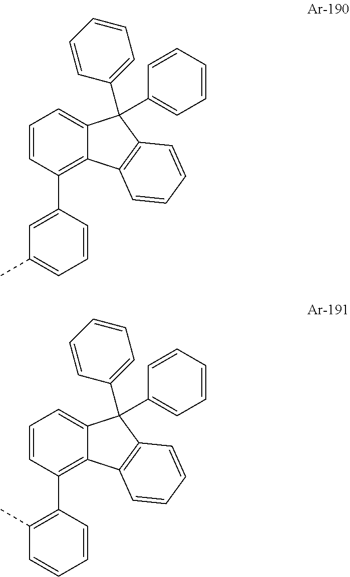

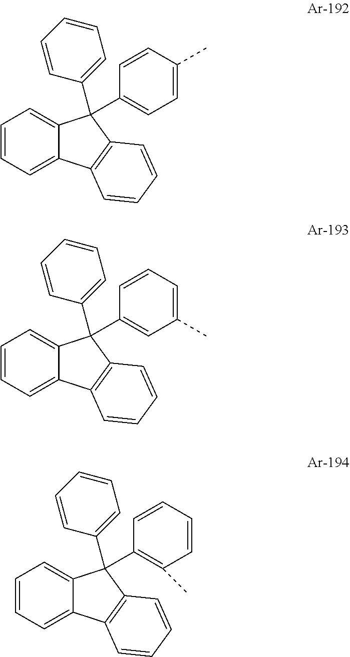

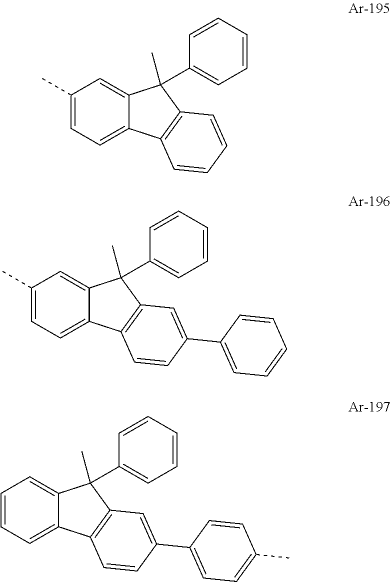

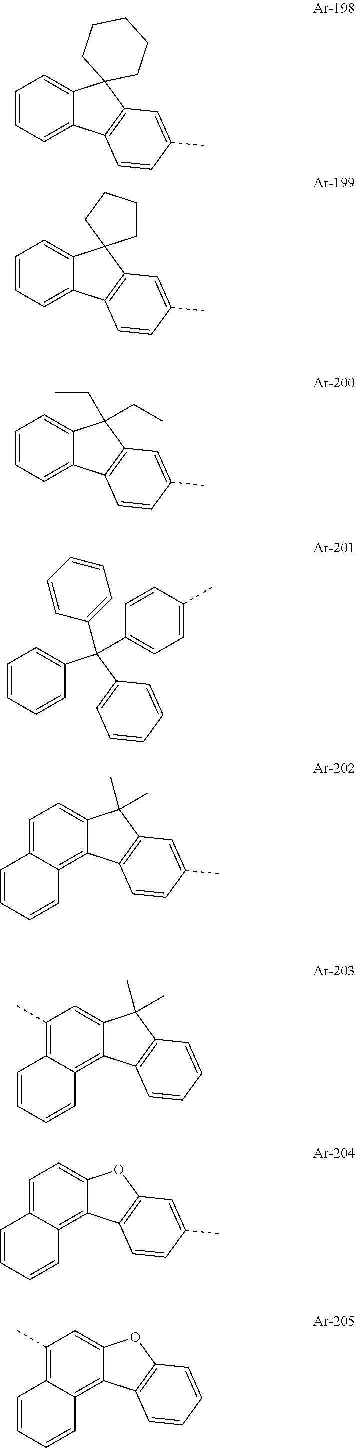

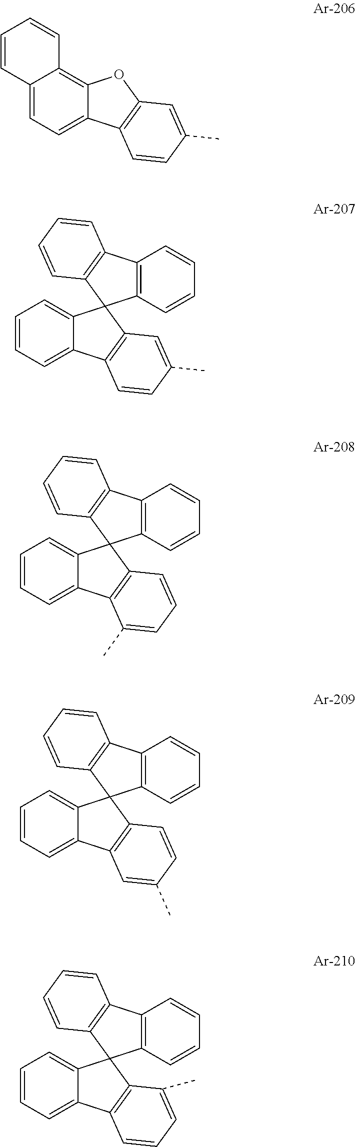

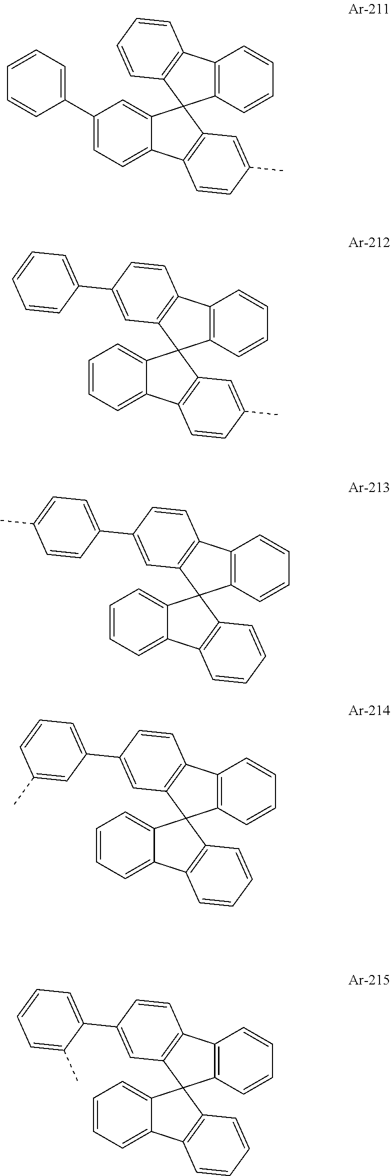

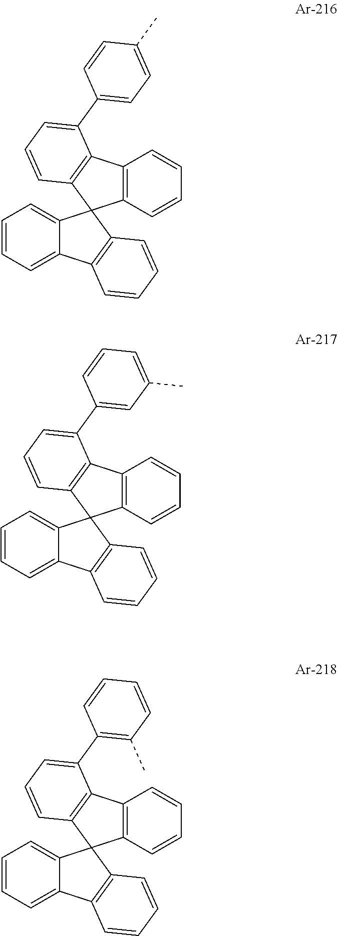

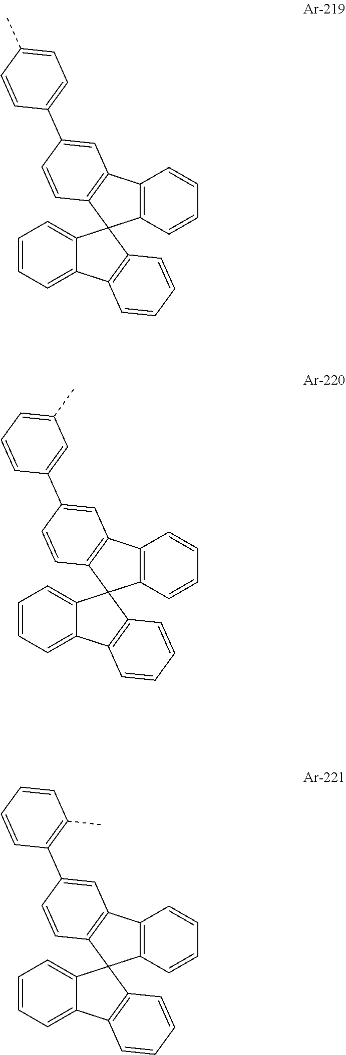

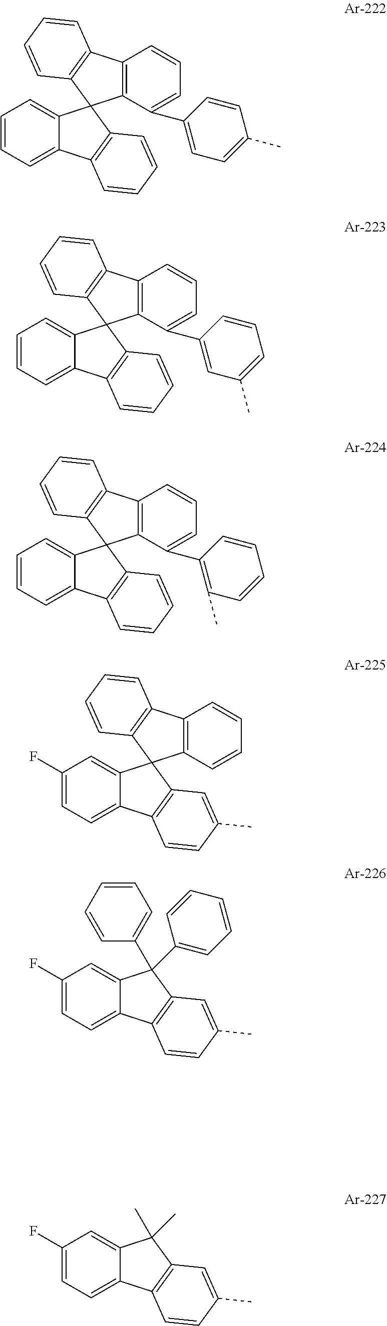

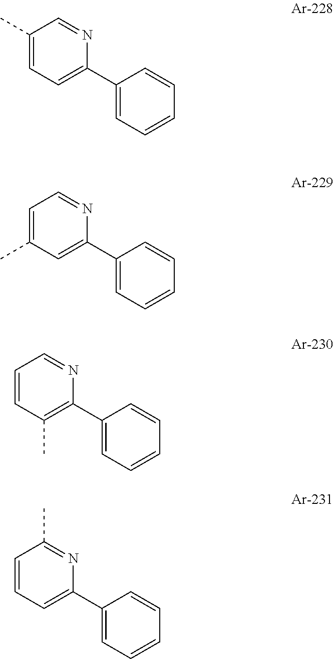

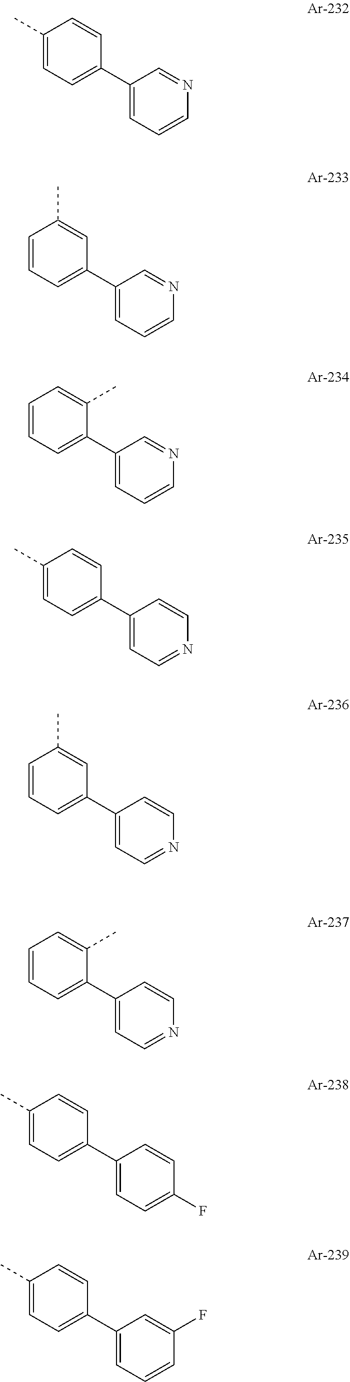

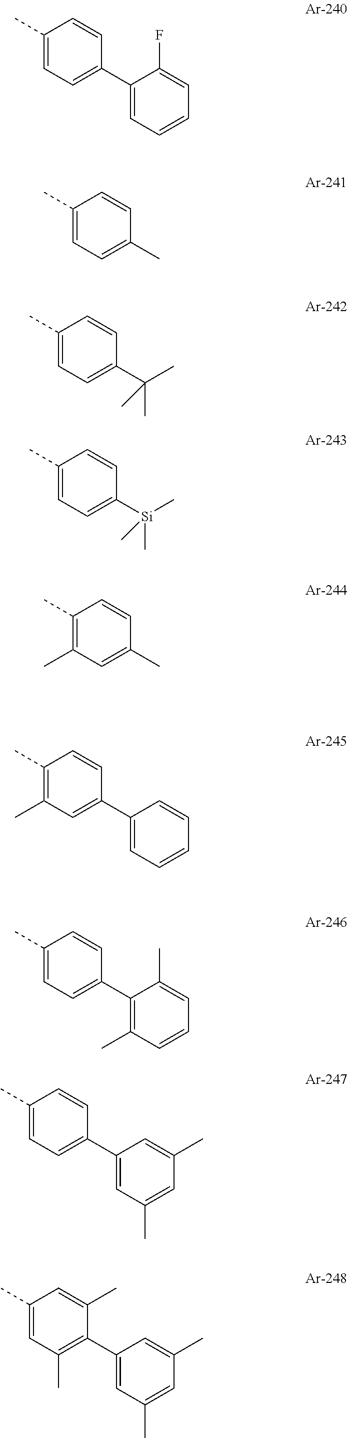

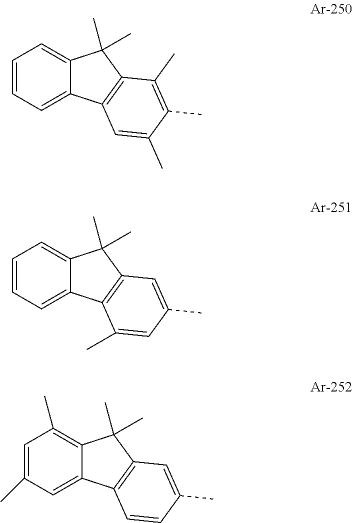

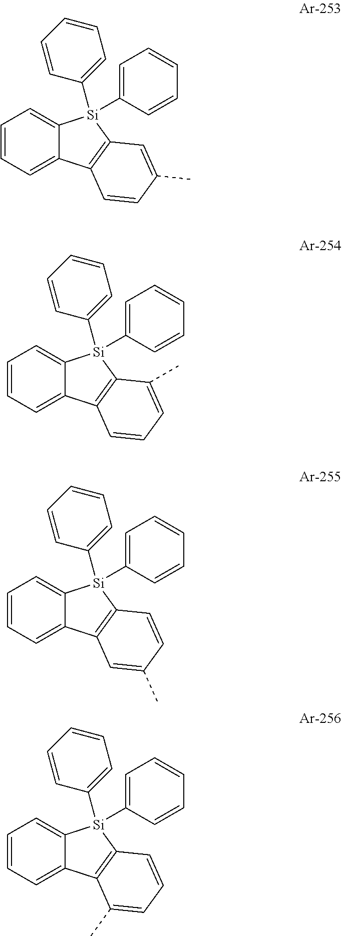

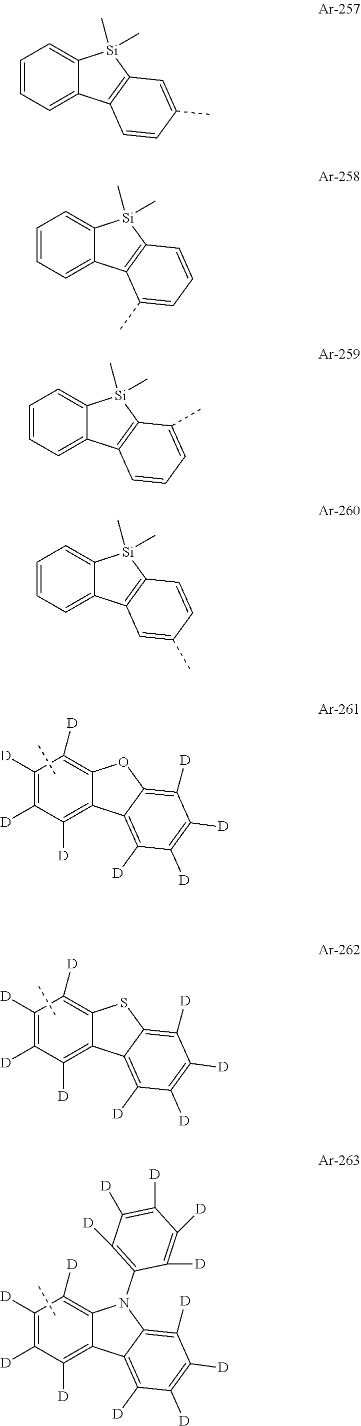

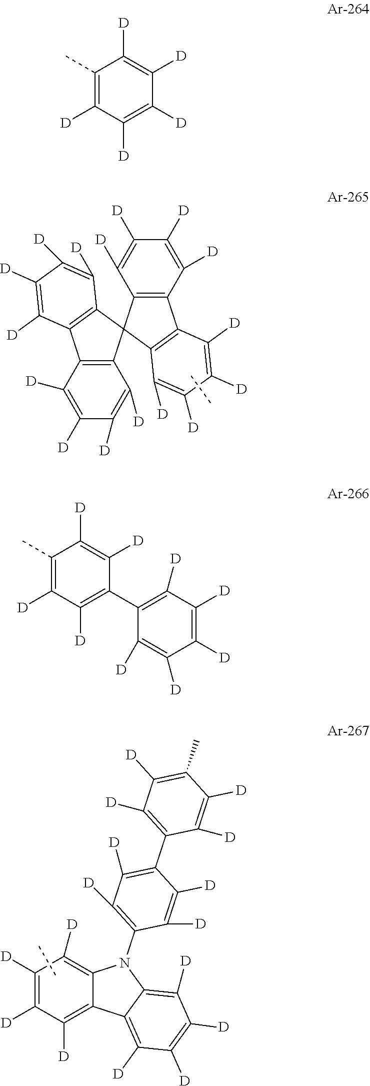

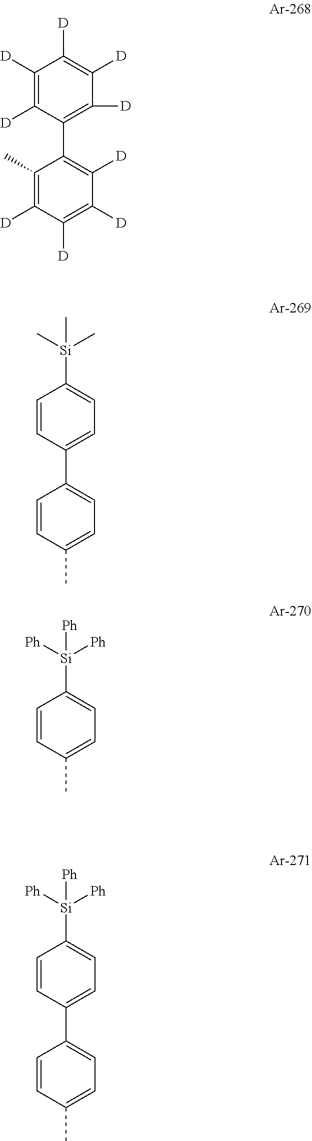

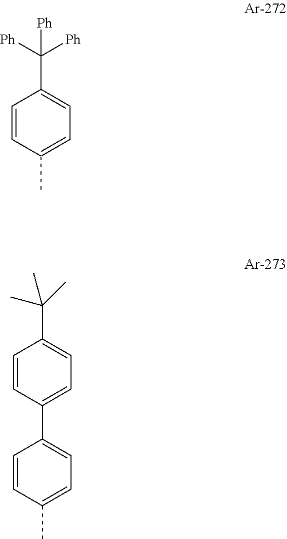

where the groups at the positions shown as unsubstituted are substituted by R³ radicals, where R³ in these positions is preferably H, and where the dotted bond is the bond to the amine nitrogen atom.

Most preferably, Ar² is the same or different at each instance and is selected from formulae Ar-1, Ar-2, Ar-3, Ar-4, Ar-5, Ar-48, Ar-50, Ar-74, Ar-78, Ar-82, Ar-107, Ar-108, Ar-117, Ar-134, Ar-139 and Ar-172.

In a preferred embodiment, the two Ar² groups selected in formula (A) are different.

E is preferably a single bond.

Preferably, the sum total of the indices m and n is 0 or 1, more preferably 0.

Preferably, n=0, such that the E group in question is absent. Preferably, m is 0, such that the E group in question is absent.

In an alternative preferred embodiment, m=1 and n=0. In this case, it is preferable that the subunit

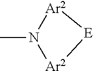

of the formula (A) is selected from the following formulae:

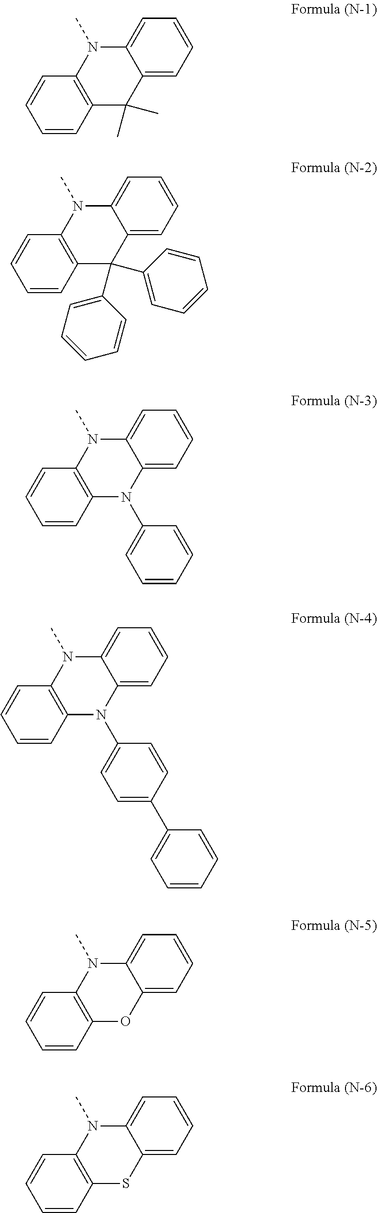

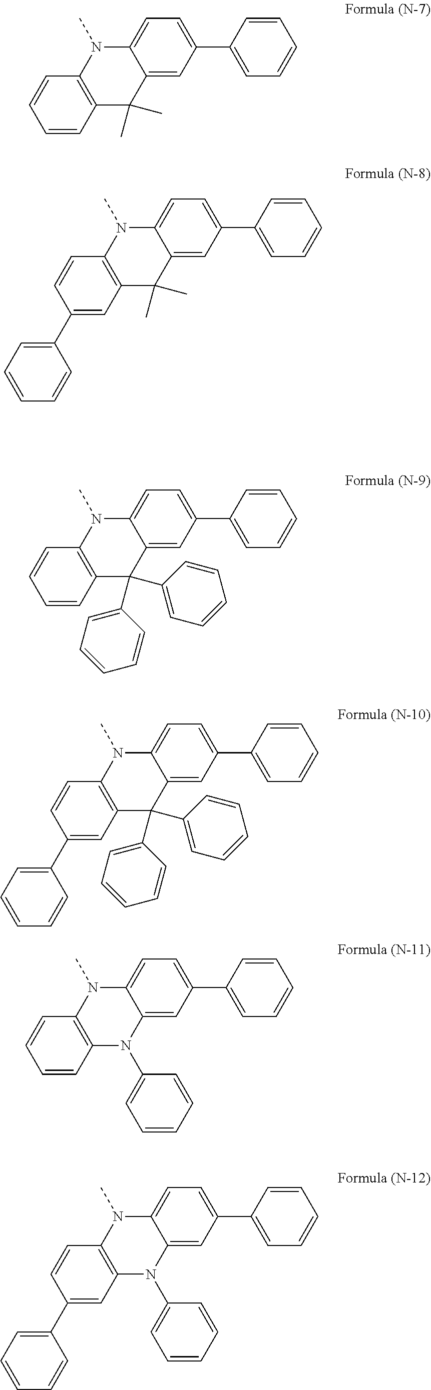

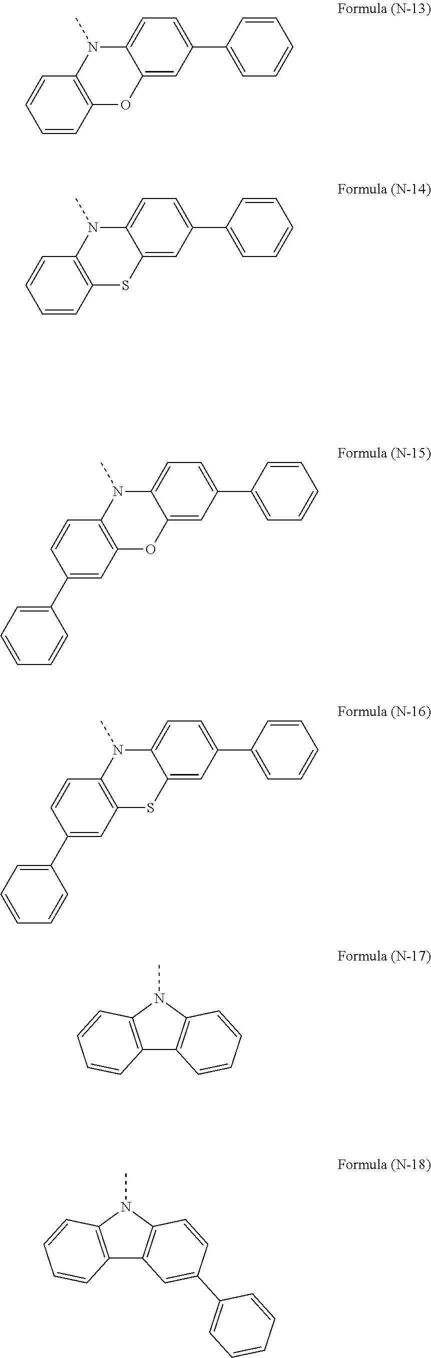

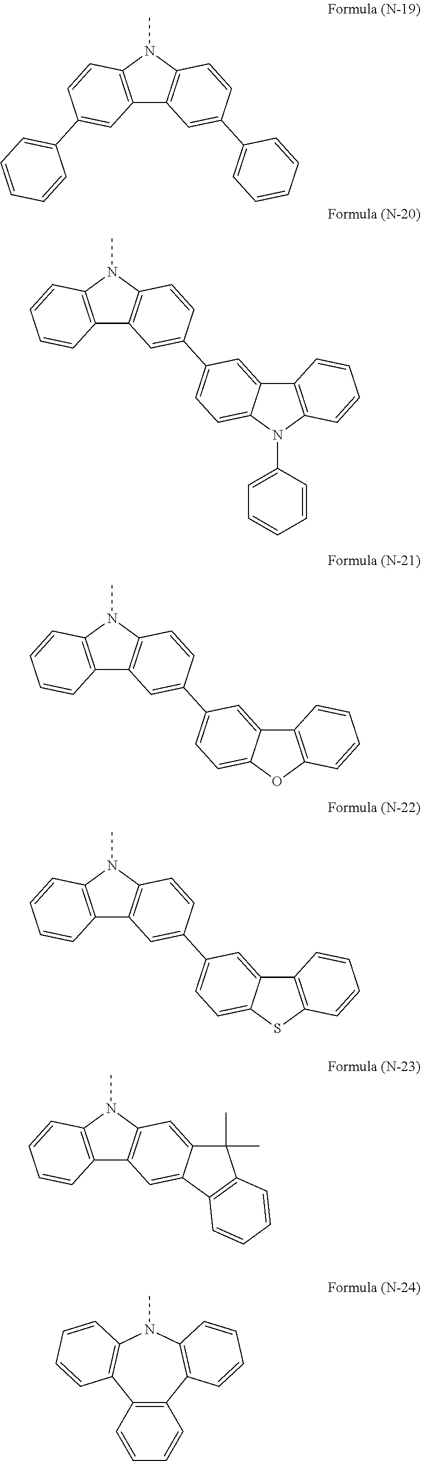

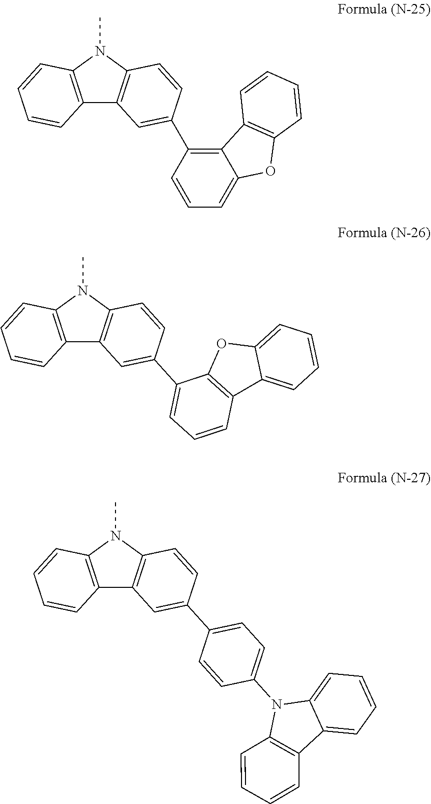

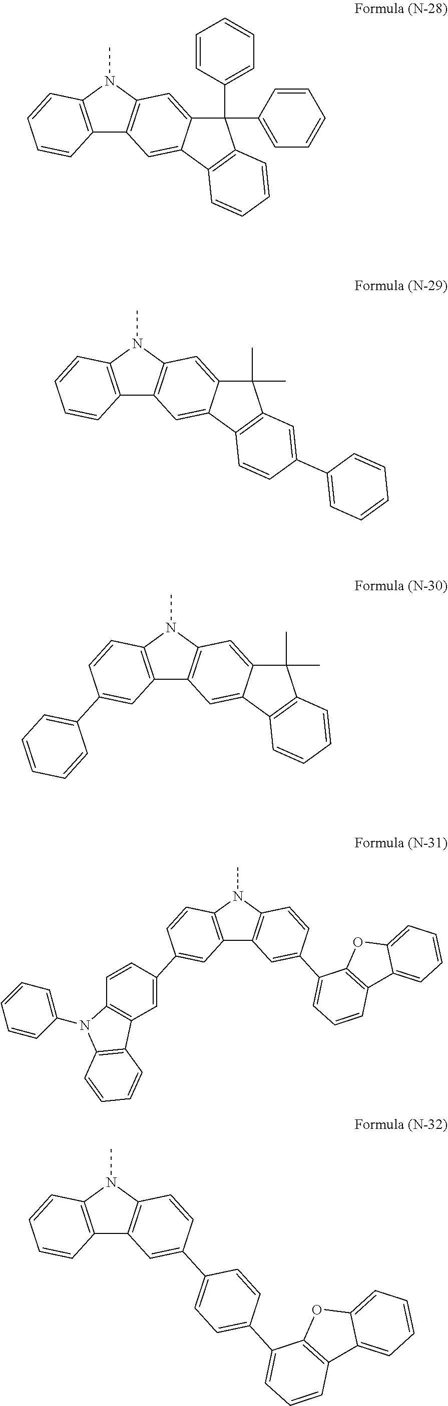

which are substituted by R³ radicals at the unoccupied positions on the rings, where these R³ radicals are preferably H.

In an alternative preferred embodiment, n=1 and m=0. In this case, it is preferable that the unit

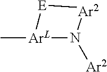

of the formula (A) is selected from the following formulae:

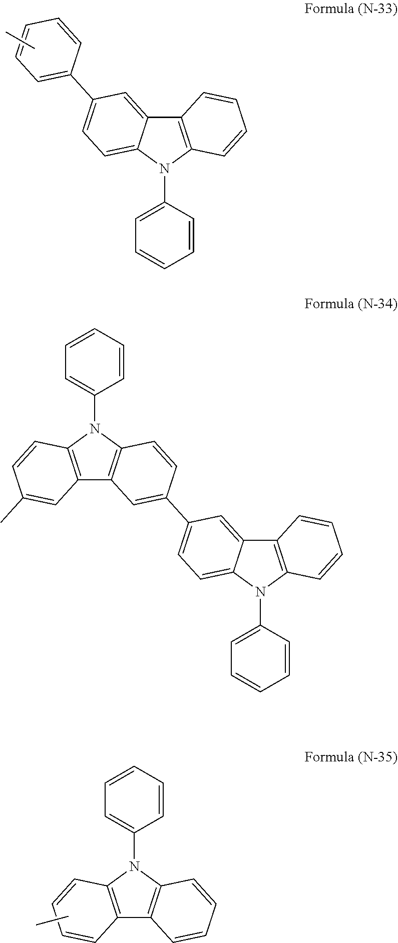

which are substituted by R³ radicals at the unoccupied positions on the rings, where these R³ radicals are preferably H.

Preferably, R³ is the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, R⁵C═CR⁵—, Si(R⁵)₂, C═0, C═NR⁵, —NR⁵—, —O—, —S—, —C(═O)O— or —C(═O)NR⁵—. More preferably, R³ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R³ is H.

Preferably, R⁴ is the same or different at each instance and is selected from H, D, F, CN, Si(R⁵)₃, N(R⁵)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁵ radicals; and where one or more CH₂ groups in the alkyl or alkoxy groups mentioned may be replaced by —C≡C—, R⁵C═CR⁵—, Si(R⁵)₂, C═0, C═NR⁵, —NR⁵—, —O—, —S—, —C(═O)O— or —C(═O)NR⁵—. More preferably, R⁴ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, which are preferably H. Even more preferably, R⁴ is H. Preferably, R⁵ is the same or different at each instance and is selected from H, D, F, CN, Si(R⁶)₃, N(R⁶)₂, straight-chain alkyl or alkoxy groups having 1 to 20 carbon atoms, branched or cyclic alkyl or alkoxy groups having 3 to 20 carbon atoms, aromatic ring systems having 6 to 40 aromatic ring atoms and heteroaromatic ring systems having 5 to 40 aromatic ring atoms; where the alkyl and alkoxy groups mentioned, the aromatic ring systems mentioned and the heteroaromatic ring systems mentioned are each substituted by R⁶ radicals; and where one or more CH₂ groups in the alkyl or alkoxy groups mentioned may be replaced by —C≡C—, —R⁶C═CR⁶—, Si(R⁶)₂, C═0, C═NR⁶, —NR⁶—, —O—, —S—, —C(═O)O— or —C(═O)NR⁶—. More preferably, R⁵ is the same or different at each instance and is selected from H, D, Si(R⁶)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁶ radicals, which are preferably H. Even more preferably, R⁵ is H.

Preferred embodiments of the formula (I) conform to the formulae (I-A) to (I-D)

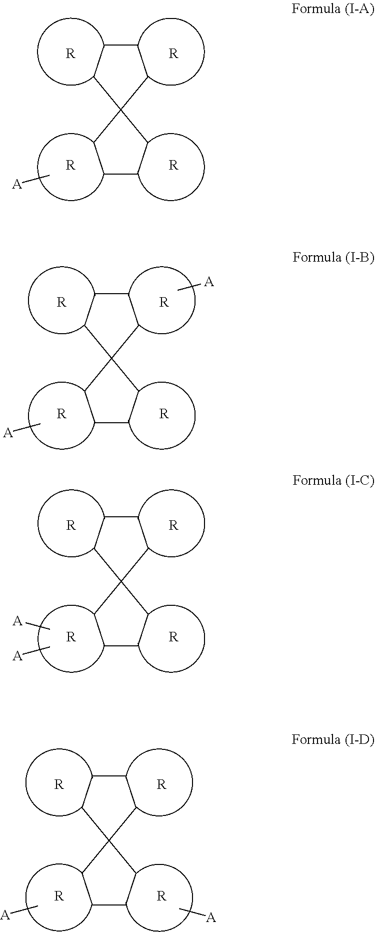

where the variables are as defined above, and the A group is either bonded directly to the ring of the unit R or bonded to an Ar¹ group that binds to one of the rings or is bonded to an Ar⁰ group that is part of an X=NAr⁰ group. Preference is given here to the formula (I-A). It is further preferable that the A group is bonded to an Ar¹ group that binds to one of the rings or is bonded to an Ar⁰ group that is part of an X=NAr⁰ group.

When the A group is bonded to a unit (R-1), it is preferably bonded in a position corresponding to the 1, 2 or 4 position on the spirobifluorene, preferably position 2 or 4. Positions 1, 2 and 4 are the following positions on the spirobifluorene base skeleton:

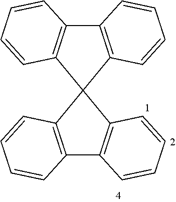 [SMILES: c1ccc2c(c1)-c1ccccc1C21c2ccccc2-c2ccccc21]

Preferred embodiments of the formula (I) therefore conform to one of the following formulae (I-a) to (I-f):

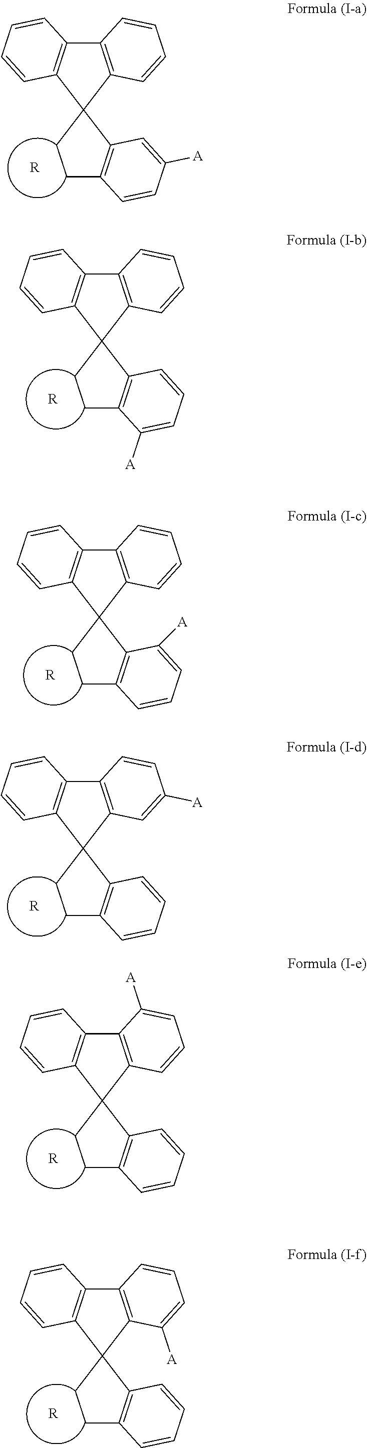

where the unit R is selected from formulae (R-2) and (R-3), preferably (R-2), and where R¹ radicals are bonded to all unoccupied positions on the rings.

It is further preferable that the compound of the formula (I) conforms to one of the following formulae (I-1) to (I-11):

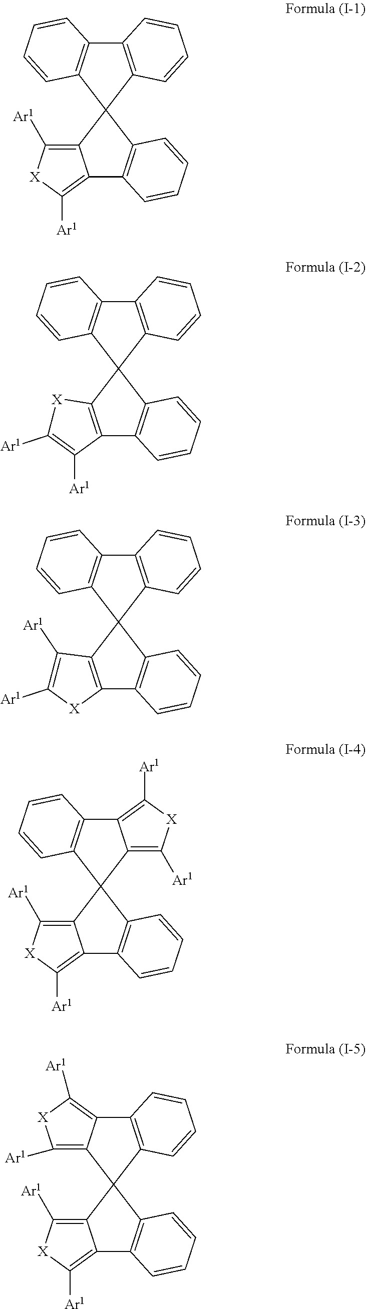

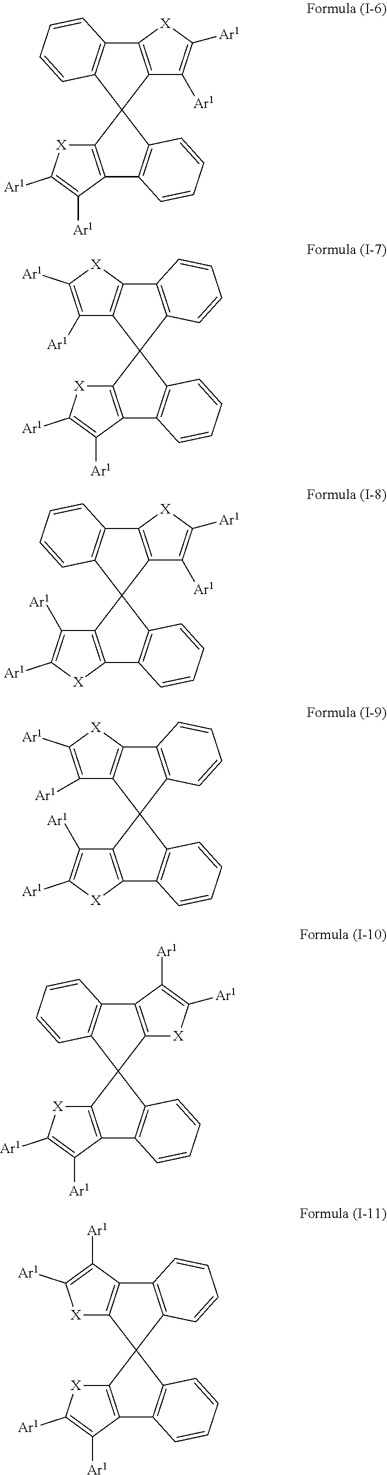

where the variables are as defined above, and where the at least one A group is either bonded directly to one of the rings or bonded to an Ar¹ group that binds to one of the rings or is bonded to an Ar⁰ group that is part of an X=NAr⁰ group. There is preferably exactly one A group bonded per formula. Further preferably, at least one A group is bonded to an Ar¹ group that binds to one of the rings or is bonded to an Ar⁰ group that is part of an X=NAr⁰ group. X in the abovementioned formulae is preferably S or O, more preferably S. Among the abovementioned formulae, preference is given to the formulae (I-1) to (I-5), particular preference to the formulae (I-1) to (I-3), and very particular preference to the formula (I-1).

Preferred embodiments of the formulae (I-1) to (I-4) conform to the formulae shown below:

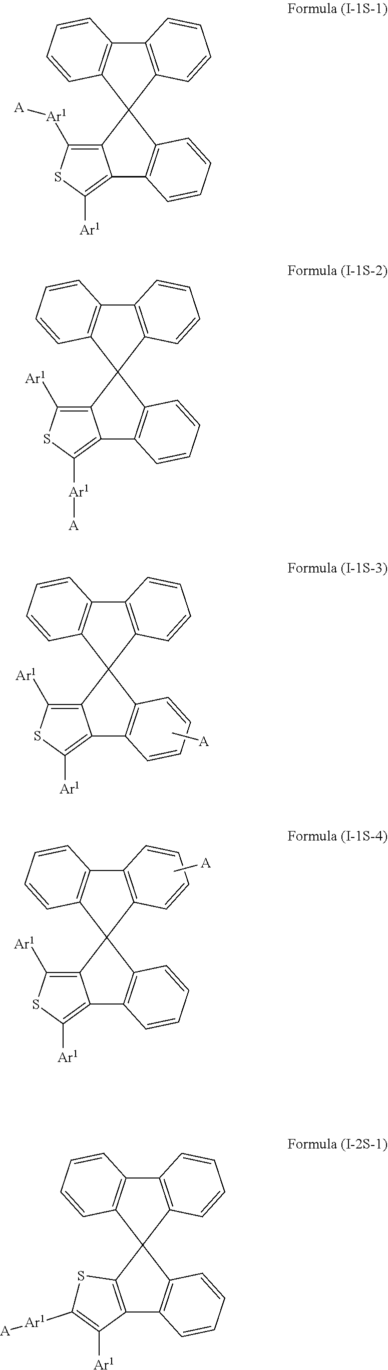

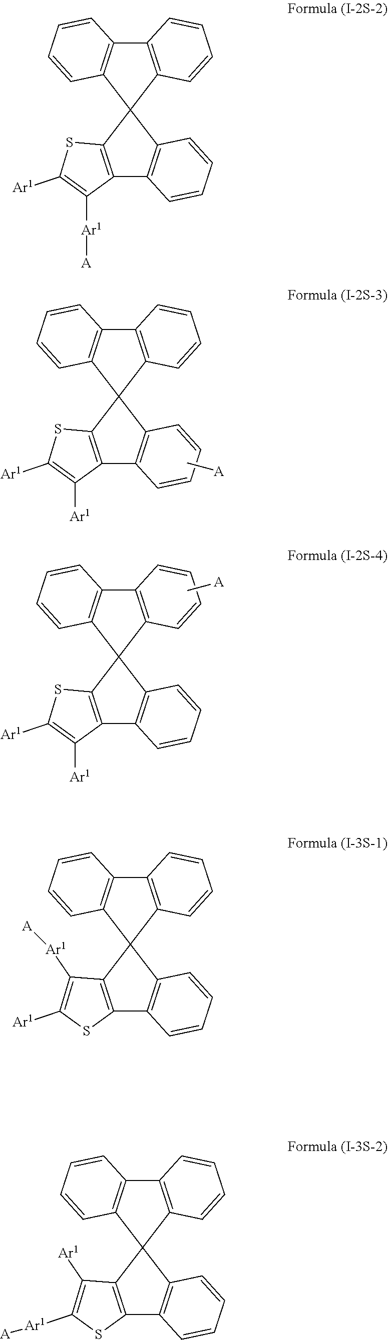

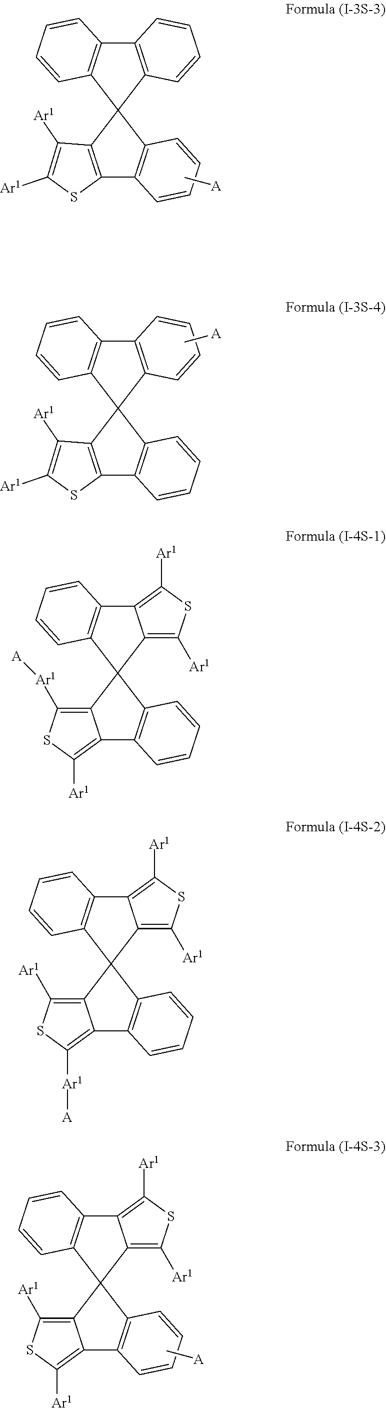

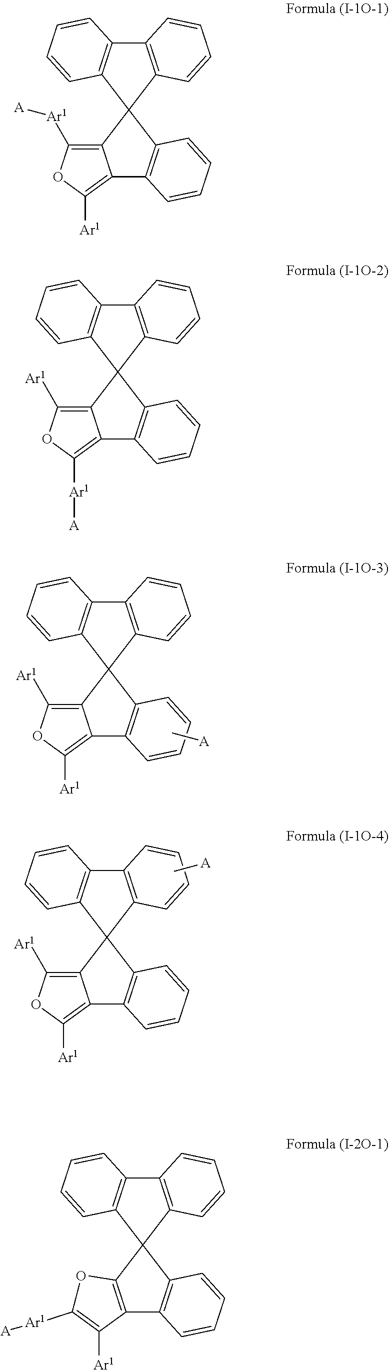

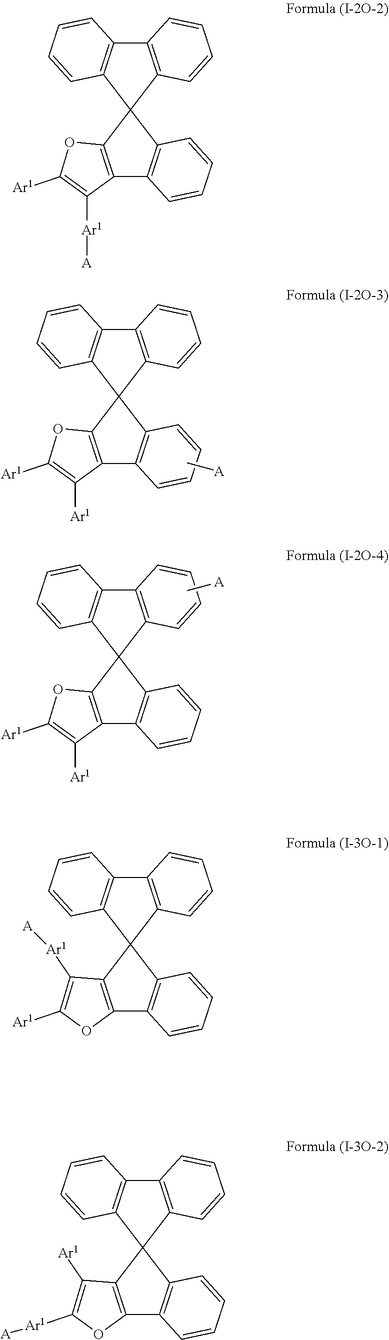

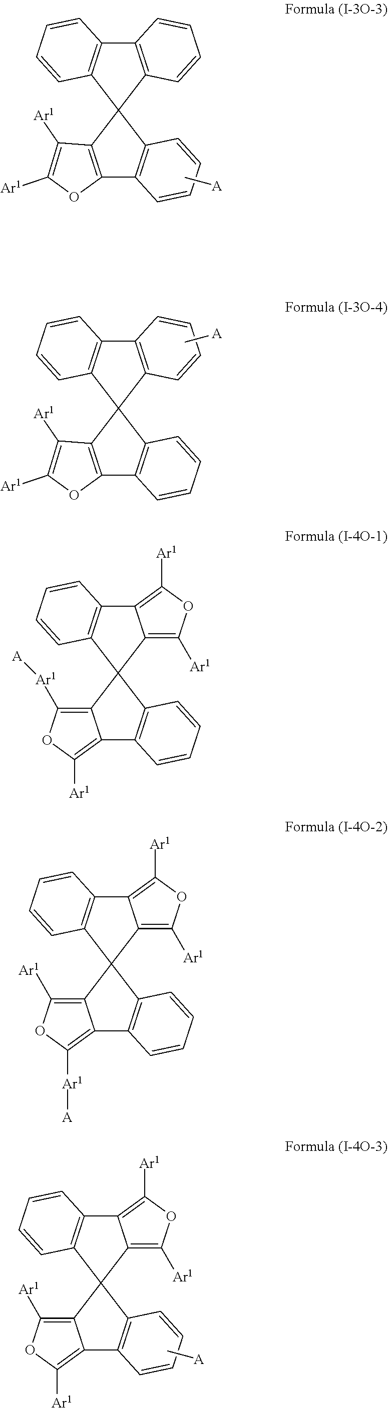

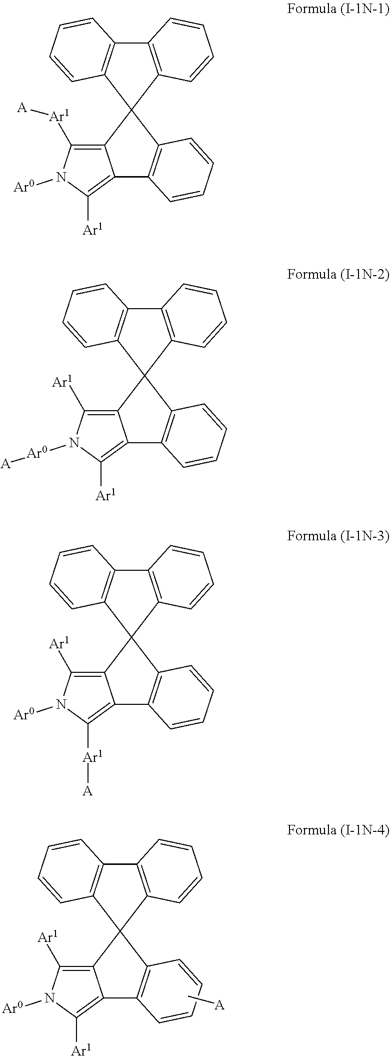

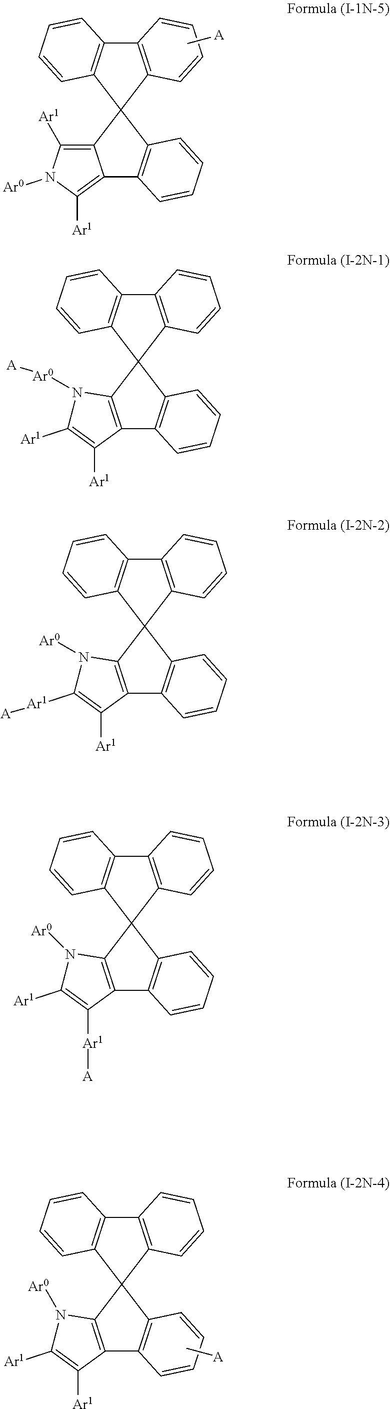

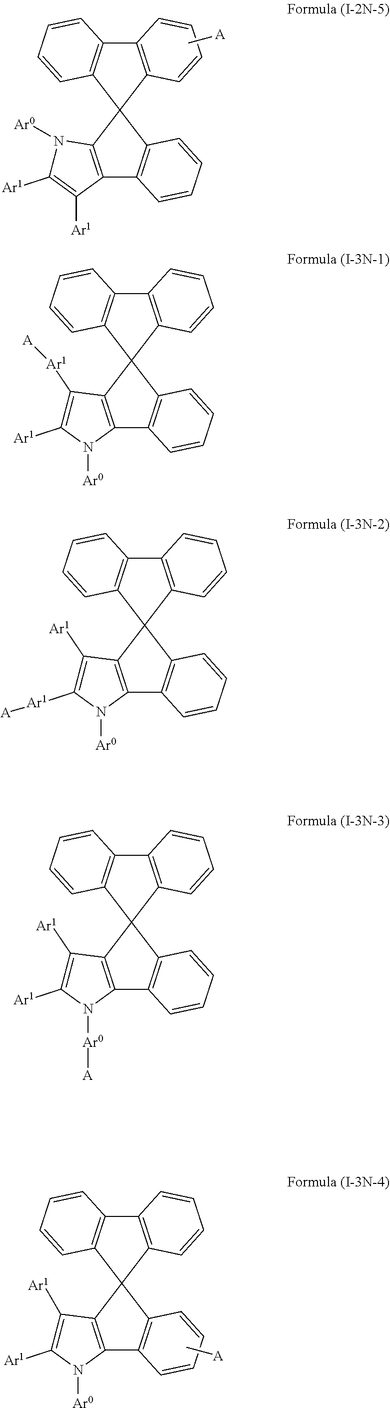

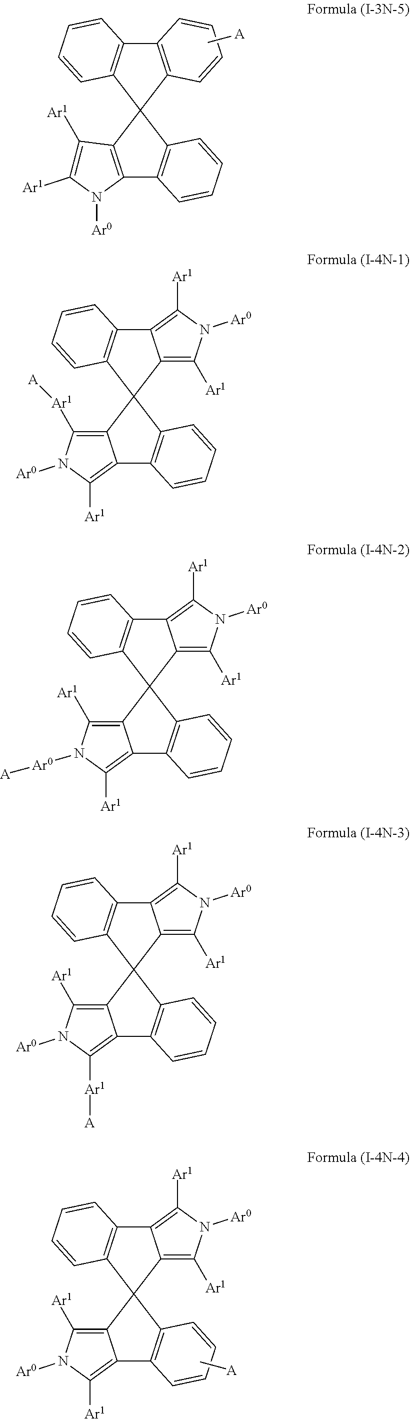

where the variables have the definitions given above.

Ar⁰ in the abovementioned formulae is preferably phenyl substituted by R², where R² in these cases is preferably H. Ar¹ in the abovementioned formulae is preferably phenyl substituted by R², where R² in these cases is preferably H.

Among the abovementioned formulae, preference is given to the formulae (I-1S-1) to (I-3S-4), (I-1O-1) to (I-3O-4) and (I-1N-1) to (I-3N-5). Among the abovementioned formulae, preference is further given to the formulae (I-1S-1) to (I-4S-3) and (I-1O-1) to (I-4O-3). Particular preference is given to the formulae (I-1S-1) to (I-3S-4) and (I-1O-1) to (I-3O-4).

Preference is further given to the formulae (I-1S-1) to (I-1 S-4), (I-1O-1) to (I-1O-4) and (I-1N-1) to (I-1N-5). Among these, particular preference is given to the formulae (I-1S-1) to (I-1S-4) and (I-1O-1) to (I-1O-4).

Preference is further given to the formulae (I-1S-1), (I-1 S-2), (I-2S-1), (I-2S-2), (I-3S-1), (I-3S-2), (I-1O-1), (I-1O-2), (I-2O-1), (I-2O-2), (I-3O-1), (I-3O-2), (I-1N-1) to (I-1N-3), (I-2N-1) to (I-2N-3), and (I-3N-1) to (I-3N-3).

Preferred embodiments of the formula (I-11S-3), (I-11S-4), (I-2S-3), (I-2S-4), (I-3S-3), (I-3S-4) are the following formulae:

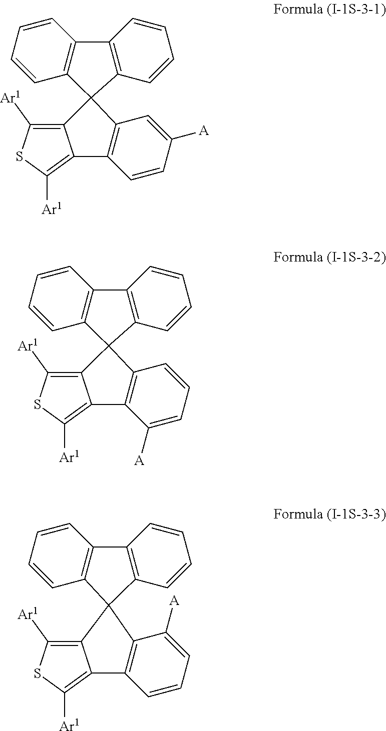

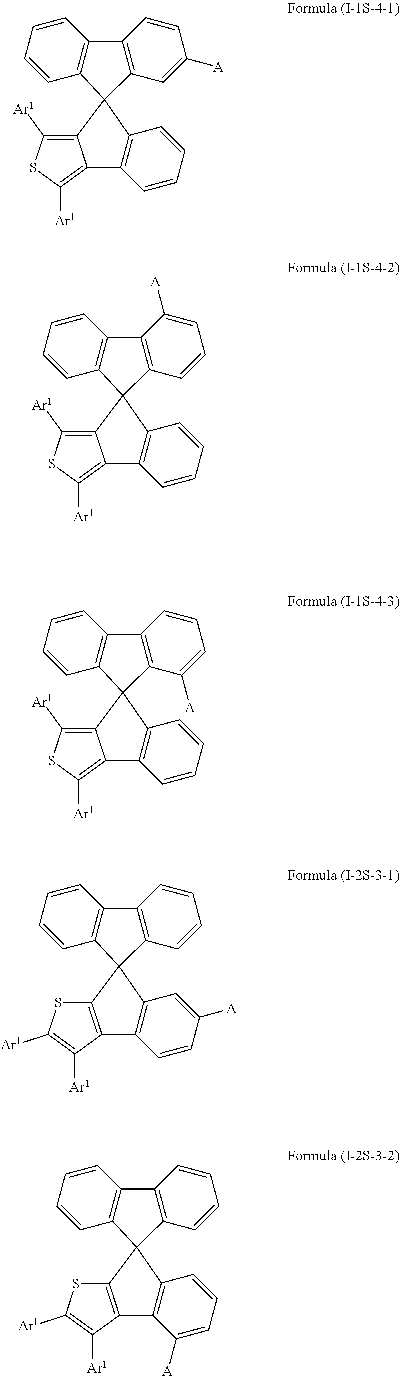

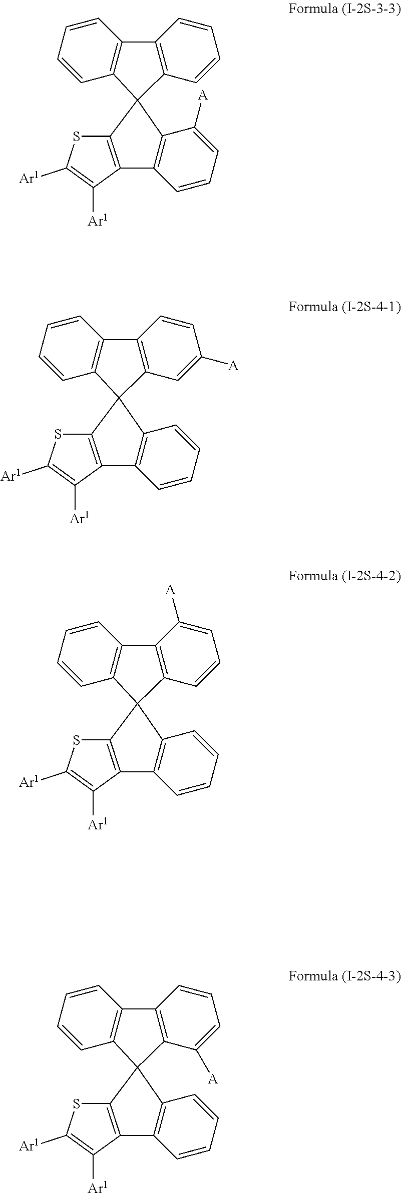

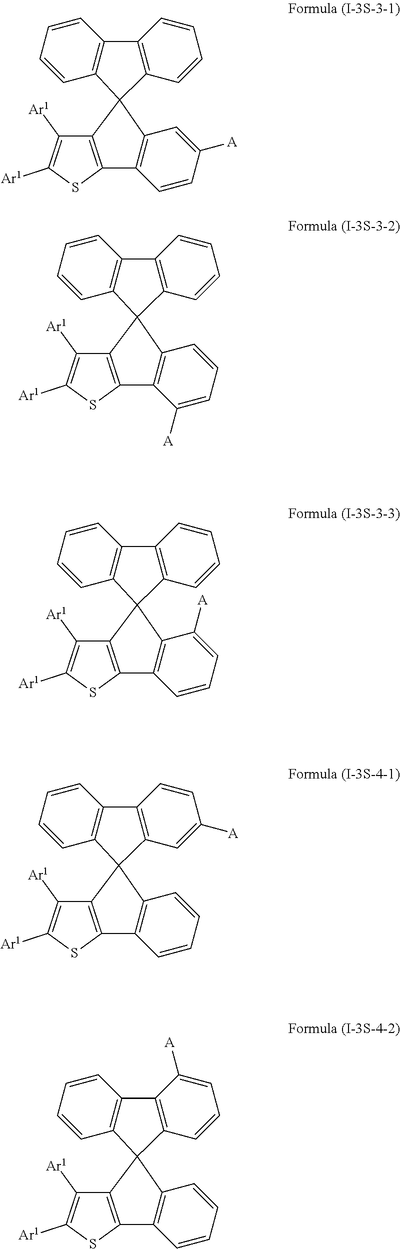

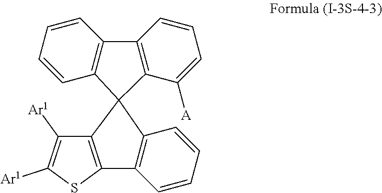

where the variables that occur are as defined above and preferably conform to the preferred embodiments, and where the unoccupied positions on the benzene rings are each substituted by R¹ radicals that are preferably H.

Preferred embodiments of the formula (I-1O-3), (I-1O-4), (I-2O-3), (I-2O-4), (I-3O-3), (I-3O-4) are the following formulae:

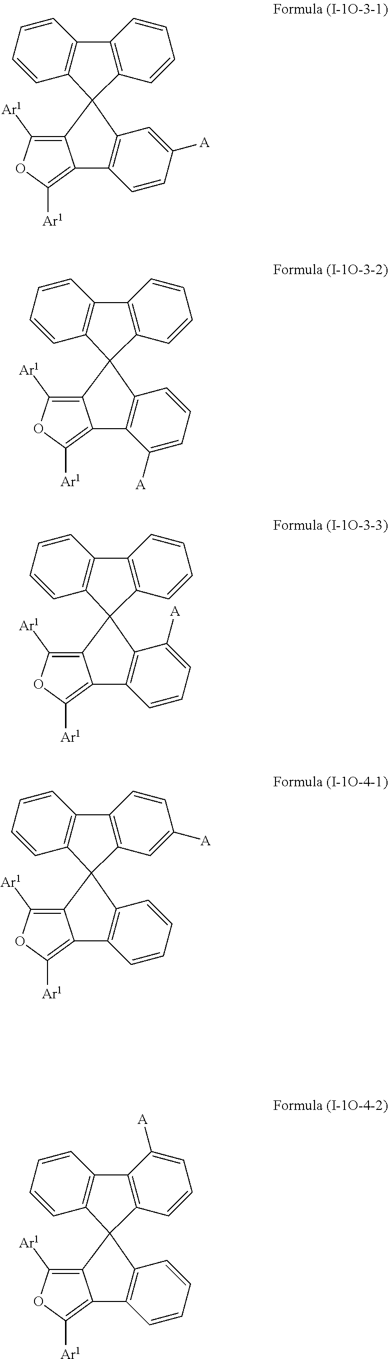

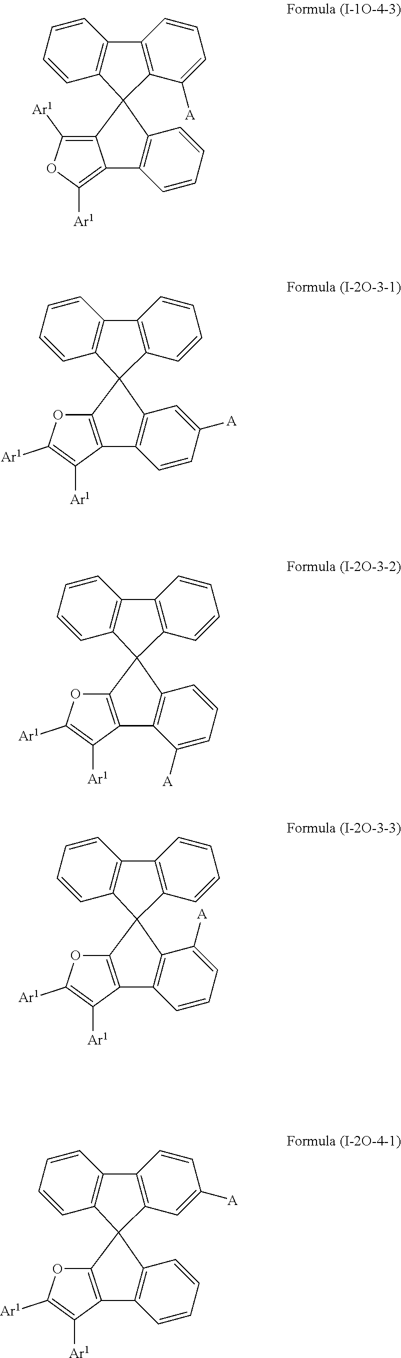

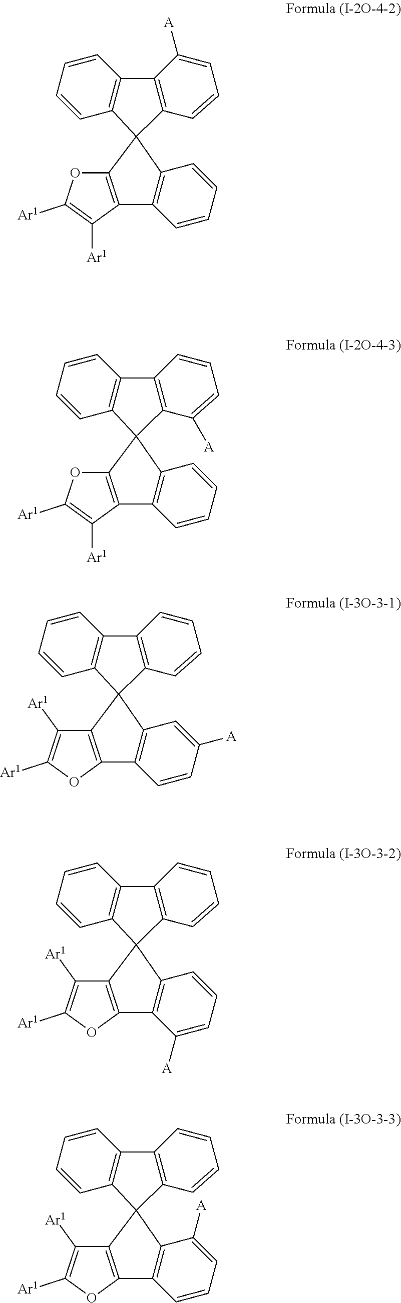

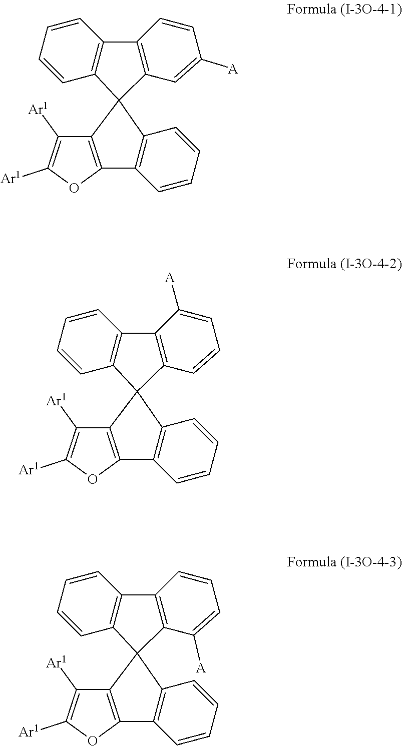

where the variables that occur are as defined above and preferably conform to the preferred embodiments, and where the unoccupied positions on the benzene rings are each substituted by R¹ radicals that are preferably H.

Among the abovementioned formulae, particular preference is given to the formulae (I-1S-3-1), (I-1S-3-2), (I-1S-3-3), (I-1S-4-1), (I-1S-4-2), (I-1 S-4-3), (I-1O-3-1), (I-1O-3-2), (I-1O-3-3), (I-1O-4-1), (I-1O-4-2) and (I-1O-4-3), very particular preference to (I-1S-3-1), (I-1 S-3-2), (I-1S-3-3), (I-1S-4-1), (I-1S-4-2) and (I-1S-4-3).

Most preferably, compounds of the formula (I) conform to one of the formulae (I-1 S-1), (I-1S-2), (I-1S-3-1), (I-1S-3-2), (I-1S-3-3), (I-1S-4-1), (I-1S-4-2), (I-1S-4-3), (I-1O-1), (I-1O-2), (I-1O-3-1), (I-1O-3-2), (I-1O-3-3), (I-1O-4-1), (I-1O-4-2) and (I-1O-4-3), where the variables in these cases preferably conform to the above-specified preferred embodiments. In these cases, Ar¹ is especially preferably phenyl substituted by R² radicals that are H.

Preferred compounds of the formula (I) are the following compounds:

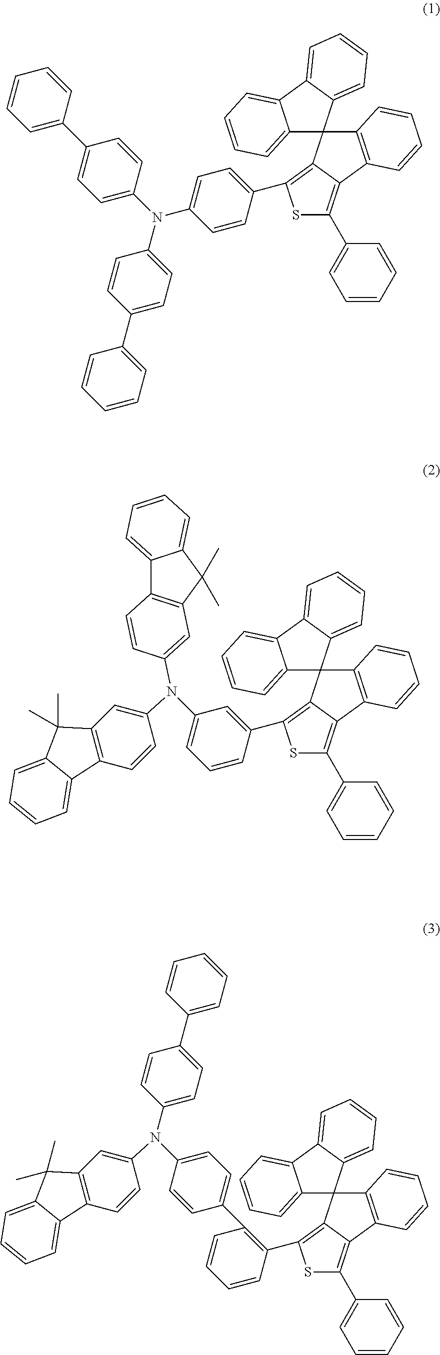 [SMILES: c1ccc(-c2ccc(N(c3ccc(-c4ccccc4)cc3)c3ccc(-c4sc(-c5ccccc5)c5c4C4(c6ccccc6-c6ccccc64)c4ccccc4-5)cc3)cc2)cc1 ; CC1(C)c2ccccc2-c2ccc(N(c3cccc(-c4sc(-c5ccccc5)c5c4C4(c6ccccc6-c6ccccc64)c4ccccc4-5)c3)c3ccc4c(c3)C(C)(C)c3ccccc3-4)cc21 ; CC1(C)c2ccccc2-c2ccc(N(c3ccc(-c4ccccc4)cc3)c3ccc(-c4ccccc4-c4sc(-c5ccccc5)c5c4C4(c6ccccc6-c6ccccc64)c4ccccc4-5)cc3)cc21]

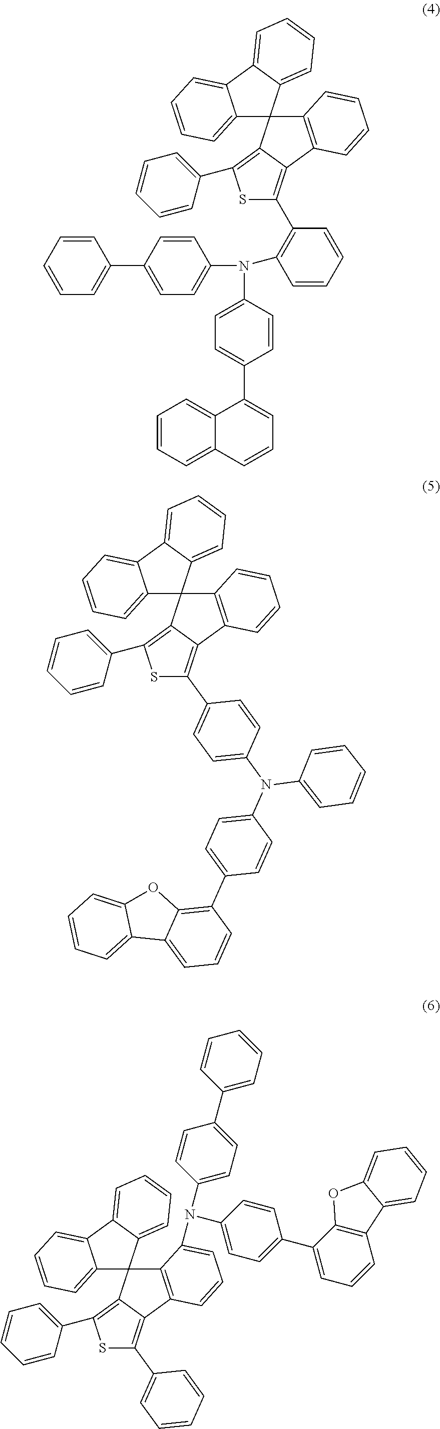

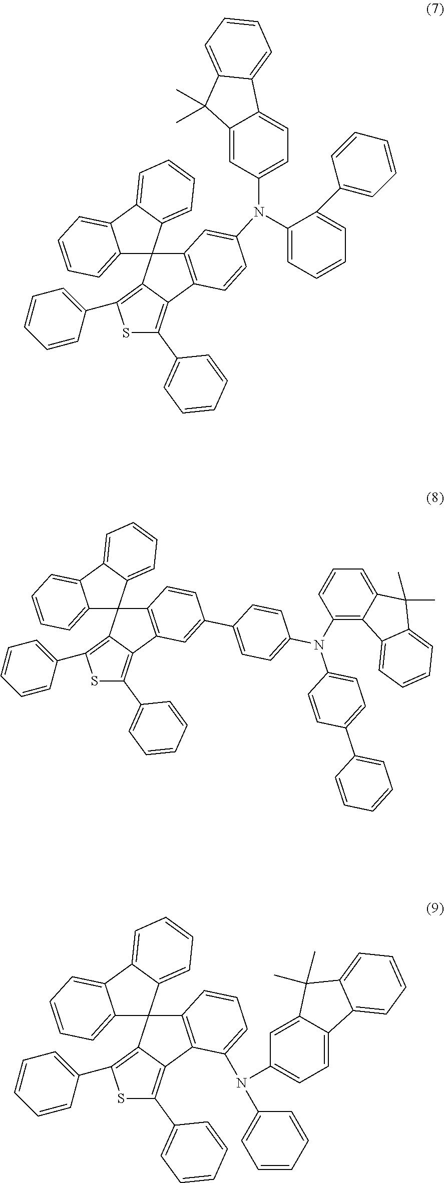 [SMILES: CC1(C)c2ccccc2-c2ccc(N(c3ccc4c(c3)C3(c5ccccc5-c5ccccc53)c3c(-c5ccccc5)sc(-c5ccccc5)c3-4)c3ccccc3-c3ccccc3)cc21 ; CC1(C)c2ccccc2-c2c(N(c3ccc(-c4ccccc4)cc3)c3ccc(-c4ccc5c(c4)-c4c(-c6ccccc6)sc(-c6ccccc6)c4C54c5ccccc5-c5ccccc54)cc3)cccc21 ; CC1(C)c2ccccc2-c2ccc(N(c3ccccc3)c3cccc4c3-c3c(-c5ccccc5)sc(-c5ccccc5)c3C43c4ccccc4-c4ccccc43)cc21]

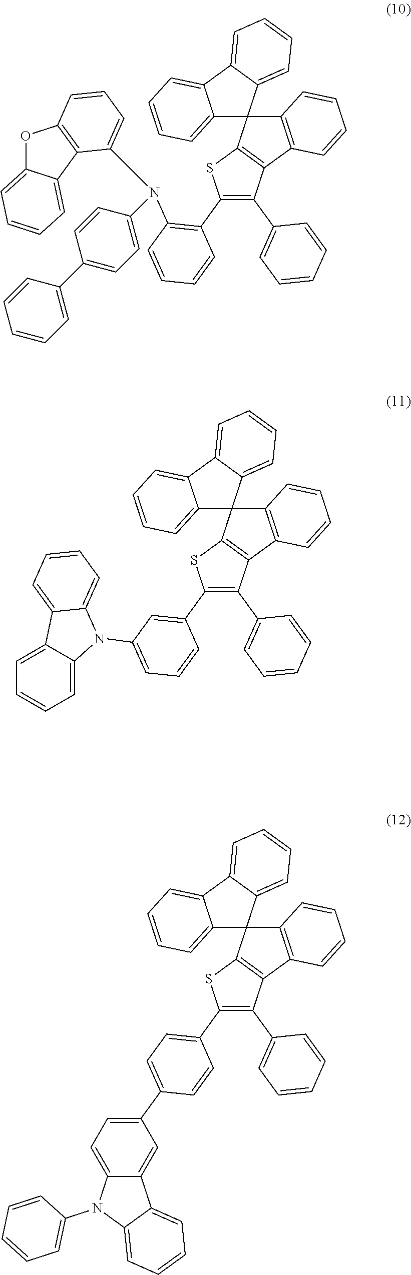 [SMILES: c1ccc(-c2ccc(N(c3ccccc3-c3sc4c(c3-c3ccccc3)-c3ccccc3C43c4ccccc4-c4ccccc43)c3cccc4oc5ccccc5c34)cc2)cc1 ; c1ccc(-c2c(-c3cccc(-n4c5ccccc5c5ccccc54)c3)sc3c2-c2ccccc2C32c3ccccc3-c3ccccc32)cc1 ; c1ccc(-c2c(-c3ccc(-c4ccc5c(c4)c4ccccc4n5-c4ccccc4)cc3)sc3c2-c2ccccc2C32c3ccccc3-c3ccccc32)cc1]

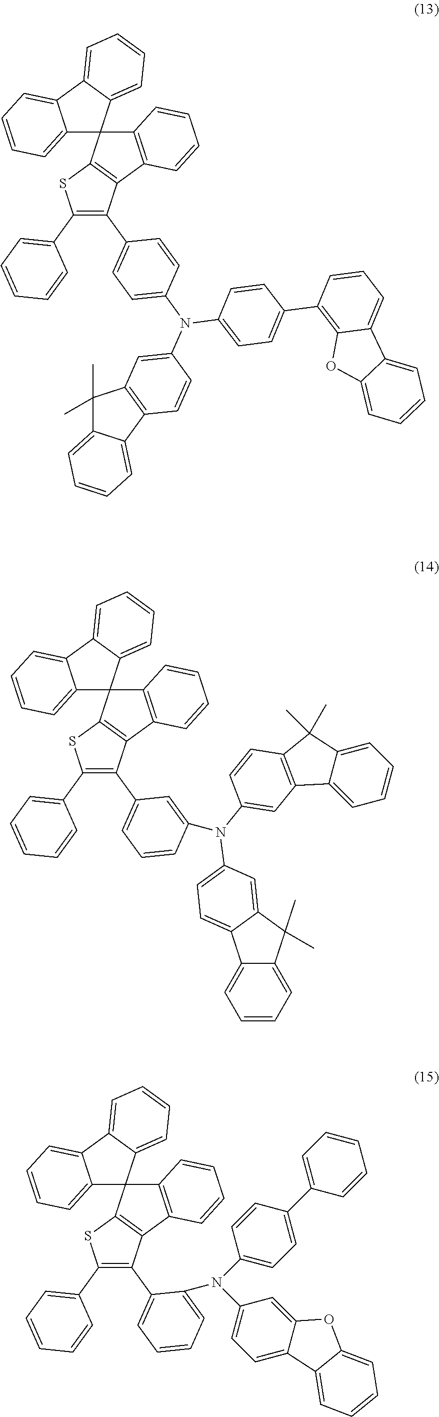 [SMILES: CC1(C)c2ccccc2-c2ccc(N(c3ccc(-c4c(-c5ccccc5)sc5c4-c4ccccc4C54c5ccccc5-c5ccccc54)cc3)c3ccc(-c4cccc5c4oc4ccccc45)cc3)cc21 ; CC1(C)c2ccccc2-c2cc(N(c3cccc(-c4c(-c5ccccc5)sc5c4-c4ccccc4C54c5ccccc5-c5ccccc54)c3)c3ccc4c(c3)C(C)(C)c3ccccc3-4)ccc21 ; c1ccc(-c2ccc(N(c3ccc4c(c3)oc3ccccc34)c3ccccc3-c3c(-c4ccccc4)sc4c3-c3ccccc3C43c4ccccc4-c4ccccc43)cc2)cc1]

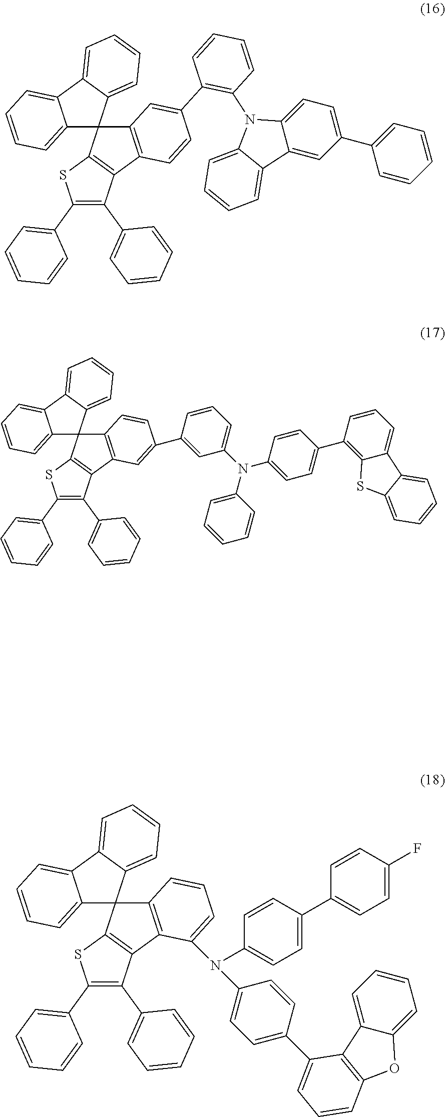 [SMILES: c1ccc(-c2ccc3c(c2)c2ccccc2n3-c2ccccc2-c2ccc3c(c2)C2(c4ccccc4-c4ccccc42)c2sc(-c4ccccc4)c(-c4ccccc4)c2-3)cc1 ; c1ccc(-c2sc3c(c2-c2ccccc2)-c2cc(-c4cccc(N(c5ccccc5)c5ccc(-c6cccc7c6sc6ccccc67)cc5)c4)ccc2C32c3ccccc3-c3ccccc32)cc1 ; Fc1ccc(-c2ccc(N(c3ccc(-c4cccc5oc6ccccc6c45)cc3)c3cccc4c3-c3c(sc(-c5ccccc5)c3-c3ccccc3)C43c4ccccc4-c4ccccc43)cc2)cc1]

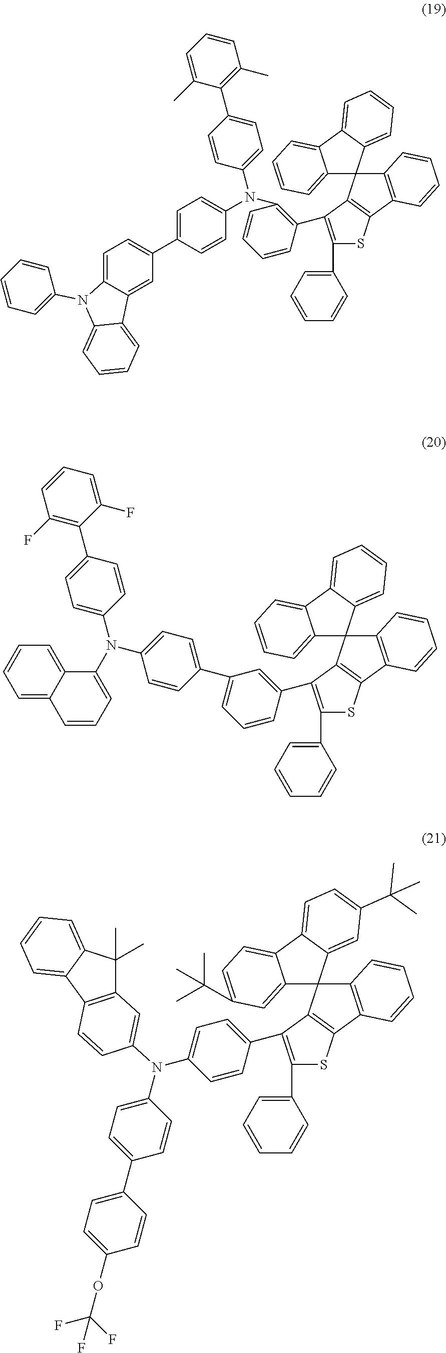 [SMILES: Cc1cccc(C)c1-c1ccc(N(c2ccc(-c3ccc4c(c3)c3ccccc3n4-c3ccccc3)cc2)c2ccccc2-c2c(-c3ccccc3)sc3c2C2(c4ccccc4-c4ccccc42)c2ccccc2-3)cc1 ; Fc1cccc(F)c1-c1ccc(N(c2ccc(-c3cccc(-c4c(-c5ccccc5)sc5c4C4(c6ccccc6-c6ccccc64)c4ccccc4-5)c3)cc2)c2cccc3ccccc23)cc1 ; CC(C)(C)c1ccc2c(c1)C1(c3cc(C(C)(C)C)ccc3-2)c2ccccc2-c2sc(-c3ccccc3)c(-c3ccc(N(c4ccc(-c5ccc(OC(F)(F)F)cc5)cc4)c4ccc5c(c4)C(C)(C)c4ccccc4-5)cc3)c21]

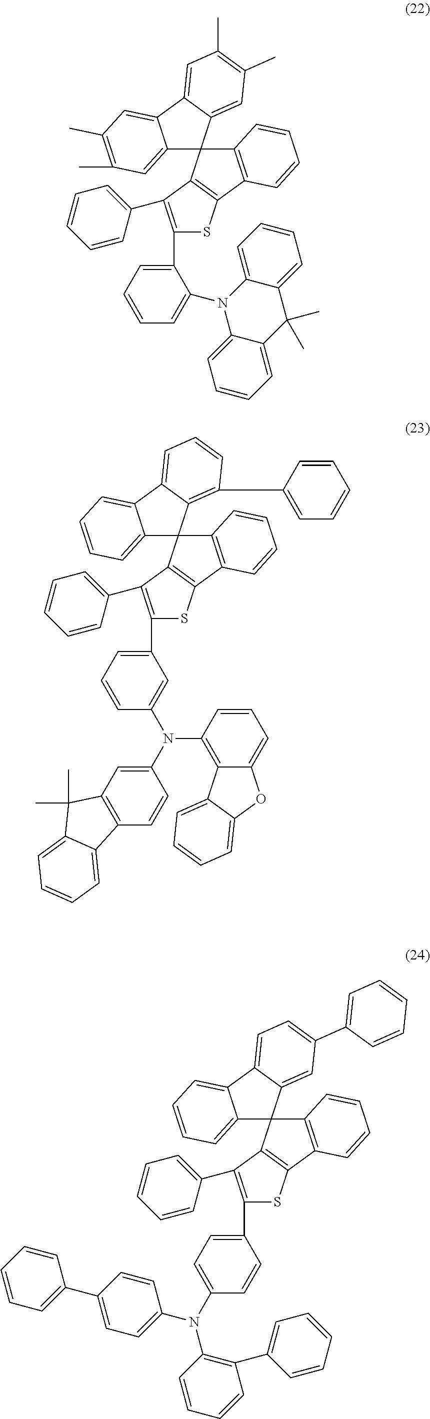

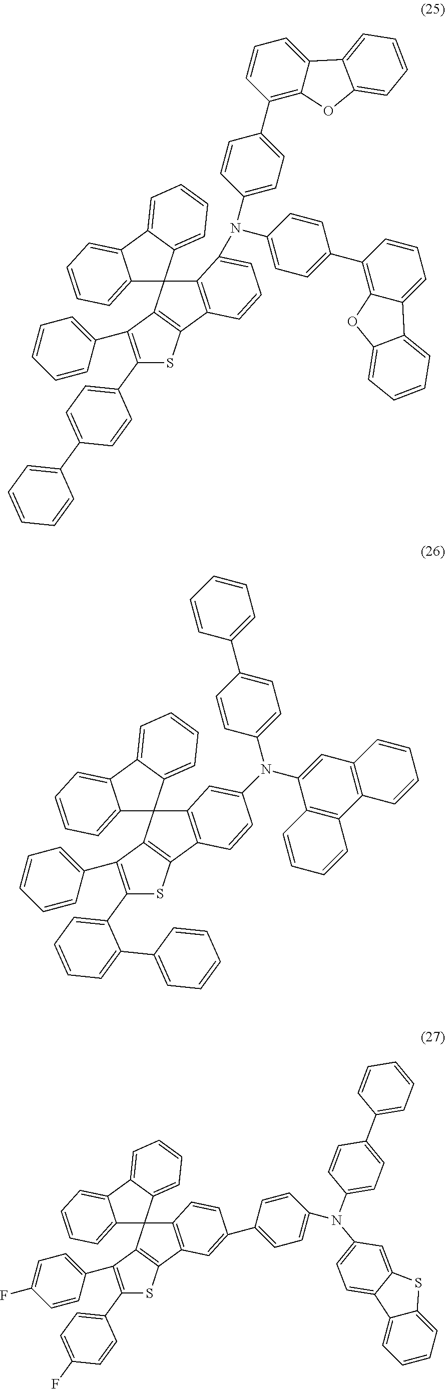 [SMILES: c1ccc(-c2ccc(-c3sc4c(c3-c3ccccc3)C3(c5ccccc5-c5ccccc53)c3c-4cccc3N(c3ccc(-c4cccc5c4oc4ccccc45)cc3)c3ccc(-c4cccc5c4oc4ccccc45)cc3)cc2)cc1 ; c1ccc(-c2ccc(N(c3ccc4c(c3)C3(c5ccccc5-c5ccccc53)c3c-4sc(-c4ccccc4-c4ccccc4)c3-c3ccccc3)c3cc4ccccc4c4ccccc34)cc2)cc1 ; Fc1ccc(-c2sc3c(c2-c2ccc(F)cc2)C2(c4ccccc4-c4ccccc42)c2ccc(-c4ccc(N(c5ccc(-c6ccccc6)cc5)c5ccc6c(c5)sc5ccccc56)cc4)cc2-3)cc1]

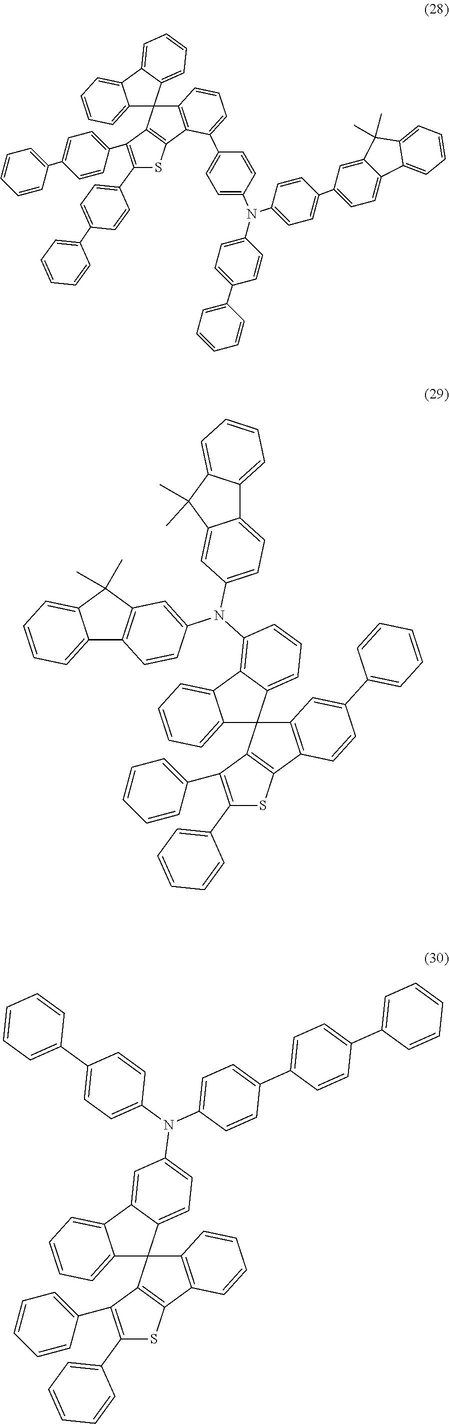 [SMILES: CC1(C)c2ccccc2-c2ccc(-c3ccc(N(c4ccc(-c5ccccc5)cc4)c4ccc(-c5cccc6c5-c5sc(-c7ccc(-c8ccccc8)cc7)c(-c7ccc(-c8ccccc8)cc7)c5C65c6ccccc6-c6ccccc65)cc4)cc3)cc21 ; CC1(C)c2ccccc2-c2ccc(N(c3ccc4c(c3)C(C)(C)c3ccccc3-4)c3cccc4c3-c3ccccc3C43c4cc(-c5ccccc5)ccc4-c4sc(-c5ccccc5)c(-c5ccccc5)c43)cc21 ; c1ccc(-c2ccc(-c3ccc(N(c4ccc(-c5ccccc5)cc4)c4ccc5c(c4)-c4ccccc4C54c5ccccc5-c5sc(-c6ccccc6)c(-c6ccccc6)c54)cc3)cc2)cc1]

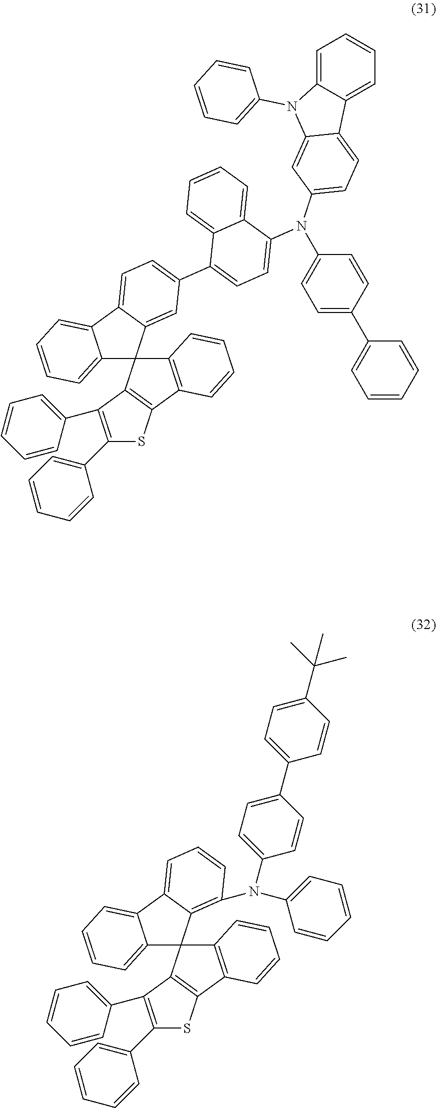 [SMILES: c1ccc(-c2ccc(N(c3ccc4c5ccccc5n(-c5ccccc5)c4c3)c3ccc(-c4ccc5c(c4)C4(c6ccccc6-5)c5ccccc5-c5sc(-c6ccccc6)c(-c6ccccc6)c54)c4ccccc34)cc2)cc1 ; CC(C)(C)c1ccc(-c2ccc(N(c3ccccc3)c3cccc4c3C3(c5ccccc5-4)c4ccccc4-c4sc(-c5ccccc5)c(-c5ccccc5)c43)cc2)cc1]

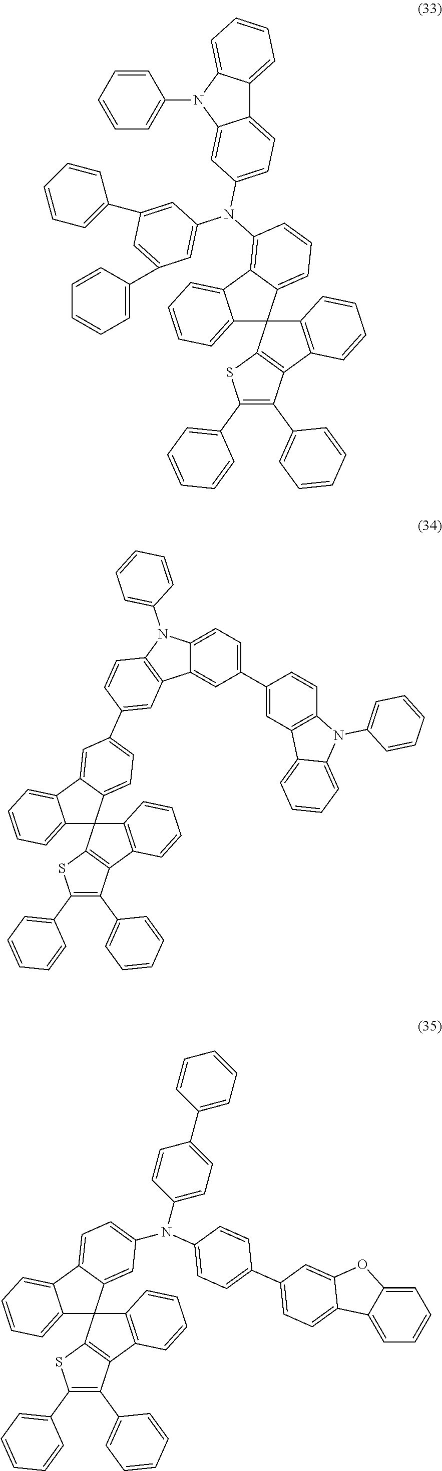 [SMILES: c1ccc(-c2cc(-c3ccccc3)cc(N(c3ccc4c5ccccc5n(-c5ccccc5)c4c3)c3cccc4c3-c3ccccc3C43c4ccccc4-c4c3sc(-c3ccccc3)c4-c3ccccc3)c2)cc1 ; c1ccc(-c2sc3c(c2-c2ccccc2)-c2ccccc2C32c3ccccc3-c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(N(c3ccc(-c4ccc5c(c4)oc4ccccc45)cc3)c3ccc4c(c3)C3(c5ccccc5-4)c4ccccc4-c4c3sc(-c3ccccc3)c4-c3ccccc3)cc2)cc1]

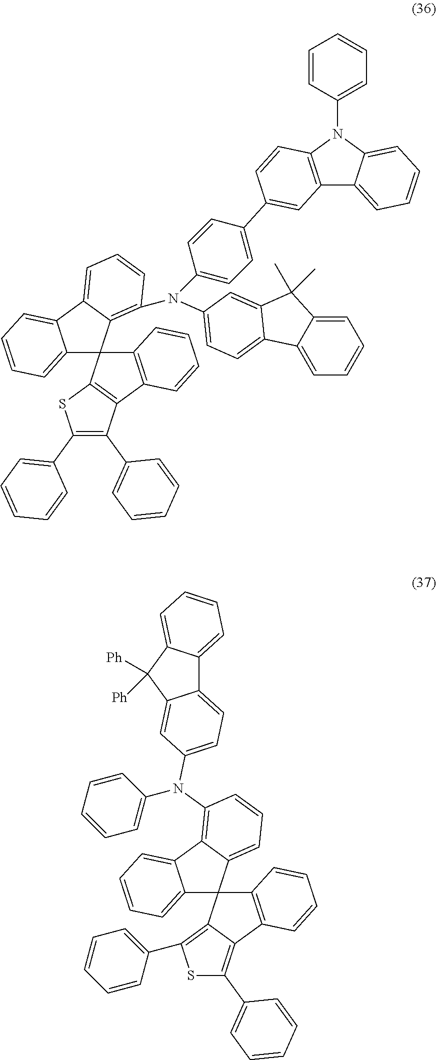 [SMILES: CC1(C)c2ccccc2-c2ccc(N(c3ccc(-c4ccc5c(c4)c4ccccc4n5-c4ccccc4)cc3)c3cccc4c3C3(c5ccccc5-4)c4ccccc4-c4c3sc(-c3ccccc3)c4-c3ccccc3)cc21 ; c1ccc(-c2sc(-c3ccccc3)c3c2-c2ccccc2C32c3ccccc3-c3c(N(c4ccccc4)c4ccc5c(c4)C(c4ccccc4)(c4ccccc4)c4ccccc4-5)cccc32)cc1]

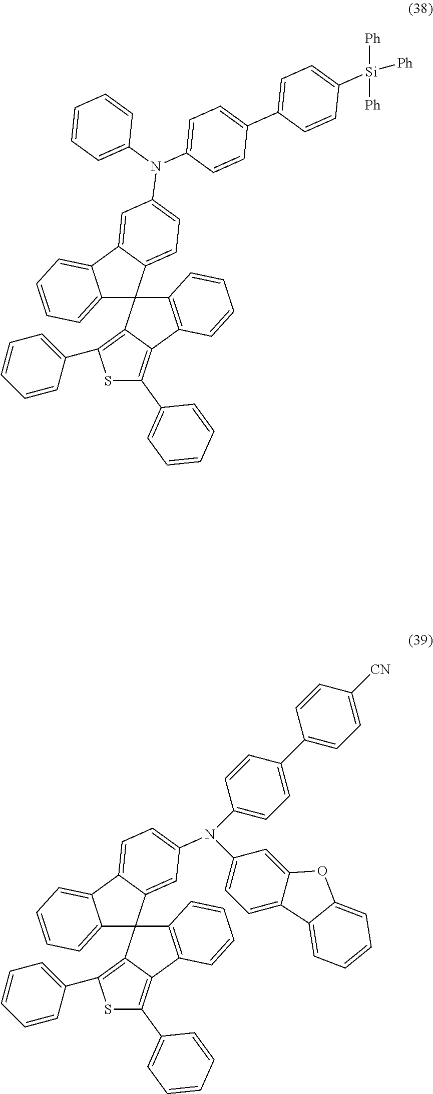 [SMILES: c1ccc(-c2sc(-c3ccccc3)c3c2-c2ccccc2C32c3ccccc3-c3cc(N(c4ccccc4)c4ccc(-c5ccc([Si](c6ccccc6)(c6ccccc6)c6ccccc6)cc5)cc4)ccc32)cc1 ; N#Cc1ccc(-c2ccc(N(c3ccc4c(c3)C3(c5ccccc5-4)c4ccccc4-c4c(-c5ccccc5)sc(-c5ccccc5)c43)c3ccc4c(c3)oc3ccccc34)cc2)cc1]

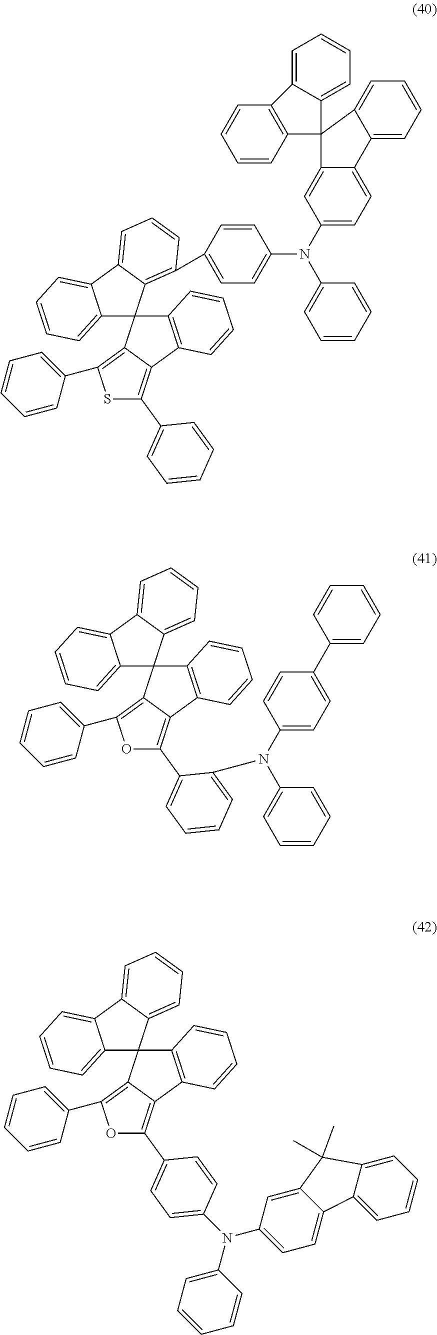 [SMILES: c1ccc(-c2sc(-c3ccccc3)c3c2-c2ccccc2C32c3ccccc3-c3cccc(-c4ccc(N(c5ccccc5)c5ccc6c(c5)C5(c7ccccc7-c7ccccc75)c5ccccc5-6)cc4)c32)cc1 ; c1ccc(-c2ccc(N(c3ccccc3)c3ccccc3-c3oc(-c4ccccc4)c4c3-c3ccccc3C43c4ccccc4-c4ccccc43)cc2)cc1 ; CC1(C)c2ccccc2-c2ccc(N(c3ccccc3)c3ccc(-c4oc(-c5ccccc5)c5c4-c4ccccc4C54c5ccccc5-c5ccccc54)cc3)cc21]

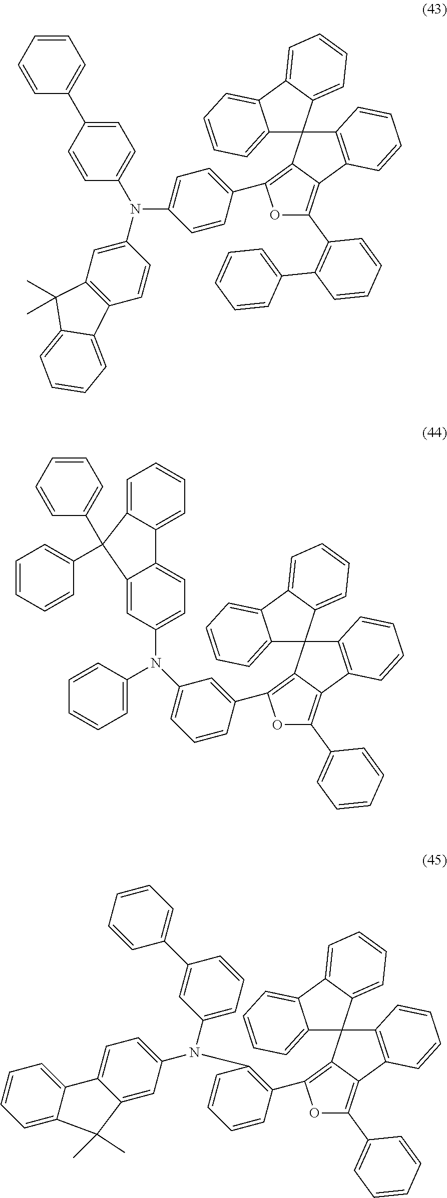 [SMILES: CC1(C)c2ccccc2-c2ccc(N(c3ccc(-c4ccccc4)cc3)c3ccc(-c4oc(-c5ccccc5-c5ccccc5)c5c4C4(c6ccccc6-c6ccccc64)c4ccccc4-5)cc3)cc21 ; c1ccc(-c2oc(-c3cccc(N(c4ccccc4)c4ccc5c(c4)C(c4ccccc4)(c4ccccc4)c4ccccc4-5)c3)c3c2-c2ccccc2C32c3ccccc3-c3ccccc32)cc1 ; CC1(C)c2ccccc2-c2ccc(N(c3cccc(-c4ccccc4)c3)c3ccccc3-c3oc(-c4ccccc4)c4c3C3(c5ccccc5-c5ccccc53)c3ccccc3-4)cc21]

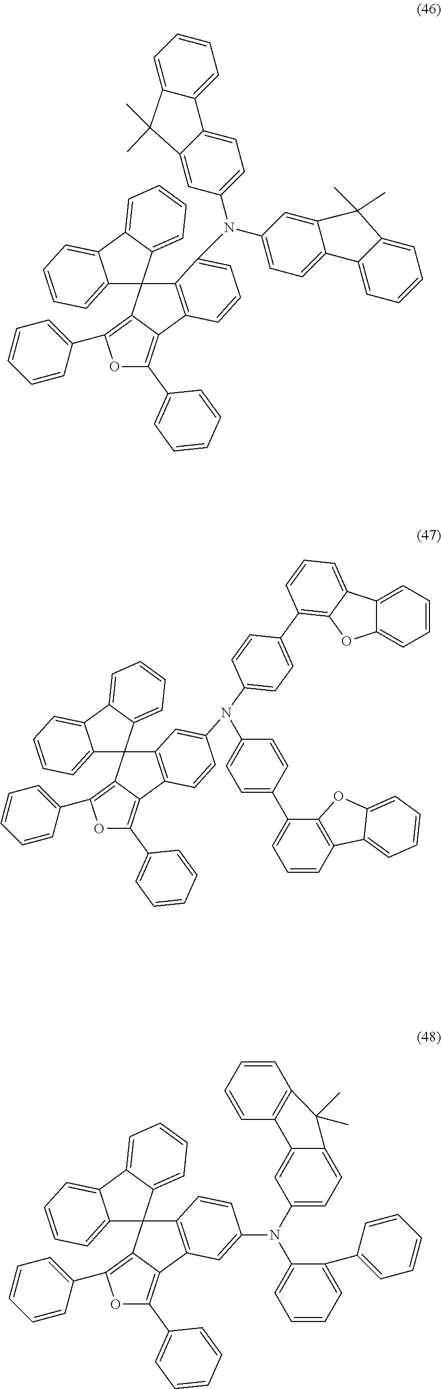 [SMILES: CC1(C)c2ccccc2-c2ccc(N(c3ccc4c(c3)C(C)(C)c3ccccc3-4)c3cccc4c3C3(c5ccccc5-c5ccccc53)c3c(-c5ccccc5)oc(-c5ccccc5)c3-4)cc21 ; c1ccc(-c2oc(-c3ccccc3)c3c2-c2ccc(N(c4ccc(-c5cccc6c5oc5ccccc56)cc4)c4ccc(-c5cccc6c5oc5ccccc56)cc4)cc2C32c3ccccc3-c3ccccc32)cc1 ; CC1(C)c2ccccc2-c2cc(N(c3ccc4c(c3)-c3c(-c5ccccc5)oc(-c5ccccc5)c3C43c4ccccc4-c4ccccc43)c3ccccc3-c3ccccc3)ccc21]

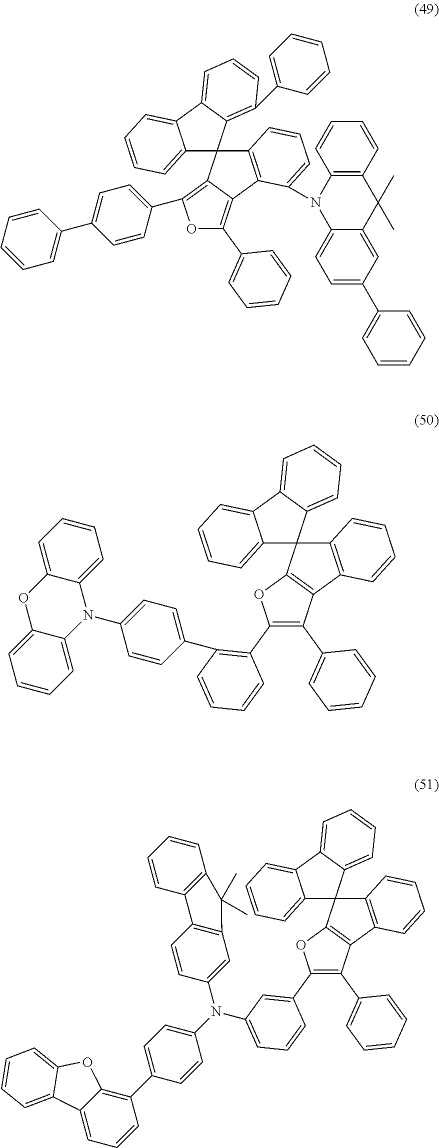 [SMILES: CC1(C)c2ccccc2N(c2cccc3c2-c2c(-c4ccccc4)oc(-c4ccc(-c5ccccc5)cc4)c2C32c3ccccc3-c3cccc(-c4ccccc4)c32)c2ccc(-c3ccccc3)cc21 ; c1ccc(-c2c(-c3ccccc3-c3ccc(N4c5ccccc5Oc5ccccc54)cc3)oc3c2-c2ccccc2C32c3ccccc3-c3ccccc32)cc1 ; CC1(C)c2ccccc2-c2ccc(N(c3ccc(-c4cccc5c4oc4ccccc45)cc3)c3cccc(-c4oc5c(c4-c4ccccc4)-c4ccccc4C54c5ccccc5-c5ccccc54)c3)cc21]

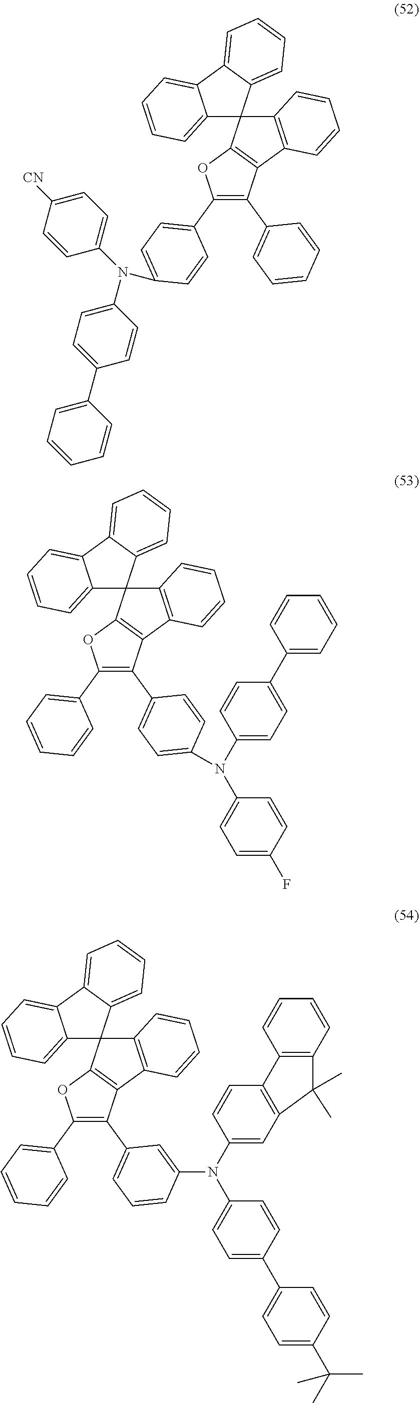 [SMILES: N#Cc1ccc(N(c2ccc(-c3ccccc3)cc2)c2ccc(-c3oc4c(c3-c3ccccc3)-c3ccccc3C43c4ccccc4-c4ccccc43)cc2)cc1 ; Fc1ccc(N(c2ccc(-c3ccccc3)cc2)c2ccc(-c3c(-c4ccccc4)oc4c3-c3ccccc3C43c4ccccc4-c4ccccc43)cc2)cc1 ; CC(C)(C)c1ccc(-c2ccc(N(c3cccc(-c4c(-c5ccccc5)oc5c4-c4ccccc4C54c5ccccc5-c5ccccc54)c3)c3ccc4c(c3)C(C)(C)c3ccccc3-4)cc2)cc1]

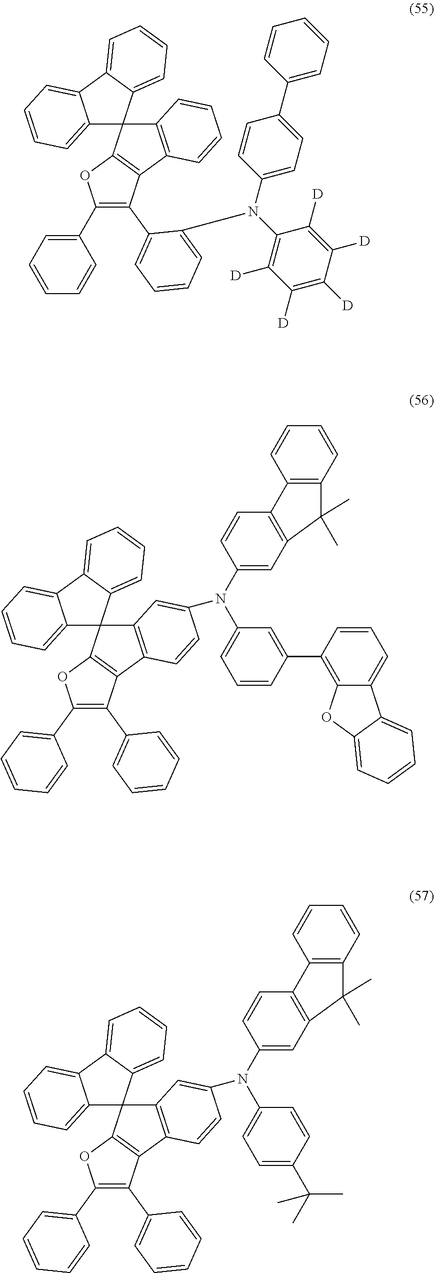 [SMILES: [2H]c1c([2H])c([2H])c(N(c2ccc(-c3ccccc3)cc2)c2ccccc2-c2c(-c3ccccc3)oc3c2-c2ccccc2C32c3ccccc3-c3ccccc32)c([2H])c1[2H] ; CC1(C)c2ccccc2-c2ccc(N(c3cccc(-c4cccc5c4oc4ccccc45)c3)c3ccc4c(c3)C3(c5ccccc5-c5ccccc53)c3oc(-c5ccccc5)c(-c5ccccc5)c3-4)cc21 ; CC(C)(C)c1ccc(N(c2ccc3c(c2)C(C)(C)c2ccccc2-3)c2ccc3c(c2)C2(c4ccccc4-c4ccccc42)c2oc(-c4ccccc4)c(-c4ccccc4)c2-3)cc1]

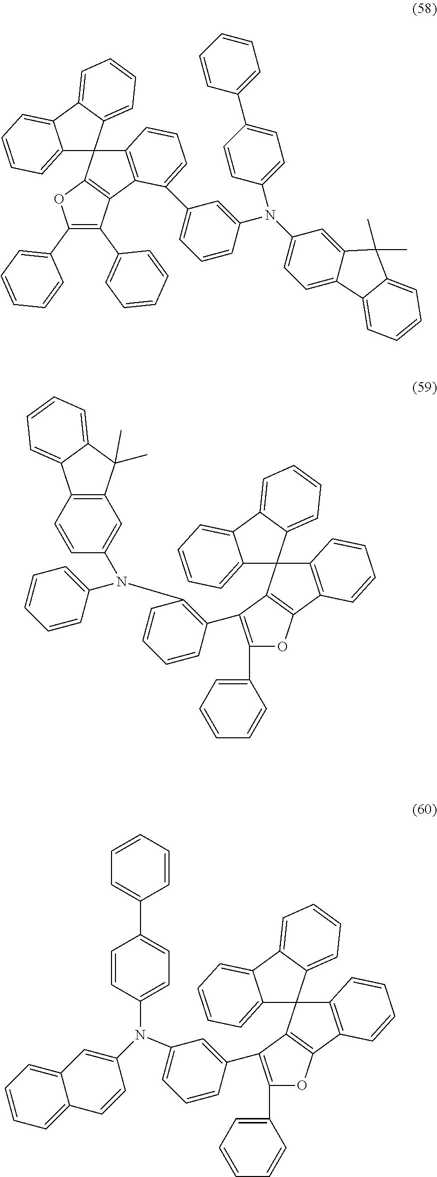 [SMILES: CC1(C)c2ccccc2-c2ccc(N(c3ccc(-c4ccccc4)cc3)c3cccc(-c4cccc5c4-c4c(oc(-c6ccccc6)c4-c4ccccc4)C54c5ccccc5-c5ccccc54)c3)cc21 ; CC1(C)c2ccccc2-c2ccc(N(c3ccccc3)c3ccccc3-c3c(-c4ccccc4)oc4c3C3(c5ccccc5-c5ccccc53)c3ccccc3-4)cc21 ; c1ccc(-c2ccc(N(c3cccc(-c4c(-c5ccccc5)oc5c4C4(c6ccccc6-c6ccccc64)c4ccccc4-5)c3)c3ccc4ccccc4c3)cc2)cc1]

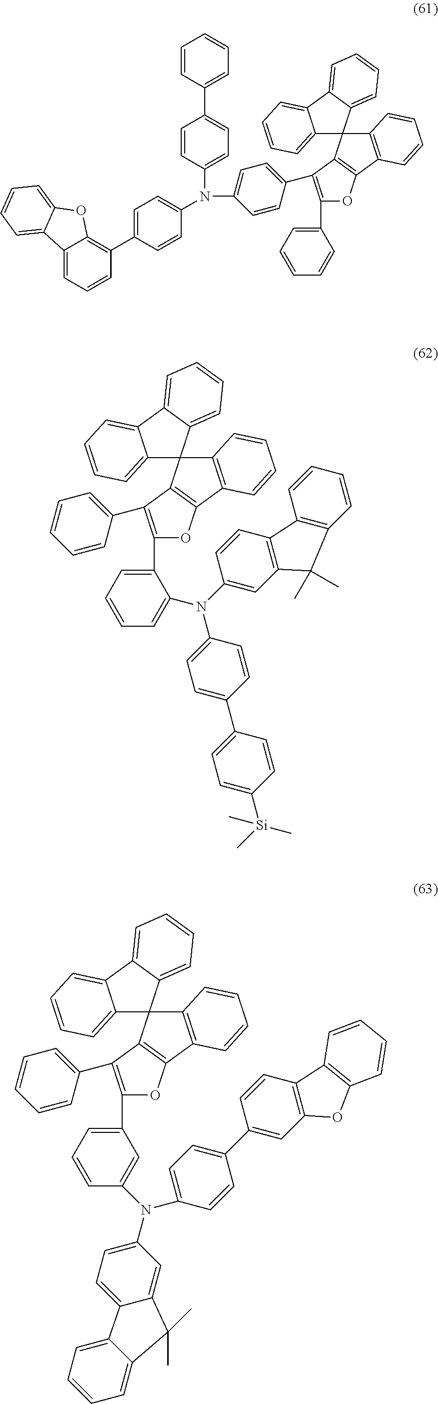 [SMILES: c1ccc(-c2ccc(N(c3ccc(-c4c(-c5ccccc5)oc5c4C4(c6ccccc6-c6ccccc64)c4ccccc4-5)cc3)c3ccc(-c4cccc5c4oc4ccccc45)cc3)cc2)cc1 ; CC1(C)c2ccccc2-c2ccc(N(c3ccc(-c4ccc([Si](C)(C)C)cc4)cc3)c3ccccc3-c3oc4c(c3-c3ccccc3)C3(c5ccccc5-c5ccccc53)c3ccccc3-4)cc21 ; CC1(C)c2ccccc2-c2ccc(N(c3ccc(-c4ccc5c(c4)oc4ccccc45)cc3)c3cccc(-c4oc5c(c4-c4ccccc4)C4(c6ccccc6-c6ccccc64)c4ccccc4-5)c3)cc21]

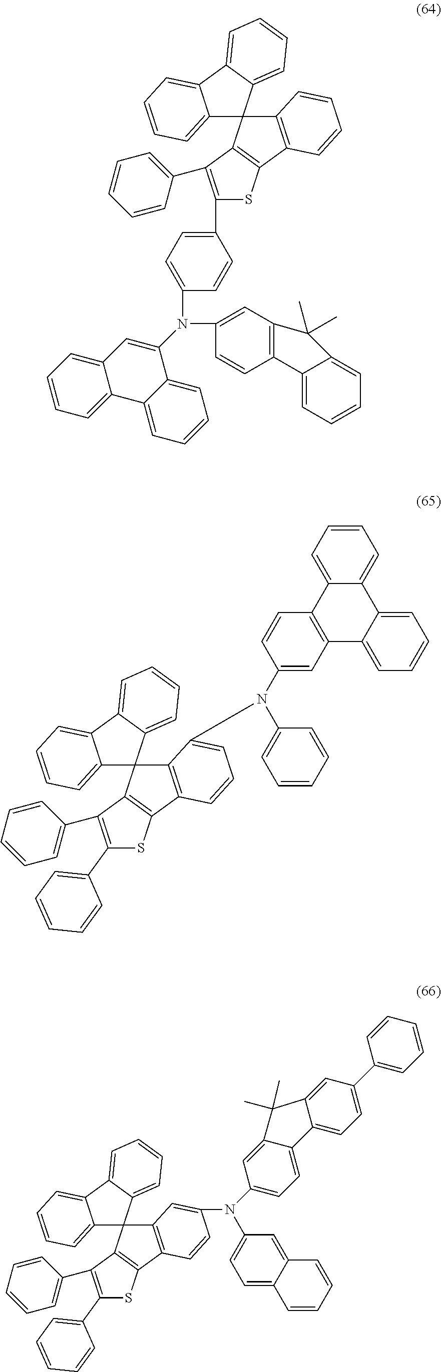 [SMILES: CC1(C)c2ccccc2-c2ccc(N(c3ccc(-c4sc5c(c4-c4ccccc4)C4(c6ccccc6-c6ccccc64)c4ccccc4-5)cc3)c3cc4ccccc4c4ccccc34)cc21 ; c1ccc(-c2sc3c(c2-c2ccccc2)C2(c4ccccc4-c4ccccc42)c2c-3cccc2N(c2ccccc2)c2ccc3c4ccccc4c4ccccc4c3c2)cc1 ; CC1(C)c2cc(-c3ccccc3)ccc2-c2ccc(N(c3ccc4c(c3)C3(c5ccccc5-c5ccccc53)c3c-4sc(-c4ccccc4)c3-c3ccccc3)c3ccc4ccccc4c3)cc21]

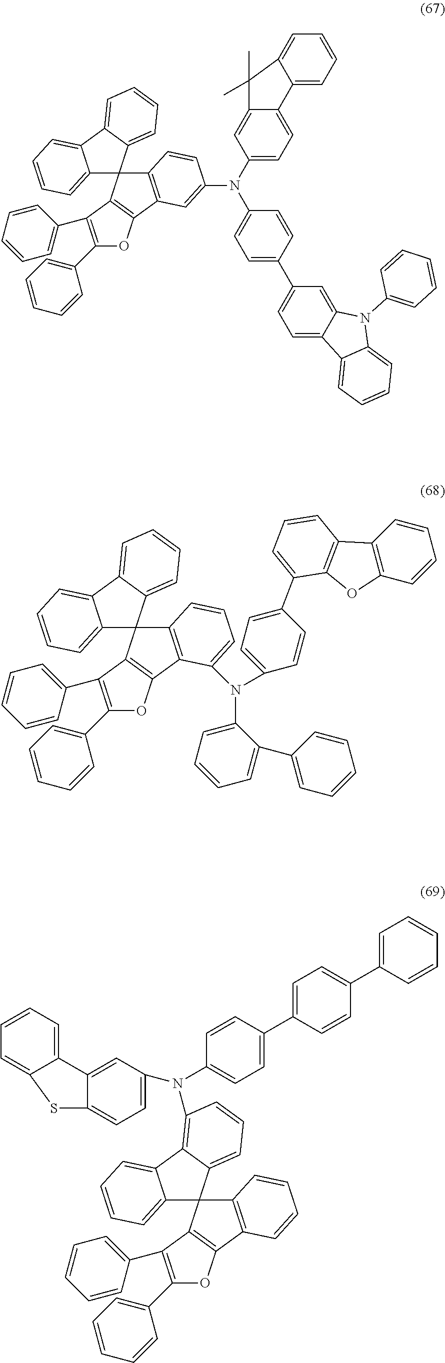 [SMILES: CC1(C)c2ccccc2-c2ccc(N(c3ccc(-c4ccc5c6ccccc6n(-c6ccccc6)c5c4)cc3)c3ccc4c(c3)-c3oc(-c5ccccc5)c(-c5ccccc5)c3C43c4ccccc4-c4ccccc43)cc21 ; c1ccc(-c2ccccc2N(c2ccc(-c3cccc4c3oc3ccccc34)cc2)c2cccc3c2-c2oc(-c4ccccc4)c(-c4ccccc4)c2C32c3ccccc3-c3ccccc32)cc1 ; c1ccc(-c2ccc(-c3ccc(N(c4ccc5sc6ccccc6c5c4)c4cccc5c4-c4ccccc4C54c5ccccc5-c5oc(-c6ccccc6)c(-c6ccccc6)c54)cc3)cc2)cc1]

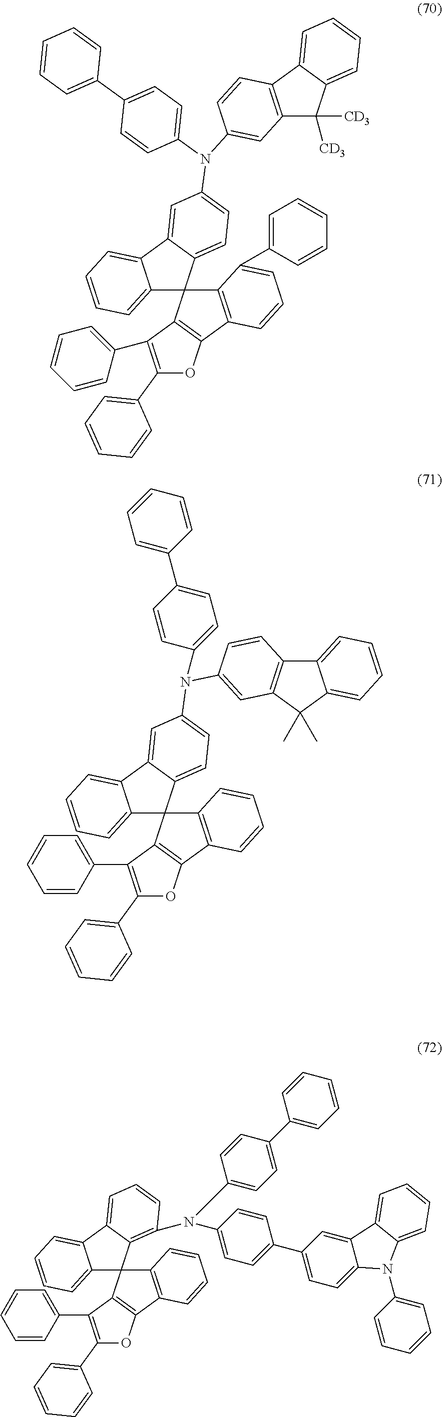

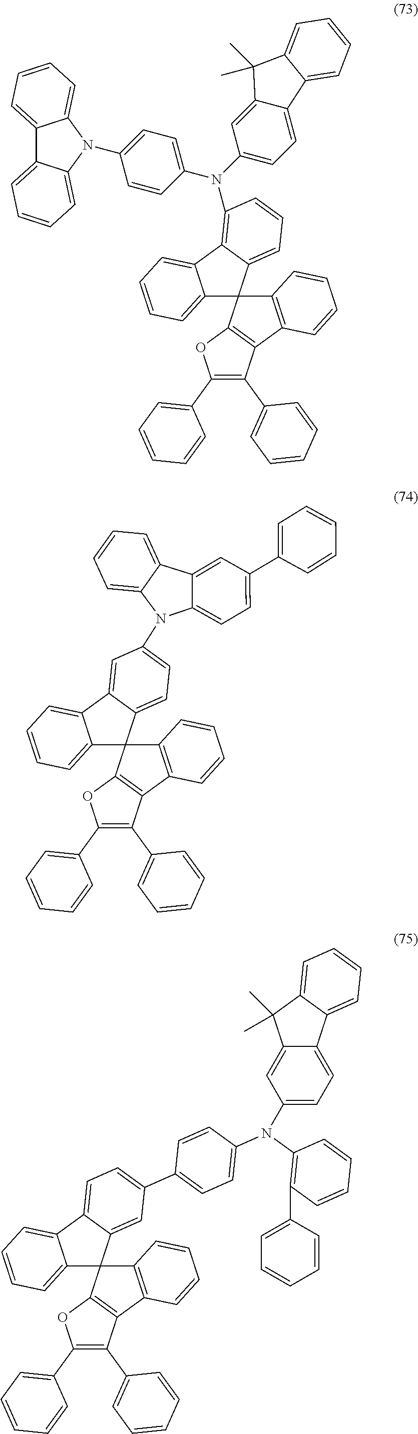

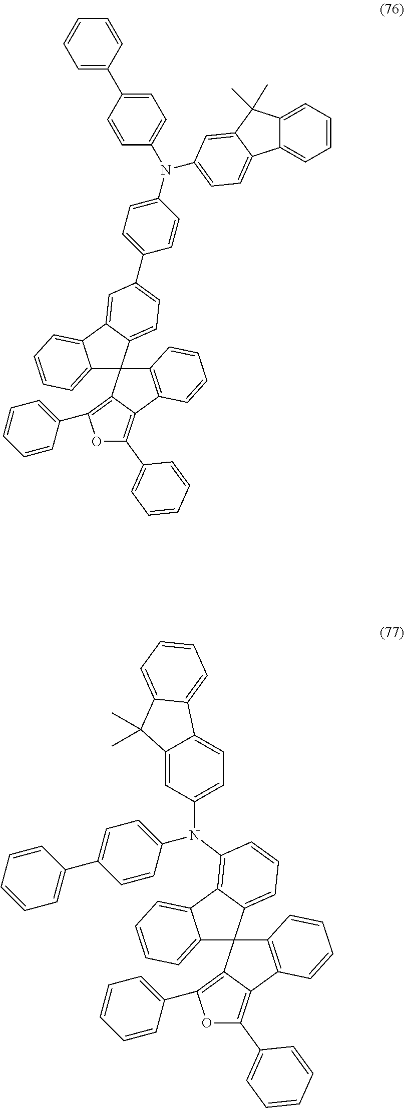 [SMILES: CC1(C)c2ccccc2-c2ccc(N(c3ccc(-c4ccccc4)cc3)c3ccc(-c4ccc5c(c4)-c4ccccc4C54c5ccccc5-c5c(-c6ccccc6)oc(-c6ccccc6)c54)cc3)cc21 ; CC1(C)c2ccccc2-c2ccc(N(c3ccc(-c4ccccc4)cc3)c3cccc4c3-c3ccccc3C43c4ccccc4-c4c(-c5ccccc5)oc(-c5ccccc5)c43)cc21]

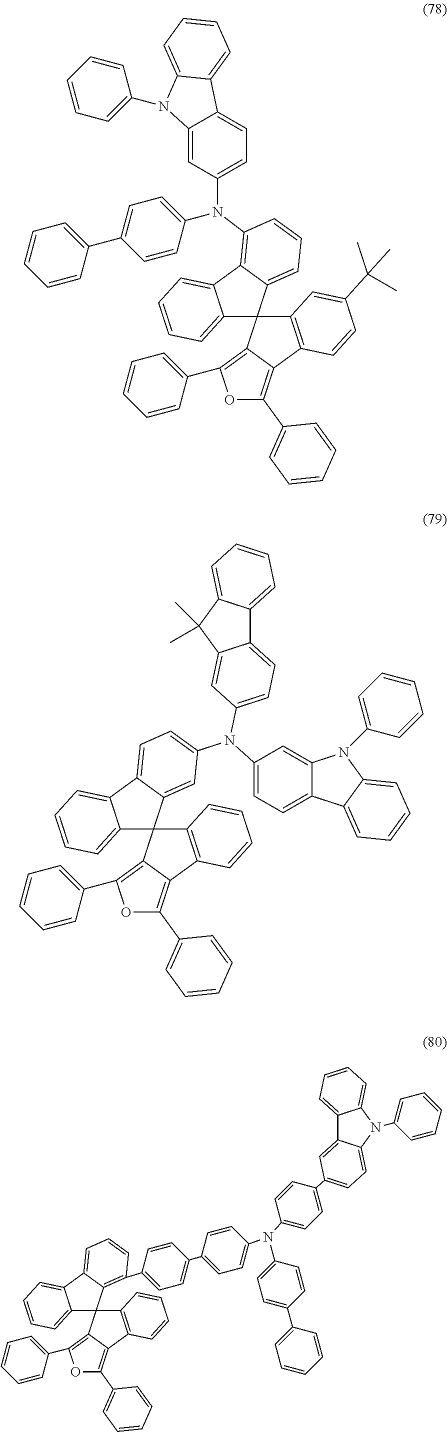 [SMILES: CC(C)(C)c1ccc2c(c1)C1(c3ccccc3-c3c(N(c4ccc(-c5ccccc5)cc4)c4ccc5c6ccccc6n(-c6ccccc6)c5c4)cccc31)c1c(-c3ccccc3)oc(-c3ccccc3)c1-2 ; CC1(C)c2ccccc2-c2ccc(N(c3ccc4c(c3)C3(c5ccccc5-4)c4ccccc4-c4c(-c5ccccc5)oc(-c5ccccc5)c43)c3ccc4c5ccccc5n(-c5ccccc5)c4c3)cc21 ; c1ccc(-c2ccc(N(c3ccc(-c4ccc(-c5cccc6c5C5(c7ccccc7-6)c6ccccc6-c6c(-c7ccccc7)oc(-c7ccccc7)c65)cc4)cc3)c3ccc(-c4ccc5c(c4)c4ccccc4n5-c4ccccc4)cc3)cc2)cc1]

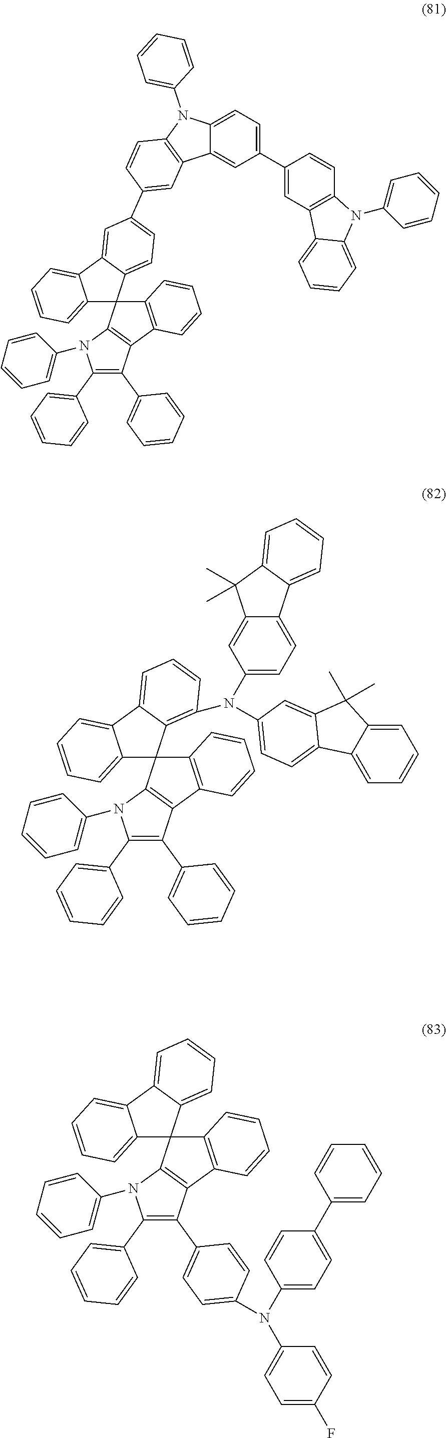 [SMILES: c1ccc(-c2c3c(n(-c4ccccc4)c2-c2ccccc2)C2(c4ccccc4-c4cc(-c5ccc6c(c5)c5cc(-c7ccc8c(c7)c7ccccc7n8-c7ccccc7)ccc5n6-c5ccccc5)ccc42)c2ccccc2-3)cc1 ; CC1(C)c2ccccc2-c2ccc(N(c3ccc4c(c3)C(C)(C)c3ccccc3-4)c3cccc4c3C3(c5ccccc5-4)c4ccccc4-c4c(-c5ccccc5)c(-c5ccccc5)n(-c5ccccc5)c43)cc21 ; Fc1ccc(N(c2ccc(-c3ccccc3)cc2)c2ccc(-c3c4c(n(-c5ccccc5)c3-c3ccccc3)C3(c5ccccc5-c5ccccc53)c3ccccc3-4)cc2)cc1]

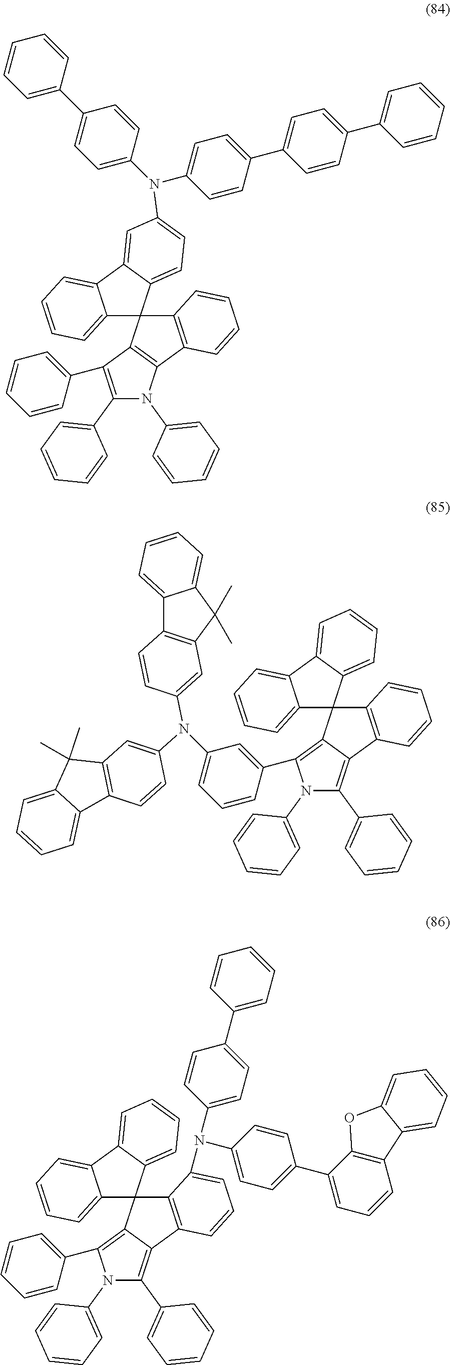 [SMILES: c1ccc(-c2ccc(-c3ccc(N(c4ccc(-c5ccccc5)cc4)c4ccc5c(c4)-c4ccccc4C54c5ccccc5-c5c4c(-c4ccccc4)c(-c4ccccc4)n5-c4ccccc4)cc3)cc2)cc1 ; CC1(C)c2ccccc2-c2ccc(N(c3cccc(-c4c5c(c(-c6ccccc6)n4-c4ccccc4)-c4ccccc4C54c5ccccc5-c5ccccc54)c3)c3ccc4c(c3)C(C)(C)c3ccccc3-4)cc21 ; c1ccc(-c2ccc(N(c3ccc(-c4cccc5c4oc4ccccc45)cc3)c3cccc4c3C3(c5ccccc5-c5ccccc53)c3c-4c(-c4ccccc4)n(-c4ccccc4)c3-c3ccccc3)cc2)cc1]

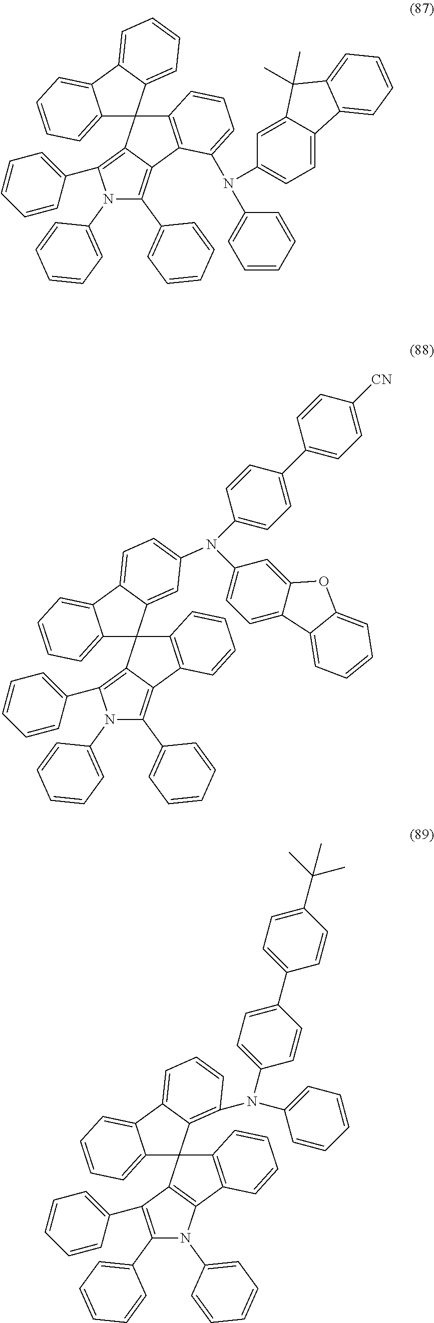 [SMILES: CC1(C)c2ccccc2-c2ccc(N(c3ccccc3)c3cccc4c3-c3c(c(-c5ccccc5)n(-c5ccccc5)c3-c3ccccc3)C43c4ccccc4-c4ccccc43)cc21 ; N#Cc1ccc(-c2ccc(N(c3ccc4c(c3)C3(c5ccccc5-4)c4ccccc4-c4c3c(-c3ccccc3)n(-c3ccccc3)c4-c3ccccc3)c3ccc4c(c3)oc3ccccc34)cc2)cc1 ; CC(C)(C)c1ccc(-c2ccc(N(c3ccccc3)c3cccc4c3C3(c5ccccc5-4)c4ccccc4-c4c3c(-c3ccccc3)c(-c3ccccc3)n4-c3ccccc3)cc2)cc1]

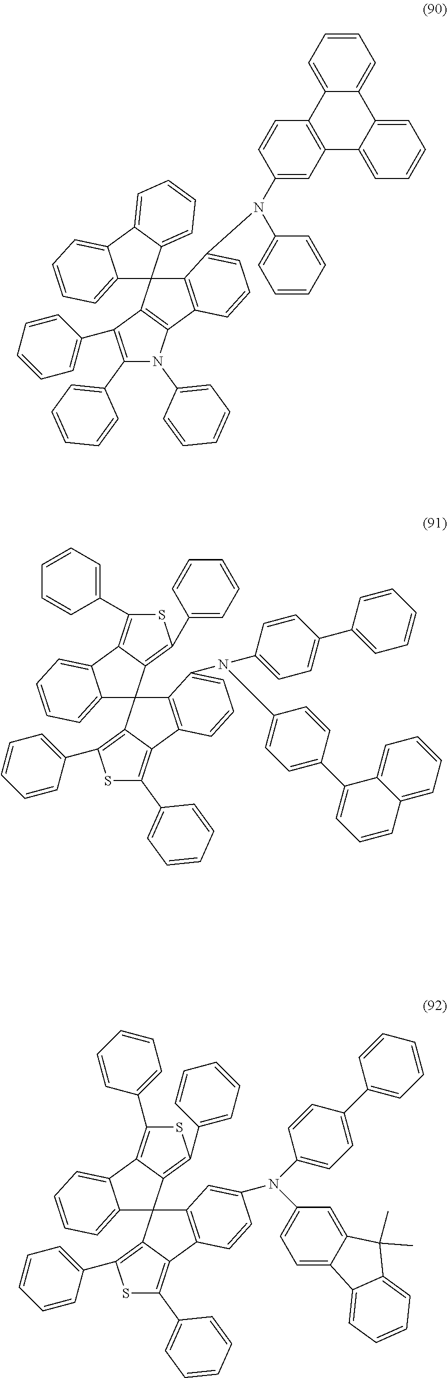 [SMILES: c1ccc(-c2c3c(n(-c4ccccc4)c2-c2ccccc2)-c2cccc(N(c4ccccc4)c4ccc5c6ccccc6c6ccccc6c5c4)c2C32c3ccccc3-c3ccccc32)cc1 ; c1ccc(-c2ccc(N(c3ccc(-c4cccc5ccccc45)cc3)c3cccc4c3C3(c5ccccc5-c5c(-c6ccccc6)sc(-c6ccccc6)c53)c3c(-c5ccccc5)sc(-c5ccccc5)c3-4)cc2)cc1 ; CC1(C)c2ccccc2-c2ccc(N(c3ccc(-c4ccccc4)cc3)c3ccc4c(c3)C3(c5ccccc5-c5c(-c6ccccc6)sc(-c6ccccc6)c53)c3c(-c5ccccc5)sc(-c5ccccc5)c3-4)cc21]

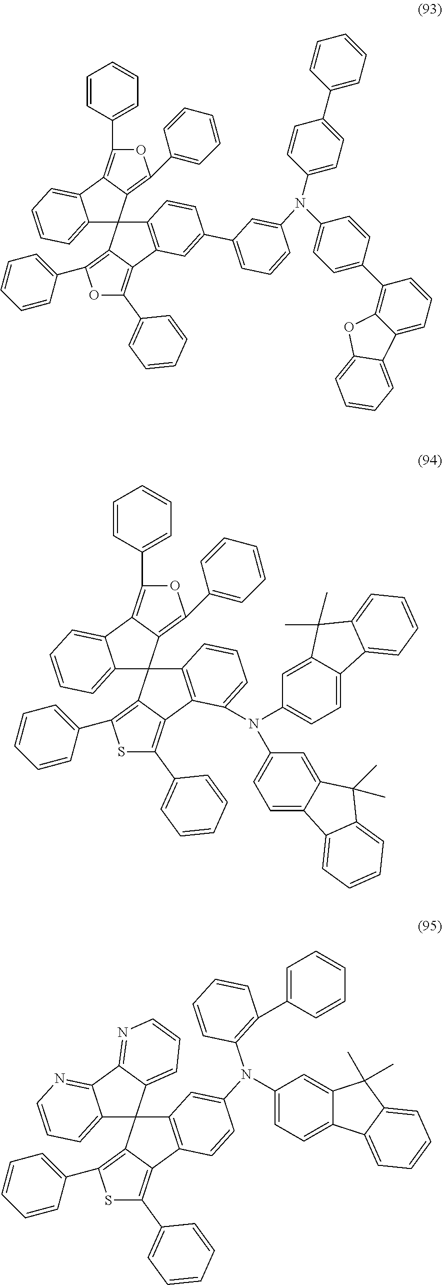 [SMILES: c1ccc(-c2ccc(N(c3ccc(-c4cccc5c4oc4ccccc45)cc3)c3cccc(-c4ccc5c(c4)-c4c(-c6ccccc6)oc(-c6ccccc6)c4C54c5ccccc5-c5c(-c6ccccc6)oc(-c6ccccc6)c54)c3)cc2)cc1 ; CC1(C)c2ccccc2-c2ccc(N(c3ccc4c(c3)C(C)(C)c3ccccc3-4)c3cccc4c3-c3c(-c5ccccc5)sc(-c5ccccc5)c3C43c4ccccc4-c4c(-c5ccccc5)oc(-c5ccccc5)c43)cc21 ; CC1(C)c2ccccc2-c2ccc(N(c3ccc4c(c3)C3(c5cccnc5-c5ncccc53)c3c(-c5ccccc5)sc(-c5ccccc5)c3-4)c3ccccc3-c3ccccc3)cc21]

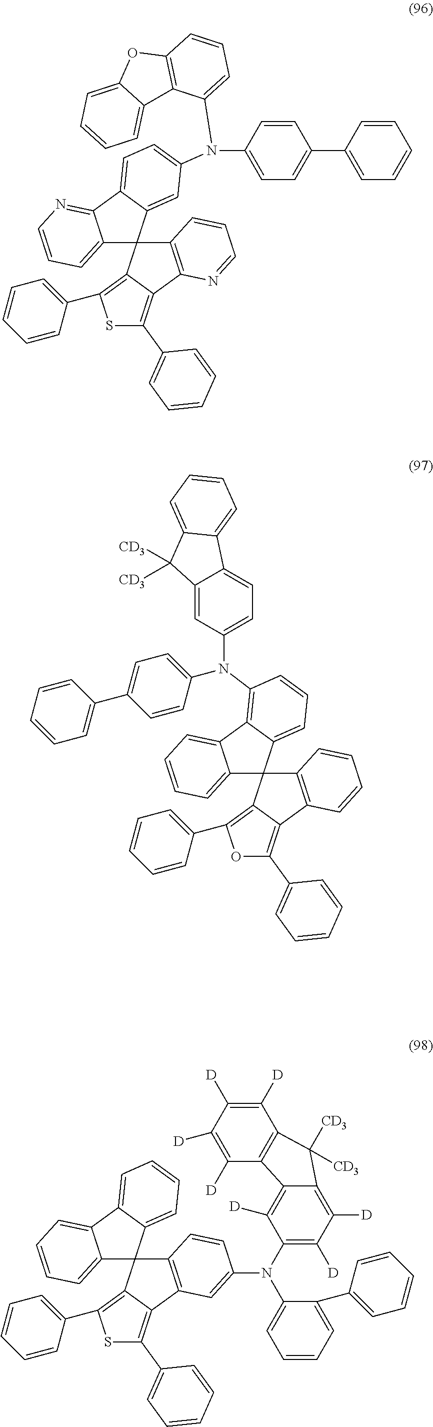 [SMILES: c1ccc(-c2ccc(N(c3ccc4c(c3)C3(c5cccnc5-4)c4cccnc4-c4c(-c5ccccc5)sc(-c5ccccc5)c43)c3cccc4oc5ccccc5c34)cc2)cc1 ; [2H]C([2H])([2H])C1(C([2H])([2H])[2H])c2ccccc2-c2ccc(N(c3ccc(-c4ccccc4)cc3)c3cccc4c3-c3ccccc3C43c4ccccc4-c4c(-c5ccccc5)oc(-c5ccccc5)c43)cc21 ; [2H]c1c([2H])c([2H])c2c(c1[2H])-c1c([2H])c(N(c3ccc4c(c3)-c3c(-c5ccccc5)sc(-c5ccccc5)c3C43c4ccccc4-c4ccccc43)c3ccccc3-c3ccccc3)c([2H])c([2H])c1C2(C([2H])([2H])[2H])C([2H])([2H])[2H]]

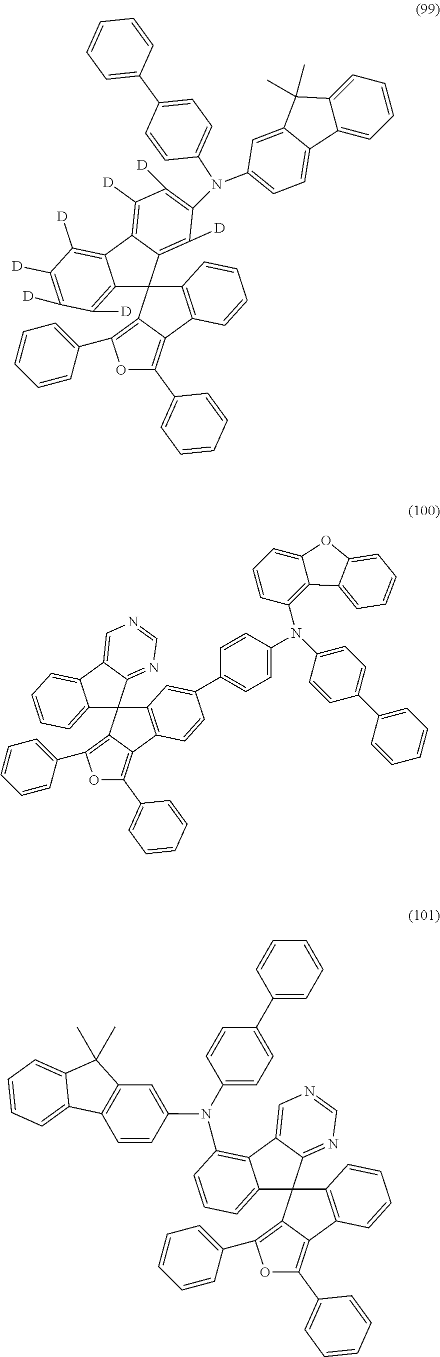 [SMILES: [2H]c1c([2H])c([2H])c2c(c1[2H])-c1c([2H])c([2H])c(N(c3ccc(-c4ccccc4)cc3)c3ccc4c(c3)C(C)(C)c3ccccc3-4)c([2H])c1C21c2ccccc2-c2c(-c3ccccc3)oc(-c3ccccc3)c21 ; c1ccc(-c2ccc(N(c3ccc(-c4ccc5c(c4)C4(c6ccccc6-c6cncnc64)c4c(-c6ccccc6)oc(-c6ccccc6)c4-5)cc3)c3cccc4oc5ccccc5c34)cc2)cc1 ; CC1(C)c2ccccc2-c2ccc(N(c3ccc(-c4ccccc4)cc3)c3cccc4c3-c3cncnc3C43c4ccccc4-c4c(-c5ccccc5)oc(-c5ccccc5)c43)cc21]

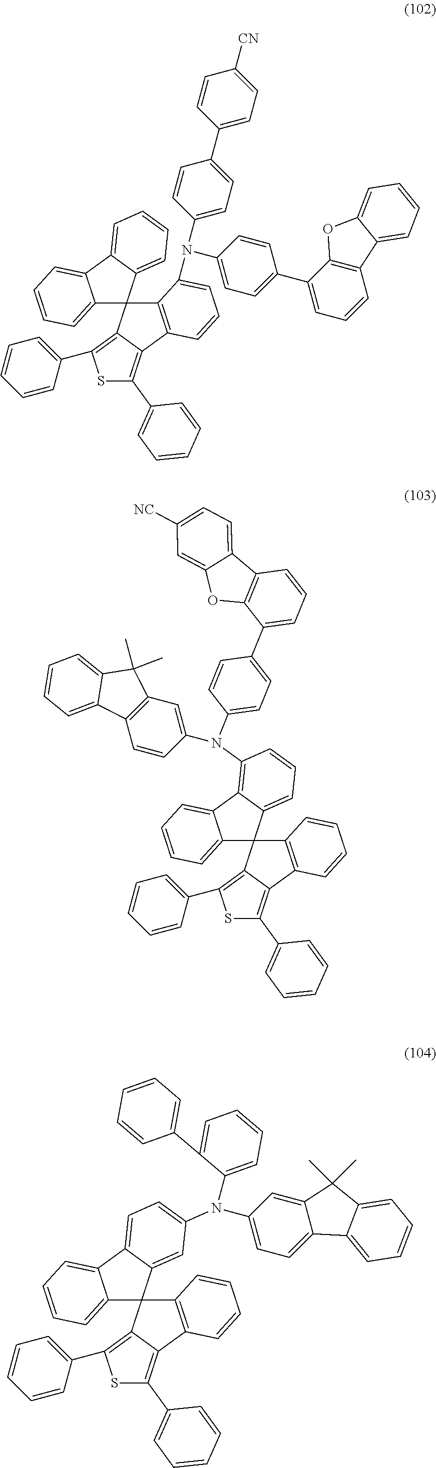 [SMILES: N#Cc1ccc(-c2ccc(N(c3ccc(-c4cccc5c4oc4ccccc45)cc3)c3cccc4c3C3(c5ccccc5-c5ccccc53)c3c(-c5ccccc5)sc(-c5ccccc5)c3-4)cc2)cc1 ; CC1(C)c2ccccc2-c2ccc(N(c3ccc(-c4cccc5c4oc4cc(C#N)ccc45)cc3)c3cccc4c3-c3ccccc3C43c4ccccc4-c4c(-c5ccccc5)sc(-c5ccccc5)c43)cc21 ; CC1(C)c2ccccc2-c2ccc(N(c3ccc4c(c3)C3(c5ccccc5-4)c4ccccc4-c4c(-c5ccccc5)sc(-c5ccccc5)c43)c3ccccc3-c3ccccc3)cc21]

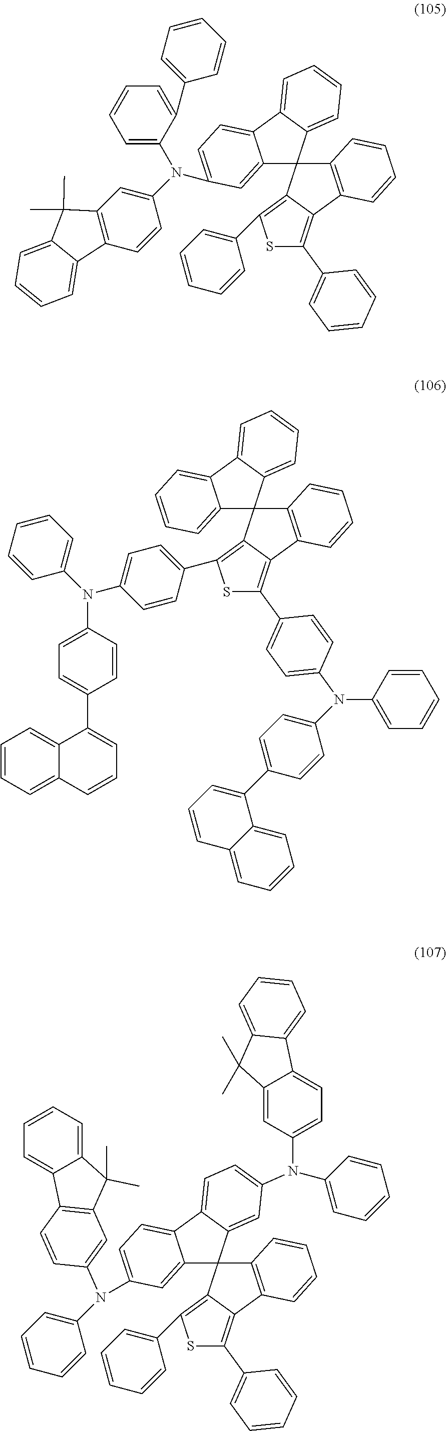 [SMILES: CC1(C)c2ccccc2-c2ccc(N(c3ccc4c(c3)C3(c5ccccc5-4)c4ccccc4-c4c(-c5ccccc5)sc(-c5ccccc5)c43)c3ccccc3-c3ccccc3)cc21 ; c1ccc(N(c2ccc(-c3sc(-c4ccc(N(c5ccccc5)c5ccc(-c6cccc7ccccc67)cc5)cc4)c4c3-c3ccccc3C43c4ccccc4-c4ccccc43)cc2)c2ccc(-c3cccc4ccccc34)cc2)cc1 ; CC1(C)c2ccccc2-c2ccc(N(c3ccccc3)c3ccc4c(c3)C3(c5cc(N(c6ccccc6)c6ccc7c(c6)C(C)(C)c6ccccc6-7)ccc5-4)c4ccccc4-c4c(-c5ccccc5)sc(-c5ccccc5)c43)cc21]

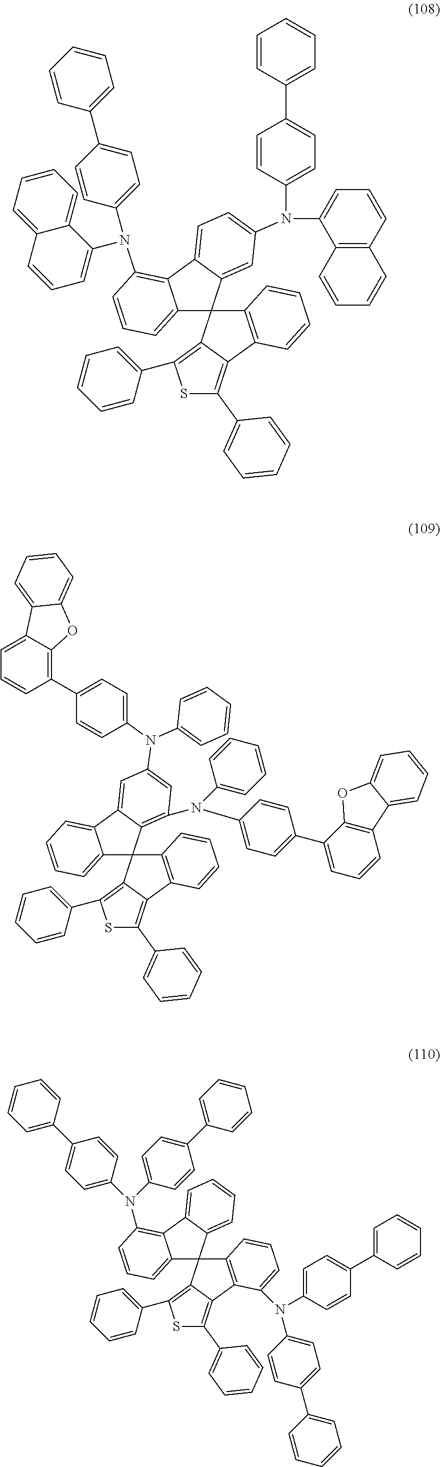 [SMILES: c1ccc(-c2ccc(N(c3ccc4c(c3)C3(c5ccccc5-c5c(-c6ccccc6)sc(-c6ccccc6)c53)c3cccc(N(c5ccc(-c6ccccc6)cc5)c5cccc6ccccc56)c3-4)c3cccc4ccccc34)cc2)cc1 ; c1ccc(-c2sc(-c3ccccc3)c3c2-c2ccccc2C32c3ccccc3-c3cc(N(c4ccccc4)c4ccc(-c5cccc6c5oc5ccccc56)cc4)cc(N(c4ccccc4)c4ccc(-c5cccc6c5oc5ccccc56)cc4)c32)cc1 ; c1ccc(-c2ccc(N(c3ccc(-c4ccccc4)cc3)c3cccc4c3-c3ccccc3C43c4cccc(N(c5ccc(-c6ccccc6)cc5)c5ccc(-c6ccccc6)cc5)c4-c4c(-c5ccccc5)sc(-c5ccccc5)c43)cc2)cc1]

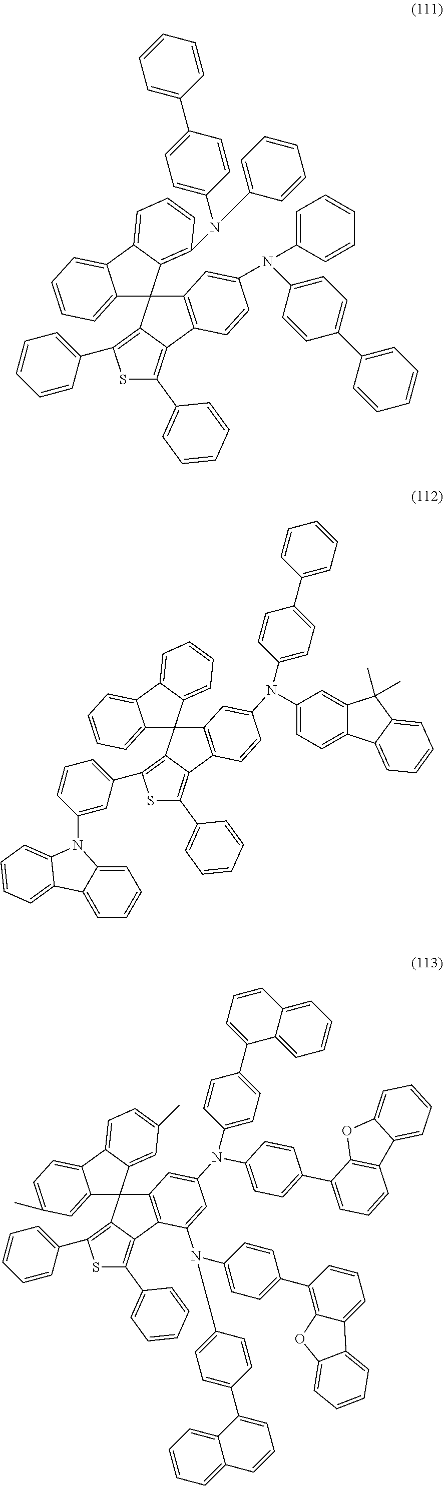 [SMILES: c1ccc(-c2ccc(N(c3ccccc3)c3ccc4c(c3)C3(c5ccccc5-c5cccc(N(c6ccccc6)c6ccc(-c7ccccc7)cc6)c53)c3c(-c5ccccc5)sc(-c5ccccc5)c3-4)cc2)cc1 ; CC1(C)c2ccccc2-c2ccc(N(c3ccc(-c4ccccc4)cc3)c3ccc4c(c3)C3(c5ccccc5-c5ccccc53)c3c(-c5cccc(-n6c7ccccc7c7ccccc76)c5)sc(-c5ccccc5)c3-4)cc21 ; Cc1ccc2c(c1)C1(c3cc(C)ccc3-2)c2cc(N(c3ccc(-c4cccc5ccccc45)cc3)c3ccc(-c4cccc5c4oc4ccccc45)cc3)cc(N(c3ccc(-c4cccc5ccccc45)cc3)c3ccc(-c4cccc5c4oc4ccccc45)cc3)c2-c2c(-c3ccccc3)sc(-c3ccccc3)c21]

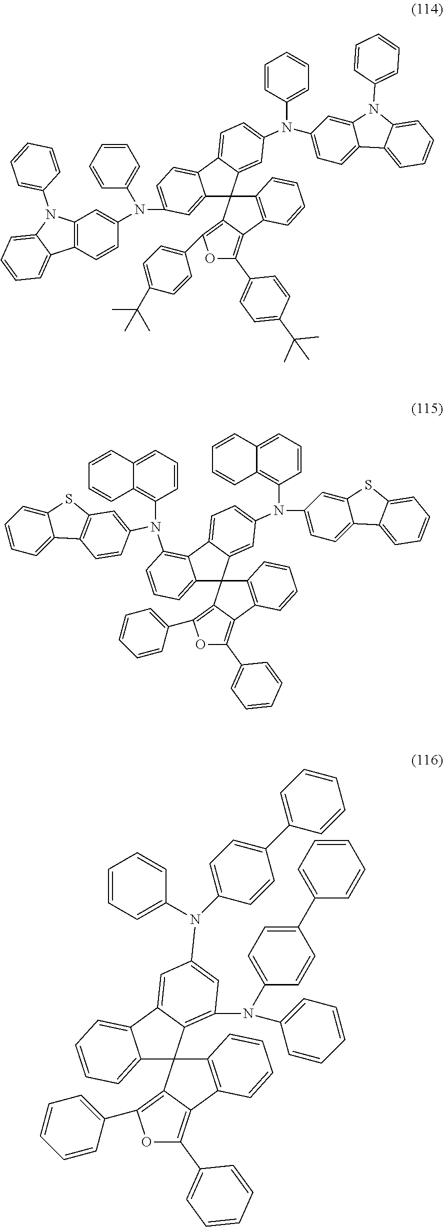 [SMILES: CC(C)(C)c1ccc(-c2oc(-c3ccc(C(C)(C)C)cc3)c3c2-c2ccccc2C32c3cc(N(c4ccccc4)c4ccc5c6ccccc6n(-c6ccccc6)c5c4)ccc3-c3ccc(N(c4ccccc4)c4ccc5c6ccccc6n(-c6ccccc6)c5c4)cc32)cc1 ; c1ccc(-c2oc(-c3ccccc3)c3c2-c2ccccc2C32c3cc(N(c4ccc5c(c4)sc4ccccc45)c4cccc5ccccc45)ccc3-c3c(N(c4ccc5c(c4)sc4ccccc45)c4cccc5ccccc45)cccc32)cc1 ; c1ccc(-c2ccc(N(c3ccccc3)c3cc4c(c(N(c5ccccc5)c5ccc(-c6ccccc6)cc5)c3)C3(c5ccccc5-4)c4ccccc4-c4c(-c5ccccc5)oc(-c5ccccc5)c43)cc2)cc1]

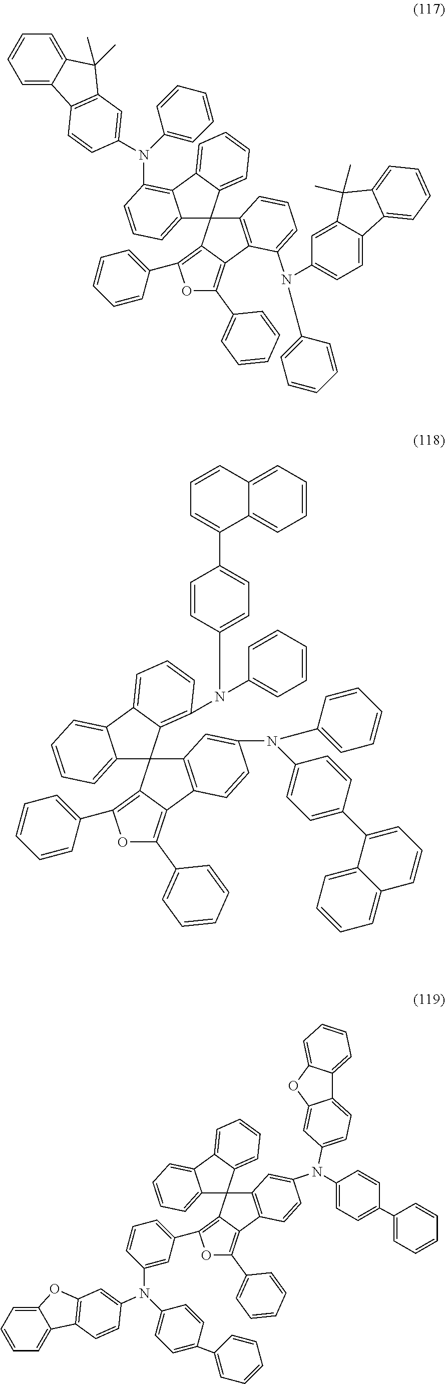 [SMILES: CC1(C)c2ccccc2-c2ccc(N(c3ccccc3)c3cccc4c3-c3ccccc3C43c4cccc(N(c5ccccc5)c5ccc6c(c5)C(C)(C)c5ccccc5-6)c4-c4c(-c5ccccc5)oc(-c5ccccc5)c43)cc21 ; c1ccc(-c2oc(-c3ccccc3)c3c2-c2ccc(N(c4ccccc4)c4ccc(-c5cccc6ccccc56)cc4)cc2C32c3ccccc3-c3cccc(N(c4ccccc4)c4ccc(-c5cccc6ccccc56)cc4)c32)cc1 ; c1ccc(-c2ccc(N(c3cccc(-c4oc(-c5ccccc5)c5c4C4(c6ccccc6-c6ccccc64)c4cc(N(c6ccc(-c7ccccc7)cc6)c6ccc7c(c6)oc6ccccc67)ccc4-5)c3)c3ccc4c(c3)oc3ccccc34)cc2)cc1]

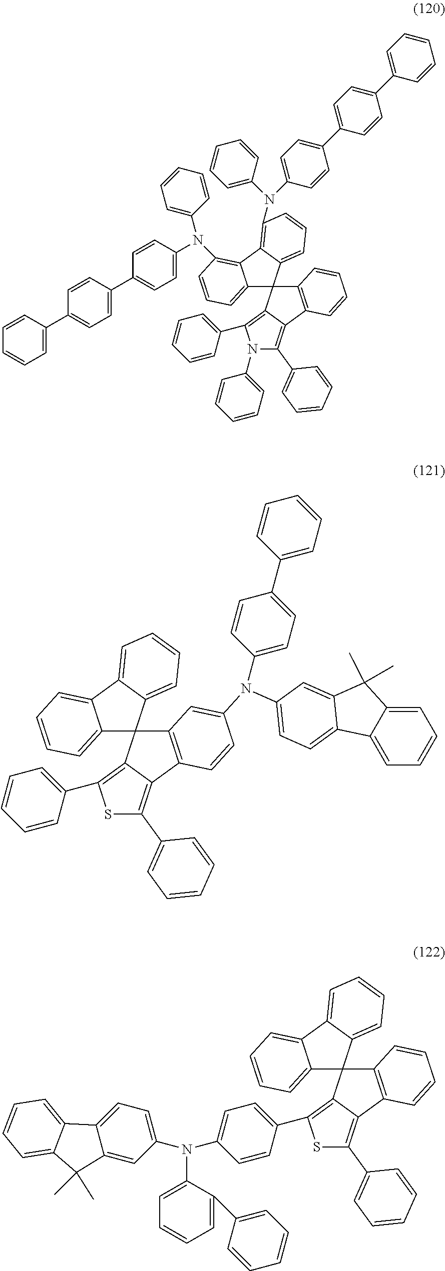 [SMILES: c1ccc(-c2ccc(-c3ccc(N(c4ccccc4)c4cccc5c4-c4c(N(c6ccccc6)c6ccc(-c7ccc(-c8ccccc8)cc7)cc6)cccc4C54c5ccccc5-c5c4c(-c4ccccc4)n(-c4ccccc4)c5-c4ccccc4)cc3)cc2)cc1 ; CC1(C)c2ccccc2-c2ccc(N(c3ccc(-c4ccccc4)cc3)c3ccc4c(c3)C3(c5ccccc5-c5ccccc53)c3c(-c5ccccc5)sc(-c5ccccc5)c3-4)cc21 ; CC1(C)c2ccccc2-c2ccc(N(c3ccc(-c4sc(-c5ccccc5)c5c4C4(c6ccccc6-c6ccccc64)c4ccccc4-5)cc3)c3ccccc3-c3ccccc3)cc21]

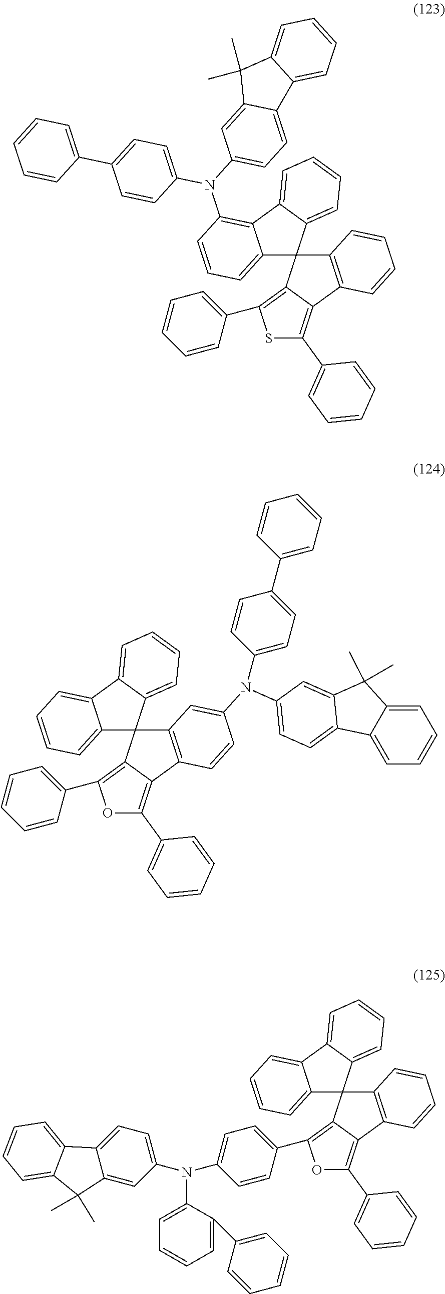 [SMILES: CC1(C)c2ccccc2-c2ccc(N(c3ccc(-c4ccccc4)cc3)c3cccc4c3-c3ccccc3C43c4ccccc4-c4c(-c5ccccc5)sc(-c5ccccc5)c43)cc21 ; CC1(C)c2ccccc2-c2ccc(N(c3ccc(-c4ccccc4)cc3)c3ccc4c(c3)C3(c5ccccc5-c5ccccc53)c3c(-c5ccccc5)oc(-c5ccccc5)c3-4)cc21 ; CC1(C)c2ccccc2-c2ccc(N(c3ccc(-c4oc(-c5ccccc5)c5c4C4(c6ccccc6-c6ccccc64)c4ccccc4-5)cc3)c3ccccc3-c3ccccc3)cc21]

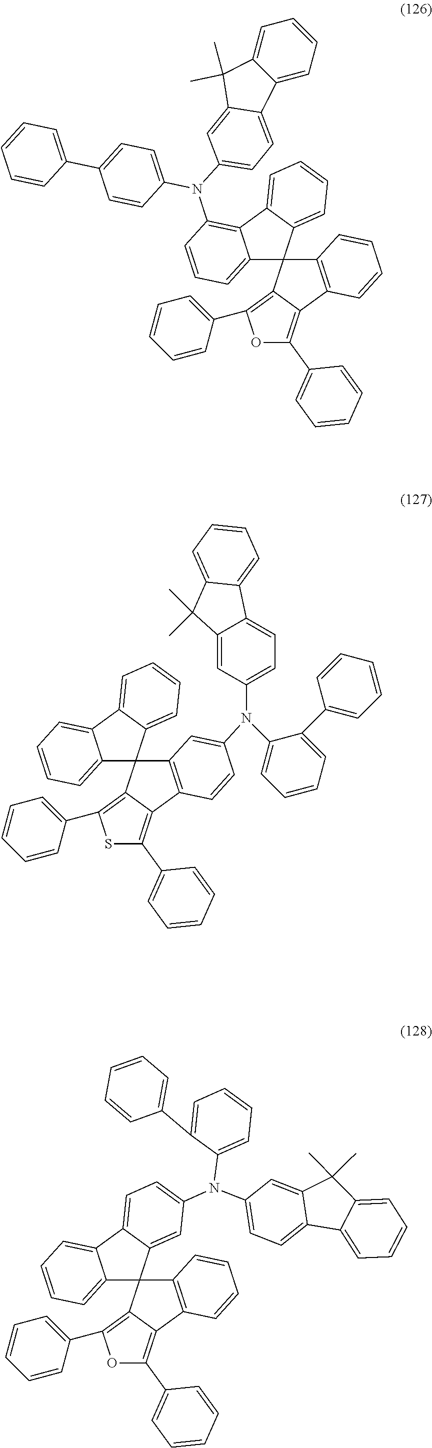 [SMILES: CC1(C)c2ccccc2-c2ccc(N(c3ccc(-c4ccccc4)cc3)c3cccc4c3-c3ccccc3C43c4ccccc4-c4c(-c5ccccc5)oc(-c5ccccc5)c43)cc21 ; CC1(C)c2ccccc2-c2ccc(N(c3ccc4c(c3)C3(c5ccccc5-c5ccccc53)c3c(-c5ccccc5)sc(-c5ccccc5)c3-4)c3ccccc3-c3ccccc3)cc21 ; CC1(C)c2ccccc2-c2ccc(N(c3ccc4c(c3)C3(c5ccccc5-4)c4ccccc4-c4c(-c5ccccc5)oc(-c5ccccc5)c43)c3ccccc3-c3ccccc3)cc21]

This includes disclosure of the corresponding configuration isomers, especially diastereomers or enantiomers, of the compounds shown above.

The compounds of formula (I) can be prepared by means of organic chemistry synthesis steps known to the person skilled in the art, for example by means of metallation, addition of nucleophiles onto carbonyl groups, Suzuki reaction and Hartwig-Buchwald reaction.

A preferred process for preparing compounds of the formula (I) is shown below. The process should be understood in an illustrative and non-limiting manner. The person skilled in the art will be able to depart from the illustrative process shown and make changes within the scope of his common art knowledge, if this is technically advantageous, in order to arrive at compounds of the formula (I).

In a first step, proceeding from a carboxylic ester-substituted thiophene, furan or pyrrole compound of the formula (Int-1), an intermediate of a formula (Int-2) is prepared by ring closure. This can be converted further in an arylation reaction to an intermediate of a formula (Int-3) (scheme 1).

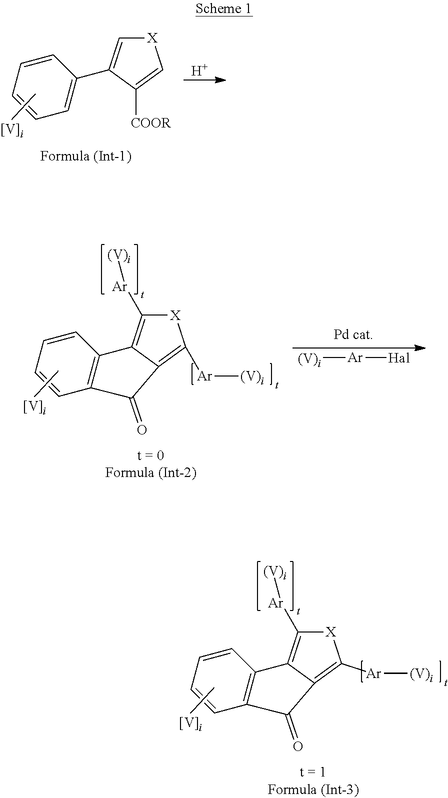

The variables here are defined as follows:
V is the same or different at each instance and is selected from reactive groups, preferably Cl, Br or I;
X is as defined above for formula (I);
Ar is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Hal is Cl, Br or I;
R is an alkyl group having 1 to 20 carbon atoms, or a substituted or unsubstituted aromatic ring system having 6 to 40 aromatic ring atoms, or a substituted or unsubstituted heteroaromatic ring system having 5 to 40 aromatic ring atoms;
i is 0, 1 or 2;
where the compounds are each substituted at the unoccupied positions on the benzene ring by an R¹ radical, as defined above for formula (I).

As shown in scheme 1b below, it is possible by an analogous route to that shown in scheme 1 also to prepare regioisomers of the formula (Int-3) in which the X group of the five-membered heteroaromatic ring is in an adjacent position (formula (Int-3b)).

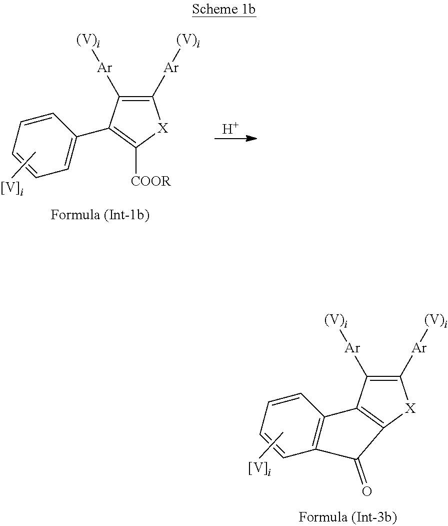

The variables here are as defined for scheme 1.

As shown in scheme 1c below, it is possible by an analogous route to that shown in scheme 1 also to prepare regioisomers of the formula (Int-3) in which the X group of the five-membered heteroaromatic ring is in an adjacent position (formula (Int-3c)).

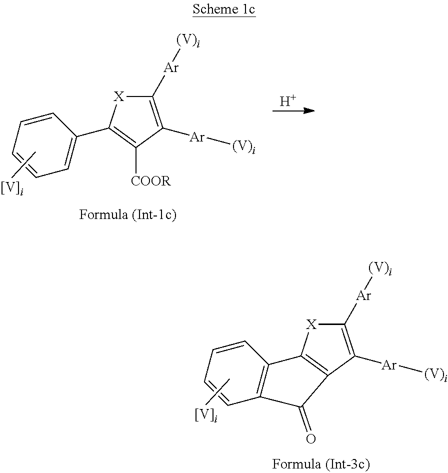

The variables here are as defined for scheme 1.

The compounds of the formula (Int-2) or (Int-3) are converted in a subsequent step to spirobifluorene derivatives (scheme 2). This is accomplished by addition of ortho-metallated bisaryl onto the carbonyl function of the compounds of the formula (Int-2) or (Int-3) and subsequent acid-catalysed ring closure, giving compounds of the formulae (Int-4) and (Int-5).

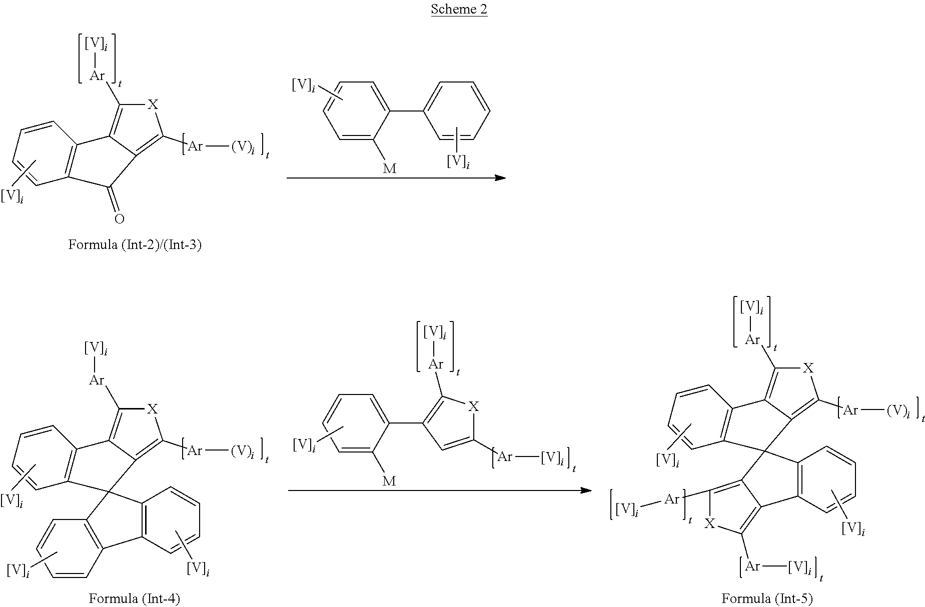

Alternatively, it is also possible to prepare compounds of the formula (Int-4) by reacting a fluorenone derivative with an ortho-metallated heteroaryl-aryl derivative (Int-A1) (scheme 2-1):

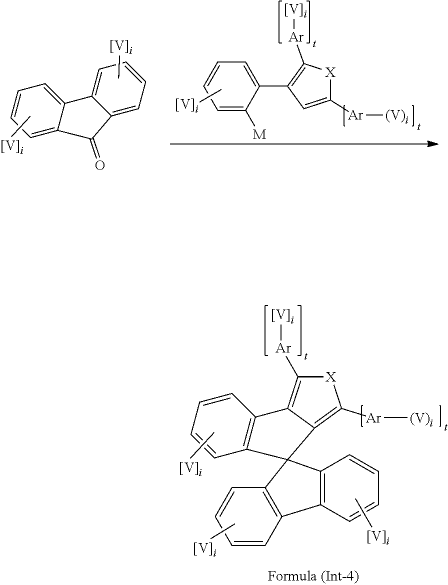

The variable groups in the compounds of the formulae (Int-4), (Int-A1) and (Int-5) are as defined above, where index t is 0 or 1 and is preferably 1, and where at least one index i equal to 1 is present, and where the formulae are each substituted at the unoccupied positions on the benzene ring by an R¹ radical.

This can be effected analogously for compounds of the formula (Int-3b), resulting in compounds of the formula (Int-4b); see scheme 2b

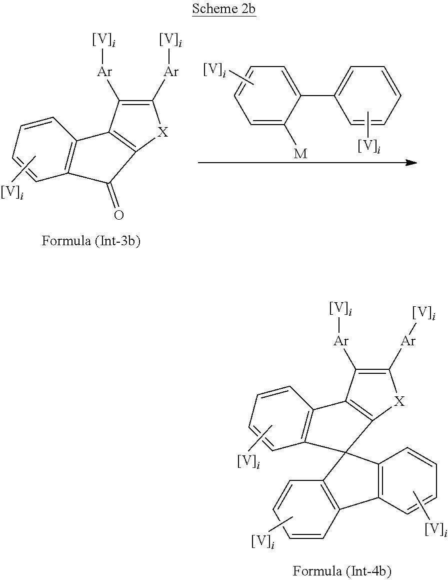

where the variables that occur are as defined for scheme 2.

Alternatively, it is also possible to prepare compounds of the formula (Int-4b) by reacting a fluorenone derivative with an ortho-metallated heteroaryl-aryl derivative (Int-A2) (scheme 2b-1):

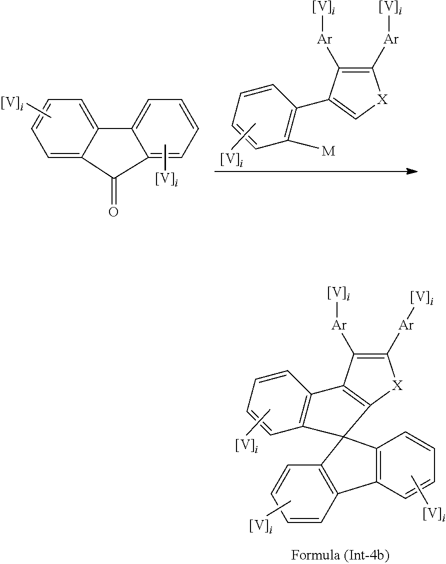

This can be effected analogously for compounds of the formula (Int-3c), resulting in compounds of the formula (Int-4c); see scheme 2c

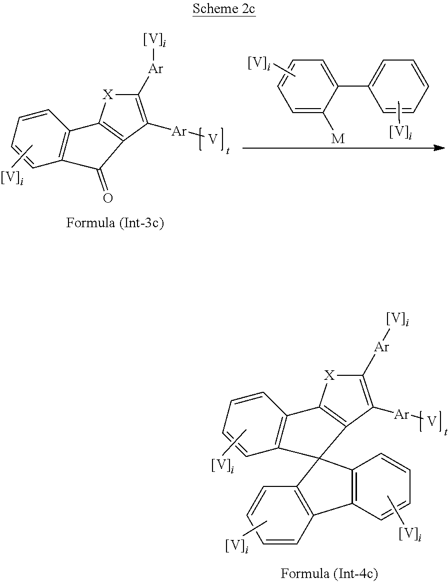

where the variables that occur are as defined for scheme 2.

Alternatively, it is also possible to prepare compounds of the formula (Int-4c) by reacting a fluorenone derivative with an ortho-metallated heteroaryl-aryl derivative (Int-A3) (scheme 2c-1):

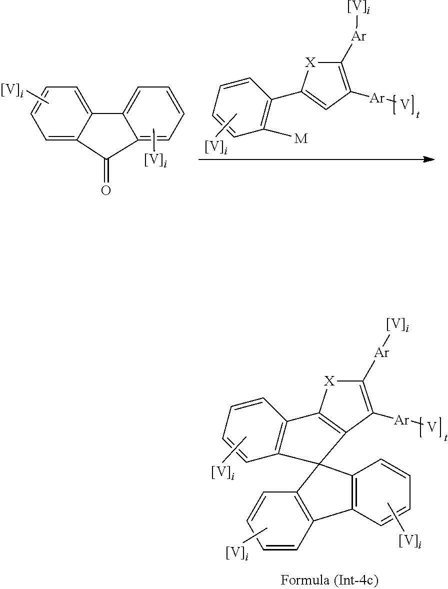

Compounds of the formulae (Int-4), (Int-4b), (Int-4c) and (Int-5) are important intermediates for the preparation of compounds of the formula (I) and hence likewise form part of the subject-matter of the present application.

The ortho-metallated bisaryl compounds that are used in the reactions may be prepared, for example, by lithiation or Grignard reaction from the corresponding ortho-halogenated bisaryls, as shown in the synthesis examples.

As shown in scheme 3a, 3b, 3c or 3d, the intermediates of the formulae (Int-4) or (Int-4b) or (Int-4c) or (Int-5) can be converted either via a Buchwald coupling with an amine, or via a Suzuki coupling with an amino-substituted aryl or heteroaryl compound. This affords compounds of formula (I).

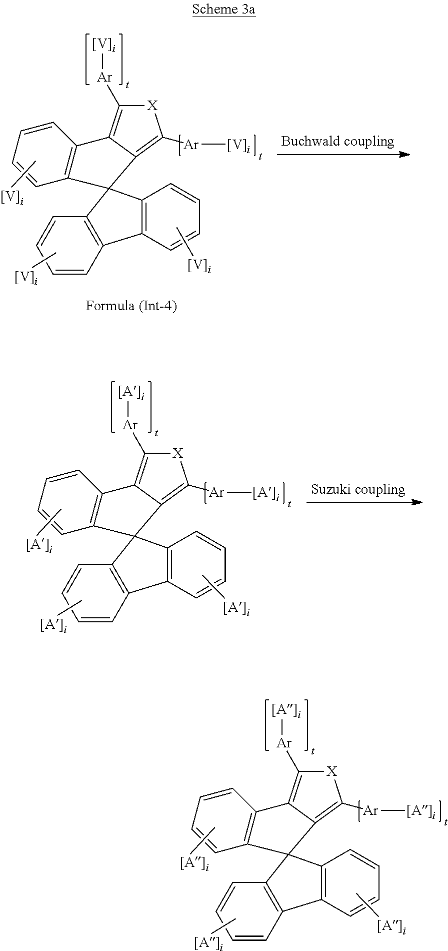

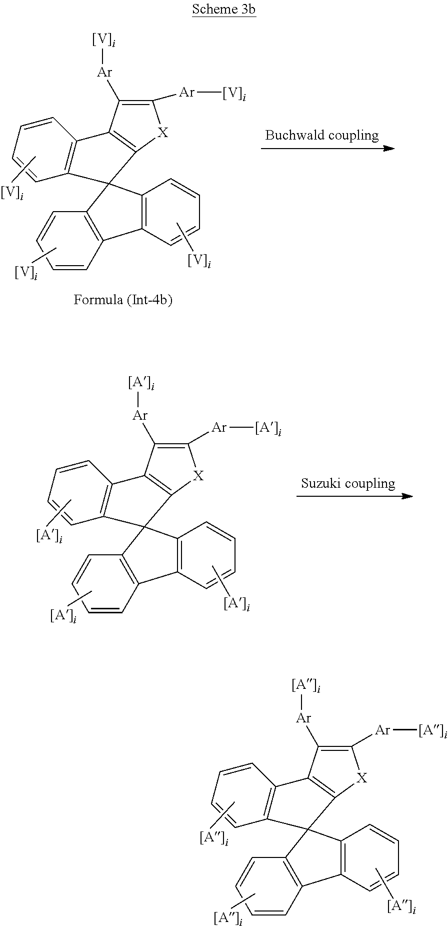

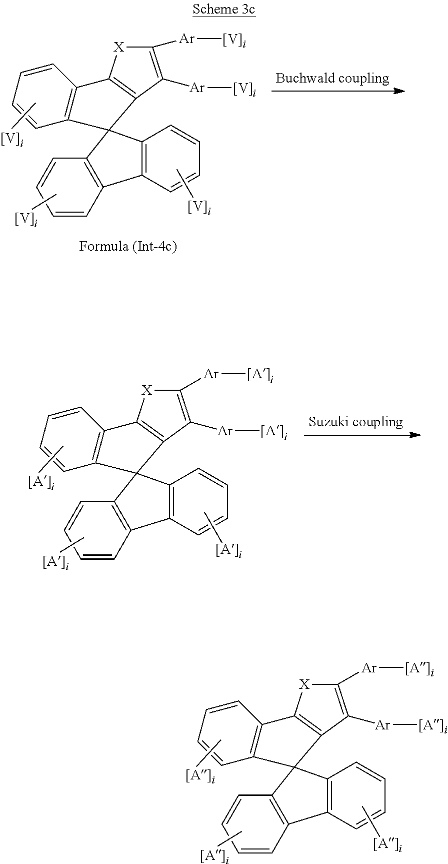

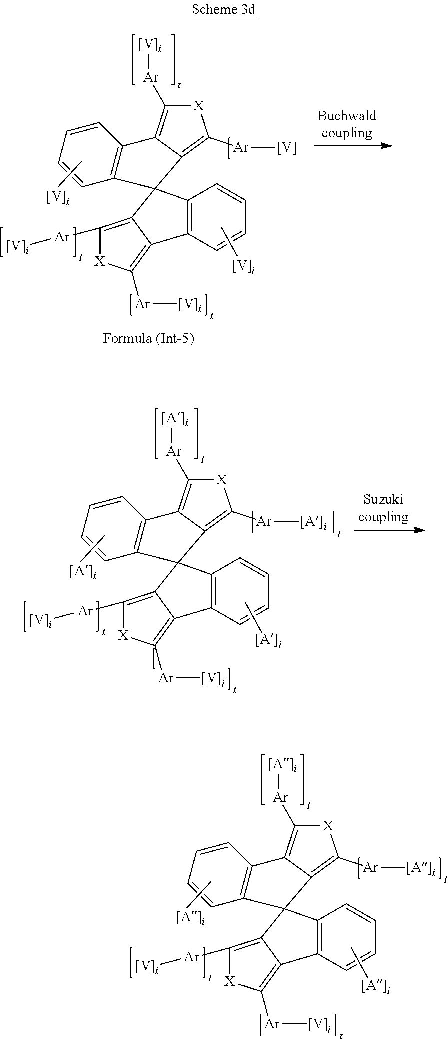

The variables here are as defined above, at least one index i equal to 1 is present, and A′ is a unit of formula (A) where k is 0, and A″ is a unit of formula (A) where k is 1.

The present application thus provides a process for preparing a compound of a formula (I), characterized in that, in a first step via a ring closure reaction, by action of acid, a compound (Int-1) is converted to a compound (Int-2), and further characterized in that, in a further step, an ortho-metallated bisaryl is added on and a further ring closure reaction is conducted, forming a compound (Int-4) or (Int-5), and further characterized in that, in a further step, a Suzuki coupling or a Hartwig-Buchwald coupling is conducted, giving a compound of a formula (I).

In a preferred embodiment, after the first step, a transition metal-catalysed arylation is performed, converting the compound (Int-2) to a compound (Int-3).

The present application likewise provides a process for preparing a compound of a formula (I), characterized in that, in a first step via a ring closure reaction, by action of acid, a compound (Int-1b) is converted to a compound (Int-3b) or a compound (Int-1c) to a compound (Int-3c), and further characterized in that, in a further step, an ortho-metallated bisaryl is added on and a further ring closure reaction is conducted, forming a compound (Int-4b) from the compound (Int-3b) and a compound (Int-4c) from the compound (Int-3c), and further characterized in that, in a further step, a Suzuki coupling or a Hartwig-Buchwald coupling is conducted, giving a compound of a formula (I).

The reaction steps take place here in the sequence specified.

The present application likewise provides a process for preparing a compound of a formula (I), characterized in that, in a first step, an ortho-metallated heteroaryl-aryl derivative (Int-A1), (Int-A2) or (Int-A3) is reacted with a fluorenone derivative and a ring closure reaction is conducted, forming a compound selected from compounds of the formulae (Int-4), (Int-4b) and (Int-4c), and further characterized in that, in a further step, a Suzuki coupling or a Hartwig-Buchwald coupling is conducted, giving a compound of a formula (I).

The above-described compounds of the invention, especially compounds substituted by reactive leaving groups, such as bromine, iodine, chlorine, boronic acid or boronic ester, may find use as monomers for production of corresponding oligomers, dendrimers or polymers. Suitable reactive leaving groups are, for example, bromine, iodine, chlorine, boronic acids, boronic esters, amines, alkenyl or alkynyl groups having a terminal C—C double bond or C—C triple bond, oxiranes, oxetanes, groups which enter into a cycloaddition, for example a 1,3-dipolar cycloaddition, for example dienes or azides, carboxylic acid derivatives, alcohols and silanes.

The invention therefore further provides oligomers, polymers or dendrimers containing one or more compounds of formula (I), wherein the bond(s) to the polymer, oligomer or dendrimer may be localized at any desired positions substituted by R¹, R², R³ or R⁴ in formula (I). According to the linkage of the compound of formula (I), the compound is part of a side chain of the oligomer or polymer or part of the main chain. An oligomer in the context of this invention is understood to mean a compound formed from at least three monomer units. A polymer in the context of the invention is understood to mean a compound formed from at least ten monomer units. The polymers, oligomers or dendrimers of the invention may be conjugated, partly conjugated or nonconjugated. The oligomers or polymers of the invention may be linear, branched or dendritic. In the structures having linear linkage, the units of formula (I) may be joined directly to one another, or they may be joined to one another via a bivalent group, for example via a substituted or unsubstituted alkylene group, via a heteroatom or via a bivalent aromatic or heteroaromatic group. In branched and dendritic structures, it is possible, for example, for three or more units of formula (I) to be joined via a trivalent or higher-valency group, for example via a trivalent or higher-valency aromatic or heteroaromatic group, to give a branched or dendritic oligomer or polymer.

For the repeat units of formula (I) in oligomers, dendrimers and polymers, the same preferences apply as described above for compounds of formula (I).

For preparation of the oligomers or polymers, the monomers of the invention are homopolymerized or copolymerized with further monomers. Suitable and preferred comonomers are selected from fluorenes, spirobifluorenes, paraphenylenes, carbazoles, thiophenes, dihydrophenanthrenes, cis- and trans-indenofluorenes, ketones, phenanthrenes or else two or more of these units. The polymers, oligomers and dendrimers typically contain still further units, for example emitting (fluorescent or phosphorescent) units, for example vinyltriarylamines or phosphorescent metal complexes, and/or charge transport units, especially those based on triarylamines.

The polymers, oligomers and dendrimers of the invention have advantageous properties, especially high lifetimes, high efficiencies and good colour coordinates.

The polymers and oligomers of the invention are generally prepared by polymerization of one or more monomer types, of which at least one monomer leads to repeat units of the formula (I) in the polymer. Suitable polymerization reactions are known to those skilled in the art and are described in the literature. Particularly suitable and preferred polymerization reactions which lead to C—C and C—N couplings are as follows:
(A) SUZUKI polymerization;
(B) YAMAMOTO polymerization;
(C) STILLE polymerization; and
(D) HARTWIG-BUCHWALD polymerization.

How the polymerization can be conducted by these methods and how the polymers can then be separated from the reaction medium and purified is known to those skilled in the art and is described in detail in the literature.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, alpha-terpineol, benzothiazole, butyl benzoate, cumene, cyclohexanol, cyclohexanone, cyclohexylbenzene, decalin, dodecylbenzene, ethyl benzoate, indane, methyl benzoate, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, or mixtures of these solvents.

The invention therefore further provides a formulation, especially a solution, dispersion or emulsion, comprising at least one compound of formula (I) or at least one polymer, oligomer or dendrimer containing at least one unit of formula (I) and at least one solvent, preferably an organic solvent. The way in which such solutions can be prepared is known to those skilled in the art.

The compound of formula (I) is suitable for use in an electronic device, especially an organic electroluminescent device (OLED). Depending on the substitution, the compound of the formula (I) can be used in different functions and layers. Preference is given to use as a hole-transporting material in a hole-transporting layer and/or as matrix material in an emitting layer, more preferably in combination with a phosphorescent emitter.

The invention therefore further provides for the use of a compound of formula (I) in an electronic device. This electronic device is preferably selected from the group consisting of organic integrated circuits (OICs), organic field-effect transistors (OFETs), organic thin-film transistors (OTFTs), organic light-emitting transistors (OLETs), organic solar cells (OSCs), organic optical detectors, organic photoreceptors, organic field-quench devices (OFQDs), organic light-emitting electrochemical cells (OLECs), organic laser diodes (O-lasers) and more preferably organic electroluminescent devices (OLEDs).

The invention further provides an electronic device comprising at least one compound of formula (I). This electronic device is preferably selected from the abovementioned devices.

Particular preference is given to an organic electroluminescent device comprising anode, cathode and at least one emitting layer, characterized in that at least one organic layer comprising at least one compound of formula (I) is present in the device. Preference is given to an organic electroluminescent device comprising anode, cathode and at least one emitting layer, characterized in that at least one organic layer in the device, selected from hole-transporting and emitting layers, comprises at least one compound of formula (I).

A hole-transporting layer is understood here to mean all layers disposed between anode and emitting layer, preferably hole injection layer, hole transport layer and electron blocker layer. A hole injection layer is understood here to mean a layer that directly adjoins the anode. A hole transport layer is understood here to mean a layer which is between the anode and emitting layer but does not directly adjoin the anode, and preferably does not directly adjoin the emitting layer either. An electron blocker layer is understood here to mean a layer which is between the anode and emitting layer and directly adjoins the emitting layer. An electron blocker layer preferably has a high-energy LUMO and hence prevents electrons from exiting from the emitting layer.

Apart from the cathode, anode and emitting layer, the electronic device may comprise further layers. These are selected, for example, from in each case one or more hole injection layers, hole transport layers, hole blocker layers, electron transport layers, electron injection layers, electron blocker layers, exciton blocker layers, interlayers, charge generation layers and/or organic or inorganic p/n junctions. However, it should be pointed out that not every one of these layers need necessarily be present and the choice of layers always depends on the compounds used and especially also on whether the device is a fluorescent or phosphorescent electroluminescent device.

The sequence of layers in the electronic device is preferably as follows:
-anode-
-hole injection layer-
-hole transport layer-
-optionally further hole transport layers-
-emitting layer-
-optionally hole blocker layer-
-electron transport layer-
-electron injection layer-
-cathode-.

At the same time, it should be pointed out again that not all the layers mentioned need be present and/or that further layers may additionally be present.

The organic electroluminescent device of the invention may contain two or more emitting layers. More preferably, these emission layers have several emission maxima between 380 nm and 750 nm overall, such that the overall result is white emission; in other words, various emitting compounds which may fluoresce or phosphoresce and which emit blue, green yellow, orange or red light are used in the emitting layers. Especially preferred are three-layer systems, i.e. systems having three emitting layers, wherein one of the three layers in each case shows blue emission, one of the three layers in each case shows green emission, and one of the three layers in each case shows orange or red emission. The compounds of the invention here are preferably present in a hole-transporting layer or in the emitting layer. It should be noted that, for the production of white light, rather than a plurality of colour-emitting emitter compounds, an emitter compound used individually which emits over a broad wavelength range may also be suitable.

It is preferable that the compound of the formula (I) is used as hole transport material. The emitting layer here may be a fluorescent emitting layer, or it may be a phosphorescent emitting layer. The emitting layer is preferably a blue-fluorescing layer or a green-phosphorescing layer.

When the device containing the compound of the formula (I) contains a phosphorescent emitting layer, it is preferable that this layer contains two or more, preferably exactly two, different matrix materials (mixed matrix system). Preferred embodiments of mixed matrix systems are described in detail further down.

If the compound of formula (I) is used as hole transport material in a hole transport layer, a hole injection layer or an electron blocker layer, the compound can be used as pure material, i.e. in a proportion of 100%, in the hole transport layer, or it can be used in combination with one or more further compounds.

In a preferred embodiment, a hole-transporting layer comprising the compound of the formula (I) additionally comprises one or more further hole-transporting compounds. These further hole-transporting compounds are preferably selected from triarylamine compounds, more preferably from monotriarylamine compounds. With very particular preference they are selected from the preferred embodiments of hole transport materials that are indicated later on below. In the preferred embodiment described, the compound of the formula (I) and the one or more further hole-transporting compounds are preferably each present in a proportion of at least 10%, more preferably each in a proportion of at least 20%.

In a preferred embodiment, a hole-transporting layer comprising the compound of the formula (I) additionally contains one or more p-dopants. p-Dopants used according to the present invention are preferably those organic electron acceptor compounds capable of oxidizing one or more of the other compounds in the mixture.

Particularly preferred as p-dopants are quinodimethane compounds, azaindenofluorenediones, azaphenalenes, azatriphenylenes, 12, metal halides, preferably transition metal halides, metal oxides, preferably metal oxides comprising at least one transition metal or a metal from main group 3, and transition metal complexes, preferably complexes of Cu, Co, Ni, Pd and Pt with ligands containing at least one oxygen atom as binding site.

Preference is further given to transition metal oxides as dopants, preferably oxides of rhenium, molybdenum and tungsten, more preferably Re₂O₇, MoO₃, WO₃ and ReO₃. Still further preference is given to complexes of bismuth in the (Ill) oxidation state, more particularly bismuth(III) complexes with electron-deficient ligands, more particularly carboxylate ligands.

The p-dopants are preferably in substantially homogeneous distribution in the p-doped layers. This can be achieved, for example, by co-evaporation of the p-dopant and the hole transport material matrix. The p-dopant is preferably present in a proportion of 1% to 10% in the p-doped layer.

Preferred p-dopants are especially the following compounds:

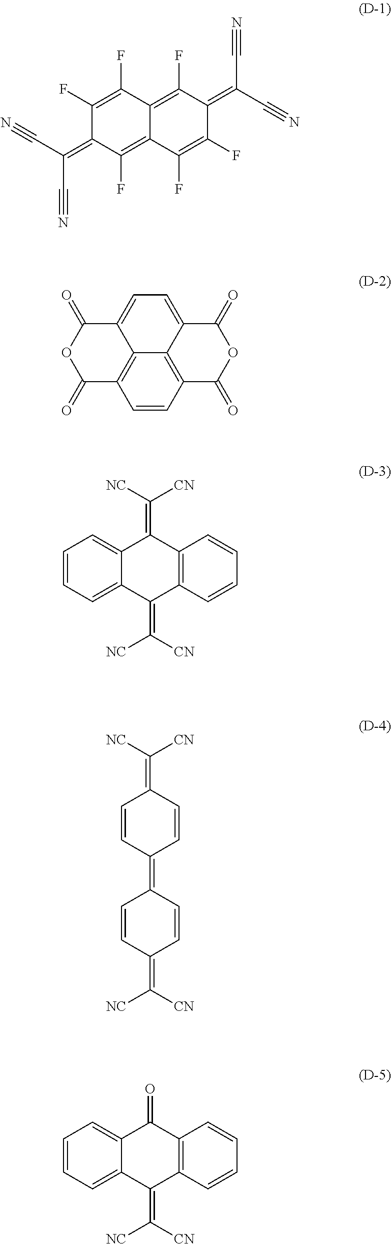 [SMILES: N#CC(C#N)=c1c(F)c(F)c2c(F)c(=C(C#N)C#N)c(F)c(F)c2c1F ; O=C1OC(=O)c2ccc3c4c(ccc1c24)C(=O)OC3=O ; N#CC(C#N)=c1c2ccccc2c(=C(C#N)C#N)c2ccccc12 ; N#CC(C#N)=c1ccc(=c2ccc(=C(C#N)C#N)cc2)cc1 ; N#CC(C#N)=C1c2ccccc2C(=O)c2ccccc21]

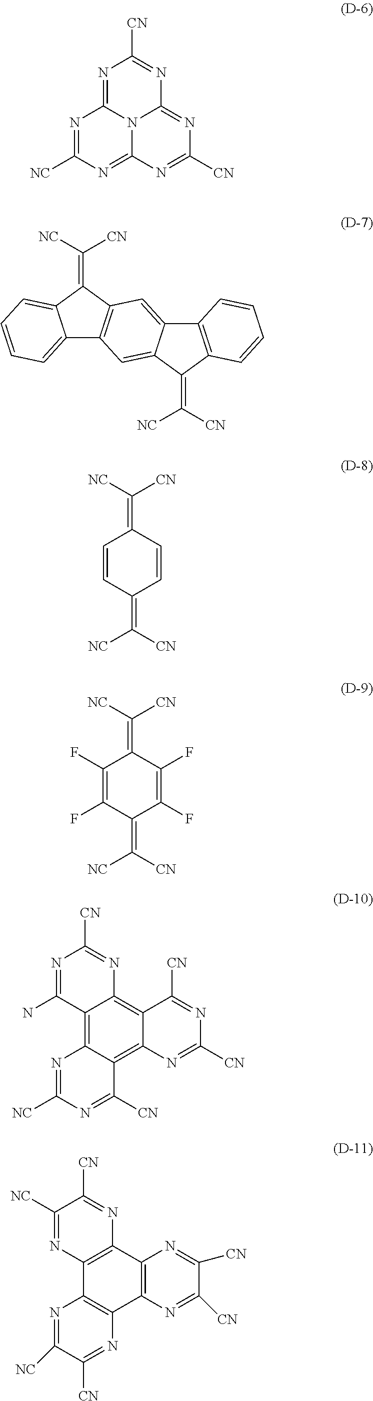 [SMILES: N#CC1=NC2=NC(C#N)=NC3=NC(C#N)=NC(=N1)N23 ; N#CC(C#N)=C1c2ccccc2-c2cc3c(cc21)-c1ccccc1C3=C(C#N)C#N ; N#CC(C#N)=c1ccc(=C(C#N)C#N)cc1 ; N#CC(C#N)=c1c(F)c(F)c(=C(C#N)C#N)c(F)c1F ; N#Cc1nc(N)c2c(n1)c1c(C#N)nc(C#N)nc1c1c(C#N)nc(C#N)nc21 ; N#Cc1nc2c3nc(C#N)c(C#N)nc3c3nc(C#N)c(C#N)nc3c2nc1C#N]

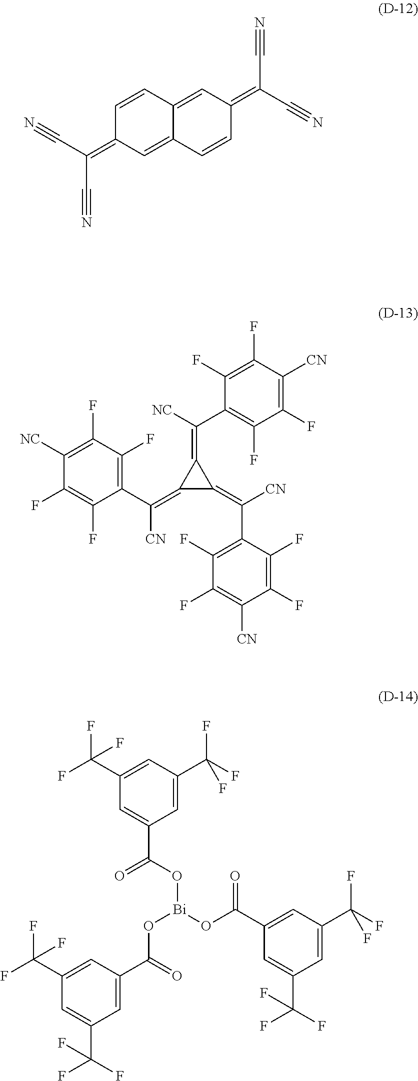

In a preferred embodiment, a hole injection layer that conforms to one of the following embodiments is present in the device: a) it contains a triarylamine and a p-dopant; or b) it contains a single electron-deficient material (electron acceptor). In a preferred embodiment of embodiment a), the triarylamine is a monotriarylamine, especially one of the preferred triarylamine derivatives mentioned further down. In a preferred embodiment of embodiment b), the electron-deficient material is a hexaazatriphenylene derivative as described in US 2007/0092755.

The compound of the formula (I) may be present in a hole injection layer, in a hole transport layer and/or in an electron blocker layer of the device. When the compound is present in a hole injection layer or in a hole transport layer, it has preferably been p-doped, meaning that it is in mixed form with a p-dopant, as described above, in the layer.

The compound of the formula (I) is preferably present in an electron blocker layer. In this case, it is preferably not p-doped. Further preferably, in this case, it is preferably in the form of a single compound in the layer without addition of a further compound.

In an alternative preferred embodiment, the compound of the formula (I) is used in an emitting layer as matrix material in combination with one or more emitting compounds, preferably phosphorescent emitting compounds. The phosphorescent emitting compounds here are preferably selected from red-phosphorescing and green-phosphorescing compounds.

The proportion of the matrix material in the emitting layer in this case is between 50.0% and 99.9% by volume, preferably between 80.0% and 99.5% by volume, and more preferably between 85.0% and 97.0% by volume.

Correspondingly, the proportion of the emitting compound is between 0.1% and 50.0% by volume, preferably between 0.5% and 20.0% by volume, and more preferably between 3.0% and 15.0% by volume.

An emitting layer of an organic electroluminescent device may also contain systems comprising a plurality of matrix materials (mixed matrix systems) and/or a plurality of emitting compounds. In this case too, the emitting compounds are generally those compounds having the smaller proportion in the system and the matrix materials are those compounds having the greater proportion in the system. In individual cases, however, the proportion of a single matrix material in the system may be less than the proportion of a single emitting compound.

It is preferable that the compounds of formula (I) are used as a component of mixed matrix systems, preferably for phosphorescent emitters. The mixed matrix systems preferably comprise two or three different matrix materials, more preferably two different matrix materials. Preferably, in this case, one of the two materials is a material having hole-transporting properties and the other material is a material having electron-transporting properties. It is further preferable when one of the materials is selected from compounds having a large energy differential between HOMO and LUMO (wide-bandgap materials). The compound of the formula (I) in a mixed matrix system is preferably the matrix material having hole-transporting properties. Correspondingly, when the compound of the formula (I) is used as matrix material for a phosphorescent emitter in the emitting layer of an OLED, a second matrix compound having electron-transporting properties is present in the emitting layer. The two different matrix materials may be present here in a ratio of 1:50 to 1:1, preferably 1:20 to 1:1, more preferably 1:10 to 1:1 and most preferably 1:4 to 1:1.

The desired electron-transporting and hole-transporting properties of the mixed matrix components may, however, also be combined mainly or entirely in a single mixed matrix component, in which case the further mixed matrix component(s) fulfil(s) other functions.

Preference is given to using the following material classes in the abovementioned layers of the device:

Phosphorescent emitters:

The term “phosphorescent emitters” typically encompasses compounds where the emission of light is effected through a spin-forbidden transition, for example a transition from an excited triplet state or a state having a higher spin quantum number, for example a quintet state.

Suitable phosphorescent emitters are especially compounds which, when suitably excited, emit light, preferably in the visible region, and also contain at least one atom of atomic number greater than 20, preferably greater than 38, and less than 84, more preferably greater than 56 and less than 80. Preference is given to using, as phosphorescent emitters, compounds containing copper, molybdenum, tungsten, rhenium, ruthenium, osmium, rhodium, iridium, palladium, platinum, silver, gold or europium, especially compounds containing iridium, platinum or copper.

In the context of the present invention, all luminescent iridium, platinum or copper complexes are considered to be phosphorescent compounds.

In general, all phosphorescent complexes as used for phosphorescent OLEDs according to the prior art and as known to those skilled in the art in the field of organic electroluminescent devices are suitable for use in the devices of the invention. Further examples of suitable phosphorescent emitters are shown in the following table:

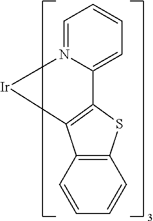

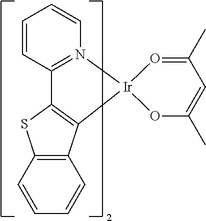

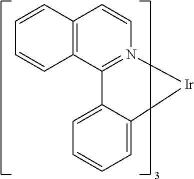

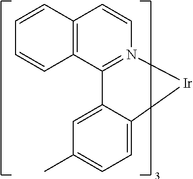

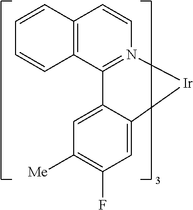

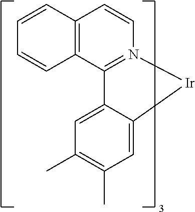

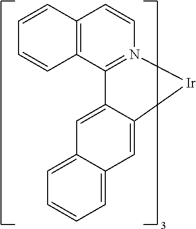

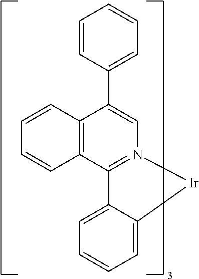

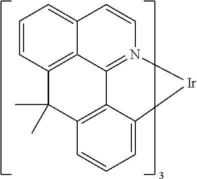

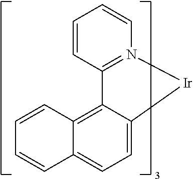

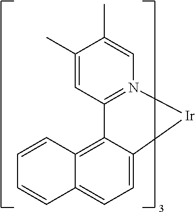

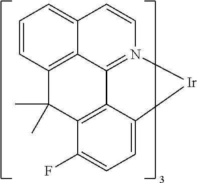

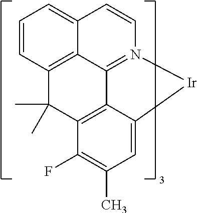

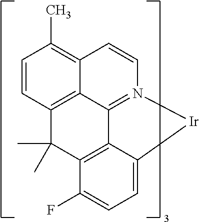

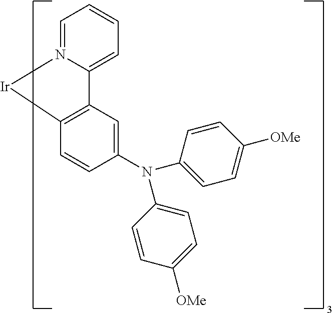

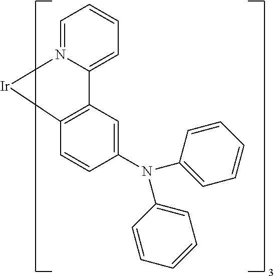

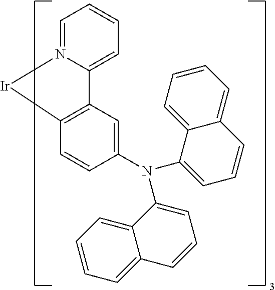

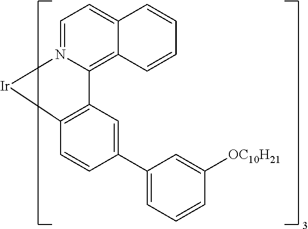

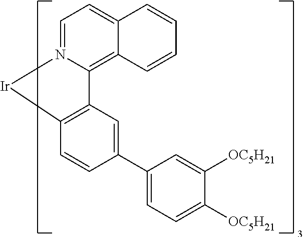

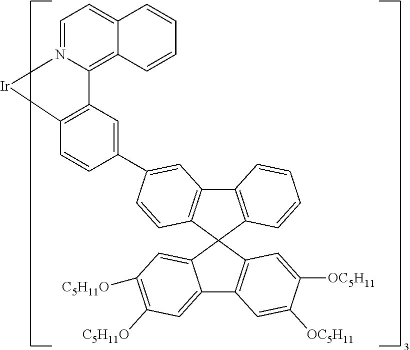

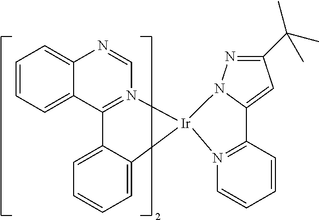

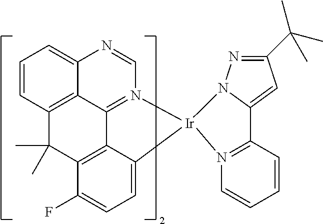

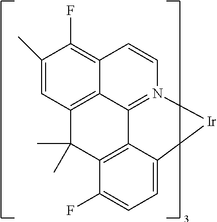

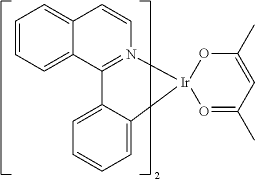

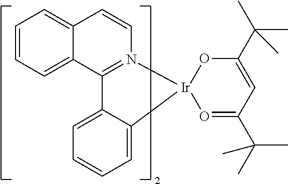

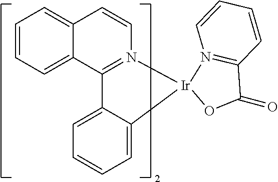

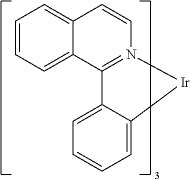

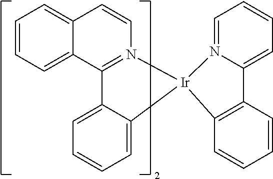

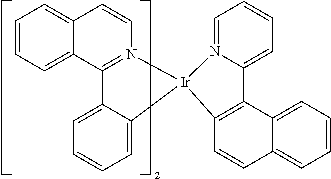

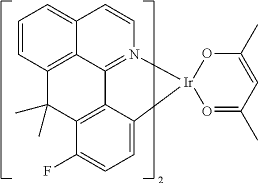

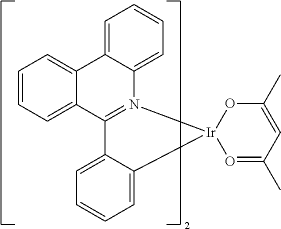

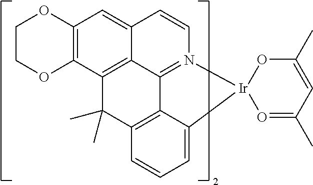

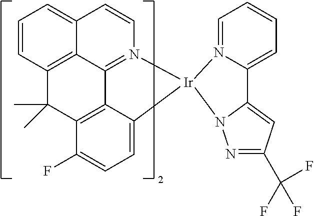

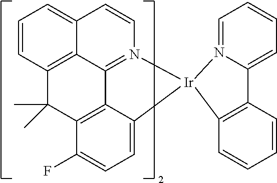

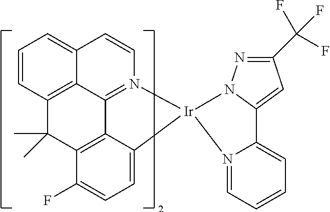

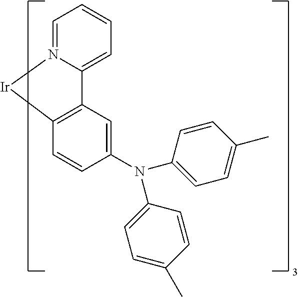

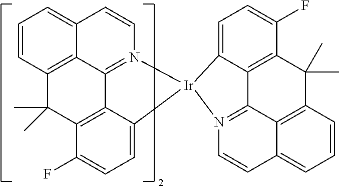

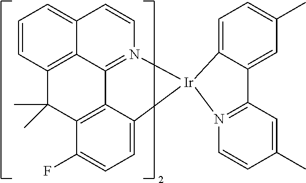

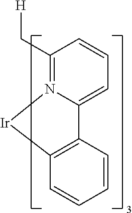

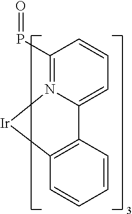

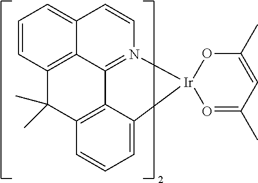

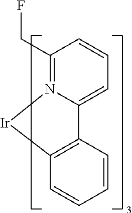

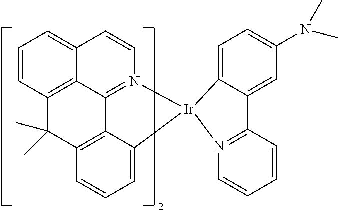

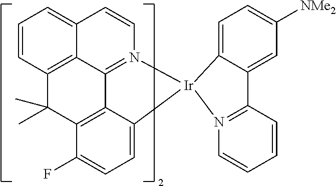

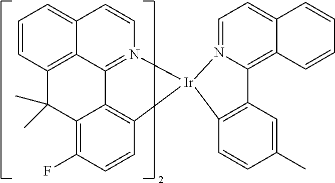

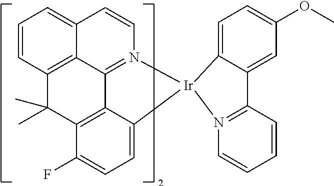

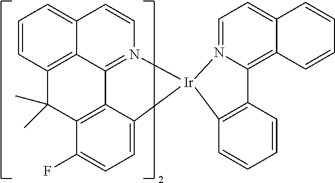

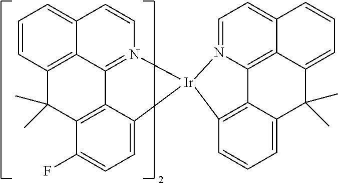

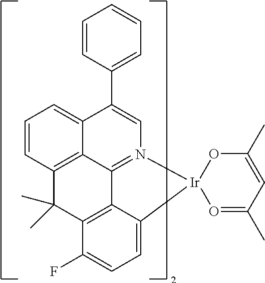

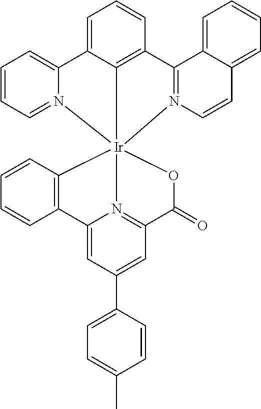

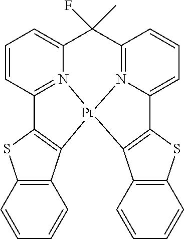

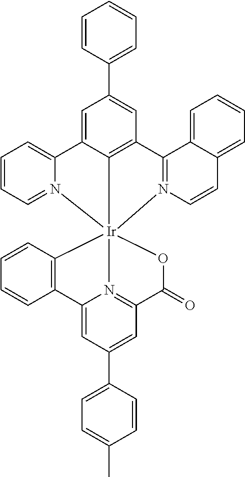

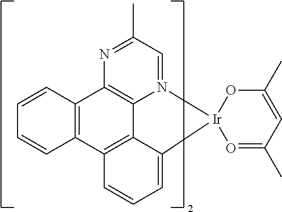

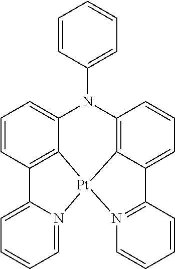

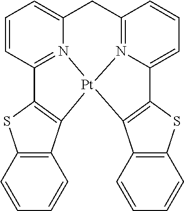

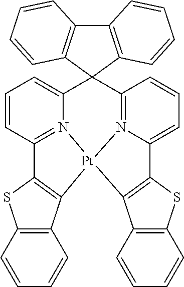

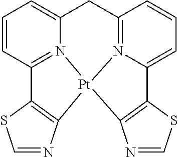

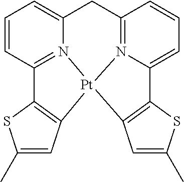

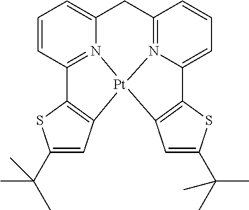

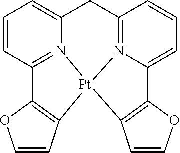

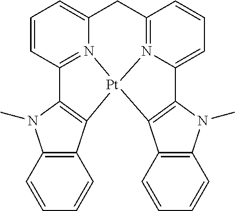

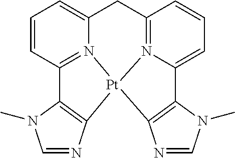

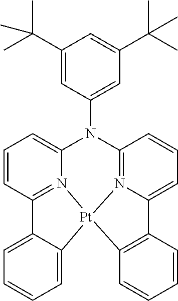

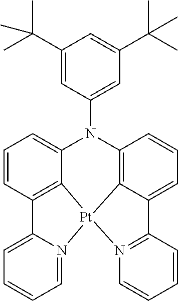

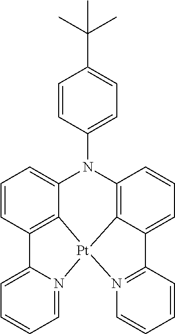

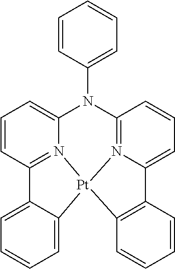

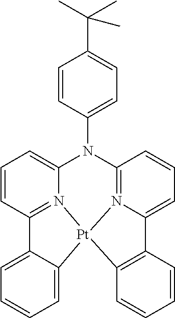

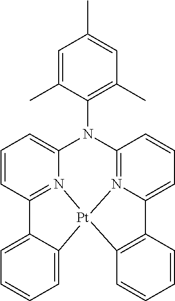

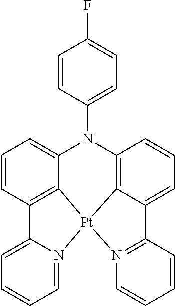

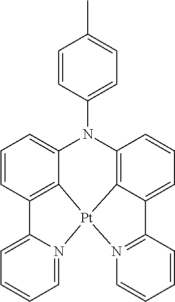

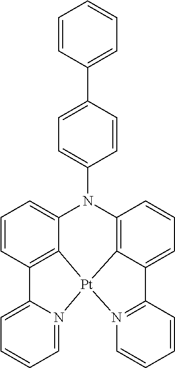

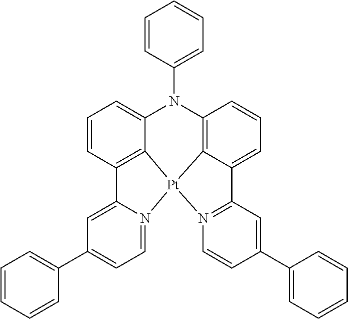

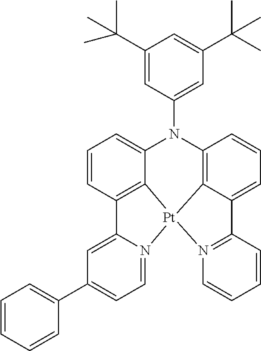

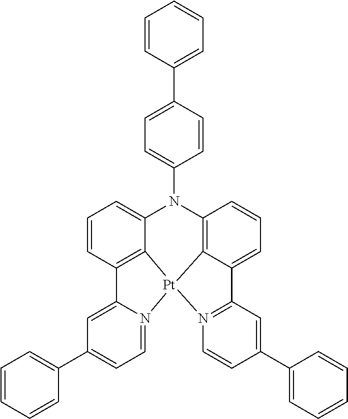

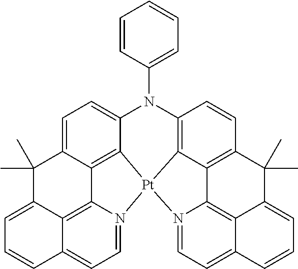

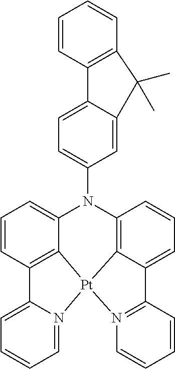

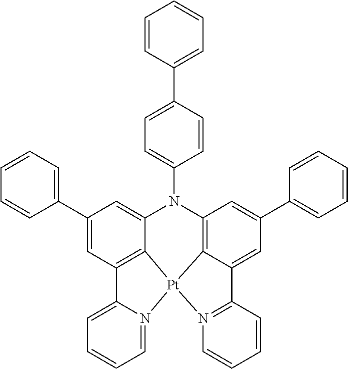

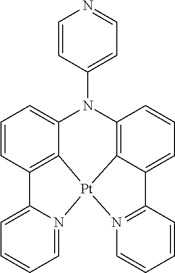

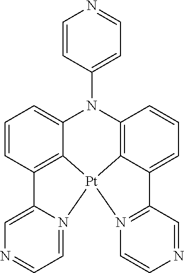

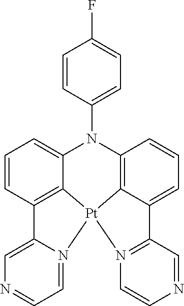

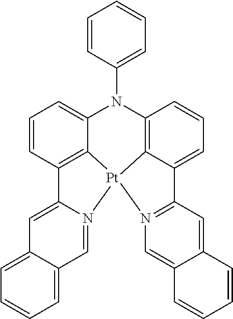

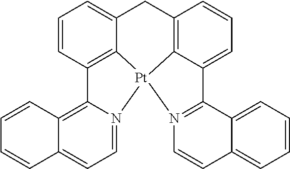

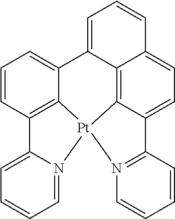

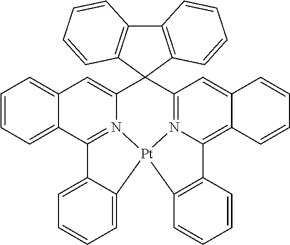

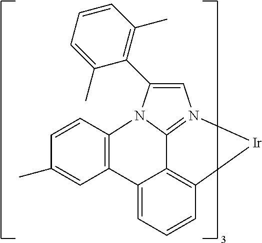

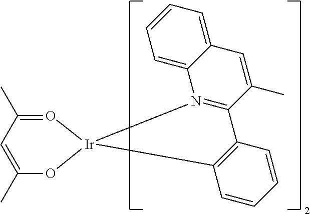

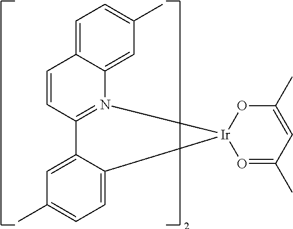

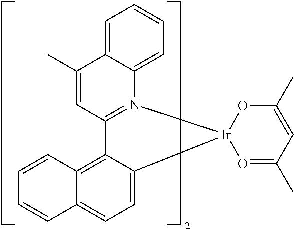

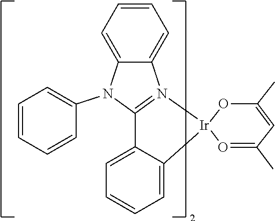

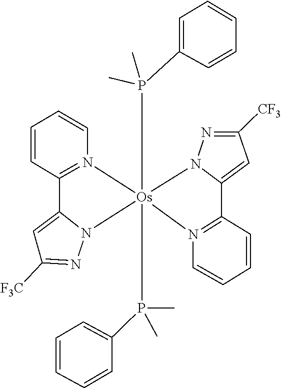

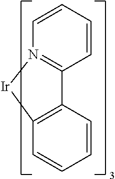

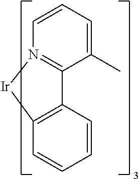

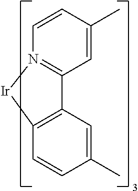

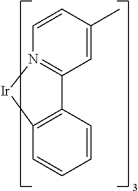

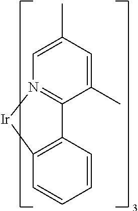

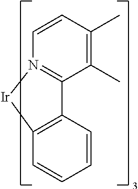

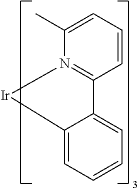

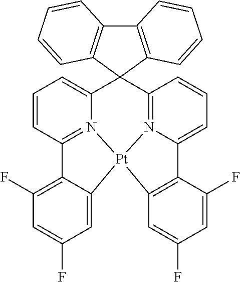

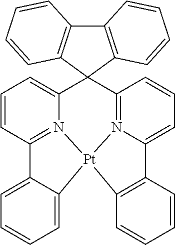

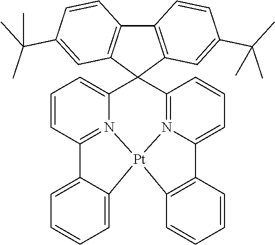

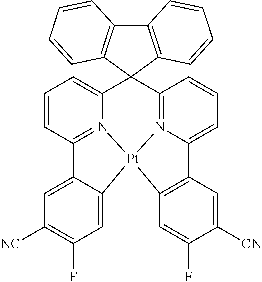

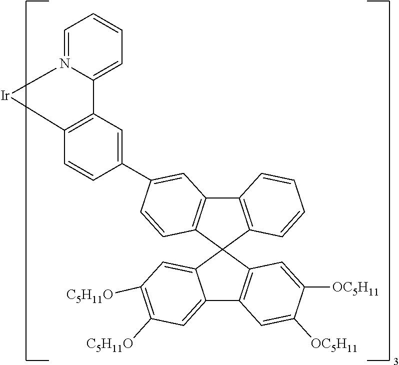

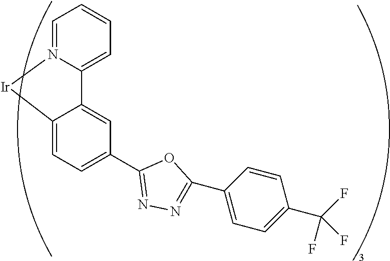

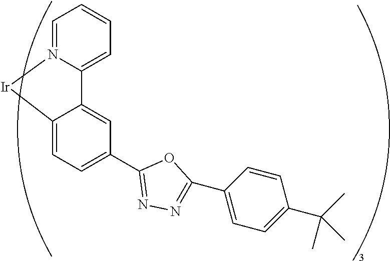

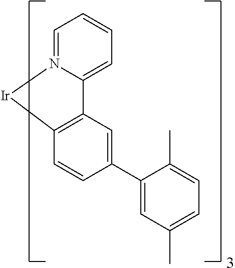

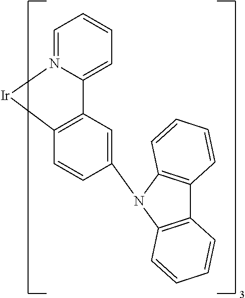

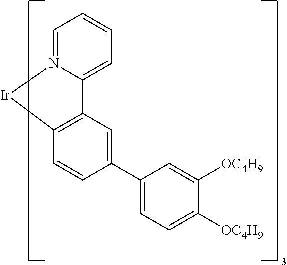

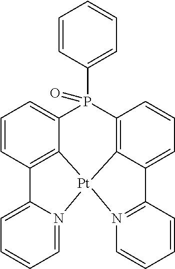

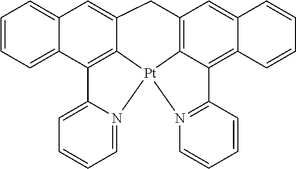

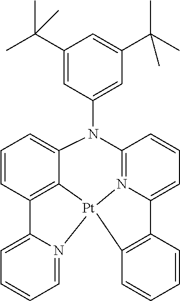

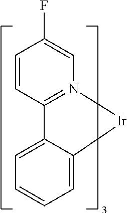

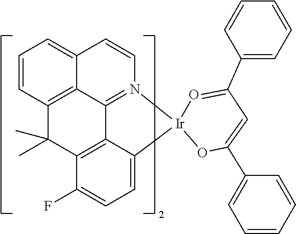

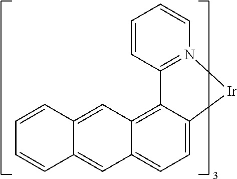

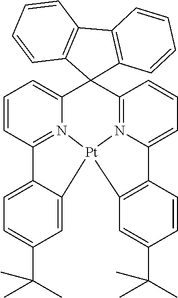

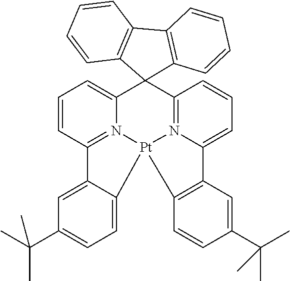

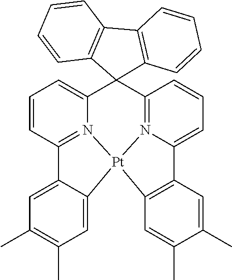

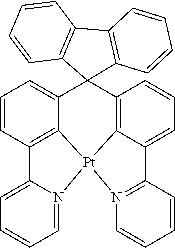

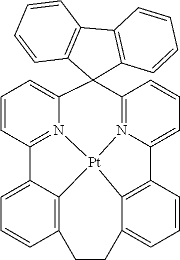

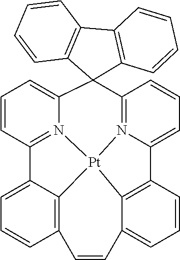

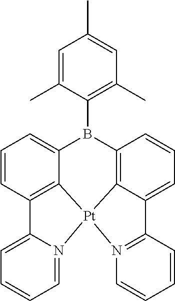

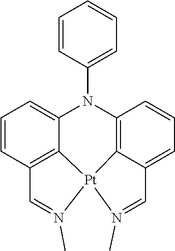

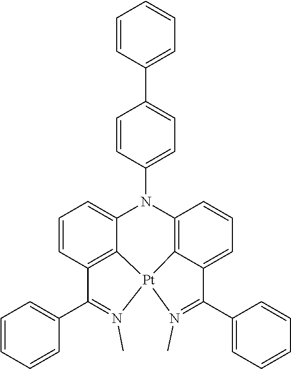

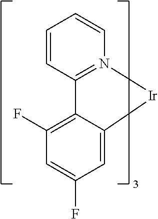

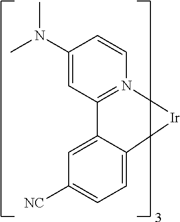

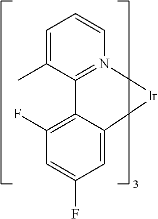

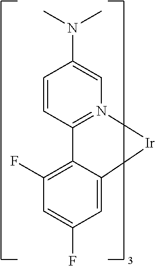

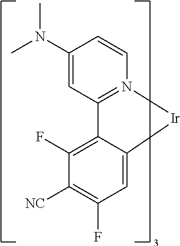

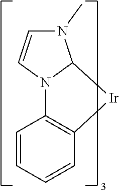

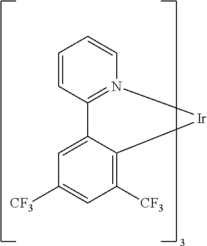

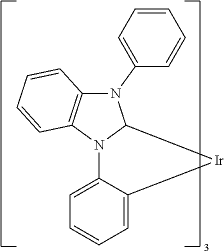

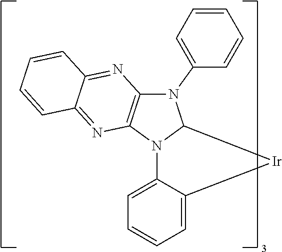

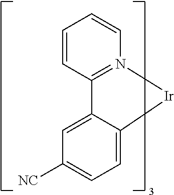

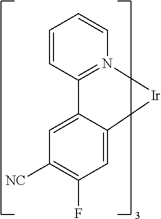

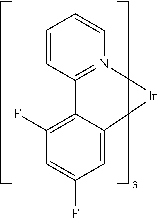

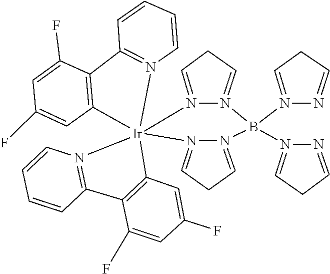

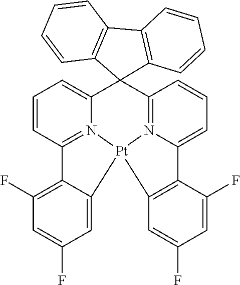

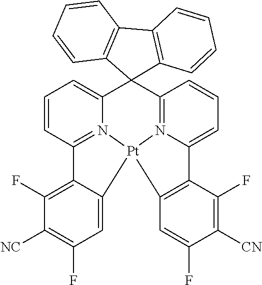

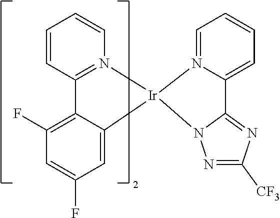

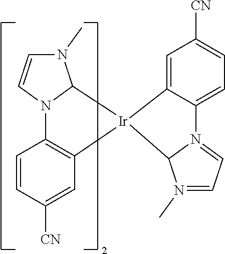

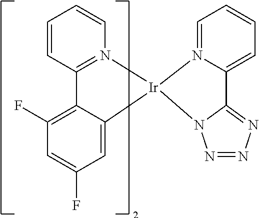

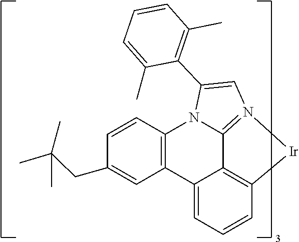

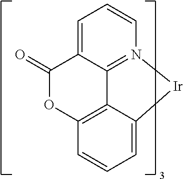

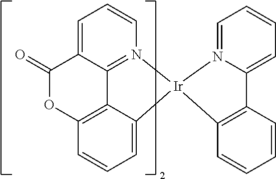

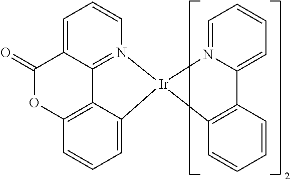

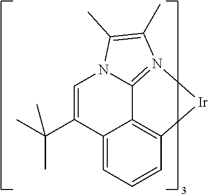

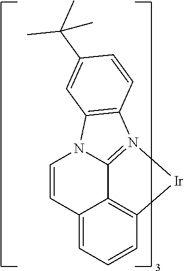

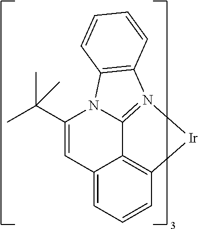

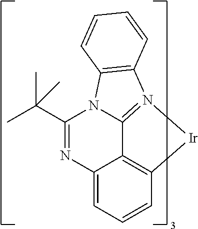

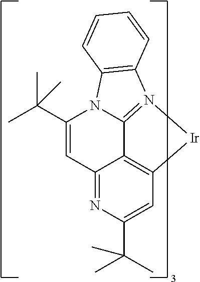

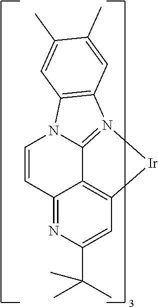

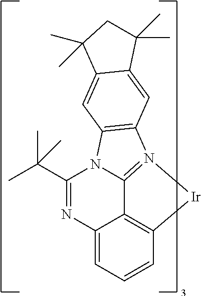

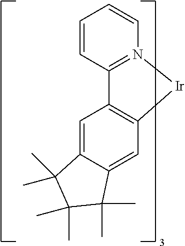

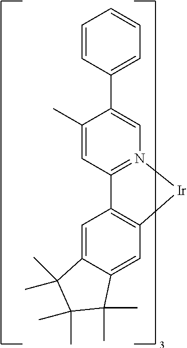

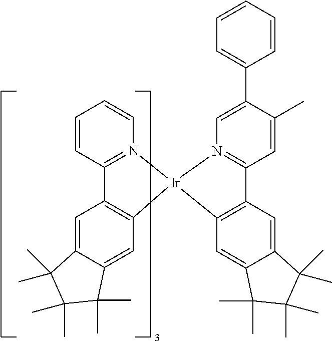

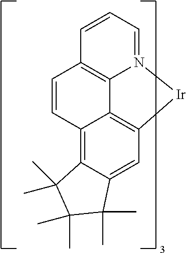

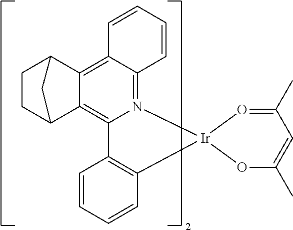

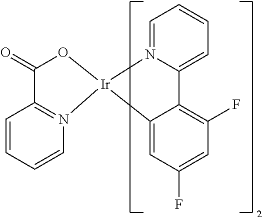

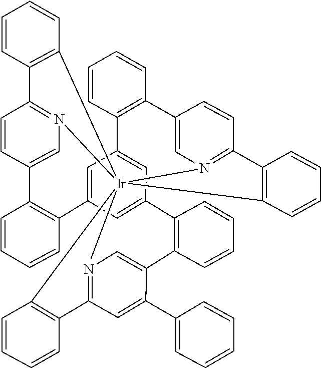

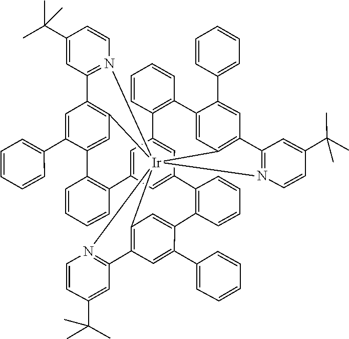

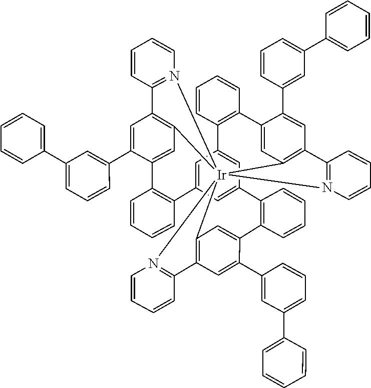

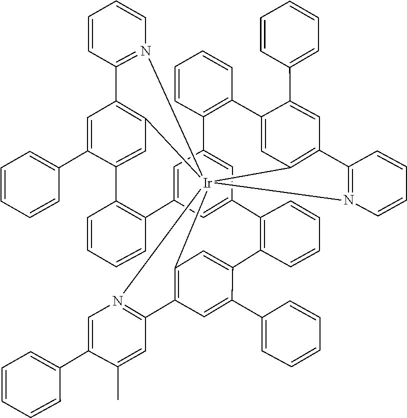

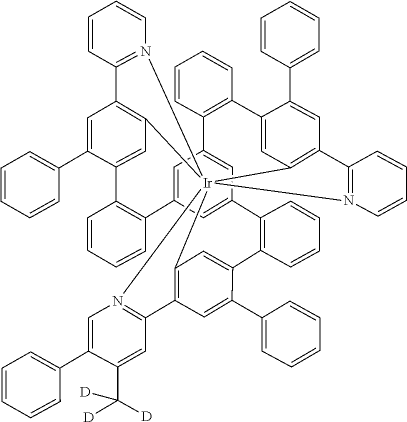

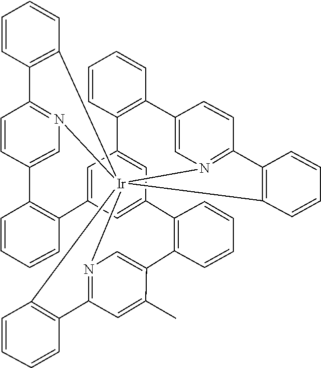

Fluorescent emitters:

Preferred fluorescent emitting compounds are selected from the class of the arylamines. An arylamine or an aromatic amine in the context of this invention is understood to mean a compound containing three substituted or unsubstituted aromatic or heteroaromatic ring systems bonded directly to the nitrogen. Preferably, at least one of these aromatic or heteroaromatic ring systems is a fused ring system, more preferably having at least 14 aromatic ring atoms. Preferred examples of these are aromatic anthraceneamines, aromatic anthracenediamines, aromatic pyreneamines, aromatic pyrenediamines, aromatic chryseneamines or aromatic chrysenediamines. An aromatic anthraceneamine is understood to mean a compound in which a diarylamino group is bonded directly to an anthracene group, preferably in the 9 position. An aromatic anthracenediamine is understood to mean a compound in which two diarylamino groups are bonded directly to an anthracene group, preferably in the 9,10 positions. Aromatic pyreneamines, pyrenediamines, chryseneamines and chrvsenediamines are defined analogouslv, where the diarvlamino aroups are bonded to the pyrene preferably in the 1 position or 1,6 positions. Further preferred emitting compounds are indenofluoreneamines or -diamines, benzoindenofluoreneamines or -diamines, and dibenzoindenofluoreneamines or -diamines, and indenofluorene derivatives having fused aryl groups. Likewise preferred are pyrenearylamines. Likewise preferred are benzoindenofluoreneamines, benzofluoreneamines, extended benzoindenofluorenes, phenoxazines, and fluorene derivatives joined to furan units or to thiophene units.

Matrix materials for fluorescent emitters:

Preferred matrix materials for fluorescent emitters are selected from the classes of the oligoarylenes (e.g. 2,2′,7,7′-tetraphenylspirobifluorene), especially the oligoarylenes containing fused aromatic groups, the oligoarylenevinylenes, the polypodal metal complexes, the hole-conducting compounds, the electron-conducting compounds, especially ketones, phosphine oxides and sulfoxides; the atropisomers, the boronic acid derivatives or the benzanthracenes. Particularly preferred matrix materials are selected from the classes of the oligoarylenes comprising naphthalene, anthracene, benzanthracene and/or pyrene or atropisomers of these compounds, the oligoarylenevinylenes, the ketones, the phosphine oxides and the sulfoxides. Very particularly preferred matrix materials are selected from the classes of the oligoarylenes comprising anthracene, benzanthracene, benzophenanthrene and/or pyrene or atropisomers of these compounds. An oligoarylene in the context of this invention shall be understood to mean a compound in which at least three aryl or arylene groups are bonded to one another.

Matrix materials for phosphorescent emitters:

Preferred matrix materials for phosphorescent emitters are, as well as the compounds of the formula (I), aromatic ketones, aromatic phosphine oxides or aromatic sulfoxides or sulfones, triarylamines, carbazole derivatives, e.g. CBP (N,N-biscarbazolylbiphenyl), indolocarbazole derivatives, indenocarbazole derivatives, azacarbazole derivatives, bipolar matrix materials, silanes, azaboroles or boronic esters, triazine derivatives, zinc complexes, diazasilole or tetraazasilole derivatives, diazaphosphole derivatives, bridged carbazole derivatives, triphenylene derivatives, or lactams.

Electron-transporting materials:

Suitable electron-transporting materials are, for example, the compounds disclosed in Y. Shirota et al., Chem. Rev. 2007, 107(4), 953-1010, or other materials used in these layers according to the prior art.

Materials used for the electron transport layer may be any materials that are used as electron transport materials in the electron transport layer according to the prior art. Especially suitable are aluminium complexes, for example Alq₃, zirconium complexes, for example Zrq₄, lithium complexes, for example Liq, benzimidazole derivatives, triazine derivatives, pyrimidine derivatives, pyridine derivatives, pyrazine derivatives, quinoxaline derivatives, quinoline derivatives, oxadiazole derivatives, aromatic ketones, lactams, boranes, diazaphosphole derivatives and phosphine oxide derivatives. Preferred electron-transporting compounds are shown in the following table:

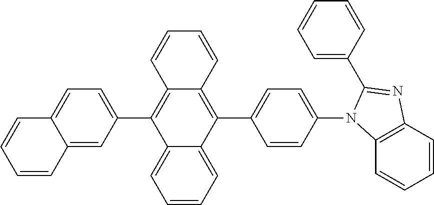 [SMILES: c1ccc(-c2nc3ccccc3n2-c2ccc(-c3c4ccccc4c(-c4ccc5ccccc5c4)c4ccccc34)cc2)cc1]

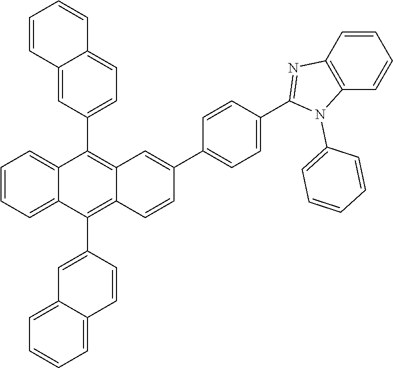 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

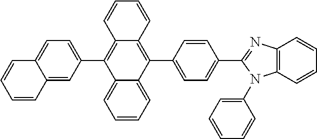 [SMILES: c1ccc(-n2c(-c3ccc(-c4c5ccccc5c(-c5ccc6ccccc6c5)c5ccccc45)cc3)nc3ccccc32)cc1]

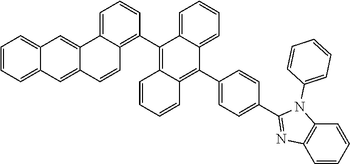 [SMILES: c1ccc(-n2c(-c3ccc(-c4c5ccccc5c(-c5cccc6c5ccc5cc7ccccc7cc56)c5ccccc45)cc3)nc3ccccc32)cc1]

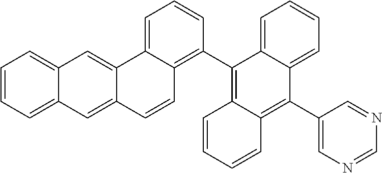 [SMILES: c1ccc2cc3c(ccc4c(-c5c6ccccc6c(-c6cncnc6)c6ccccc56)cccc43)cc2c1]

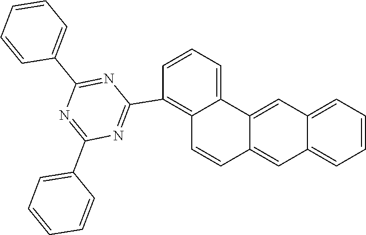 [SMILES: c1ccc(-c2nc(-c3ccccc3)nc(-c3cccc4c3ccc3cc5ccccc5cc34)n2)cc1]

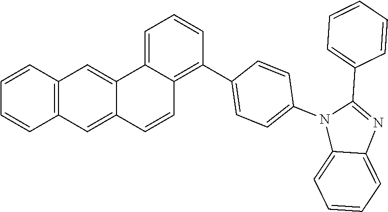 [SMILES: c1ccc(-c2nc3ccccc3n2-c2ccc(-c3cccc4c3ccc3cc5ccccc5cc34)cc2)cc1]

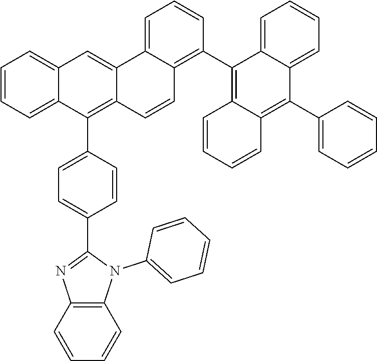 [SMILES: c1ccc(-c2c3ccccc3c(-c3cccc4c3ccc3c(-c5ccc(-c6nc7ccccc7n6-c6ccccc6)cc5)c5ccccc5cc34)c3ccccc23)cc1]

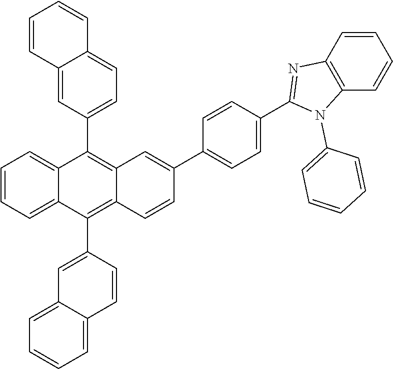 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

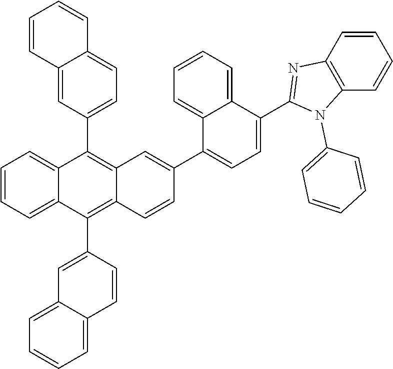 [SMILES: c1ccc(-n2c(-c3ccc(-c4ccc5c(-c6ccc7ccccc7c6)c6ccccc6c(-c6ccc7ccccc7c6)c5c4)c4ccccc34)nc3ccccc32)cc1]

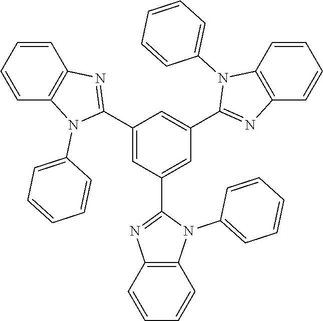 [SMILES: c1ccc(-n2c(-c3cc(-c4nc5ccccc5n4-c4ccccc4)cc(-c4nc5ccccc5n4-c4ccccc4)c3)nc3ccccc32)cc1]

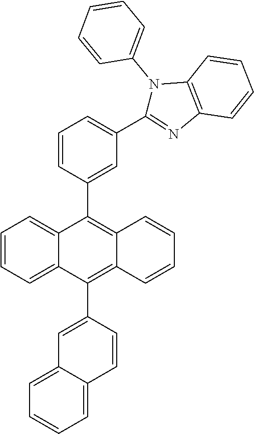 [SMILES: c1ccc(-n2c(-c3cccc(-c4c5ccccc5c(-c5ccc6ccccc6c5)c5ccccc45)c3)nc3ccccc32)cc1]

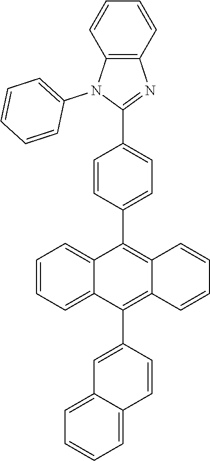 [SMILES: c1ccc(-n2c(-c3ccc(-c4c5ccccc5c(-c5ccc6ccccc6c5)c5ccccc45)cc3)nc3ccccc32)cc1]

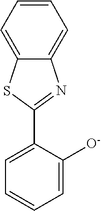 [SMILES: [O-]c1ccccc1-c1nc2ccccc2s1]

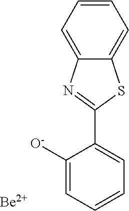

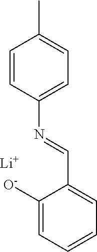 [SMILES: [Li+] ; Cc1ccc(/N=C/c2ccccc2[O-])cc1]

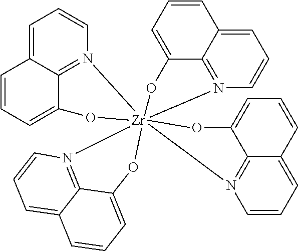

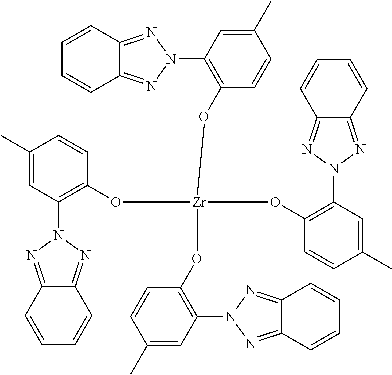

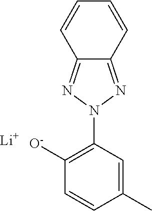 [SMILES: [Li+] ; Cc1ccc([O-])c(-n2nc3ccccc3n2)c1]

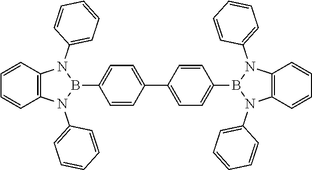 [SMILES: c1ccc(N2B(c3ccc(-c4ccc(B5N(c6ccccc6)c6ccccc6N5c5ccccc5)cc4)cc3)N(c3ccccc3)c3ccccc32)cc1]

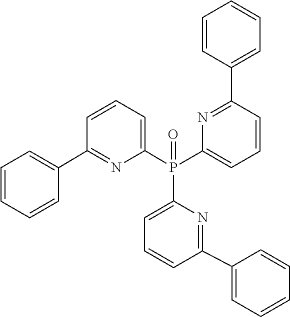 [SMILES: O=P(c1cccc(-c2ccccc2)n1)(c1cccc(-c2ccccc2)n1)c1cccc(-c2ccccc2)n1]

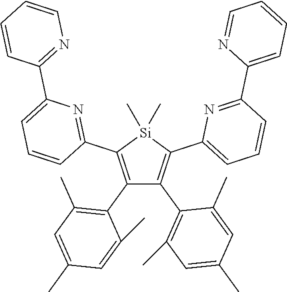 [SMILES: Cc1cc(C)c(C2=C(c3cccc(-c4ccccn4)n3)[Si](C)(C)C(c3cccc(-c4ccccn4)n3)=C2c2c(C)cc(C)cc2C)c(C)c1]

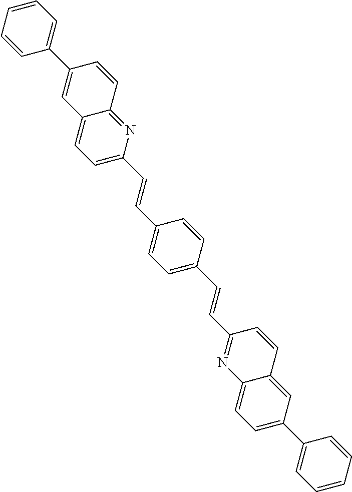 [SMILES: C(=C\c1ccc2cc(-c3ccccc3)ccc2n1)/c1ccc(/C=C/c2ccc3cc(-c4ccccc4)ccc3n2)cc1]

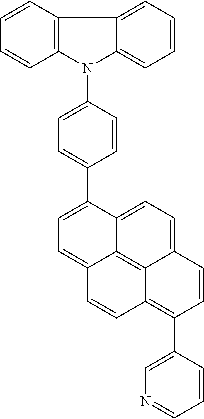 [SMILES: c1cncc(-c2ccc3ccc4c(-c5ccc(-n6c7ccccc7c7ccccc76)cc5)ccc5ccc2c3c54)c1]

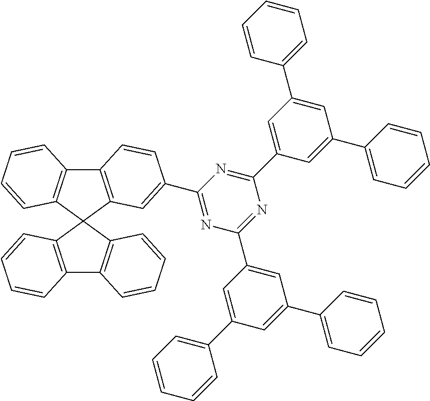 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

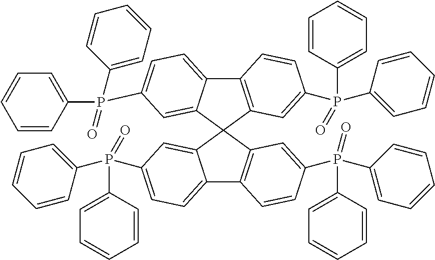 [SMILES: O=P(c1ccccc1)(c1ccccc1)c1ccc2c(c1)C1(c3cc(P(=O)(c4ccccc4)c4ccccc4)ccc3-2)c2cc(P(=O)(c3ccccc3)c3ccccc3)ccc2-c2ccc(P(=O)(c3ccccc3)c3ccccc3)cc21]

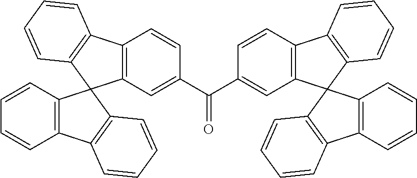 [SMILES: O=C(c1ccc2c(c1)C1(c3ccccc3-c3ccccc31)c1ccccc1-2)c1ccc2c(c1)C1(c3ccccc3-c3ccccc31)c1ccccc1-2]

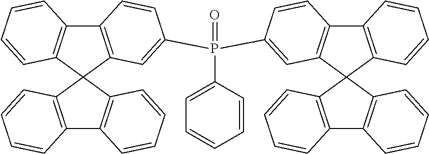 [SMILES: O=P(c1ccccc1)(c1ccc2c(c1)C1(c3ccccc3-c3ccccc31)c1ccccc1-2)c1ccc2c(c1)C1(c3ccccc3-c3ccccc31)c1ccccc1-2]

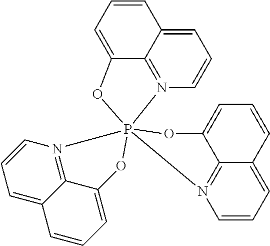

Hole-transporting materials:

Further compounds which, in addition to the compounds of the formula (I), are preferably used in hole-transporting layers of the OLEDs of the invention are indenofluoreneamine derivatives, amine derivatives, hexaazatriphenylene derivatives, amine derivatives with fused aromatic systems, monobenzoindenofluoreneamines, dibenzoindenofluoreneamines, spirobifluoreneamines, fluoreneamines, spirodibenzopyranamines, dihydroacridine derivatives, spirodibenzofurans and spirodibenzothiophenes, phenanthrenediarylamines, spirotribenzotropolones, spirobifluorenes having meta-phenyldiamine groups, spirobisacridines, xanthenediarylamines, and 9,10-dihydroanthracene spiro compounds having diarylamino groups. Preferred hole-transporting compounds are shown in the following table:

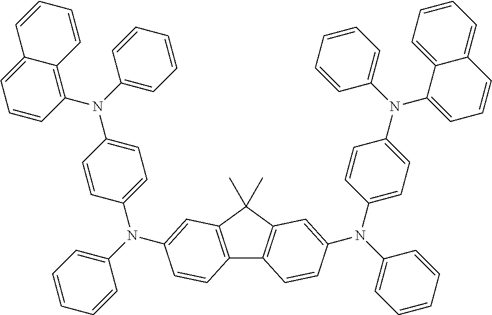 [SMILES: CC1(C)c2cc(N(c3ccccc3)c3ccc(N(c4ccccc4)c4cccc5ccccc45)cc3)ccc2-c2ccc(N(c3ccccc3)c3ccc(N(c4ccccc4)c4cccc5ccccc45)cc3)cc21]

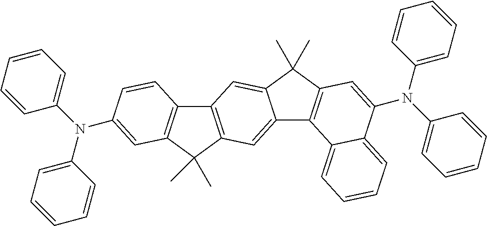 [SMILES: CC1(C)c2cc(N(c3ccccc3)c3ccccc3)ccc2-c2cc3c(cc21)-c1c(cc(N(c2ccccc2)c2ccccc2)c2ccccc12)C3(C)C]

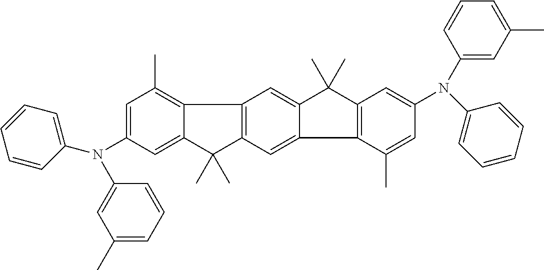 [SMILES: Cc1cccc(N(c2ccccc2)c2cc(C)c3c(c2)C(C)(C)c2cc4c(cc2-3)C(C)(C)c2cc(N(c3ccccc3)c3cccc(C)c3)cc(C)c2-4)c1]

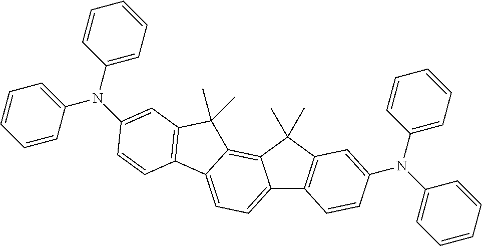 [SMILES: CC1(C)c2cc(N(c3ccccc3)c3ccccc3)ccc2-c2ccc3c(c21)C(C)(C)c1cc(N(c2ccccc2)c2ccccc2)ccc1-3]

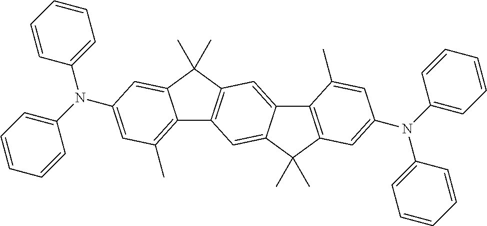 [SMILES: Cc1cc(N(c2ccccc2)c2ccccc2)cc2c1-c1cc3c(cc1C2(C)C)-c1c(C)cc(N(c2ccccc2)c2ccccc2)cc1C3(C)C]

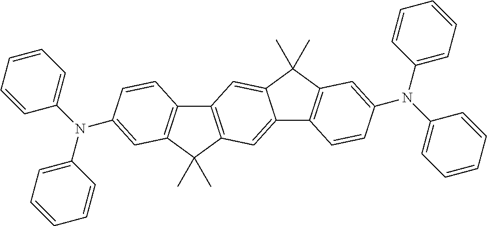 [SMILES: CC1(C)c2cc(N(c3ccccc3)c3ccccc3)ccc2-c2cc3c(cc21)-c1ccc(N(c2ccccc2)c2ccccc2)cc1C3(C)C]

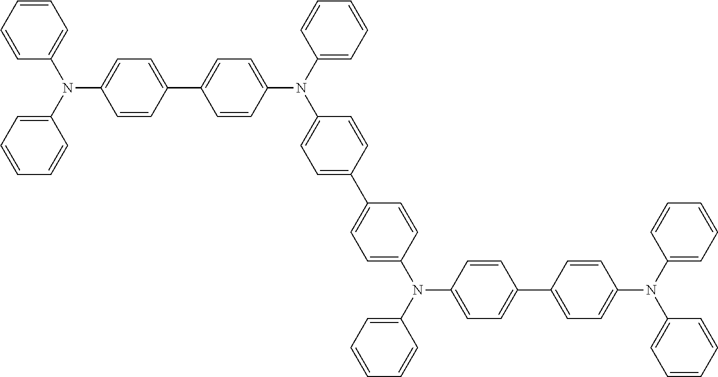 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

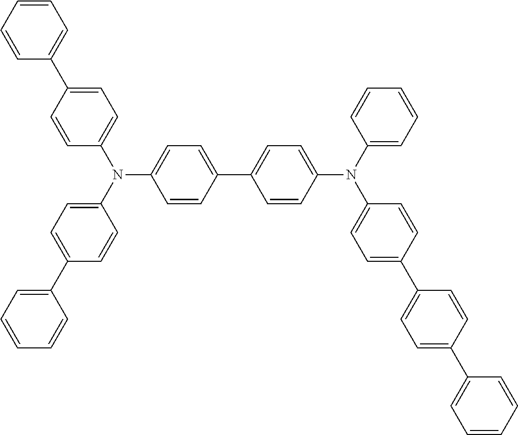 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

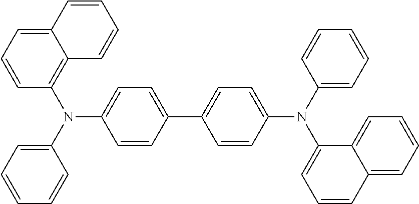 [SMILES: c1ccc(N(c2ccc(-c3ccc(N(c4ccccc4)c4cccc5ccccc45)cc3)cc2)c2cccc3ccccc23)cc1]

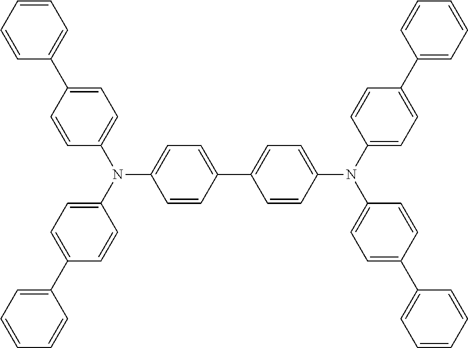 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

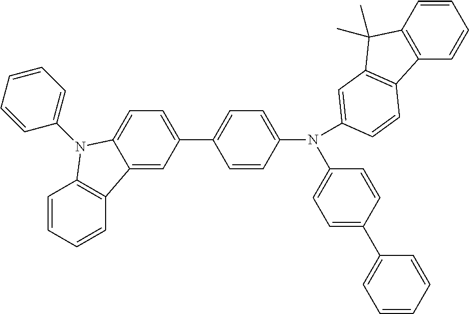 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

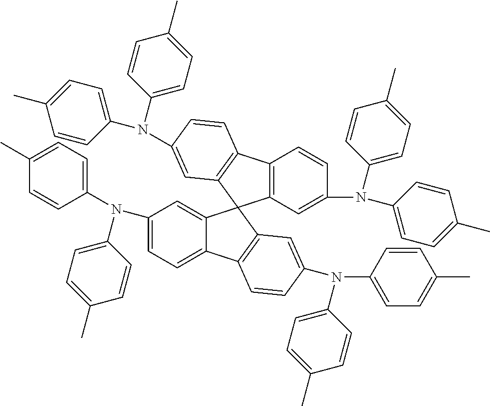

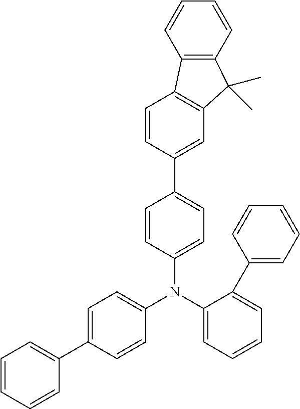 [SMILES: CC1(C)c2ccccc2-c2ccc(-c3ccc(N(c4ccc(-c5ccccc5)cc4)c4ccccc4-c4ccccc4)cc3)cc21]

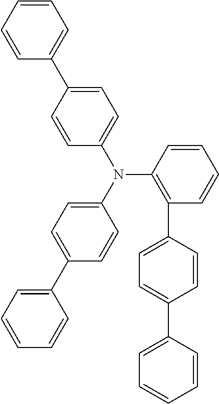 [SMILES: c1ccc(-c2ccc(-c3ccccc3N(c3ccc(-c4ccccc4)cc3)c3ccc(-c4ccccc4)cc3)cc2)cc1]

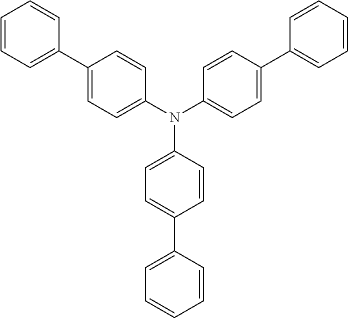 [SMILES: c1ccc(-c2ccc(N(c3ccc(-c4ccccc4)cc3)c3ccc(-c4ccccc4)cc3)cc2)cc1]

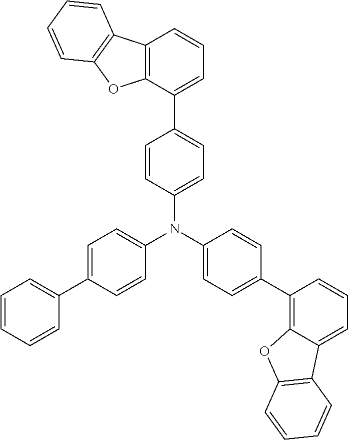 [SMILES: c1ccc(-c2ccc(N(c3ccc(-c4cccc5c4oc4ccccc45)cc3)c3ccc(-c4cccc5c4oc4ccccc45)cc3)cc2)cc1]

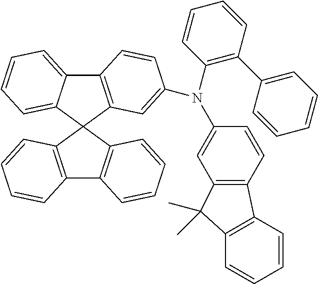 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

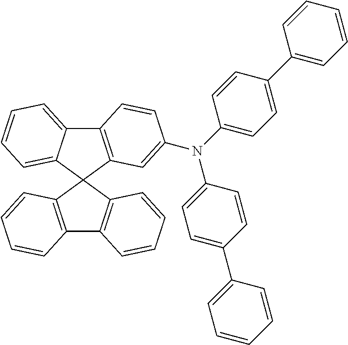 [SMILES: c1ccc(-c2ccc(N(c3ccc(-c4ccccc4)cc3)c3ccc4c(c3)C3(c5ccccc5-c5ccccc53)c3ccccc3-4)cc2)cc1]

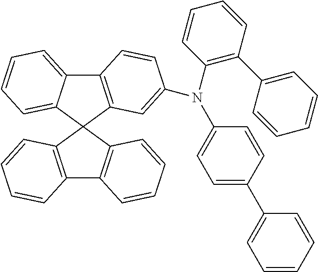 [SMILES: c1ccc(-c2ccc(N(c3ccc4c(c3)C3(c5ccccc5-c5ccccc53)c3ccccc3-4)c3ccccc3-c3ccccc3)cc2)cc1]

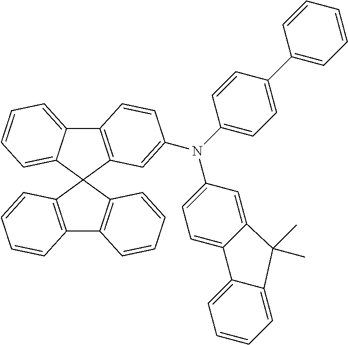 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21]

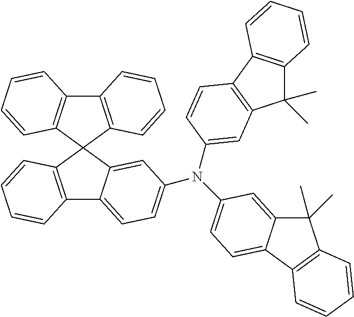 [SMILES: CC1(C)c2ccccc2-c2ccc(N(c3ccc4c(c3)C(C)(C)c3ccccc3-4)c3ccc4c(c3)C3(c5ccccc5-c5ccccc53)c3ccccc3-4)cc21]

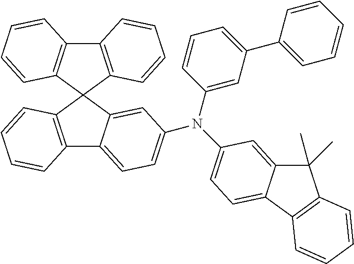 [SMILES: CC1(C)c2ccccc2-c2ccc(N(c3cccc(-c4ccccc4)c3)c3ccc4c(c3)C3(c5ccccc5-c5ccccc53)c3ccccc3-4)cc21]

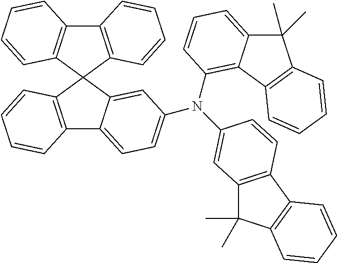 [SMILES: CC1(C)c2ccccc2-c2ccc(N(c3ccc4c(c3)C3(c5ccccc5-c5ccccc53)c3ccccc3-4)c3cccc4c3-c3ccccc3C4(C)C)cc21]

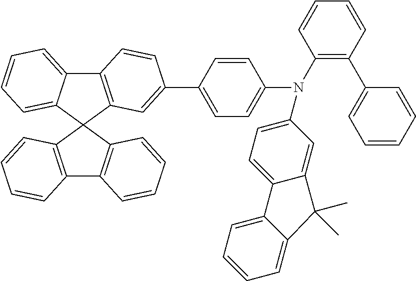 [SMILES: CC1(C)c2ccccc2-c2ccc(N(c3ccc(-c4ccc5c(c4)C4(c6ccccc6-c6ccccc64)c4ccccc4-5)cc3)c3ccccc3-c3ccccc3)cc21]

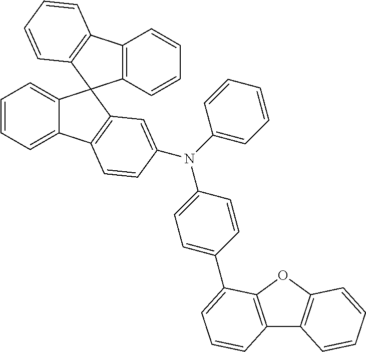 [SMILES: c1ccc(N(c2ccc(-c3cccc4c3oc3ccccc34)cc2)c2ccc3c(c2)C2(c4ccccc4-c4ccccc42)c2ccccc2-3)cc1]

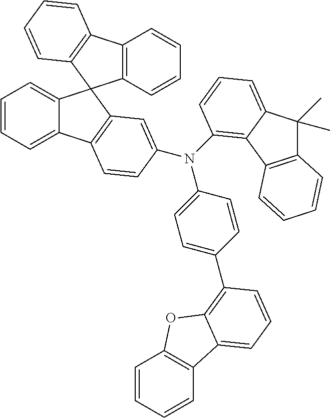 [SMILES: CC1(C)c2ccccc2-c2c(N(c3ccc(-c4cccc5c4oc4ccccc45)cc3)c3ccc4c(c3)C3(c5ccccc5-c5ccccc53)c3ccccc3-4)cccc21]

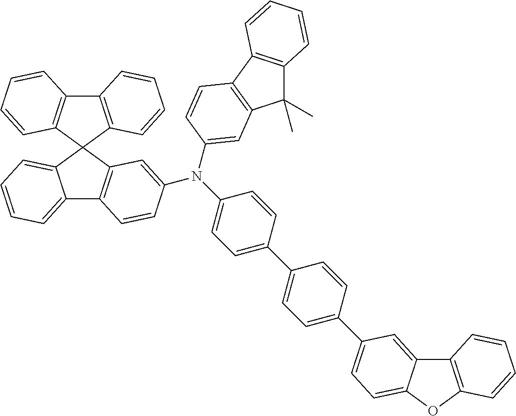 [SMILES: CC1(C)c2ccccc2-c2ccc(N(c3ccc(-c4ccc(-c5ccc6oc7ccccc7c6c5)cc4)cc3)c3ccc4c(c3)C3(c5ccccc5-c5ccccc53)c3ccccc3-4)cc21]

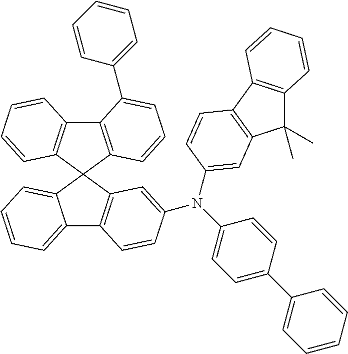 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)cccc43)cc21]

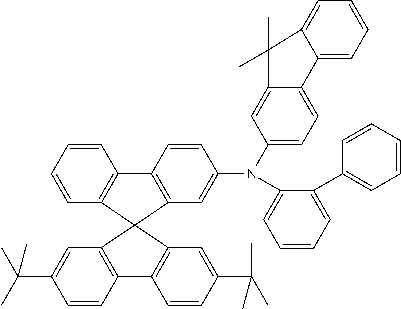 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

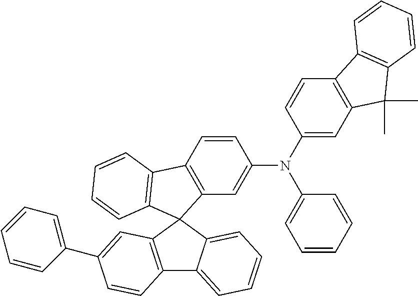 [SMILES: CC1(C)c2ccccc2-c2ccc(N(c3ccccc3)c3ccc4c(c3)C3(c5ccccc5-c5ccc(-c6ccccc6)cc53)c3ccccc3-4)cc21]

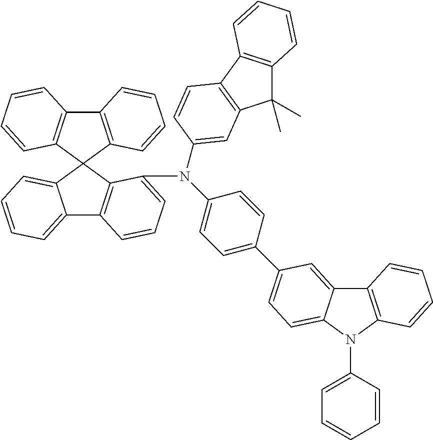 [SMILES: CC1(C)c2ccccc2-c2ccc(N(c3ccc(-c4ccc5c(c4)c4ccccc4n5-c4ccccc4)cc3)c3cccc4c3C3(c5ccccc5-c5ccccc53)c3ccccc3-4)cc21]

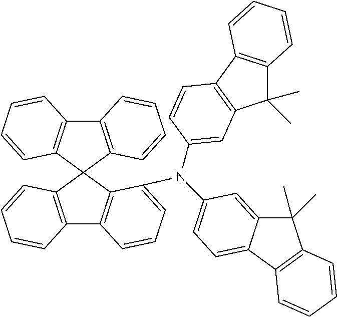 [SMILES: CC1(C)c2ccccc2-c2ccc(N(c3ccc4c(c3)C(C)(C)c3ccccc3-4)c3cccc4c3C3(c5ccccc5-c5ccccc53)c3ccccc3-4)cc21]

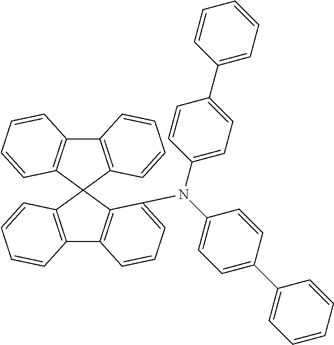 [SMILES: c1ccc(-c2ccc(N(c3ccc(-c4ccccc4)cc3)c3cccc4c3C3(c5ccccc5-c5ccccc53)c3ccccc3-4)cc2)cc1]

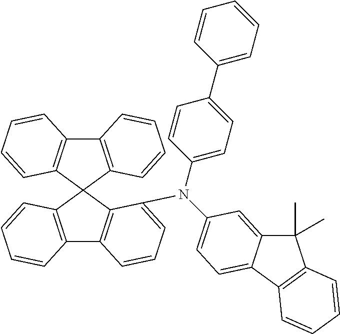 [SMILES: CC1(C)c2ccccc2-c2ccc(N(c3ccc(-c4ccccc4)cc3)c3cccc4c3C3(c5ccccc5-c5ccccc53)c3ccccc3-4)cc21]

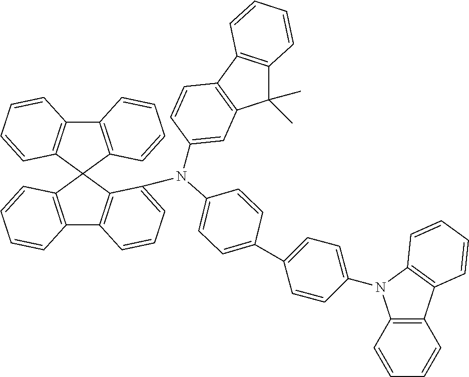 [SMILES: CC1(C)c2ccccc2-c2ccc(N(c3ccc(-c4ccc(-n5c6ccccc6c6ccccc65)cc4)cc3)c3cccc4c3C3(c5ccccc5-c5ccccc53)c3ccccc3-4)cc21]

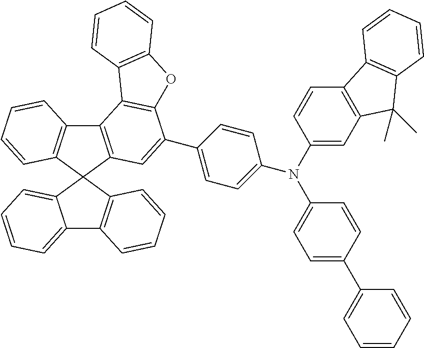 [SMILES: CC1(C)c2ccccc2-c2ccc(N(c3ccc(-c4ccccc4)cc3)c3ccc(-c4cc5c(c6c4oc4ccccc46)-c4ccccc4C54c5ccccc5-c5ccccc54)cc3)cc21]

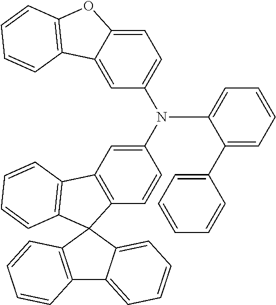 [SMILES: c1ccc(-c2ccccc2N(c2ccc3c(c2)-c2ccccc2C32c3ccccc3-c3ccccc32)c2ccc3oc4ccccc4c3c2)cc1]

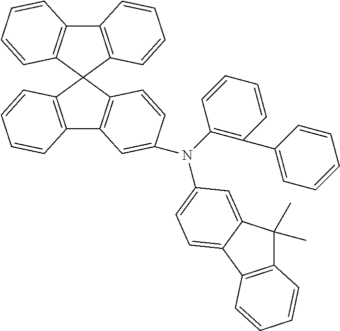 [SMILES: CC1(C)c2ccccc2-c2ccc(N(c3ccc4c(c3)-c3ccccc3C43c4ccccc4-c4ccccc43)c3ccccc3-c3ccccc3)cc21]

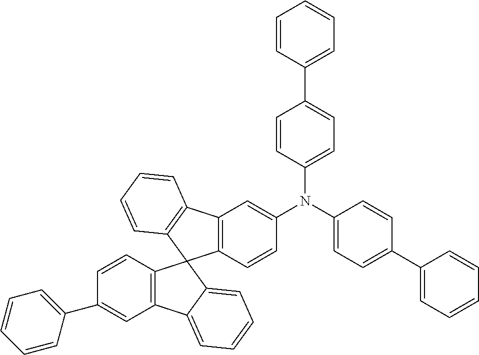 [SMILES: c1ccc(-c2ccc(N(c3ccc(-c4ccccc4)cc3)c3ccc4c(c3)-c3ccccc3C43c4ccccc4-c4cc(-c5ccccc5)ccc43)cc2)cc1]

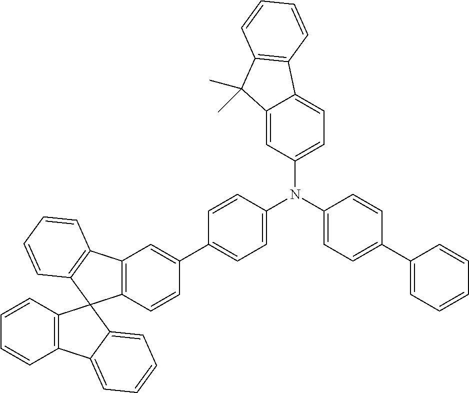 [SMILES: CC1(C)c2ccccc2-c2ccc(N(c3ccc(-c4ccccc4)cc3)c3ccc(-c4ccc5c(c4)-c4ccccc4C54c5ccccc5-c5ccccc54)cc3)cc21]

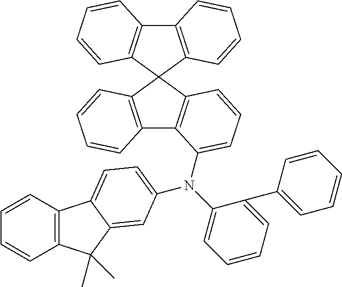 [SMILES: CC1(C)c2ccccc2-c2ccc(N(c3ccccc3-c3ccccc3)c3cccc4c3-c3ccccc3C43c4ccccc4-c4ccccc43)cc21]

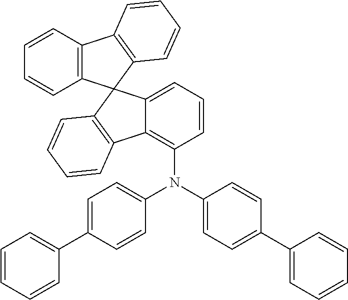 [SMILES: c1ccc(-c2ccc(N(c3ccc(-c4ccccc4)cc3)c3cccc4c3-c3ccccc3C43c4ccccc4-c4ccccc43)cc2)cc1]

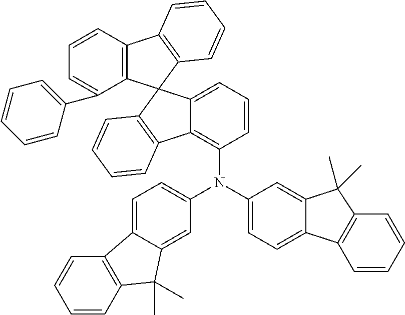 [SMILES: CC1(C)c2ccccc2-c2ccc(N(c3ccc4c(c3)C(C)(C)c3ccccc3-4)c3cccc4c3-c3ccccc3C43c4ccccc4-c4cccc(-c5ccccc5)c43)cc21]

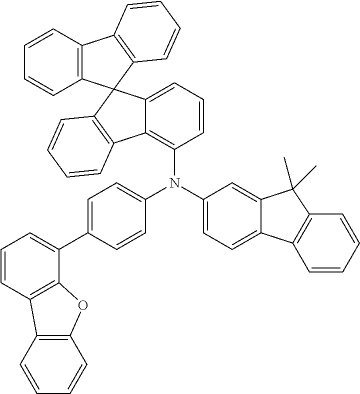 [SMILES: CC1(C)c2ccccc2-c2ccc(N(c3ccc(-c4cccc5c4oc4ccccc45)cc3)c3cccc4c3-c3ccccc3C43c4ccccc4-c4ccccc43)cc21]

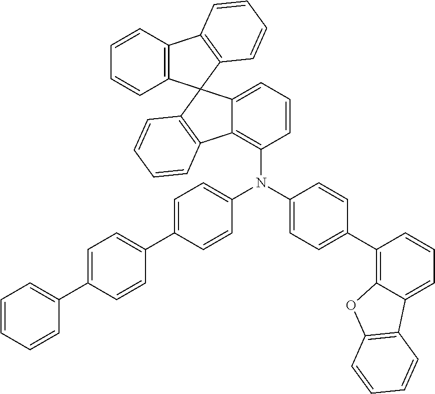 [SMILES: c1ccc(-c2ccc(-c3ccc(N(c4ccc(-c5cccc6c5oc5ccccc56)cc4)c4cccc5c4-c4ccccc4C54c5ccccc5-c5ccccc54)cc3)cc2)cc1]

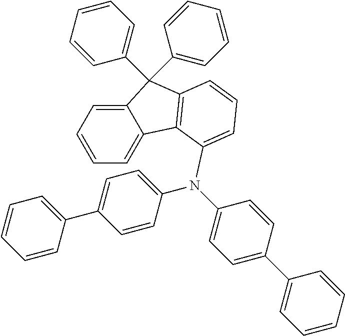 [SMILES: c1ccc(-c2ccc(N(c3ccc(-c4ccccc4)cc3)c3cccc4c3-c3ccccc3C4(c3ccccc3)c3ccccc3)cc2)cc1]

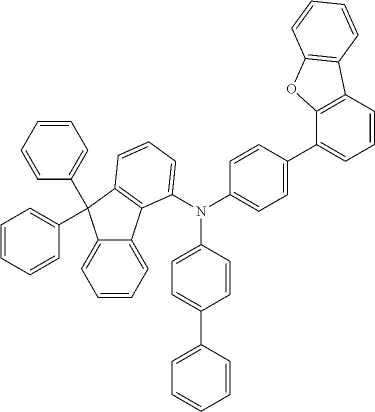 [SMILES: c1ccc(-c2ccc(N(c3ccc(-c4cccc5c4oc4ccccc45)cc3)c3cccc4c3-c3ccccc3C4(c3ccccc3)c3ccccc3)cc2)cc1]

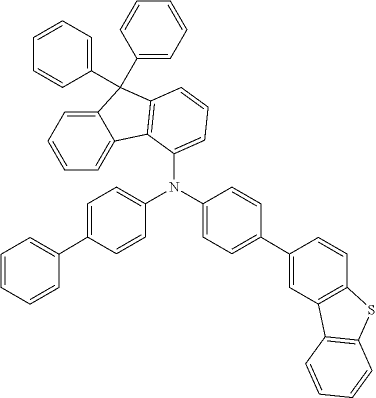 [SMILES: c1ccc(-c2ccc(N(c3ccc(-c4ccc5sc6ccccc6c5c4)cc3)c3cccc4c3-c3ccccc3C4(c3ccccc3)c3ccccc3)cc2)cc1]

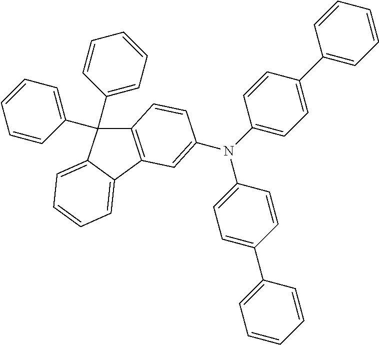 [SMILES: c1ccc(-c2ccc(N(c3ccc(-c4ccccc4)cc3)c3ccc4c(c3)-c3ccccc3C4(c3ccccc3)c3ccccc3)cc2)cc1]

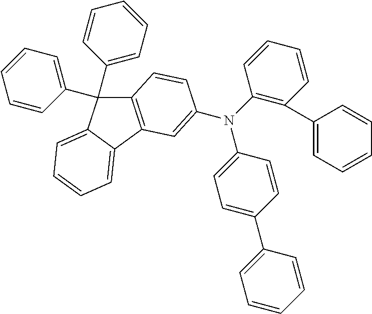 [SMILES: c1ccc(-c2ccc(N(c3ccc4c(c3)-c3ccccc3C4(c3ccccc3)c3ccccc3)c3ccccc3-c3ccccc3)cc2)cc1]

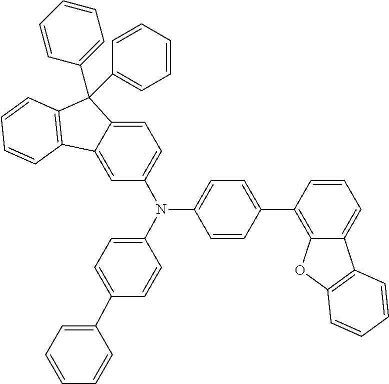 [SMILES: c1ccc(-c2ccc(N(c3ccc(-c4cccc5c4oc4ccccc45)cc3)c3ccc4c(c3)-c3ccccc3C4(c3ccccc3)c3ccccc3)cc2)cc1]

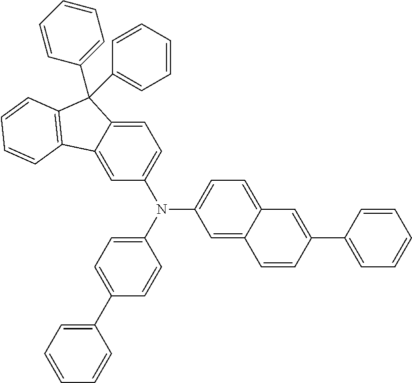 [SMILES: c1ccc(-c2ccc(N(c3ccc4c(c3)-c3ccccc3C4(c3ccccc3)c3ccccc3)c3ccc4cc(-c5ccccc5)ccc4c3)cc2)cc1]

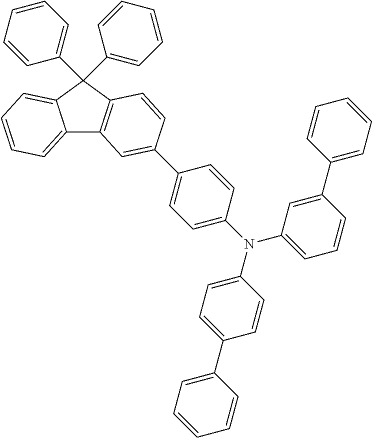 [SMILES: c1ccc(-c2ccc(N(c3ccc(-c4ccc5c(c4)-c4ccccc4C5(c4ccccc4)c4ccccc4)cc3)c3cccc(-c4ccccc4)c3)cc2)cc1]

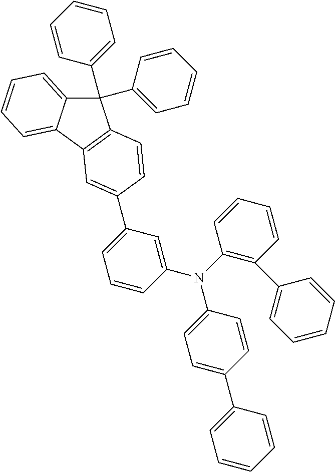 [SMILES: c1ccc(-c2ccc(N(c3cccc(-c4ccc5c(c4)-c4ccccc4C5(c4ccccc4)c4ccccc4)c3)c3ccccc3-c3ccccc3)cc2)cc1]

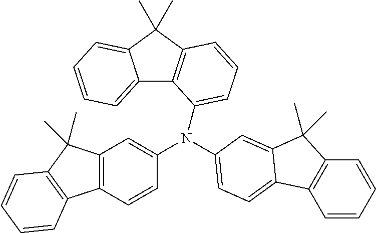 [SMILES: CC1(C)c2ccccc2-c2ccc(N(c3ccc4c(c3)C(C)(C)c3ccccc3-4)c3cccc4c3-c3ccccc3C4(C)C)cc21]

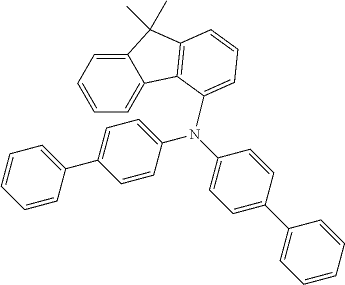 [SMILES: CC1(C)c2ccccc2-c2c(N(c3ccc(-c4ccccc4)cc3)c3ccc(-c4ccccc4)cc3)cccc21]

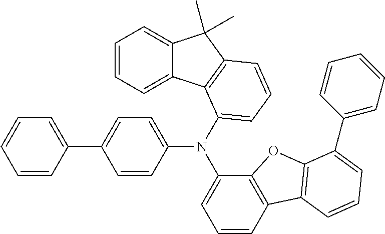 [SMILES: CC1(C)c2ccccc2-c2c(N(c3ccc(-c4ccccc4)cc3)c3cccc4c3oc3c(-c5ccccc5)cccc34)cccc21]

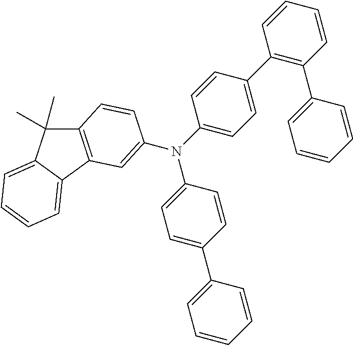 [SMILES: CC1(C)c2ccccc2-c2cc(N(c3ccc(-c4ccccc4)cc3)c3ccc(-c4ccccc4-c4ccccc4)cc3)ccc21]

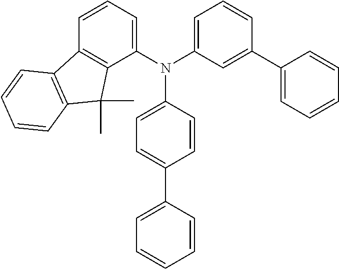 [SMILES: CC1(C)c2ccccc2-c2cccc(N(c3ccc(-c4ccccc4)cc3)c3cccc(-c4ccccc4)c3)c21]

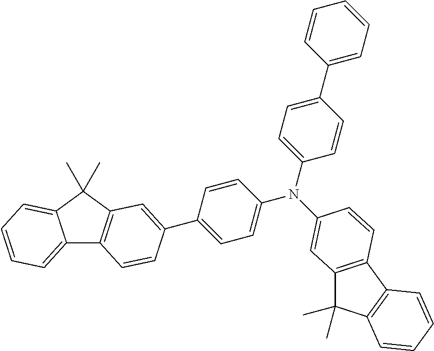 [SMILES: CC1(C)c2ccccc2-c2ccc(-c3ccc(N(c4ccc(-c5ccccc5)cc4)c4ccc5c(c4)C(C)(C)c4ccccc4-5)cc3)cc21]

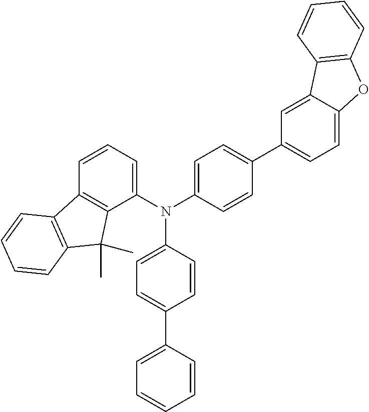 [SMILES: CC1(C)c2ccccc2-c2cccc(N(c3ccc(-c4ccccc4)cc3)c3ccc(-c4ccc5oc6ccccc6c5c4)cc3)c21]

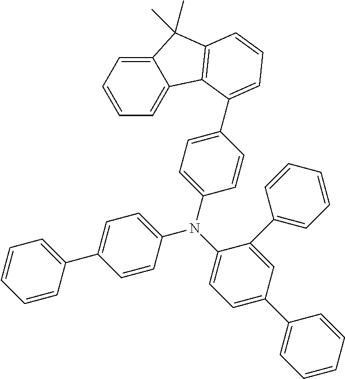 [SMILES: CC1(C)c2ccccc2-c2c(-c3ccc(N(c4ccc(-c5ccccc5)cc4)c4ccc(-c5ccccc5)cc4-c4ccccc4)cc3)cccc21]

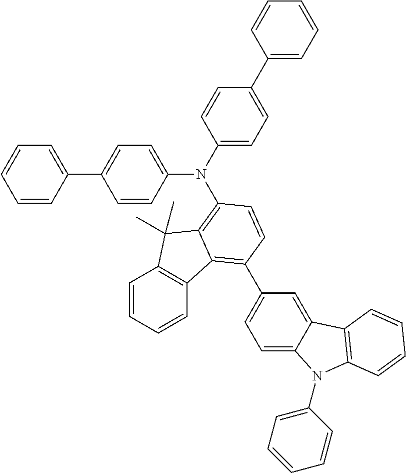 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

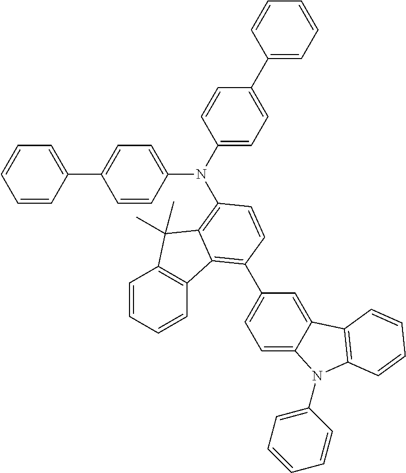 [SMILES: CC1(C)c2ccccc2-c2c(-c3ccc4c(c3)c3ccccc3n4-c3ccccc3)ccc(N(c3ccc(-c4ccccc4)cc3)c3ccc(-c4ccccc4)cc3)c21]

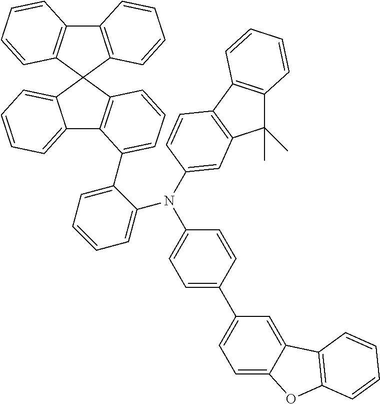 [SMILES: CC1(C)c2ccccc2-c2ccc(N(c3ccc(-c4ccc5oc6ccccc6c5c4)cc3)c3ccccc3-c3cccc4c3-c3ccccc3C43c4ccccc4-c4ccccc43)cc21]

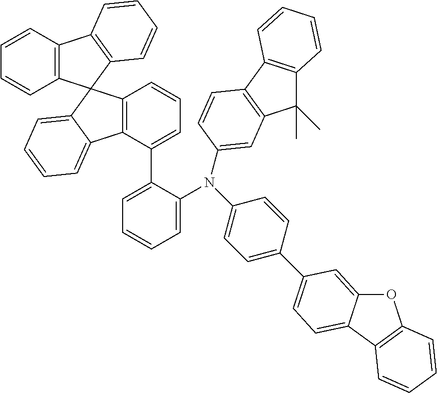 [SMILES: CC1(C)c2ccccc2-c2ccc(N(c3ccc(-c4ccc5c(c4)oc4ccccc45)cc3)c3ccccc3-c3cccc4c3-c3ccccc3C43c4ccccc4-c4ccccc43)cc21]

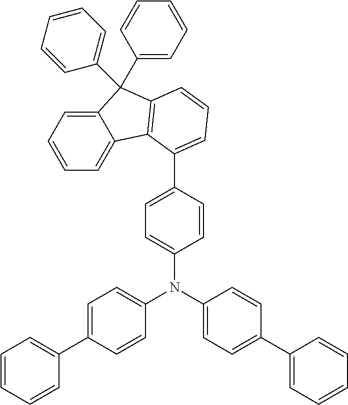 [SMILES: c1ccc(-c2ccc(N(c3ccc(-c4ccccc4)cc3)c3ccc(-c4cccc5c4-c4ccccc4C5(c4ccccc4)c4ccccc4)cc3)cc2)cc1]

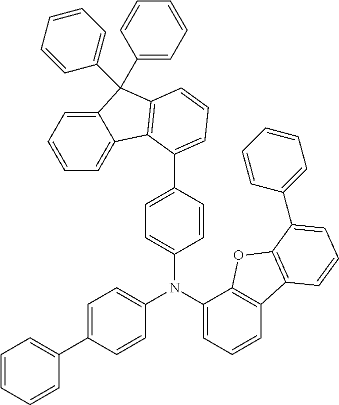 [SMILES: c1ccc(-c2ccc(N(c3ccc(-c4cccc5c4-c4ccccc4C5(c4ccccc4)c4ccccc4)cc3)c3cccc4c3oc3c(-c5ccccc5)cccc34)cc2)cc1]

In addition, the following compounds HT-1 to HT-10 are suitable for use in a layer having a hole-transporting function, especially in a hole injection layer, a hole transport layer and/or an electron blocker layer, or for use in an emitting layer as matrix material, especially as matrix material in an emitting layer comprising one or more phosphorescent emitters:

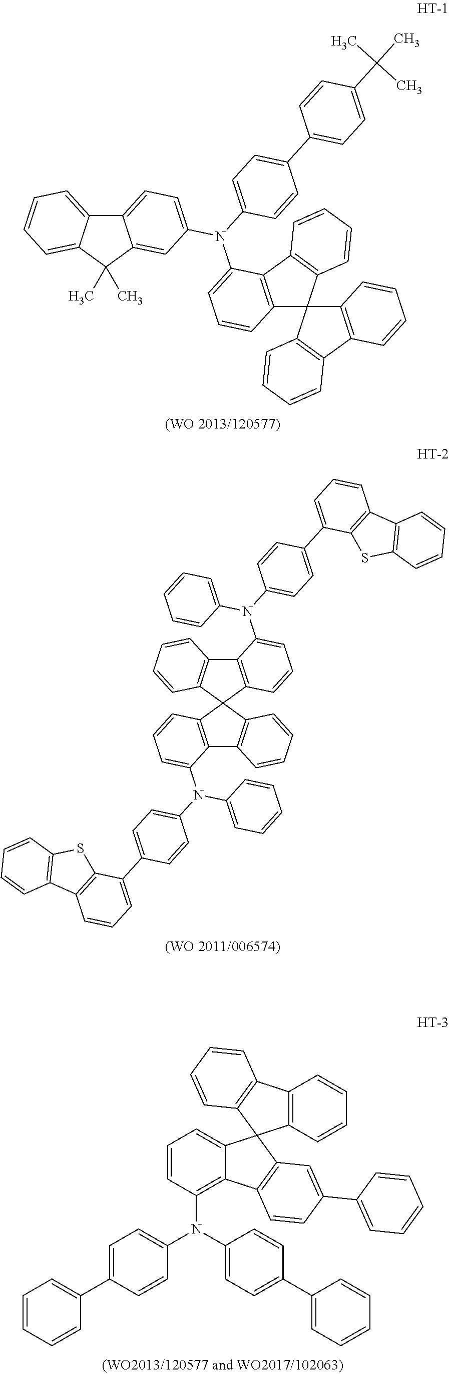 [SMILES: CC(C)(C)c1ccc(-c2ccc(N(c3ccc4c(c3)C(C)(C)c3ccccc3-4)c3cccc4c3-c3ccccc3C43c4ccccc4-c4ccccc43)cc2)cc1 ; c1ccc(N(c2ccc(-c3cccc4c3sc3ccccc34)cc2)c2cccc3c2-c2ccccc2C32c3ccccc3-c3c(N(c4ccccc4)c4ccc(-c5cccc6c5sc5ccccc56)cc4)cccc32)cc1 ; c1ccc(-c2ccc(N(c3ccc(-c4ccccc4)cc3)c3cccc4c3-c3ccc(-c5ccccc5)cc3C43c4ccccc4-c4ccccc43)cc2)cc1]

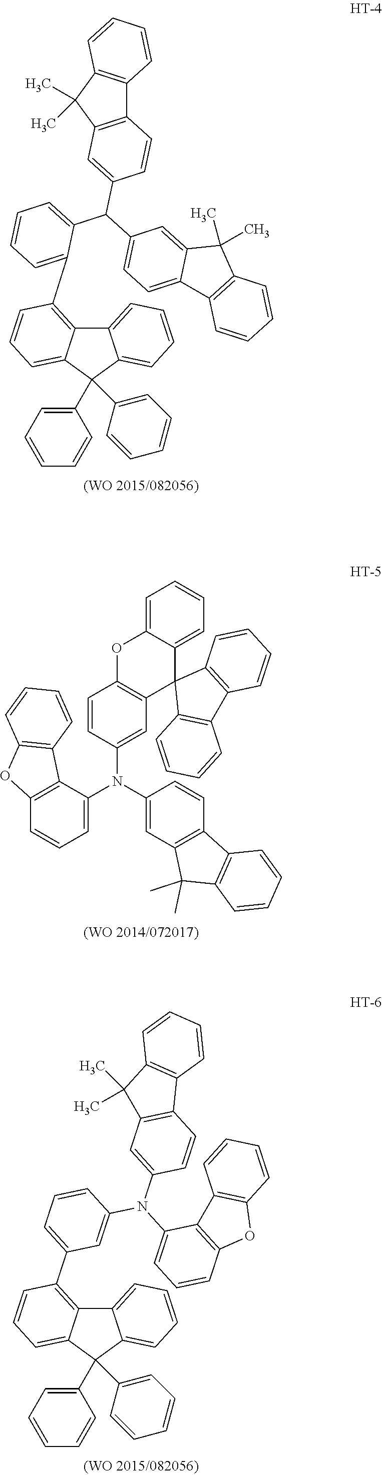

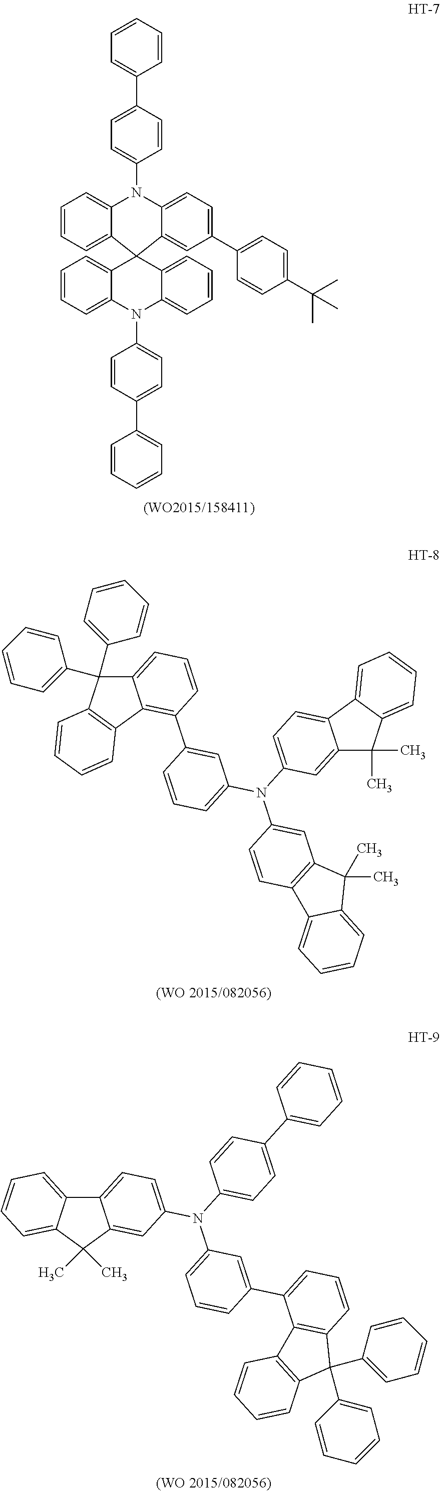 [SMILES: CC(C)(C)c1ccc(-c2ccc3c(c2)C2(c4ccccc4N(c4ccc(-c5ccccc5)cc4)c4ccccc42)c2ccccc2N3c2ccc(-c3ccccc3)cc2)cc1 ; CC1(C)c2ccccc2-c2ccc(N(c3cccc(-c4cccc5c4-c4ccccc4C5(c4ccccc4)c4ccccc4)c3)c3ccc4c(c3)C(C)(C)c3ccccc3-4)cc21 ; CC1(C)c2ccccc2-c2ccc(N(c3ccc(-c4ccccc4)cc3)c3cccc(-c4cccc5c4-c4ccccc4C5(c4ccccc4)c4ccccc4)c3)cc21]

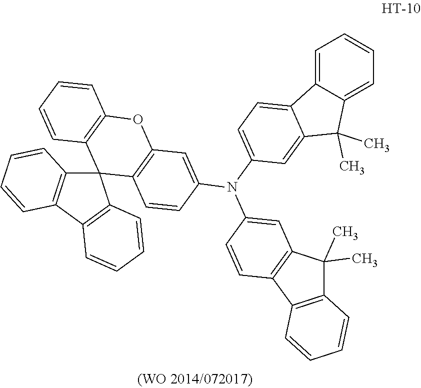 [SMILES: CC1(C)c2ccccc2-c2ccc(N(c3ccc4c(c3)Oc3ccccc3C43c4ccccc4-c4ccccc43)c3ccc4c(c3)C(C)(C)c3ccccc3-4)cc21]

The compounds HT-1 to HT-10 are generally of good suitability for the abovementioned uses in OLEDs of any design and composition, not just in OLEDs according to the present application. Processes for preparing these compounds and the further relevant disclosure relating to the use of these compounds are disclosed in the published specifications that are each cited in brackets in the table beneath the respective compounds. The compounds show good performance data in OLEDs, especially good lifetime and good efficiency.

Preferred cathodes of the electronic device are metals having a low work function, metal alloys or multilayer structures composed of various metals, for example alkaline earth metals, alkali metals, main group metals or lanthanoids (e.g. Ca, Ba, Mg, Al, In, Mg, Yb, Sm, etc.). Additionally suitable are alloys composed of an alkali metal or alkaline earth metal and silver, for example an alloy composed of magnesium and silver. In the case of multilayer structures, in addition to the metals mentioned, it is also possible to use further metals having a relatively high work function, for example Ag or Al, in which case combinations of the metals such as Ca/Ag, Mg/Ag or Ba/Ag, for example, are generally used. It may also be preferable to introduce a thin interlayer of a material having a high dielectric constant between a metallic cathode and the organic semiconductor. Examples of useful materials for this purpose are alkali metal or alkaline earth metal fluorides, but also the corresponding oxides or carbonates (e.g. LiF, Li₂O, BaF₂, MgO, NaF, CsF, Cs₂CO₃, etc.). It is also possible to use lithium quinolinate (LiQ) for this purpose. The layer thickness of this layer is preferably between 0.5 and 5 nm.

Preferred anodes are materials having a high work function. Preferably, the anode has a work function of greater than 4.5 eV versus vacuum. Firstly, metals having a high redox potential are suitable for this purpose, for example Ag, Pt or Au. Secondly, metal/metal oxide electrodes (e.g. Al/Ni/NiOₓ, Al/PtOₓ) may also be preferred. For some applications, at least one of the electrodes has to be transparent or partly transparent in order to enable either the irradiation of the organic material (organic solar cell) or the emission of light (OLED, O-LASER). Preferred anode materials here are conductive mixed metal oxides. Particular preference is given to indium tin oxide (ITO) or indium zinc oxide (IZO). Preference is further given to conductive doped organic materials, especially conductive doped polymers. In addition, the anode may also consist of two or more layers, for example of an inner layer of ITO and an outer layer of a metal oxide, preferably tungsten oxide, molybdenum oxide or vanadium oxide.

In a preferred embodiment, the electronic device is characterized in that one or more layers are coated by a sublimation process. In this case, the materials are applied by vapour deposition in vacuum sublimation systems at an initial pressure of less than 10-5 mbar, preferably less than 10⁻⁶ mbar. In this case, however, it is also possible that the initial pressure is even lower, for example less than 10-7 mbar.

Preference is likewise given to an electronic device, characterized in that one or more layers are coated by the OVPD (organic vapour phase deposition) method or with the aid of a carrier gas sublimation. In this case, the materials are applied at a pressure between 10-5 mbar and 1 bar. A special case of this method is the OVJP (organic vapour jet printing) method, in which the materials are applied directly by a nozzle and thus structured (for example M. S. Arnold et al., Appl. Phys. Lett. 2008, 92, 053301).

Preference is additionally given to an electronic device, characterized in that one or more layers are produced from solution, for example by spin-coating, or by any printing method, for example screen printing, flexographic printing, nozzle printing or offset printing, but more preferably LITI (light-induced thermal imaging, thermal transfer printing) or inkjet printing. For this purpose, soluble compounds of formula (I) are needed. High solubility can be achieved by suitable substitution of the compounds.

It is further preferable that an electronic device of the invention is produced by applying one or more layers from solution and one or more layers by a sublimation method.

After application of the layers, according to the use, the device is structured, contact-connected and finally sealed, in order to rule out damaging effects of water and air.

According to the invention, the electronic devices comprising one or more compounds of formula (I) can be used in displays, as light sources in lighting applications and as light sources in medical and/or cosmetic applications.

### EXAMPLES

### A) Synthesis Examples

### 1,3-Diphenyl-8H-indeno[1,2-c]thiophen-8-one 1a

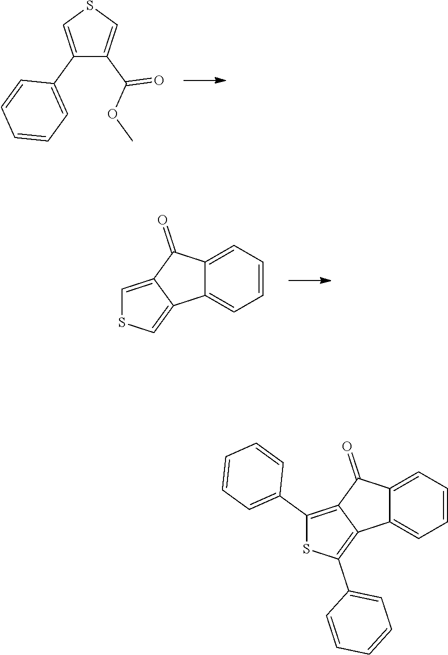 [SMILES: COC(=O)c1cscc1-c1ccccc1 ; O=C1c2ccccc2-c2cscc21 ; O=C1c2ccccc2-c2c(-c3ccccc3)sc(-c3ccccc3)c21]

Methyl 4-phenylthiophene-3-carboxylate (59.50 g, 218 mmol) is suspended in 478.48 ml of trifluoromethanesulfonic acid and stirred at room temperature overnight. The reaction mixture is poured onto ice-water. A yellow solid precipitates out. The precipitated solid is filtered off with suction and dissolved in EtOAc, and extracted by shaking first with NaHCO₃ solution and then with water. The organic phase is dried and concentrated (49.7 g, 98% yield).

An initial charge of 4A molecular sieve is heated under reduced pressure, 8H-indeno[1,2-c]thiophen-8-one (44.60 g, 239.5 mmol), Cs₂CO₃ (234.09 g, 718.5 mmol), John-Phos (7.15 g, 23.9 mmol), palladium(II) acetate (2.69 g, 11.9 mmol), dried N,N-dimethylformamide (669.00 ml) and bromobenzene (75.71 ml, 718.5 mmol) are added, and the mixture is stirred at 150° C. overnight. The mixture is then left to cool down to room temperature and admixed with water. The precipitated solid is filtered off with suction and washed with heptane (70 g, 85% yield).

The following compounds are prepared in an analogous manner:

| Ex. | Ester | Fluorenone | Product | Yield |
|---|---|---|---|---|
| 1b | 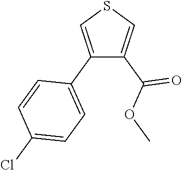 [SMILES: COC(=O)c1cscc1-c1ccc(Cl)cc1] | 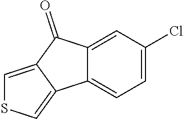 [SMILES: O=C1c2cc(Cl)ccc2-c2cscc21] | 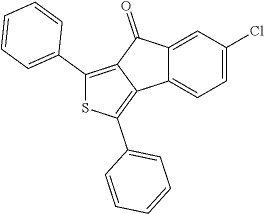 [SMILES: O=C1c2cc(Cl)ccc2-c2c(-c3ccccc3)sc(-c3ccccc3)c21] | 66% |
| 1c | 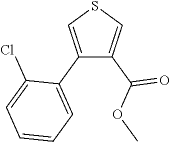 [SMILES: COC(=O)c1cscc1-c1ccccc1Cl] | 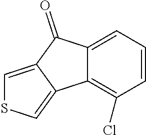 [SMILES: O=C1c2cscc2-c2c(Cl)cccc21] | 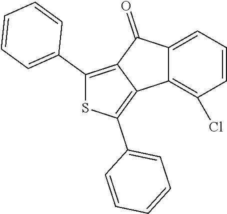 [SMILES: O=C1c2cccc(Cl)c2-c2c(-c3ccccc3)sc(-c3ccccc3)c21] | 54% |
| 1d | 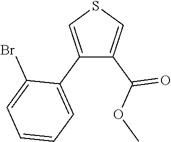 [SMILES: COC(=O)c1cscc1-c1ccccc1Br] | 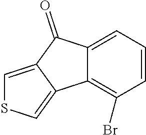 [SMILES: O=C1c2cscc2-c2c(Br)cccc21] | 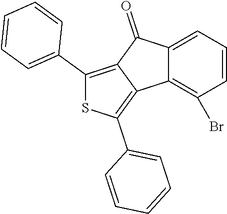 [SMILES: O=C1c2cccc(Br)c2-c2c(-c3ccccc3)sc(-c3ccccc3)c21] | 60% |
| 1f | 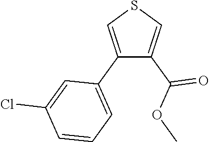 [SMILES: COC(=O)c1cscc1-c1cccc(Cl)c1] | 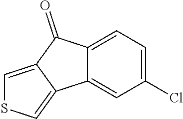 [SMILES: O=C1c2cscc2-c2cc(Cl)ccc21] | 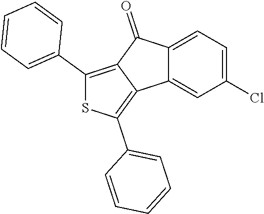 [SMILES: O=C1c2ccc(Cl)cc2-c2c(-c3ccccc3)sc(-c3ccccc3)c21] | 70% |
| 1g | 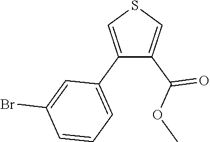 [SMILES: COC(=O)c1cscc1-c1cccc(Br)c1] | 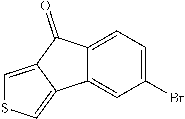 [SMILES: O=C1c2cscc2-c2cc(Br)ccc21] | 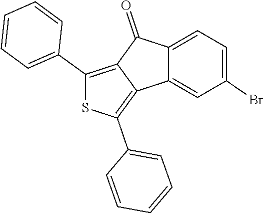 [SMILES: O=C1c2ccc(Br)cc2-c2c(-c3ccccc3)sc(-c3ccccc3)c21] | 66% |
| 1h | 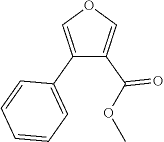 | 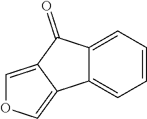 [SMILES: O=C1c2ccccc2-c2cocc21] | 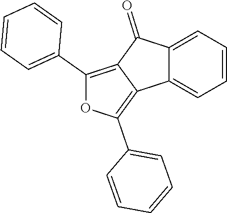 [SMILES: O=C1c2ccccc2-c2c(-c3ccccc3)oc(-c3ccccc3)c21] | 51% |
| 1i | 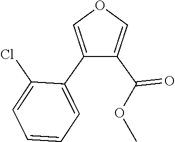 [SMILES: COC(=O)c1cocc1-c1ccccc1Cl] | 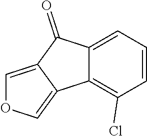 [SMILES: O=C1c2cocc2-c2c(Cl)cccc21] | 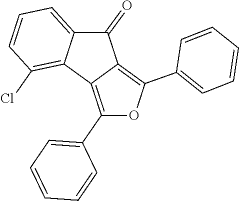 [SMILES: O=C1c2cccc(Cl)c2-c2c(-c3ccccc3)oc(-c3ccccc3)c21] | 57% |
| 1j | 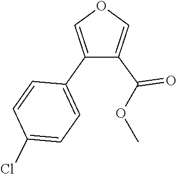 [SMILES: COC(=O)c1cocc1-c1ccc(Cl)cc1] | 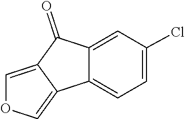 [SMILES: O=C1c2cc(Cl)ccc2-c2cocc21] | 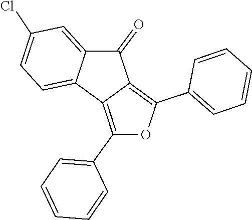 [SMILES: O=C1c2cc(Cl)ccc2-c2c(-c3ccccc3)oc(-c3ccccc3)c21] | 55% |
| 1k | 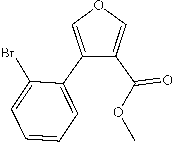 [SMILES: COC(=O)c1cocc1-c1ccccc1Br] | 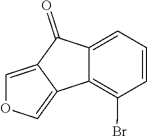 [SMILES: O=C1c2cocc2-c2c(Br)cccc21] | 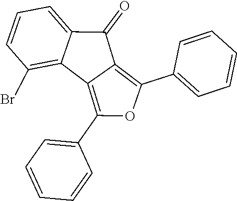 [SMILES: O=C1c2cccc(Br)c2-c2c(-c3ccccc3)oc(-c3ccccc3)c21] | 51% |
| 1l | 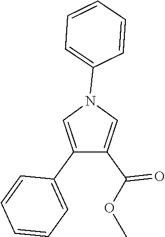 [SMILES: COC(=O)c1cn(-c2ccccc2)cc1-c1ccccc1] | 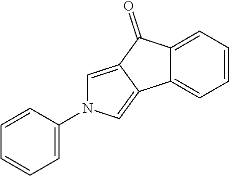 [SMILES: O=C1c2ccccc2-c2cn(-c3ccccc3)cc21] | 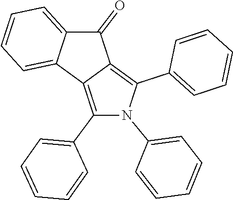 [SMILES: O=C1c2ccccc2-c2c1c(-c1ccccc1)n(-c1ccccc1)c2-c1ccccc1] | 50% |
| 1m | 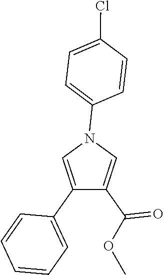 [SMILES: COC(=O)c1cn(-c2ccc(Cl)cc2)cc1-c1ccccc1] | 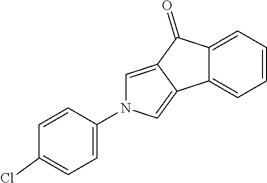 [SMILES: O=C1c2ccccc2-c2cn(-c3ccc(Cl)cc3)cc21] | 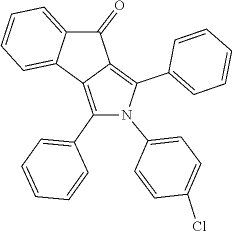 [SMILES: O=C1c2ccccc2-c2c1c(-c1ccccc1)n(-c1ccc(Cl)cc1)c2-c1ccccc1] | 56% |
| 1n | 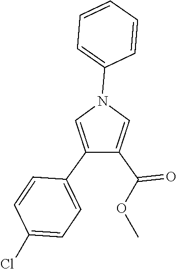 [SMILES: COC(=O)c1cn(-c2ccccc2)cc1-c1ccc(Cl)cc1] | 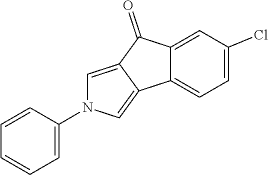 [SMILES: O=C1c2cc(Cl)ccc2-c2cn(-c3ccccc3)cc21] | 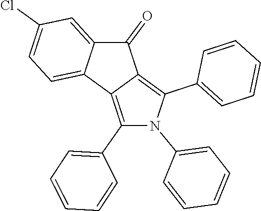 [SMILES: O=C1c2cc(Cl)ccc2-c2c1c(-c1ccccc1)n(-c1ccccc1)c2-c1ccccc1] | 47% |

### 7-Chloro-1′,3′-diphenylspiro[fluorene-9,8′-indeno[1,2-c]thiophene]2a

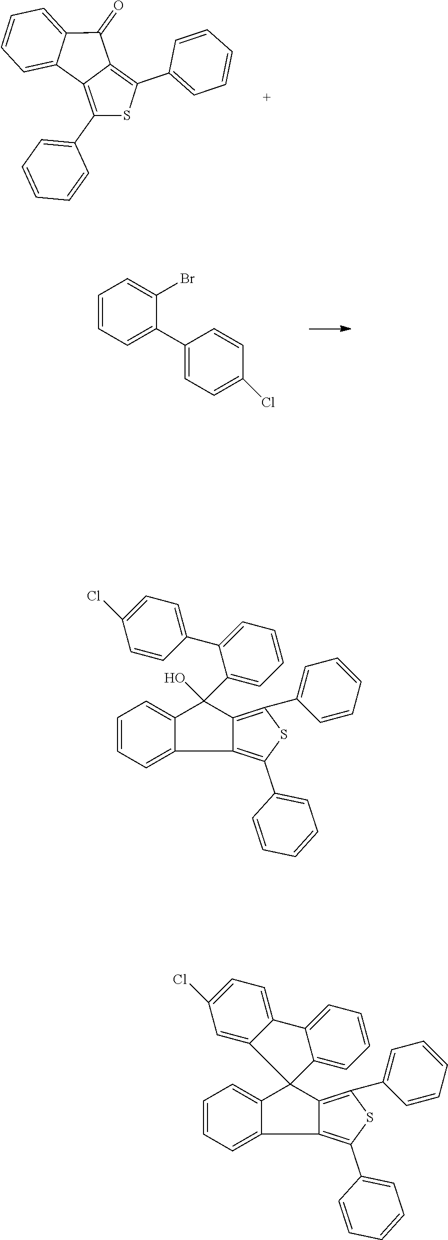 [SMILES: O=C1c2ccccc2-c2c(-c3ccccc3)sc(-c3ccccc3)c21 ; Clc1ccc(-c2ccccc2Br)cc1 ; OC1(c2ccccc2-c2ccc(Cl)cc2)c2ccccc2-c2c(-c3ccccc3)sc(-c3ccccc3)c21 ; Clc1ccc2c(c1)C1(c3ccccc3-2)c2ccccc2-c2c(-c3ccccc3)sc(-c3ccccc3)c21]

28.5 g (106.4 mmol) of 2-bromo-4′-chloro-1,1′-biphenyl is dissolved in 280 ml of dried THE in a baked-out flask. The reaction mixture is cooled to −78° C. At this temperature, 39 ml of a 2.5 M solution of n-BuLi in hexane (97.5 mmol) is slowly added dropwise. The mixture is stirred at −70° C. for a further 1 hour. Subsequently, 30 g of 1,3-diphenyl-8H-indeno[1,2-c]thiophen-8-one (88.6 mmol) is dissolved in 145 ml of THE and added dropwise at −70° C. After the addition has ended, the reaction mixture is left to warm up gradually to room temperature, the reaction is stopped with NH₄Cl, and then the mixture is concentrated on a rotary evaporator. The solid material is dissolved in 380 ml of toluene, and then 1.7 g (8.9 mmol) of p-toluenesulfonic acid is added. The mixture is heated under reflux for 6 hours, then allowed to cool down to room temperature and admixed with water. The precipitated solid is filtered off with suction and washed with heptane (30.6 g, 67% yield).

The following compounds are prepared in an analogous manner:

| Ex. | Fluorenone | Halogen | Product | Yield |
|---|---|---|---|---|
| 2b | 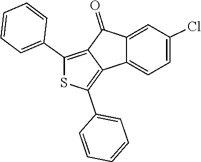 [SMILES: O=C1c2cc(Cl)ccc2-c2c(-c3ccccc3)sc(-c3ccccc3)c21] | 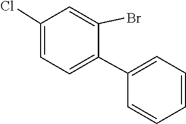 [SMILES: Clc1ccc(-c2ccccc2)c(Br)c1] | 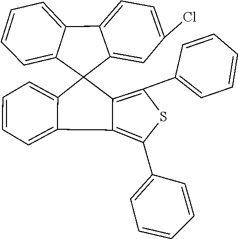 [SMILES: Clc1ccc2c(c1)C1(c3ccccc3-2)c2ccccc2-c2c(-c3ccccc3)sc(-c3ccccc3)c21] | 81% |
| 2c | 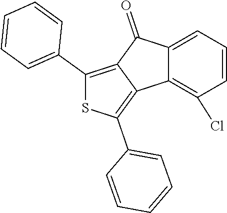 [SMILES: O=C1c2cccc(Cl)c2-c2c(-c3ccccc3)sc(-c3ccccc3)c21] | 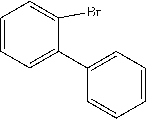 [SMILES: Brc1ccccc1-c1ccccc1] | 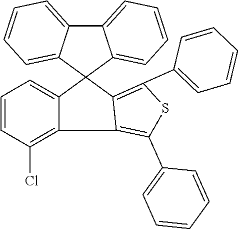 [SMILES: Clc1cccc2c1-c1c(-c3ccccc3)sc(-c3ccccc3)c1C21c2ccccc2-c2ccccc21] | 72% |
| 2d | 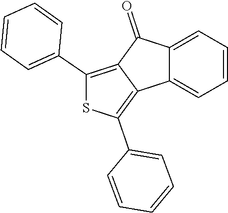 [SMILES: O=C1c2ccccc2-c2c(-c3ccccc3)sc(-c3ccccc3)c21] | 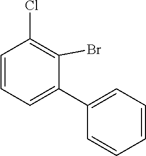 [SMILES: Clc1cccc(-c2ccccc2)c1Br] | 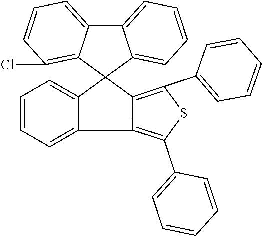 [SMILES: Clc1cccc2c1C1(c3ccccc3-2)c2ccccc2-c2c(-c3ccccc3)sc(-c3ccccc3)c21] | 33% |
| 2e | 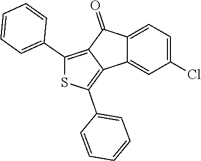 [SMILES: O=C1c2ccc(Cl)cc2-c2c(-c3ccccc3)sc(-c3ccccc3)c21] | 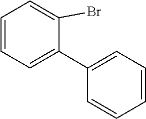 [SMILES: Brc1ccccc1-c1ccccc1] | 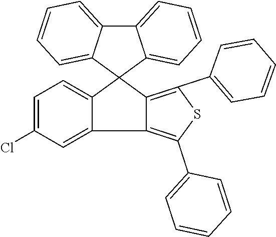 [SMILES: Clc1ccc2c(c1)-c1c(-c3ccccc3)sc(-c3ccccc3)c1C21c2ccccc2-c2ccccc21] | 82% |
| 2f | 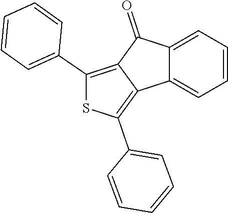 [SMILES: O=C1c2ccccc2-c2c(-c3ccccc3)sc(-c3ccccc3)c21] | 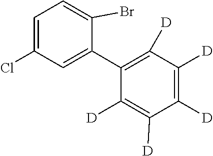 [SMILES: [2H]c1c([2H])c([2H])c(-c2cc(Cl)ccc2Br)c([2H])c1[2H]] | 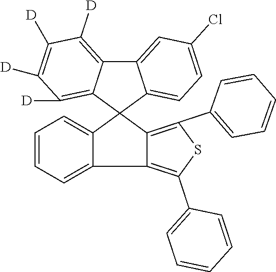 [SMILES: [2H]c1c([2H])c([2H])c2c(c1[2H])-c1cc(Cl)ccc1C21c2ccccc2-c2c(-c3ccccc3)sc(-c3ccccc3)c21] | 80% |
| 2g | 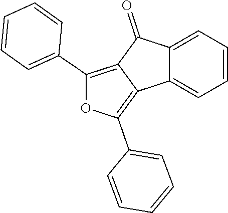 [SMILES: O=C1c2ccccc2-c2c(-c3ccccc3)oc(-c3ccccc3)c21] | 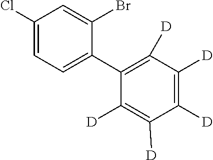 [SMILES: [2H]c1c([2H])c([2H])c(-c2ccc(Cl)cc2Br)c([2H])c1[2H]] | 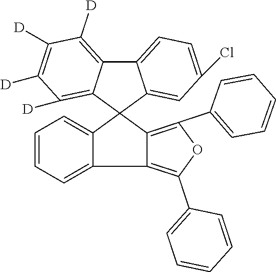 [SMILES: [2H]c1c([2H])c([2H])c2c(c1[2H])-c1ccc(Cl)cc1C21c2ccccc2-c2c(-c3ccccc3)oc(-c3ccccc3)c21] | 75% |
| 2h | 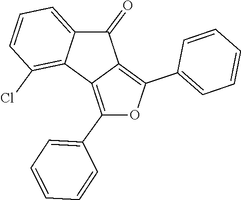 [SMILES: O=C1c2cccc(Cl)c2-c2c(-c3ccccc3)oc(-c3ccccc3)c21] | 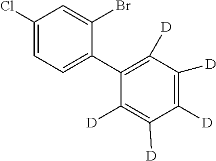 [SMILES: [2H]c1c([2H])c([2H])c(-c2ccc(Cl)cc2Br)c([2H])c1[2H]] | 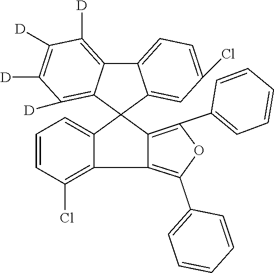 [SMILES: [2H]c1c([2H])c([2H])c2c(c1[2H])-c1ccc(Cl)cc1C21c2cccc(Cl)c2-c2c(-c3ccccc3)oc(-c3ccccc3)c21] | 75% |
| 2i | 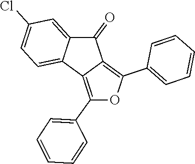 [SMILES: O=C1c2cc(Cl)ccc2-c2c(-c3ccccc3)oc(-c3ccccc3)c21] | 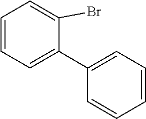 [SMILES: Brc1ccccc1-c1ccccc1] | 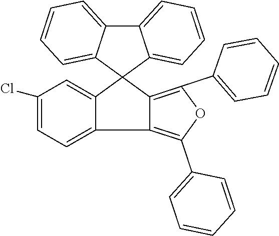 [SMILES: Clc1ccc2c(c1)C1(c3ccccc3-c3ccccc31)c1c(-c3ccccc3)oc(-c3ccccc3)c1-2] | 64% |
| 2j | 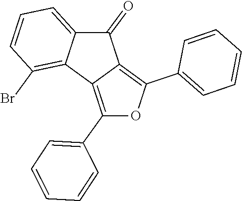 [SMILES: O=C1c2cccc(Br)c2-c2c(-c3ccccc3)oc(-c3ccccc3)c21] | 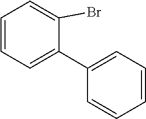 [SMILES: Brc1ccccc1-c1ccccc1] | 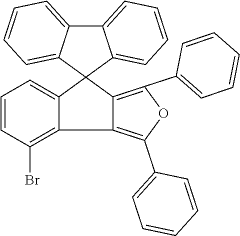 [SMILES: Brc1cccc2c1-c1c(-c3ccccc3)oc(-c3ccccc3)c1C21c2ccccc2-c2ccccc21] | 60% |
| 2k | 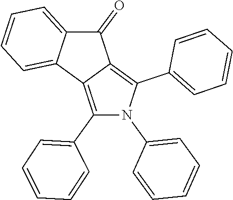 [SMILES: O=C1c2ccccc2-c2c1c(-c1ccccc1)n(-c1ccccc1)c2-c1ccccc1] | 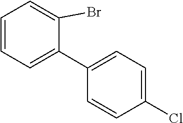 [SMILES: Clc1ccc(-c2ccccc2Br)cc1] | 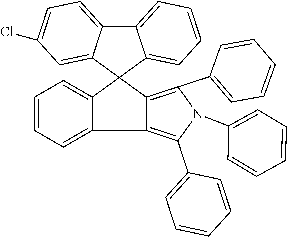 [SMILES: Clc1ccc2c(c1)C1(c3ccccc3-2)c2ccccc2-c2c1c(-c1ccccc1)n(-c1ccccc1)c2-c1ccccc1] | 70% |
| 2l | 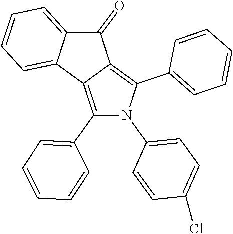 [SMILES: O=C1c2ccccc2-c2c1c(-c1ccccc1)n(-c1ccc(Cl)cc1)c2-c1ccccc1] | 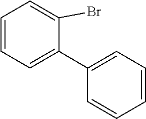 [SMILES: Brc1ccccc1-c1ccccc1] | 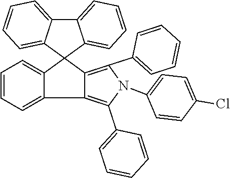 [SMILES: Clc1ccc(-n2c(-c3ccccc3)c3c(c2-c2ccccc2)C2(c4ccccc4-c4ccccc42)c2ccccc2-3)cc1] | 80% |
| 2m | 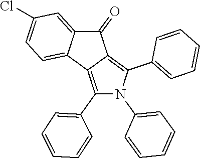 [SMILES: O=C1c2cc(Cl)ccc2-c2c1c(-c1ccccc1)n(-c1ccccc1)c2-c1ccccc1] | 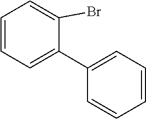 [SMILES: Brc1ccccc1-c1ccccc1] | 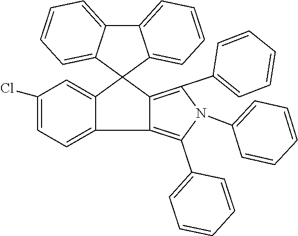 [SMILES: Clc1ccc2c(c1)C1(c3ccccc3-c3ccccc31)c1c-2c(-c2ccccc2)n(-c2ccccc2)c1-c1ccccc1] | 75% |
| 2n | 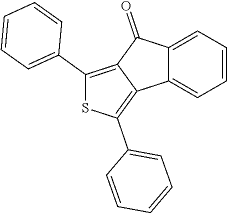 [SMILES: O=C1c2ccccc2-c2c(-c3ccccc3)sc(-c3ccccc3)c21] | 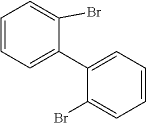 [SMILES: Brc1ccccc1-c1ccccc1Br] | 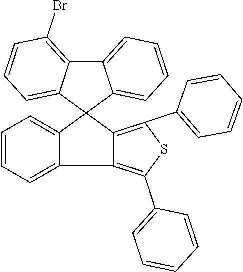 [SMILES: Brc1cccc2c1-c1ccccc1C21c2ccccc2-c2c(-c3ccccc3)sc(-c3ccccc3)c21] | 80% |
| 2o | 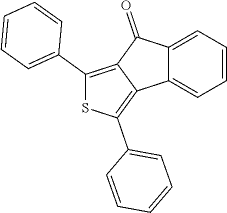 [SMILES: O=C1c2ccccc2-c2c(-c3ccccc3)sc(-c3ccccc3)c21] | 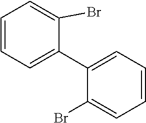 [SMILES: Brc1ccccc1-c1ccccc1Br] | 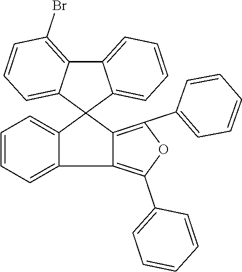 [SMILES: Brc1cccc2c1-c1ccccc1C21c2ccccc2-c2c(-c3ccccc3)oc(-c3ccccc3)c21] | 77% |
| 2p | 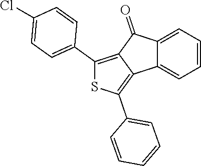 [SMILES: O=C1c2ccccc2-c2c(-c3ccccc3)sc(-c3ccc(Cl)cc3)c21] | 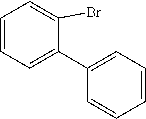 [SMILES: Brc1ccccc1-c1ccccc1] | 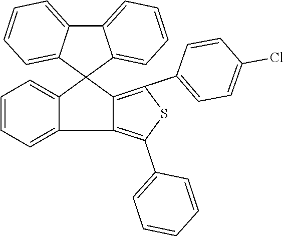 [SMILES: Clc1ccc(-c2sc(-c3ccccc3)c3c2C2(c4ccccc4-c4ccccc42)c2ccccc2-3)cc1] | 82% |
| 2q | 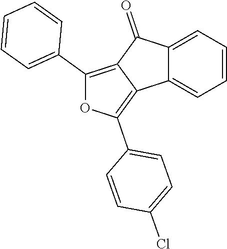 [SMILES: O=C1c2ccccc2-c2c(-c3ccc(Cl)cc3)oc(-c3ccccc3)c21] | 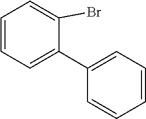 [SMILES: Brc1ccccc1-c1ccccc1] | 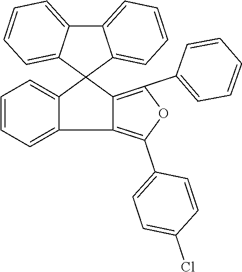 [SMILES: Clc1ccc(-c2oc(-c3ccccc3)c3c2-c2ccccc2C32c3ccccc3-c3ccccc32)cc1] | 84% |
| 2r | 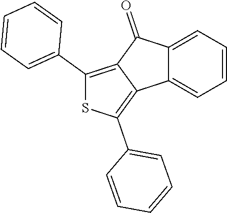 [SMILES: O=C1c2ccccc2-c2c(-c3ccccc3)sc(-c3ccccc3)c21] | 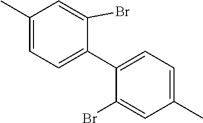 | 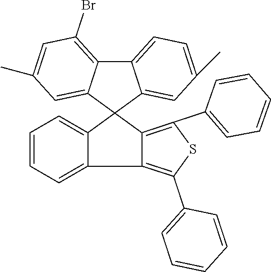 [SMILES: Cc1ccc2c(c1)C1(c3ccccc3-c3c(-c4ccccc4)sc(-c4ccccc4)c31)c1cc(C)cc(Br)c1-2] | 74% |
| 2s | 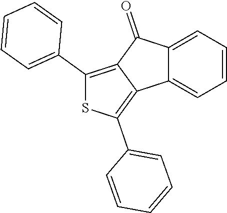 [SMILES: O=C1c2ccccc2-c2c(-c3ccccc3)sc(-c3ccccc3)c21] | 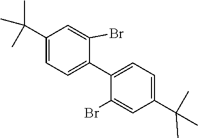 [SMILES: CC(C)(C)c1ccc(-c2ccc(C(C)(C)C)cc2Br)c(Br)c1] | 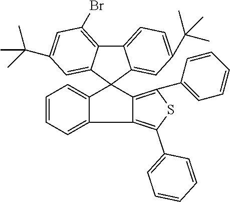 [SMILES: CC(C)(C)c1ccc2c(c1)C1(c3ccccc3-c3c(-c4ccccc4)sc(-c4ccccc4)c31)c1cc(C(C)(C)C)cc(Br)c1-2] | 70% |
| 2t | 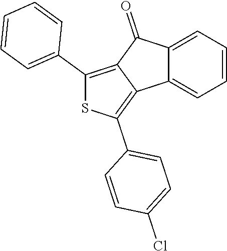 [SMILES: O=C1c2ccccc2-c2c(-c3ccc(Cl)cc3)sc(-c3ccccc3)c21] | 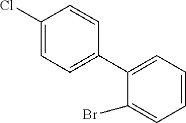 [SMILES: Clc1ccc(-c2ccccc2Br)cc1] | 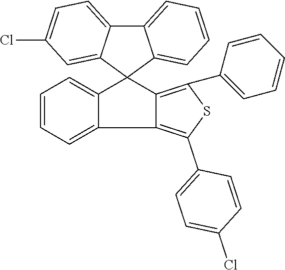 [SMILES: Clc1ccc(-c2sc(-c3ccccc3)c3c2-c2ccccc2C32c3ccccc3-c3ccc(Cl)cc32)cc1] | 68% |
| 2u | 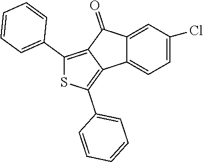 [SMILES: O=C1c2cc(Cl)ccc2-c2c(-c3ccccc3)sc(-c3ccccc3)c21] | 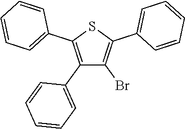 [SMILES: Brc1c(-c2ccccc2)sc(-c2ccccc2)c1-c1ccccc1] | 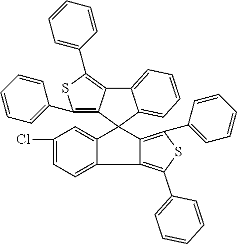 [SMILES: Clc1ccc2c(c1)C1(c3ccccc3-c3c(-c4ccccc4)sc(-c4ccccc4)c31)c1c(-c3ccccc3)sc(-c3ccccc3)c1-2] | 65% |
| 2v | 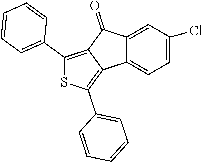 [SMILES: O=C1c2cc(Cl)ccc2-c2c(-c3ccccc3)sc(-c3ccccc3)c21] | 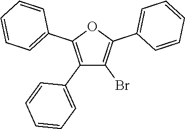 [SMILES: Brc1c(-c2ccccc2)oc(-c2ccccc2)c1-c1ccccc1] | 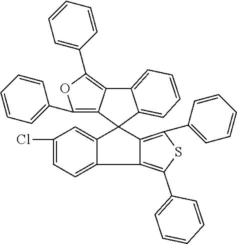 [SMILES: Clc1ccc2c(c1)C1(c3ccccc3-c3c(-c4ccccc4)oc(-c4ccccc4)c31)c1c(-c3ccccc3)sc(-c3ccccc3)c1-2] | 67% |
| 2x | 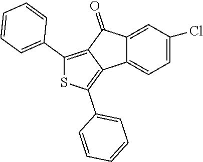 [SMILES: O=C1c2cc(Cl)ccc2-c2c(-c3ccccc3)sc(-c3ccccc3)c21] | 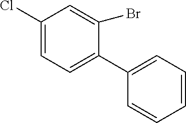 [SMILES: Clc1ccc(-c2ccccc2)c(Br)c1] | 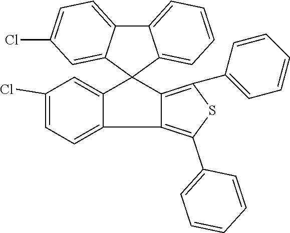 [SMILES: Clc1ccc2c(c1)C1(c3ccccc3-2)c2cc(Cl)ccc2-c2c(-c3ccccc3)sc(-c3ccccc3)c21] | 75% |
| 2y | 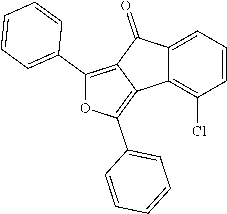 [SMILES: O=C1c2cccc(Cl)c2-c2c(-c3ccccc3)oc(-c3ccccc3)c21] | 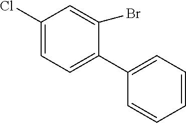 [SMILES: Clc1ccc(-c2ccccc2)c(Br)c1] | 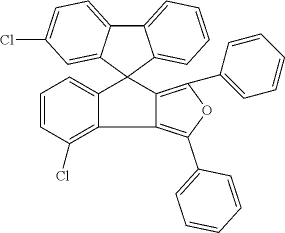 [SMILES: Clc1ccc2c(c1)C1(c3ccccc3-2)c2cccc(Cl)c2-c2c(-c3ccccc3)oc(-c3ccccc3)c21] | 80% |
| 2z | 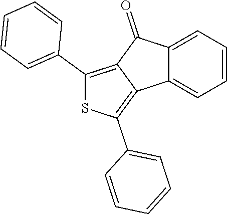 [SMILES: O=C1c2ccccc2-c2c(-c3ccccc3)sc(-c3ccccc3)c21] | 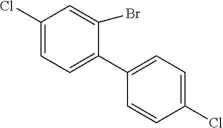 [SMILES: Clc1ccc(-c2ccc(Cl)cc2Br)cc1] | 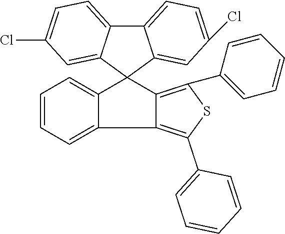 [SMILES: Clc1ccc2c(c1)C1(c3cc(Cl)ccc3-2)c2ccccc2-c2c(-c3ccccc3)sc(-c3ccccc3)c21] | 76% |
| 2aa | 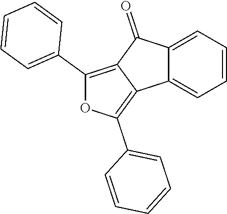 [SMILES: O=C1c2ccccc2-c2c(-c3ccccc3)oc(-c3ccccc3)c21] | 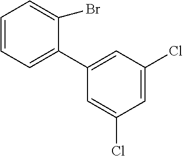 [SMILES: Clc1cc(Cl)cc(-c2ccccc2Br)c1] | 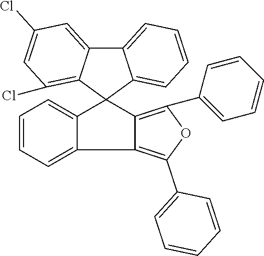 [SMILES: Clc1cc(Cl)c2c(c1)-c1ccccc1C21c2ccccc2-c2c(-c3ccccc3)oc(-c3ccccc3)c21] | 70% |
| 2ab | 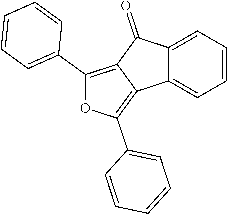 [SMILES: O=C1c2ccccc2-c2c(-c3ccccc3)oc(-c3ccccc3)c21] | 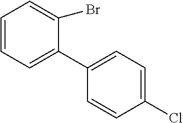 [SMILES: Clc1ccc(-c2ccccc2Br)cc1] | 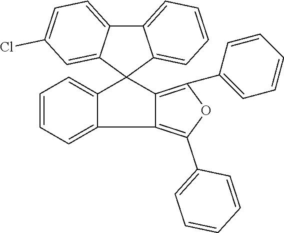 [SMILES: Clc1ccc2c(c1)C1(c3ccccc3-2)c2ccccc2-c2c(-c3ccccc3)oc(-c3ccccc3)c21] | 75% |
| 2ac | 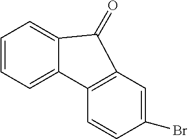 [SMILES: O=C1c2ccccc2-c2ccc(Br)cc21] | 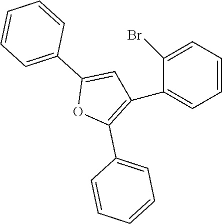 [SMILES: Brc1ccccc1-c1cc(-c2ccccc2)oc1-c1ccccc1] | 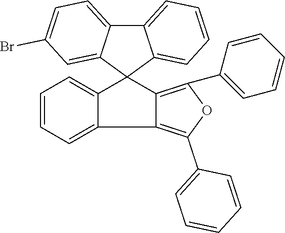 [SMILES: Brc1ccc2c(c1)C1(c3ccccc3-2)c2ccccc2-c2c(-c3ccccc3)oc(-c3ccccc3)c21] | 60% |
| 2ad | 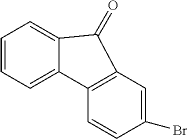 [SMILES: O=C1c2ccccc2-c2ccc(Br)cc21] | 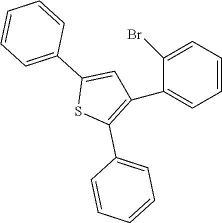 [SMILES: Brc1ccccc1-c1cc(-c2ccccc2)sc1-c1ccccc1] | 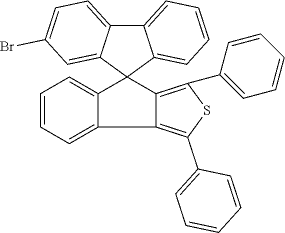 [SMILES: Brc1ccc2c(c1)C1(c3ccccc3-2)c2ccccc2-c2c(-c3ccccc3)sc(-c3ccccc3)c21] | 70% |

### N-{[1,1′-Biphenyl]-2-yl}-N-(9,9-dimethyl-9H-fluoren-2-yl)-1′,3′-diphenyispiro[fluorene-9,8′-indeno[1,2-c]thiophene]-2-amine 3a

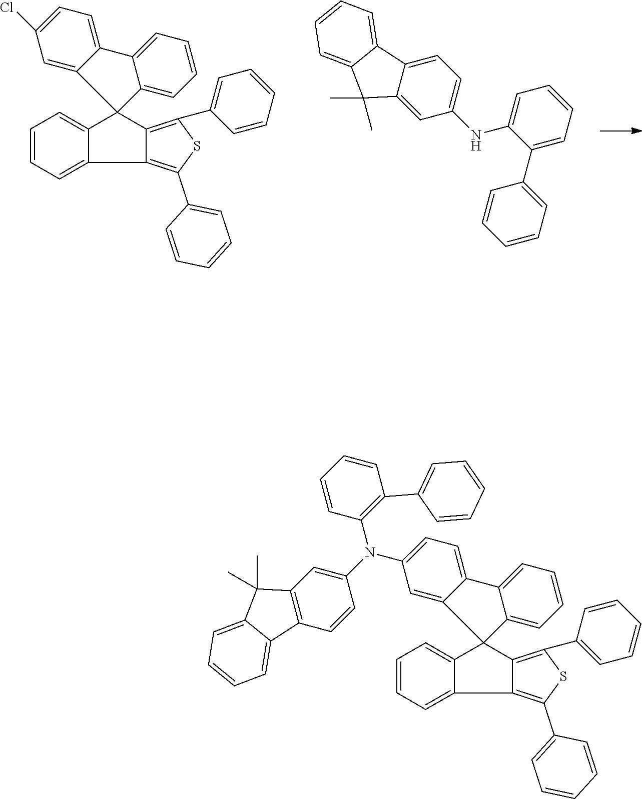 [SMILES: Clc1ccc2c(c1)C1(c3ccccc3-2)c2ccccc2-c2c(-c3ccccc3)sc(-c3ccccc3)c21 ; CC1(C)c2ccccc2-c2ccc(Nc3ccccc3-c3ccccc3)cc21 ; CC1(C)c2ccccc2-c2ccc(N(c3ccc4c(c3)C3(c5ccccc5-4)c4ccccc4-c4c(-c5ccccc5)sc(-c5ccccc5)c43)c3ccccc3-c3ccccc3)cc21]

10.6 g of N-{1,1′-biphenyl]-2-yl}-9,9-dimethylfluoren-2-amine (29.3 mmol) and 14.5 g of 2-chloro-1′,3′-diphenylspiro[fluorene-9,8′-indeno[1,2-c]thiophene] (28.5 mol) are dissolved in 250 ml of toluene. The solution is degassed and saturated with N2. Thereafter, 1 g (5.1 mmol) of S-Phos and 1.6 g (1.7 mmol) of Pd₂(dba)₃ are added thereto, and then 4.1 g of sodium tert-butoxide (42.7 mmol) is added. The reaction mixture is heated to boiling under a protective atmosphere overnight. The mixture is subsequently partitioned between toluene and water, and the organic phase is washed three times with water and dried over Na₂SO₄ and concentrated by rotary evaporation. After the crude product has been filtered through silica gel with toluene, the remaining residue is recrystallized from heptane/toluene. The substance is finally sublimed under high vacuum; purity is 99.9%. The yield is 8 g (30% of theory).

The following compounds are prepared in an analogous manner:

| Ex. | Spiro | Amine | Product | Yield |
|---|---|---|---|---|
| 3b | 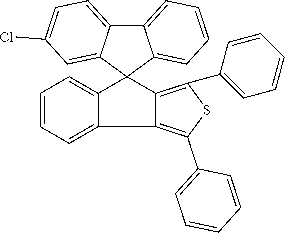 [SMILES: Clc1ccc2c(c1)C1(c3ccccc3-2)c2ccccc2-c2c(-c3ccccc3)sc(-c3ccccc3)c21] | 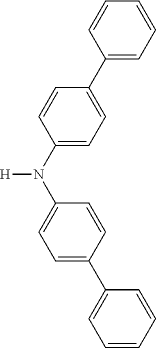 [SMILES: c1ccc(-c2ccc(Nc3ccc(-c4ccccc4)cc3)cc2)cc1] | 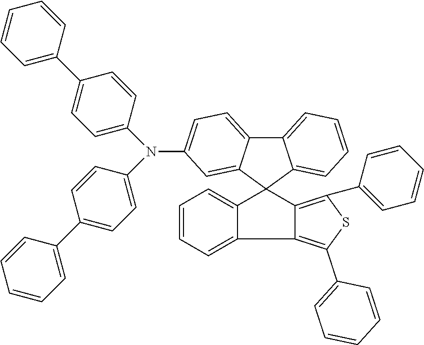 [SMILES: c1ccc(-c2ccc(N(c3ccc(-c4ccccc4)cc3)c3ccc4c(c3)C3(c5ccccc5-4)c4ccccc4-c4c(-c5ccccc5)sc(-c5ccccc5)c43)cc2)cc1] | 53% |
| 3c | 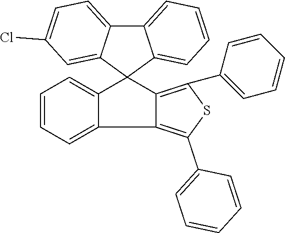 [SMILES: Clc1ccc2c(c1)C1(c3ccccc3-2)c2ccccc2-c2c(-c3ccccc3)sc(-c3ccccc3)c21] | 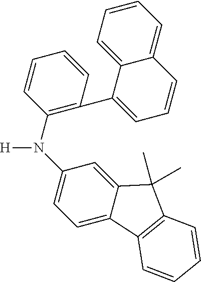 [SMILES: CC1(C)c2ccccc2-c2ccc(Nc3ccccc3-c3cccc4ccccc34)cc21] | 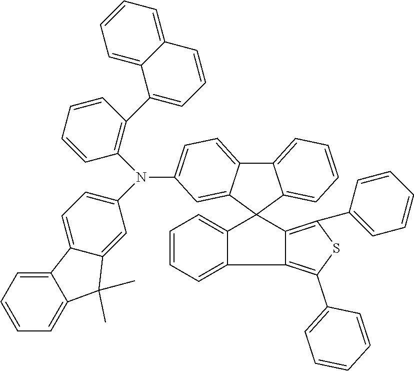 [SMILES: CC1(C)c2ccccc2-c2ccc(N(c3ccc4c(c3)C3(c5ccccc5-4)c4ccccc4-c4c(-c5ccccc5)sc(-c5ccccc5)c43)c3ccccc3-c3cccc4ccccc34)cc21] | 65% |
| 3d | 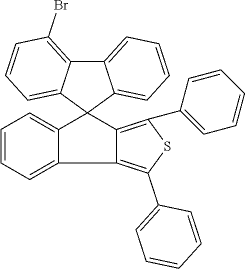 [SMILES: Brc1cccc2c1-c1ccccc1C21c2ccccc2-c2c(-c3ccccc3)sc(-c3ccccc3)c21] | 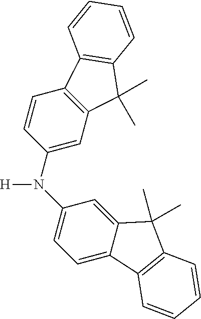 [SMILES: CC1(C)c2ccccc2-c2ccc(Nc3ccc4c(c3)C(C)(C)c3ccccc3-4)cc21] | 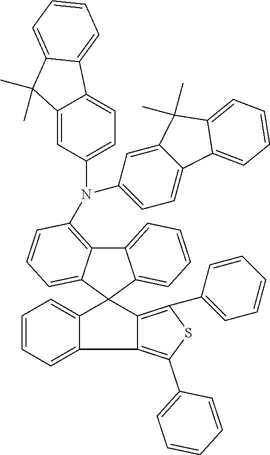 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccccc5)sc(-c5ccccc5)c43)cc21] | 51% |
| 3e | 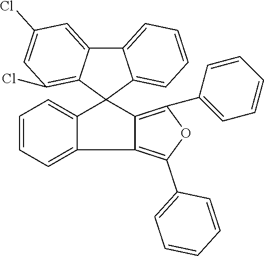 [SMILES: Clc1cc(Cl)c2c(c1)-c1ccccc1C21c2ccccc2-c2c(-c3ccccc3)oc(-c3ccccc3)c21] | 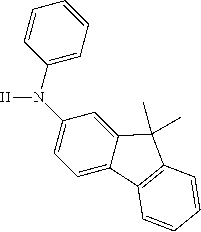 [SMILES: CC1(C)c2ccccc2-c2ccc(Nc3ccccc3)cc21] | 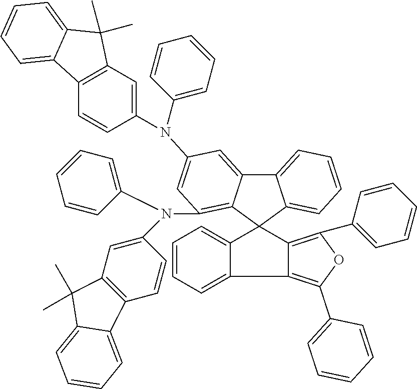 [SMILES: CC1(C)c2ccccc2-c2ccc(N(c3ccccc3)c3cc4c(c(N(c5ccccc5)c5ccc6c(c5)C(C)(C)c5ccccc5-6)c3)C3(c5ccccc5-4)c4ccccc4-c4c(-c5ccccc5)oc(-c5ccccc5)c43)cc21] | 62% |
| 3f | 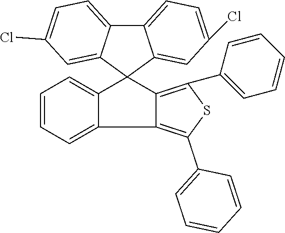 [SMILES: Clc1ccc2c(c1)C1(c3cc(Cl)ccc3-2)c2ccccc2-c2c(-c3ccccc3)sc(-c3ccccc3)c21] | 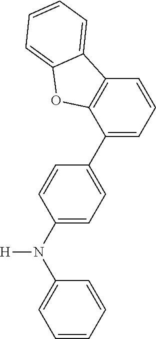 [SMILES: c1ccc(Nc2ccc(-c3cccc4c3oc3ccccc34)cc2)cc1] | 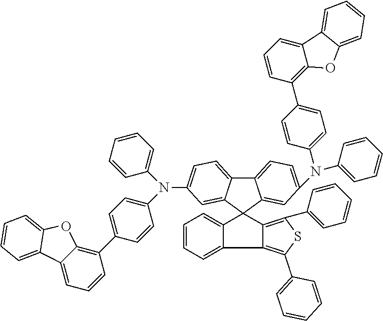 [SMILES: c1ccc(-c2sc(-c3ccccc3)c3c2-c2ccccc2C32c3cc(N(c4ccccc4)c4ccc(-c5cccc6c5oc5ccccc56)cc4)ccc3-c3ccc(N(c4ccccc4)c4ccc(-c5cccc6c5oc5ccccc56)cc4)cc32)cc1] | 58% |
| 3g | 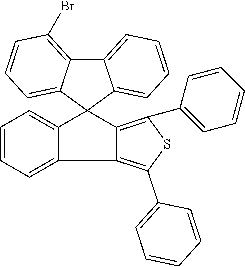 [SMILES: Brc1cccc2c1-c1ccccc1C21c2ccccc2-c2c(-c3ccccc3)sc(-c3ccccc3)c21] | 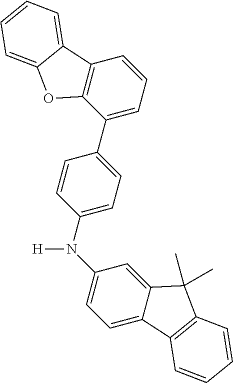 [SMILES: CC1(C)c2ccccc2-c2ccc(Nc3ccc(-c4cccc5c4oc4ccccc45)cc3)cc21] | 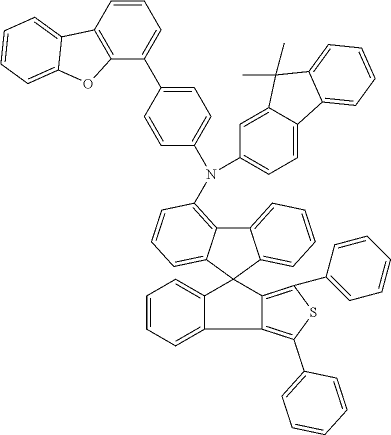 [SMILES: CC1(C)c2ccccc2-c2ccc(N(c3ccc(-c4cccc5c4oc4ccccc45)cc3)c3cccc4c3-c3ccccc3C43c4ccccc4-c4c(-c5ccccc5)sc(-c5ccccc5)c43)cc21] | 63% |
| 3h | 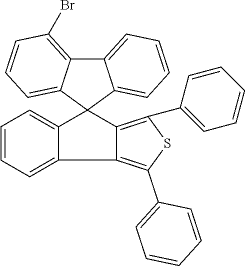 [SMILES: Brc1cccc2c1-c1ccccc1C21c2ccccc2-c2c(-c3ccccc3)sc(-c3ccccc3)c21] | 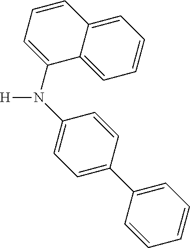 [SMILES: c1ccc(-c2ccc(Nc3cccc4ccccc34)cc2)cc1] | 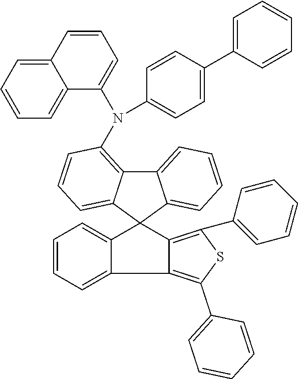 [SMILES: c1ccc(-c2ccc(N(c3cccc4c3-c3ccccc3C43c4ccccc4-c4c(-c5ccccc5)sc(-c5ccccc5)c43)c3cccc4ccccc34)cc2)cc1] | 55% |
| 3i | 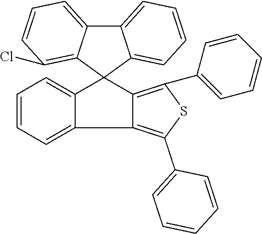 [SMILES: Clc1cccc2c1C1(c3ccccc3-2)c2ccccc2-c2c(-c3ccccc3)sc(-c3ccccc3)c21] | 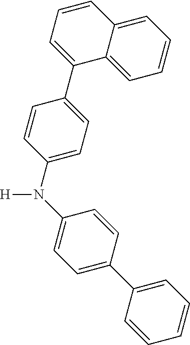 [SMILES: c1ccc(-c2ccc(Nc3ccc(-c4cccc5ccccc45)cc3)cc2)cc1] | 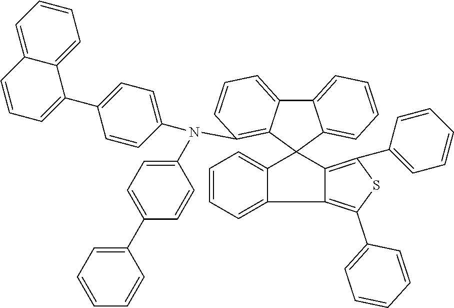 [SMILES: c1ccc(-c2ccc(N(c3ccc(-c4cccc5ccccc45)cc3)c3cccc4c3C3(c5ccccc5-4)c4ccccc4-c4c(-c5ccccc5)sc(-c5ccccc5)c43)cc2)cc1] | 77% |
| 3j | 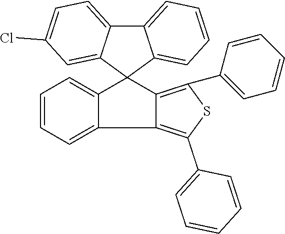 [SMILES: Clc1ccc2c(c1)C1(c3ccccc3-2)c2ccccc2-c2c(-c3ccccc3)sc(-c3ccccc3)c21] | 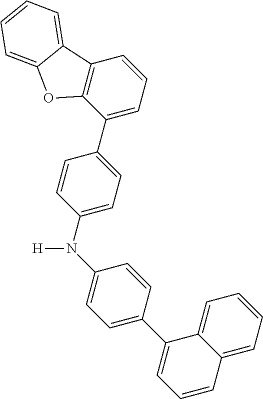 [SMILES: c1ccc2c(-c3ccc(Nc4ccc(-c5cccc6c5oc5ccccc56)cc4)cc3)cccc2c1] | 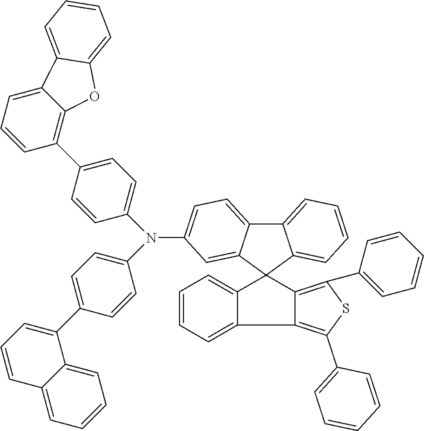 [SMILES: c1ccc(-c2sc(-c3ccccc3)c3c2-c2ccccc2C32c3ccccc3-c3ccc(N(c4ccc(-c5cccc6ccccc56)cc4)c4ccc(-c5cccc6c5oc5ccccc56)cc4)cc32)cc1] | 76% |
| 3k | 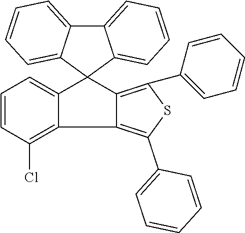 [SMILES: Clc1cccc2c1-c1c(-c3ccccc3)sc(-c3ccccc3)c1C21c2ccccc2-c2ccccc21] | 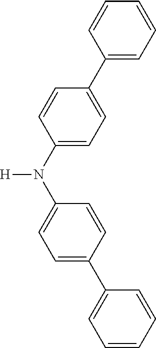 [SMILES: c1ccc(-c2ccc(Nc3ccc(-c4ccccc4)cc3)cc2)cc1] | 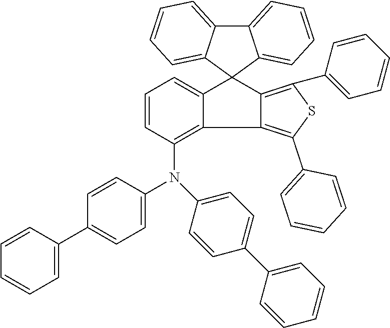 [SMILES: c1ccc(-c2ccc(N(c3ccc(-c4ccccc4)cc3)c3cccc4c3-c3c(-c5ccccc5)sc(-c5ccccc5)c3C43c4ccccc4-c4ccccc43)cc2)cc1] | 65% |
| 3l | 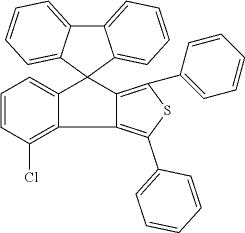 [SMILES: Clc1cccc2c1-c1c(-c3ccccc3)sc(-c3ccccc3)c1C21c2ccccc2-c2ccccc21] | 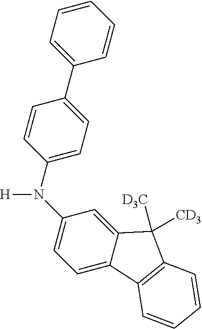 [SMILES: [2H]C([2H])([2H])C1(C([2H])([2H])[2H])c2ccccc2-c2ccc(Nc3ccc(-c4ccccc4)cc3)cc21] | 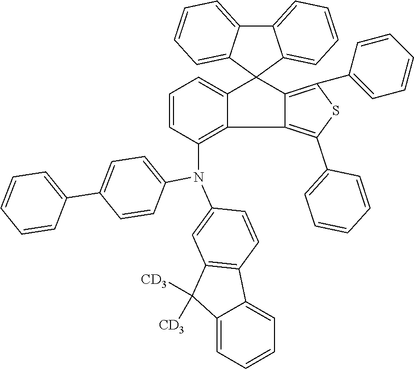 [SMILES: [2H]C([2H])([2H])C1(C([2H])([2H])[2H])c2ccccc2-c2ccc(N(c3ccc(-c4ccccc4)cc3)c3cccc4c3-c3c(-c5ccccc5)sc(-c5ccccc5)c3C43c4ccccc4-c4ccccc43)cc21] | 52% |
| 3m | 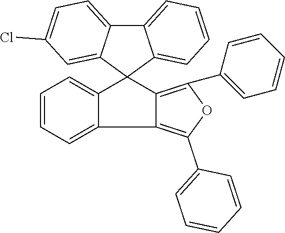 [SMILES: Clc1ccc2c(c1)C1(c3ccccc3-2)c2ccccc2-c2c(-c3ccccc3)oc(-c3ccccc3)c21] | 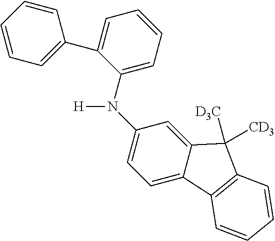 [SMILES: [2H]C([2H])([2H])C1(C([2H])([2H])[2H])c2ccccc2-c2ccc(Nc3ccccc3-c3ccccc3)cc21] | 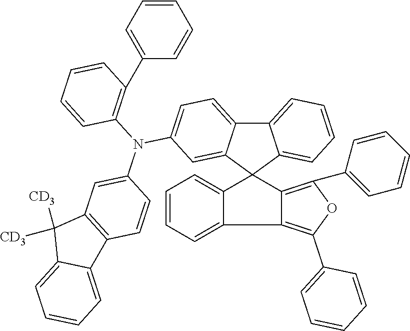 [SMILES: [2H]C([2H])([2H])C1(C([2H])([2H])[2H])c2ccccc2-c2ccc(N(c3ccc4c(c3)C3(c5ccccc5-4)c4ccccc4-c4c(-c5ccccc5)oc(-c5ccccc5)c43)c3ccccc3-c3ccccc3)cc21] | 73% |
| 3n | 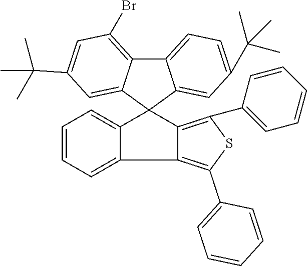 [SMILES: CC(C)(C)c1ccc2c(c1)C1(c3ccccc3-c3c(-c4ccccc4)sc(-c4ccccc4)c31)c1cc(C(C)(C)C)cc(Br)c1-2] | 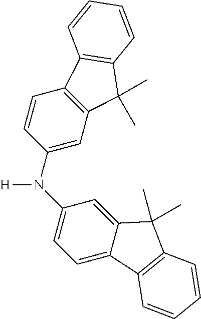 [SMILES: CC1(C)c2ccccc2-c2ccc(Nc3ccc4c(c3)C(C)(C)c3ccccc3-4)cc21] | 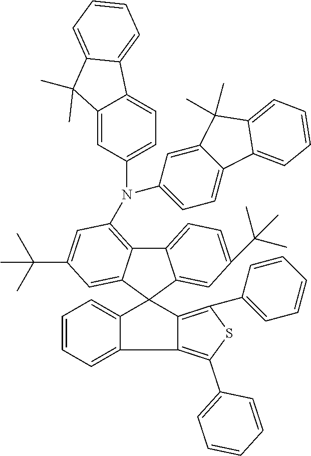 [SMILES: CC(C)(C)c1ccc2c(c1)C1(c3ccccc3-c3c(-c4ccccc4)sc(-c4ccccc4)c31)c1cc(C(C)(C)C)cc(N(c3ccc4c(c3)C(C)(C)c3ccccc3-4)c3ccc4c(c3)C(C)(C)c3ccccc3-4)c1-2] | 59% |
| 3o | 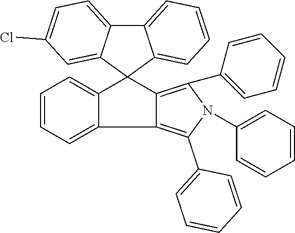 [SMILES: Clc1ccc2c(c1)C1(c3ccccc3-2)c2ccccc2-c2c1c(-c1ccccc1)n(-c1ccccc1)c2-c1ccccc1] | 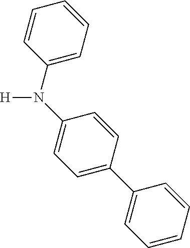 [SMILES: c1ccc(Nc2ccc(-c3ccccc3)cc2)cc1] | 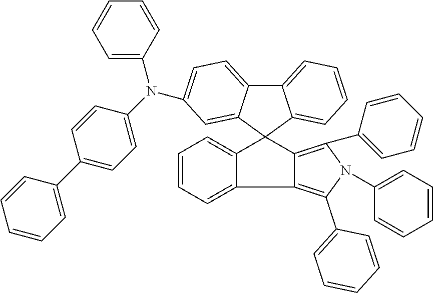 [SMILES: c1ccc(-c2ccc(N(c3ccccc3)c3ccc4c(c3)C3(c5ccccc5-4)c4ccccc4-c4c3c(-c3ccccc3)n(-c3ccccc3)c4-c3ccccc3)cc2)cc1] | 55% |
| 3p | 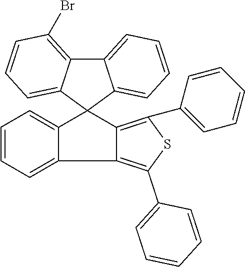 [SMILES: Brc1cccc2c1-c1ccccc1C21c2ccccc2-c2c(-c3ccccc3)sc(-c3ccccc3)c21] | 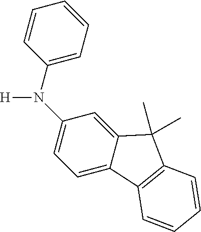 [SMILES: CC1(C)c2ccccc2-c2ccc(Nc3ccccc3)cc21] | 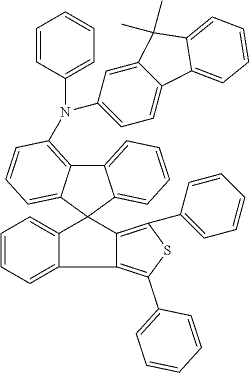 [SMILES: CC1(C)c2ccccc2-c2ccc(N(c3ccccc3)c3cccc4c3-c3ccccc3C43c4ccccc4-c4c(-c5ccccc5)sc(-c5ccccc5)c43)cc21] | 55% |
| 3q | 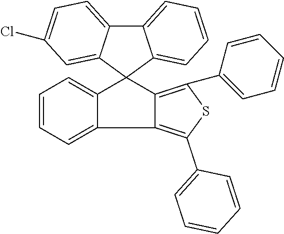 [SMILES: Clc1ccc2c(c1)C1(c3ccccc3-2)c2ccccc2-c2c(-c3ccccc3)sc(-c3ccccc3)c21] | 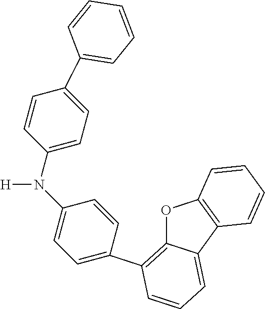 [SMILES: c1ccc(-c2ccc(Nc3ccc(-c4cccc5c4oc4ccccc45)cc3)cc2)cc1] | 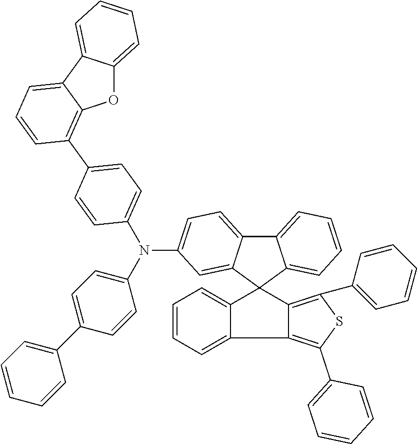 [SMILES: c1ccc(-c2ccc(N(c3ccc(-c4cccc5c4oc4ccccc45)cc3)c3ccc4c(c3)C3(c5ccccc5-4)c4ccccc4-c4c(-c5ccccc5)sc(-c5ccccc5)c43)cc2)cc1] | 75% |
| 3r | 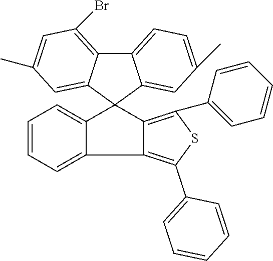 [SMILES: Cc1ccc2c(c1)C1(c3ccccc3-c3c(-c4ccccc4)sc(-c4ccccc4)c31)c1cc(C)cc(Br)c1-2] | 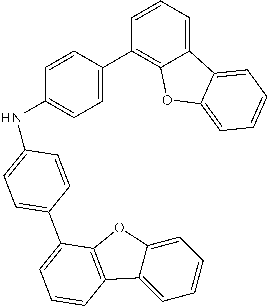 [SMILES: c1ccc2c(c1)oc1c(-c3ccc(Nc4ccc(-c5cccc6c5oc5ccccc56)cc4)cc3)cccc12] | 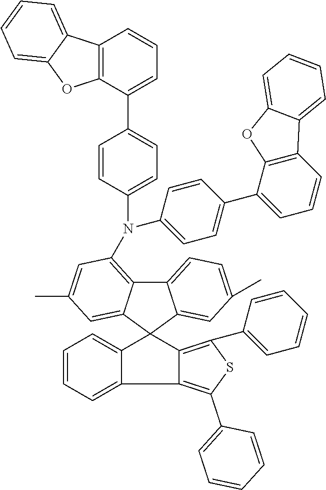 [SMILES: Cc1ccc2c(c1)C1(c3ccccc3-c3c(-c4ccccc4)sc(-c4ccccc4)c31)c1cc(C)cc(N(c3ccc(-c4cccc5c4oc4ccccc45)cc3)c3ccc(-c4cccc5c4oc4ccccc45)cc3)c1-2] | 70% |
| 3s | 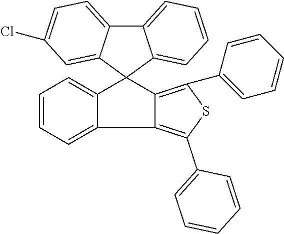 [SMILES: Clc1ccc2c(c1)C1(c3ccccc3-2)c2ccccc2-c2c(-c3ccccc3)sc(-c3ccccc3)c21] | 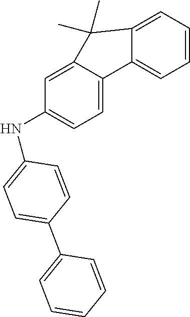 [SMILES: CC1(C)c2ccccc2-c2ccc(Nc3ccc(-c4ccccc4)cc3)cc21] | 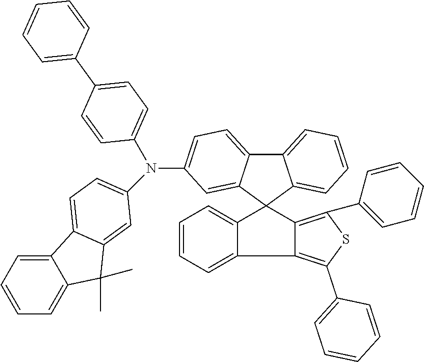 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)sc(-c5ccccc5)c43)cc21] | 72% |
| 3t | 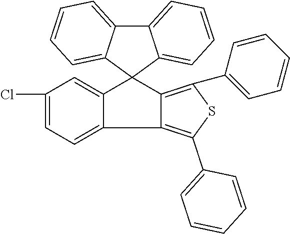 [SMILES: Clc1ccc2c(c1)C1(c3ccccc3-c3ccccc31)c1c(-c3ccccc3)sc(-c3ccccc3)c1-2] | 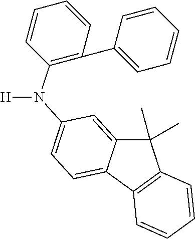 [SMILES: CC1(C)c2ccccc2-c2ccc(Nc3ccccc3-c3ccccc3)cc21] | 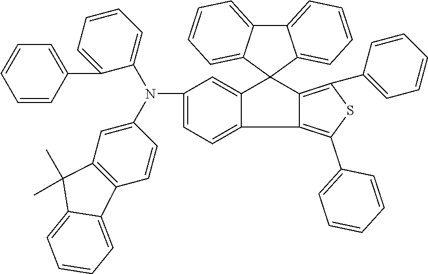 [SMILES: CC1(C)c2ccccc2-c2ccc(N(c3ccc4c(c3)C3(c5ccccc5-c5ccccc53)c3c(-c5ccccc5)sc(-c5ccccc5)c3-4)c3ccccc3-c3ccccc3)cc21] | 62% |
| 3u | 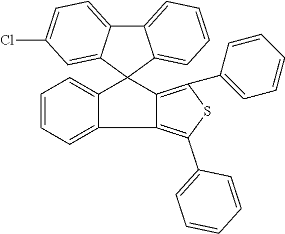 [SMILES: Clc1ccc2c(c1)C1(c3ccccc3-2)c2ccccc2-c2c(-c3ccccc3)sc(-c3ccccc3)c21] | 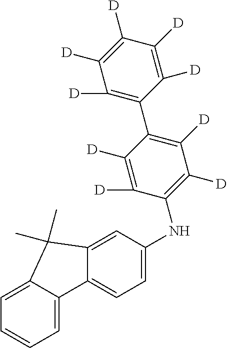 [SMILES: [2H]c1c([2H])c([2H])c(-c2c([2H])c([2H])c(Nc3ccc4c(c3)C(C)(C)c3ccccc3-4)c([2H])c2[2H])c([2H])c1[2H]] | 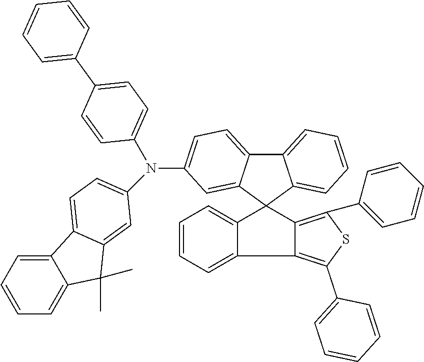 [SMILES: CC1(C)c2ccccc2-c2ccc(N(c3ccc(-c4ccccc4)cc3)c3ccc4c(c3)C3(c5ccccc5-4)c4ccccc4-c4c(-c5ccccc5)sc(-c5ccccc5)c43)cc21] | 45% |
| 3v | 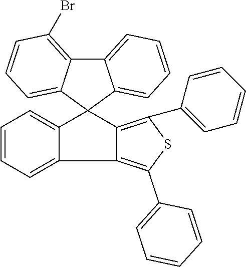 [SMILES: Brc1cccc2c1-c1ccccc1C21c2ccccc2-c2c(-c3ccccc3)sc(-c3ccccc3)c21] | 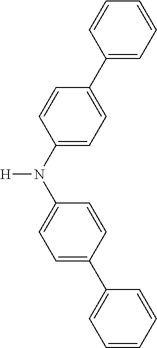 [SMILES: c1ccc(-c2ccc(Nc3ccc(-c4ccccc4)cc3)cc2)cc1] | 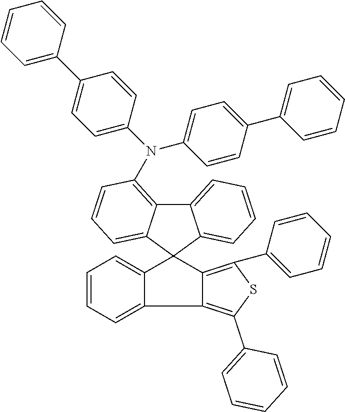 [SMILES: c1ccc(-c2ccc(N(c3ccc(-c4ccccc4)cc3)c3cccc4c3-c3ccccc3C43c4ccccc4-c4c(-c5ccccc5)sc(-c5ccccc5)c43)cc2)cc1] | 66% |
| 3w | 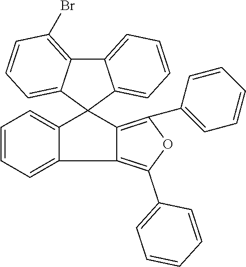 [SMILES: Brc1cccc2c1-c1ccccc1C21c2ccccc2-c2c(-c3ccccc3)oc(-c3ccccc3)c21] | 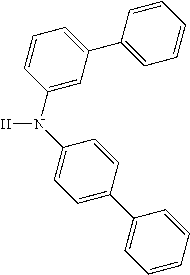 [SMILES: c1ccc(-c2ccc(Nc3cccc(-c4ccccc4)c3)cc2)cc1] | 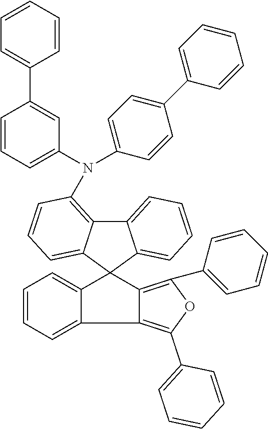 [SMILES: c1ccc(-c2ccc(N(c3cccc(-c4ccccc4)c3)c3cccc4c3-c3ccccc3C43c4ccccc4-c4c(-c5ccccc5)oc(-c5ccccc5)c43)cc2)cc1] | 66% |
| 3x | 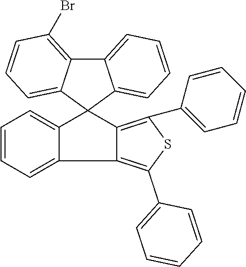 [SMILES: Brc1cccc2c1-c1ccccc1C21c2ccccc2-c2c(-c3ccccc3)sc(-c3ccccc3)c21] | 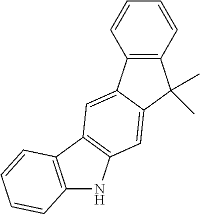 [SMILES: CC1(C)c2ccccc2-c2cc3c(cc21)[nH]c1ccccc13] | 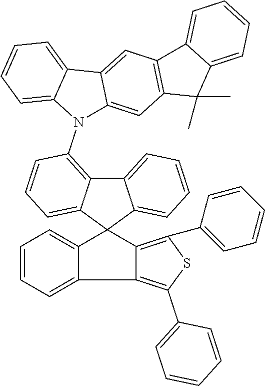 [SMILES: CC1(C)c2ccccc2-c2cc3c4ccccc4n(-c4cccc5c4-c4ccccc4C54c5ccccc5-c5c(-c6ccccc6)sc(-c6ccccc6)c54)c3cc21] | 71% |
| 3y | 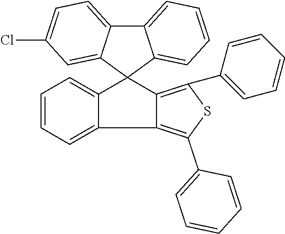 [SMILES: Clc1ccc2c(c1)C1(c3ccccc3-2)c2ccccc2-c2c(-c3ccccc3)sc(-c3ccccc3)c21] | 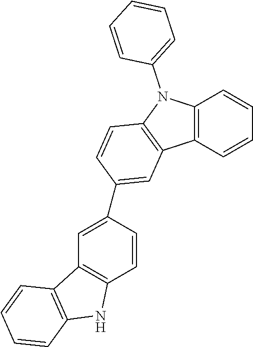 [SMILES: c1ccc(-n2c3ccccc3c3cc(-c4ccc5[nH]c6ccccc6c5c4)ccc32)cc1] | 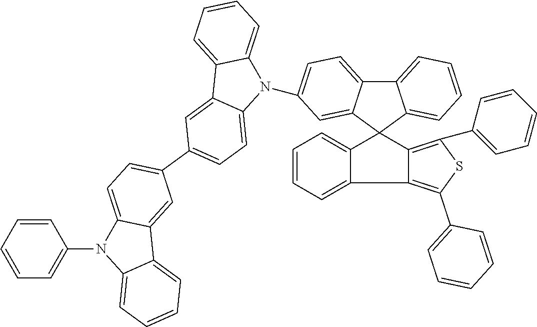 [SMILES: c1ccc(-c2sc(-c3ccccc3)c3c2-c2ccccc2C32c3ccccc3-c3ccc(-n4c5ccccc5c5cc(-c6ccc7c(c6)c6ccccc6n7-c6ccccc6)ccc54)cc32)cc1] | 49% |
| 3z | 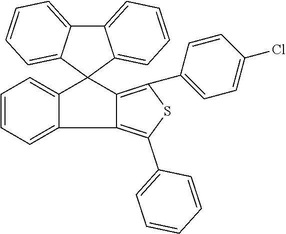 [SMILES: Clc1ccc(-c2sc(-c3ccccc3)c3c2C2(c4ccccc4-c4ccccc42)c2ccccc2-3)cc1] | 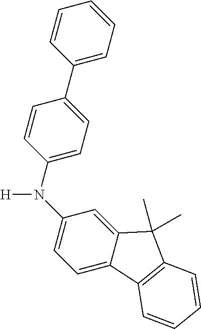 [SMILES: CC1(C)c2ccccc2-c2ccc(Nc3ccc(-c4ccccc4)cc3)cc21] | 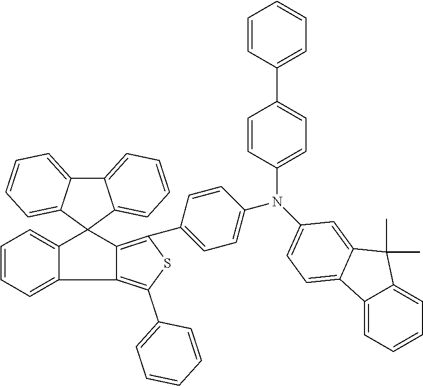 [SMILES: CC1(C)c2ccccc2-c2ccc(N(c3ccc(-c4ccccc4)cc3)c3ccc(-c4sc(-c5ccccc5)c5c4C4(c6ccccc6-c6ccccc64)c4ccccc4-5)cc3)cc21] | 65% |
| 3aa | 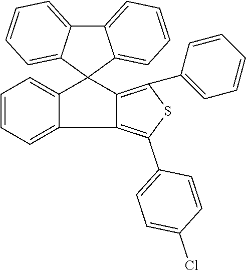 [SMILES: Clc1ccc(-c2sc(-c3ccccc3)c3c2-c2ccccc2C32c3ccccc3-c3ccccc32)cc1] | 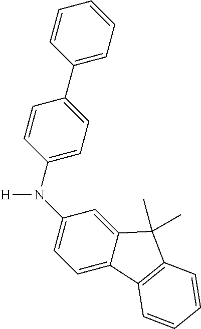 [SMILES: CC1(C)c2ccccc2-c2ccc(Nc3ccc(-c4ccccc4)cc3)cc21] | 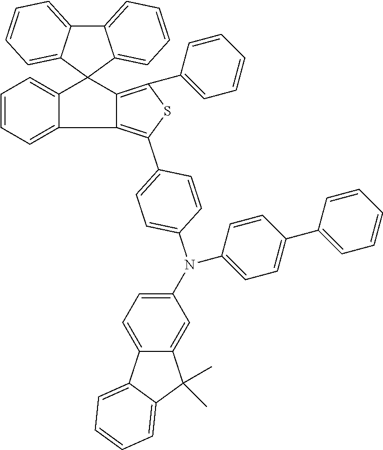 [SMILES: CC1(C)c2ccccc2-c2ccc(N(c3ccc(-c4ccccc4)cc3)c3ccc(-c4sc(-c5ccccc5)c5c4-c4ccccc4C54c5ccccc5-c5ccccc54)cc3)cc21] | 73% |
| 3ab | 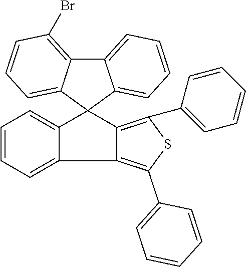 [SMILES: Brc1cccc2c1-c1ccccc1C21c2ccccc2-c2c(-c3ccccc3)sc(-c3ccccc3)c21] | 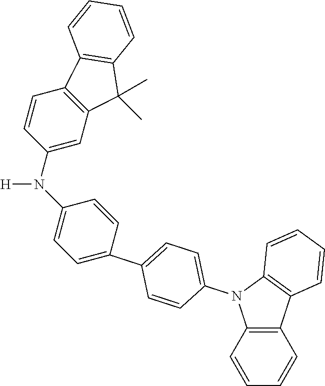 [SMILES: CC1(C)c2ccccc2-c2ccc(Nc3ccc(-c4ccc(-n5c6ccccc6c6ccccc65)cc4)cc3)cc21] | 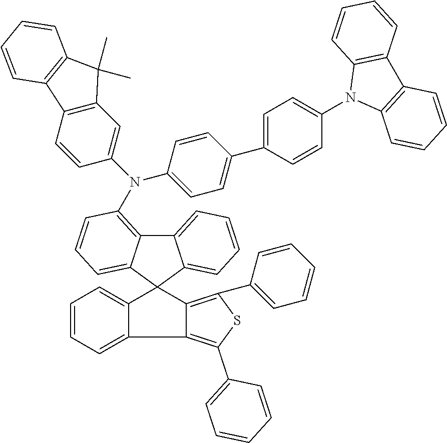 [SMILES: CC1(C)c2ccccc2-c2ccc(N(c3ccc(-c4ccc(-n5c6ccccc6c6ccccc65)cc4)cc3)c3cccc4c3-c3ccccc3C43c4ccccc4-c4c(-c5ccccc5)sc(-c5ccccc5)c43)cc21] | 70% |
| 3ac | 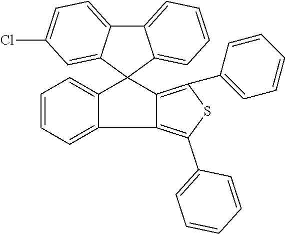 [SMILES: Clc1ccc2c(c1)C1(c3ccccc3-2)c2ccccc2-c2c(-c3ccccc3)sc(-c3ccccc3)c21] | 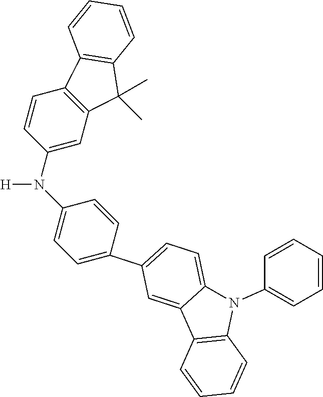 [SMILES: CC1(C)c2ccccc2-c2ccc(Nc3ccc(-c4ccc5c(c4)c4ccccc4n5-c4ccccc4)cc3)cc21] | 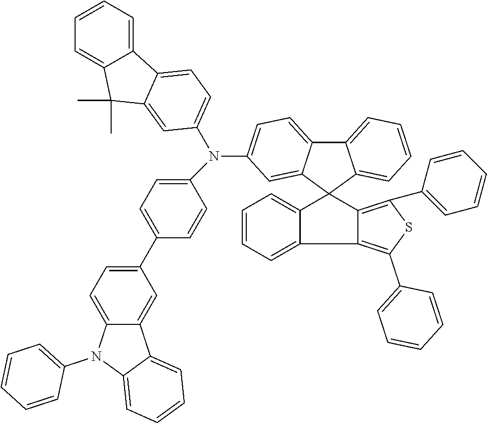 [SMILES: CC1(C)c2ccccc2-c2ccc(N(c3ccc(-c4ccc5c(c4)c4ccccc4n5-c4ccccc4)cc3)c3ccc4c(c3)C3(c5ccccc5-4)c4ccccc4-c4c(-c5ccccc5)sc(-c5ccccc5)c43)cc21] | 80% |
| 3ad | 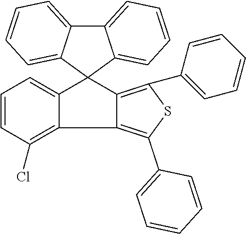 [SMILES: Clc1cccc2c1-c1c(-c3ccccc3)sc(-c3ccccc3)c1C21c2ccccc2-c2ccccc21] | 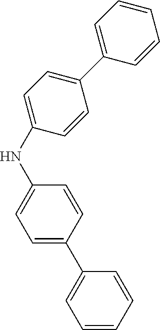 [SMILES: c1ccc(-c2ccc(Nc3ccc(-c4ccccc4)cc3)cc2)cc1] | 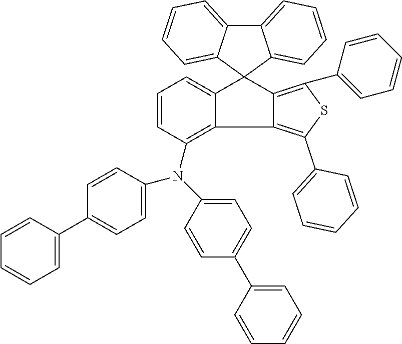 [SMILES: c1ccc(-c2ccc(N(c3ccc(-c4ccccc4)cc3)c3cccc4c3-c3c(-c5ccccc5)sc(-c5ccccc5)c3C43c4ccccc4-c4ccccc43)cc2)cc1] | 75% |
| 3ae | 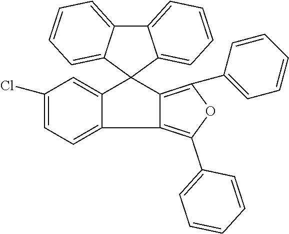 [SMILES: Clc1ccc2c(c1)C1(c3ccccc3-c3ccccc31)c1c(-c3ccccc3)oc(-c3ccccc3)c1-2] | 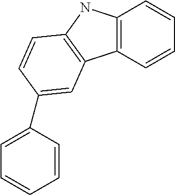 [SMILES: c1ccc(-c2ccc3[nH]c4ccccc4c3c2)cc1] | 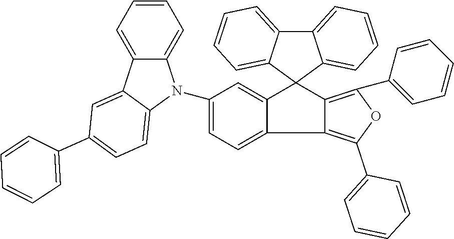 [SMILES: c1ccc(-c2ccc3c(c2)c2ccccc2n3-c2ccc3c(c2)C2(c4ccccc4-c4ccccc42)c2c(-c4ccccc4)oc(-c4ccccc4)c2-3)cc1] | 50% |
| 3af | 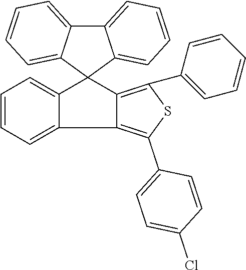 [SMILES: Clc1ccc(-c2sc(-c3ccccc3)c3c2-c2ccccc2C32c3ccccc3-c3ccccc32)cc1] | 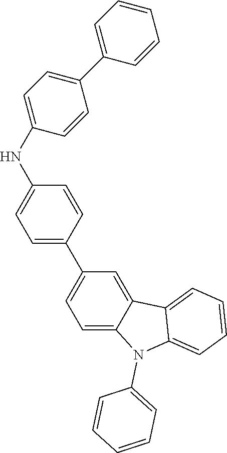 [SMILES: c1ccc(-c2ccc(Nc3ccc(-c4ccc5c(c4)c4ccccc4n5-c4ccccc4)cc3)cc2)cc1] | 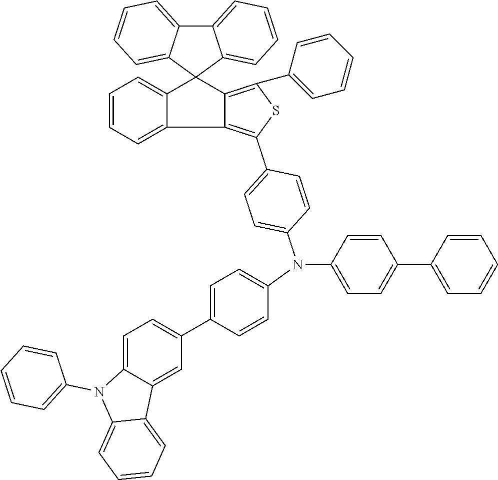 [SMILES: c1ccc(-c2ccc(N(c3ccc(-c4ccc5c(c4)c4ccccc4n5-c4ccccc4)cc3)c3ccc(-c4sc(-c5ccccc5)c5c4-c4ccccc4C54c5ccccc5-c5ccccc54)cc3)cc2)cc1] | 40% |
| 3ag | 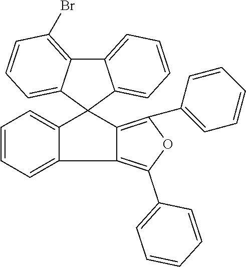 [SMILES: Brc1cccc2c1-c1ccccc1C21c2ccccc2-c2c(-c3ccccc3)oc(-c3ccccc3)c21] | 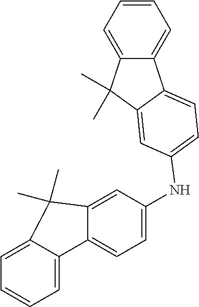 [SMILES: CC1(C)c2ccccc2-c2ccc(Nc3ccc4c(c3)C(C)(C)c3ccccc3-4)cc21] | 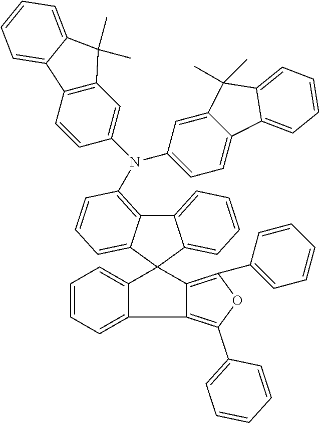 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccccc5)oc(-c5ccccc5)c43)cc21] | 76% |
| 3ah | 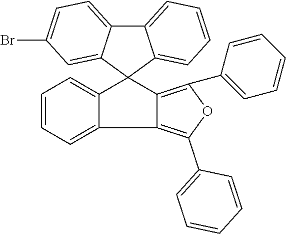 [SMILES: Brc1ccc2c(c1)C1(c3ccccc3-2)c2ccccc2-c2c(-c3ccccc3)oc(-c3ccccc3)c21] | 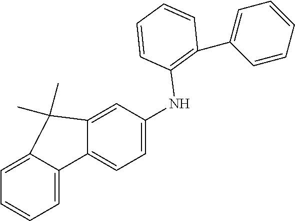 [SMILES: CC1(C)c2ccccc2-c2ccc(Nc3ccccc3-c3ccccc3)cc21] | 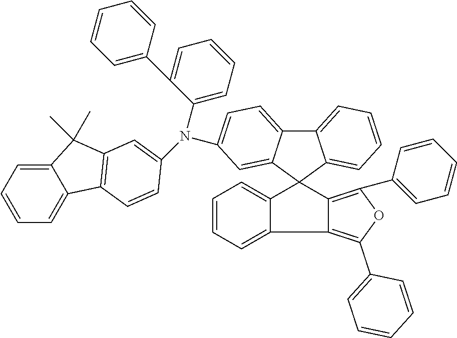 [SMILES: CC1(C)c2ccccc2-c2ccc(N(c3ccc4c(c3)C3(c5ccccc5-4)c4ccccc4-c4c(-c5ccccc5)oc(-c5ccccc5)c43)c3ccccc3-c3ccccc3)cc21] | 30% |

### N-{[1,1′-Biphenyl]-4-yl}-N-(4-{1′,3′-diphenylspiro[fluorene-9,8′-indeno[1,2-c]thiophen]-7-yl}phenyl)-9,9-dimethyl-9H-fluorene-2-amine 4a

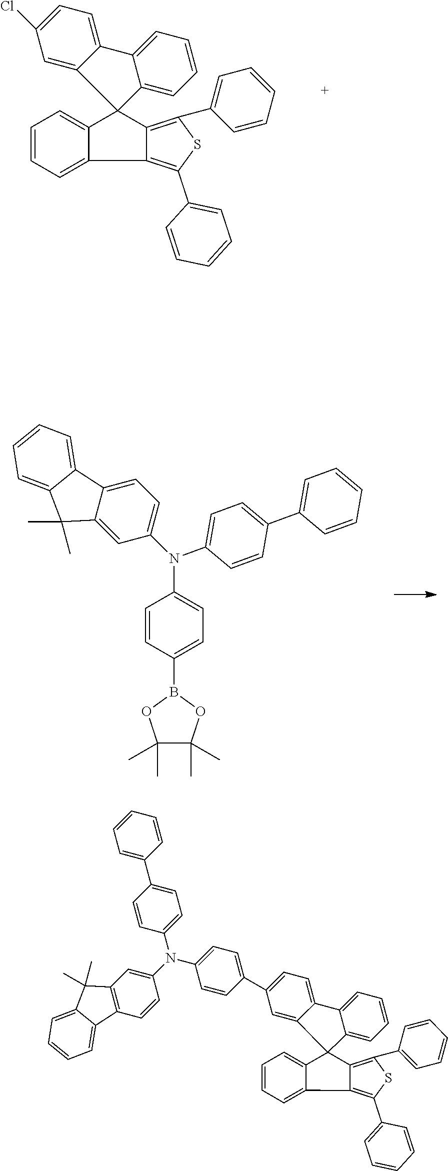

20.0 g (39 mmol) of N-{[1,1′-biphenyl]-4-yl}-9,9-dimethyl-N-[4-(4,4,5,5-tetramethyl-1,3,2-dioxaborolan-2-yl)phenyl]-9H-fluorene-2-amine and 23.5 g (42 mmol) of 2-chloro-1′,3′-diphenylspiro[fluorene-9,8′-indeno[1,2-c]thiophene] are suspended in 400 ml of dioxane and 13.7 g of caesium fluoride (90 mmol). 4.0 g (5.4 mmol) of bis(tricyclohexylphosphine)palladium dichloride are added to this suspension, and the reaction mixture is heated under reflux for 18 h. After cooling, the organic phase is removed, filtered through silica gel, washed three times with 80 ml of water and then concentrated to dryness. After the crude product has been filtered through silica gel with toluene, the remaining residue is recrystallized from heptane/toluene and finally sublimed under high vacuum; purity is 99.9%. The yield is 11 g (30% of theory).

The following compounds are prepared in an analogous manner:

| Ex. | Spiro | Amine | Product | Yield |
|---|---|---|---|---|
| 4b | 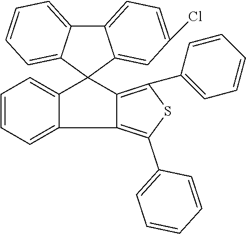 [SMILES: Clc1ccc2c(c1)C1(c3ccccc3-2)c2ccccc2-c2c(-c3ccccc3)sc(-c3ccccc3)c21] | 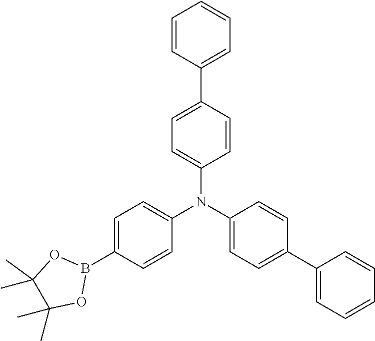 [SMILES: CC1(C)OB(c2ccc(N(c3ccc(-c4ccccc4)cc3)c3ccc(-c4ccccc4)cc3)cc2)OC1(C)C] | 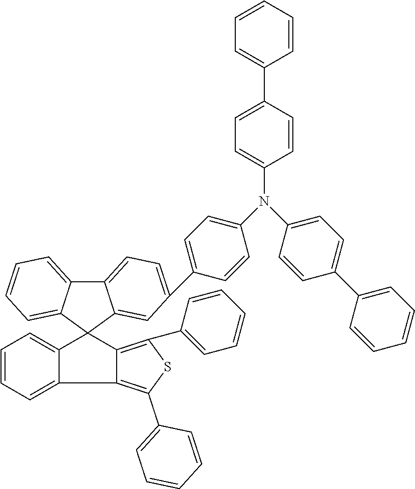 [SMILES: c1ccc(-c2ccc(N(c3ccc(-c4ccccc4)cc3)c3ccc(-c4ccc5c(c4)C4(c6ccccc6-5)c5ccccc5-c5c(-c6ccccc6)sc(-c6ccccc6)c54)cc3)cc2)cc1] | 53% |
| 4c | 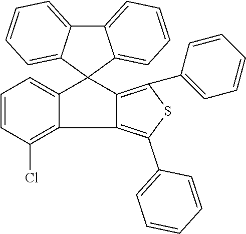 [SMILES: Clc1cccc2c1-c1c(-c3ccccc3)sc(-c3ccccc3)c1C21c2ccccc2-c2ccccc21] | 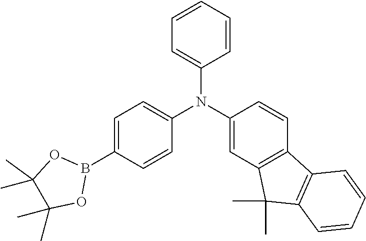 [SMILES: CC1(C)c2ccccc2-c2ccc(N(c3ccccc3)c3ccc(B4OC(C)(C)C(C)(C)O4)cc3)cc21] | 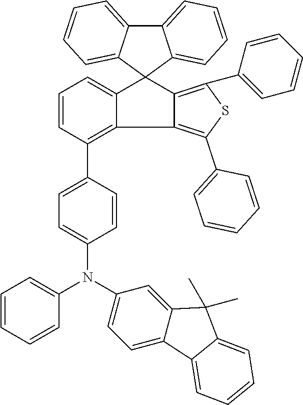 [SMILES: CC1(C)c2ccccc2-c2ccc(N(c3ccccc3)c3ccc(-c4cccc5c4-c4c(-c6ccccc6)sc(-c6ccccc6)c4C54c5ccccc5-c5ccccc54)cc3)cc21] | 65% |
| 4d | 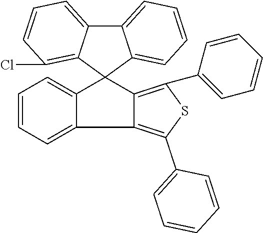 [SMILES: Clc1cccc2c1C1(c3ccccc3-2)c2ccccc2-c2c(-c3ccccc3)sc(-c3ccccc3)c21] | 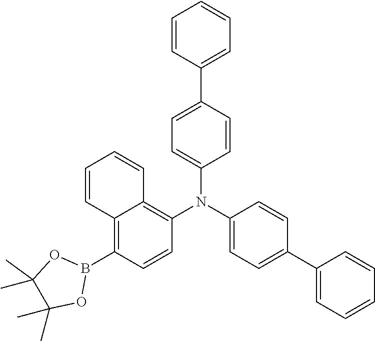 [SMILES: CC1(C)OB(c2ccc(N(c3ccc(-c4ccccc4)cc3)c3ccc(-c4ccccc4)cc3)c3ccccc23)OC1(C)C] | 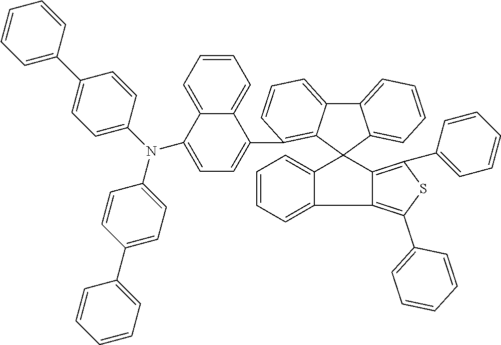 [SMILES: c1ccc(-c2ccc(N(c3ccc(-c4ccccc4)cc3)c3ccc(-c4cccc5c4C4(c6ccccc6-5)c5ccccc5-c5c(-c6ccccc6)sc(-c6ccccc6)c54)c4ccccc34)cc2)cc1] | 50% |
| 4e | 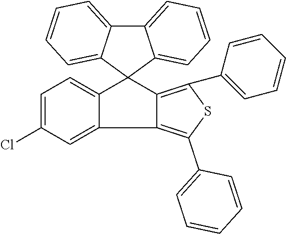 [SMILES: Clc1ccc2c(c1)-c1c(-c3ccccc3)sc(-c3ccccc3)c1C21c2ccccc2-c2ccccc21] | 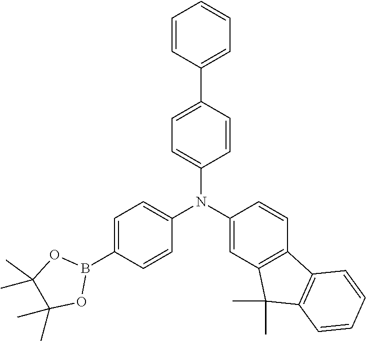 [SMILES: CC1(C)c2ccccc2-c2ccc(N(c3ccc(B4OC(C)(C)C(C)(C)O4)cc3)c3ccc(-c4ccccc4)cc3)cc21] | 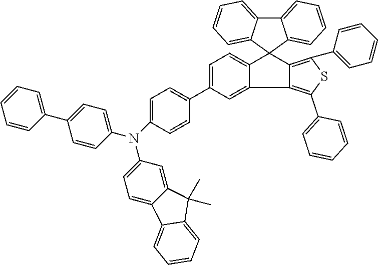 [SMILES: CC1(C)c2ccccc2-c2ccc(N(c3ccc(-c4ccccc4)cc3)c3ccc(-c4ccc5c(c4)-c4c(-c6ccccc6)sc(-c6ccccc6)c4C54c5ccccc5-c5ccccc54)cc3)cc21] | 53% |
| 4f | 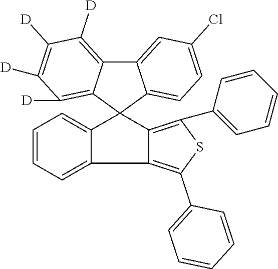 [SMILES: [2H]c1c([2H])c([2H])c2c(c1[2H])-c1cc(Cl)ccc1C21c2ccccc2-c2c(-c3ccccc3)sc(-c3ccccc3)c21] | 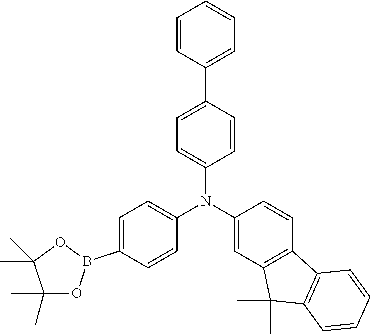 [SMILES: CC1(C)c2ccccc2-c2ccc(N(c3ccc(B4OC(C)(C)C(C)(C)O4)cc3)c3ccc(-c4ccccc4)cc3)cc21] | 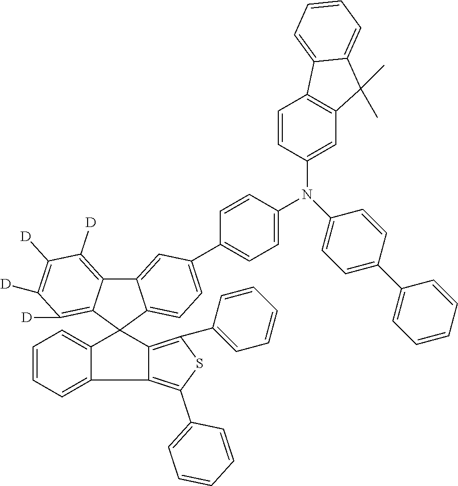 [SMILES: [2H]c1c([2H])c([2H])c2c(c1[2H])-c1cc(-c3ccc(N(c4ccc(-c5ccccc5)cc4)c4ccc5c(c4)C(C)(C)c4ccccc4-5)cc3)ccc1C21c2ccccc2-c2c(-c3ccccc3)sc(-c3ccccc3)c21] | 60% |
| 4g | 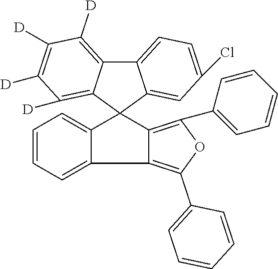 [SMILES: [2H]c1c([2H])c([2H])c2c(c1[2H])-c1ccc(Cl)cc1C21c2ccccc2-c2c(-c3ccccc3)oc(-c3ccccc3)c21] | 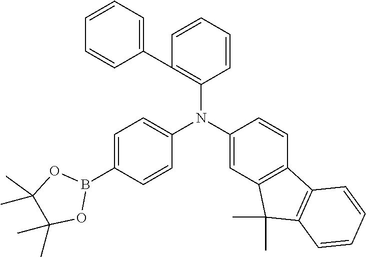 [SMILES: CC1(C)c2ccccc2-c2ccc(N(c3ccc(B4OC(C)(C)C(C)(C)O4)cc3)c3ccccc3-c3ccccc3)cc21] | 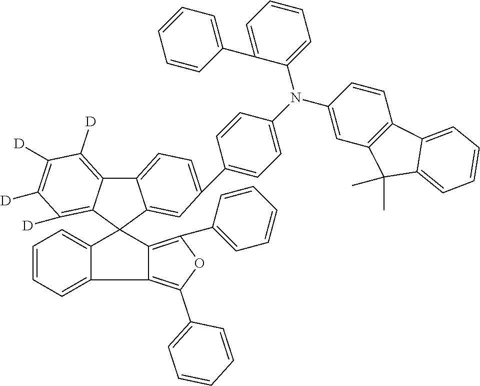 [SMILES: [2H]c1c([2H])c([2H])c2c(c1[2H])-c1ccc(-c3ccc(N(c4ccc5c(c4)C(C)(C)c4ccccc4-5)c4ccccc4-c4ccccc4)cc3)cc1C21c2ccccc2-c2c(-c3ccccc3)oc(-c3ccccc3)c21] | 55% |
| 4h | 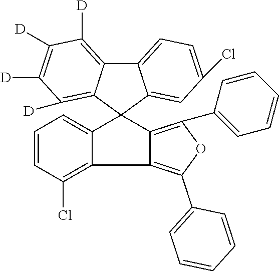 [SMILES: [2H]c1c([2H])c([2H])c2c(c1[2H])-c1ccc(Cl)cc1C21c2cccc(Cl)c2-c2c(-c3ccccc3)oc(-c3ccccc3)c21] | 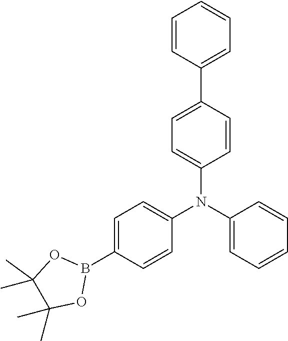 [SMILES: CC1(C)OB(c2ccc(N(c3ccccc3)c3ccc(-c4ccccc4)cc3)cc2)OC1(C)C] | 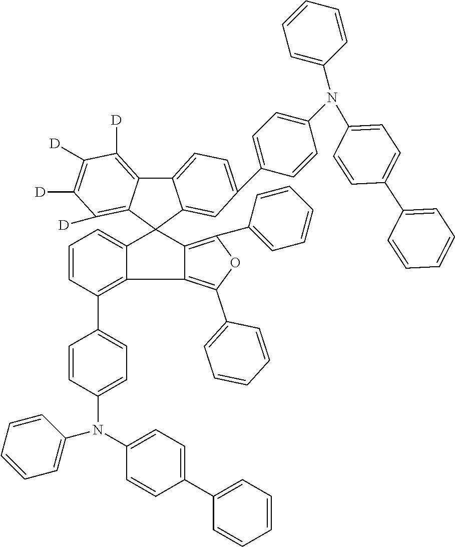 [SMILES: [2H]c1c([2H])c([2H])c2c(c1[2H])-c1ccc(-c3ccc(N(c4ccccc4)c4ccc(-c5ccccc5)cc4)cc3)cc1C21c2cccc(-c3ccc(N(c4ccccc4)c4ccc(-c5ccccc5)cc4)cc3)c2-c2c(-c3ccccc3)oc(-c3ccccc3)c21] | 65% |
| 4i | 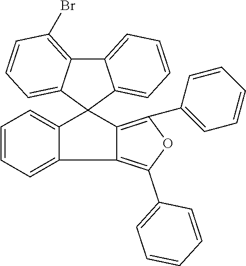 [SMILES: Brc1cccc2c1-c1ccccc1C21c2ccccc2-c2c(-c3ccccc3)oc(-c3ccccc3)c21] | 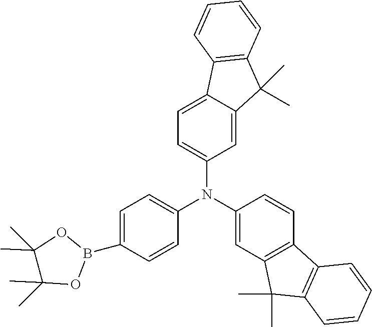 [SMILES: CC1(C)c2ccccc2-c2ccc(N(c3ccc(B4OC(C)(C)C(C)(C)O4)cc3)c3ccc4c(c3)C(C)(C)c3ccccc3-4)cc21] | 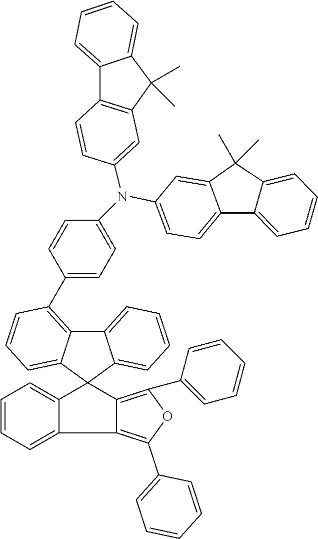 [SMILES: CC1(C)c2ccccc2-c2ccc(N(c3ccc(-c4cccc5c4-c4ccccc4C54c5ccccc5-c5c(-c6ccccc6)oc(-c6ccccc6)c54)cc3)c3ccc4c(c3)C(C)(C)c3ccccc3-4)cc21] | 54% |
| 4j | 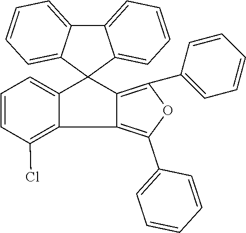 [SMILES: Clc1cccc2c1-c1c(-c3ccccc3)oc(-c3ccccc3)c1C21c2ccccc2-c2ccccc21] | 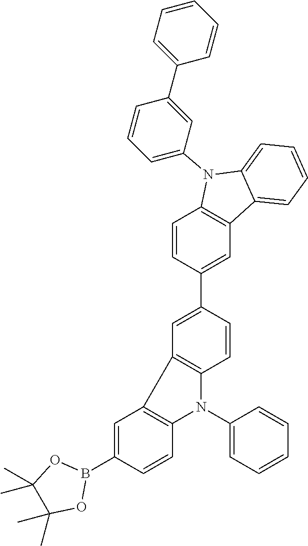 [SMILES: CC1(C)OB(c2ccc3c(c2)c2cc(-c4ccc5c(c4)c4ccccc4n5-c4cccc(-c5ccccc5)c4)ccc2n3-c2ccccc2)OC1(C)C] | 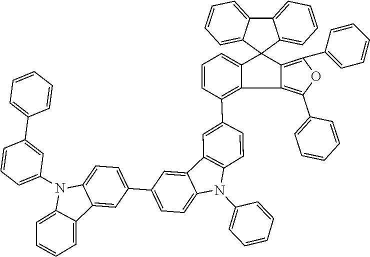 [SMILES: c1ccc(-c2cccc(-n3c4ccccc4c4cc(-c5ccc6c(c5)c5cc(-c7cccc8c7-c7c(-c9ccccc9)oc(-c9ccccc9)c7C87c8ccccc8-c8ccccc87)ccc5n6-c5ccccc5)ccc43)c2)cc1] | 65% |
| 4k | 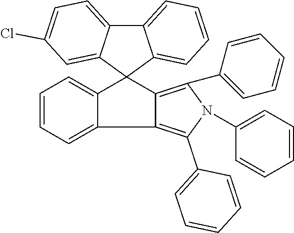 [SMILES: Clc1ccc2c(c1)C1(c3ccccc3-2)c2ccccc2-c2c1c(-c1ccccc1)n(-c1ccccc1)c2-c1ccccc1] | 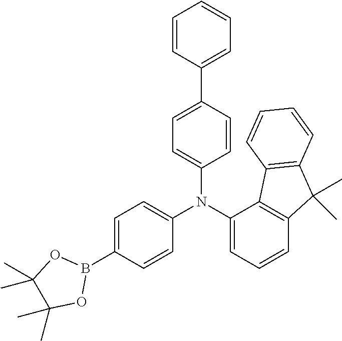 | 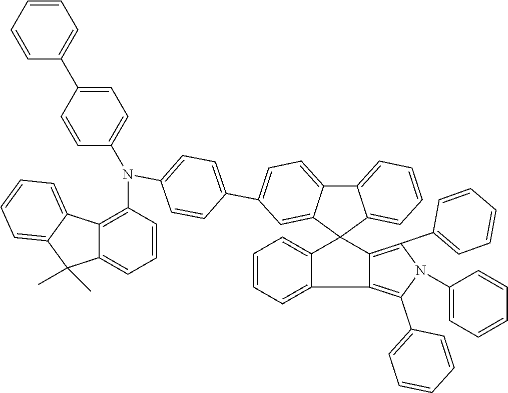 [SMILES: CC1(C)c2ccccc2-c2c(N(c3ccc(-c4ccccc4)cc3)c3ccc(-c4ccc5c(c4)C4(c6ccccc6-5)c5ccccc5-c5c4c(-c4ccccc4)n(-c4ccccc4)c5-c4ccccc4)cc3)cccc21] | 45% |
| 4l | 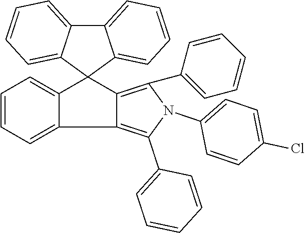 [SMILES: Clc1ccc(-n2c(-c3ccccc3)c3c(c2-c2ccccc2)C2(c4ccccc4-c4ccccc42)c2ccccc2-3)cc1] | 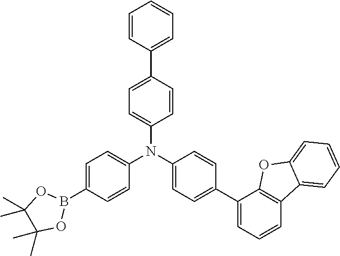 [SMILES: CC1(C)OB(c2ccc(N(c3ccc(-c4ccccc4)cc3)c3ccc(-c4cccc5c4oc4ccccc45)cc3)cc2)OC1(C)C] | 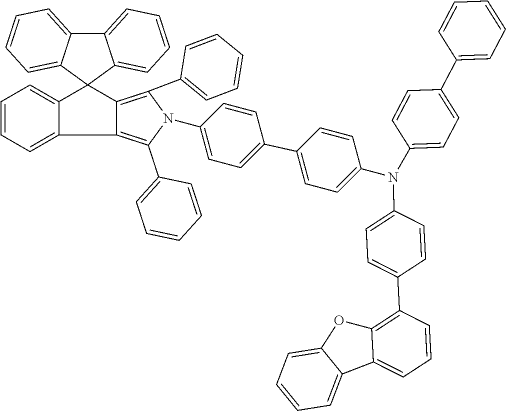 [SMILES: c1ccc(-c2ccc(N(c3ccc(-c4ccc(-n5c(-c6ccccc6)c6c(c5-c5ccccc5)C5(c7ccccc7-c7ccccc75)c5ccccc5-6)cc4)cc3)c3ccc(-c4cccc5c4oc4ccccc45)cc3)cc2)cc1] | 80% |
| 4m | 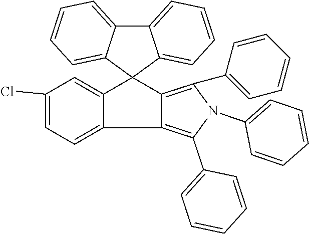 [SMILES: Clc1ccc2c(c1)C1(c3ccccc3-c3ccccc31)c1c-2c(-c2ccccc2)n(-c2ccccc2)c1-c1ccccc1] | 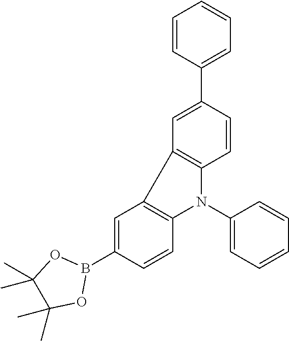 [SMILES: CC1(C)OB(c2ccc3c(c2)c2cc(-c4ccccc4)ccc2n3-c2ccccc2)OC1(C)C] | 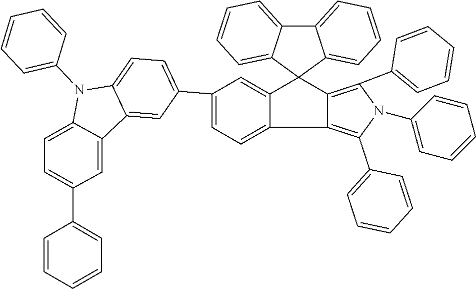 [SMILES: c1ccc(-c2ccc3c(c2)c2cc(-c4ccc5c(c4)C4(c6ccccc6-c6ccccc64)c4c-5c(-c5ccccc5)n(-c5ccccc5)c4-c4ccccc4)ccc2n3-c2ccccc2)cc1] | 67% |
| 4n | 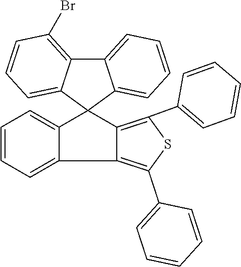 [SMILES: Brc1cccc2c1-c1ccccc1C21c2ccccc2-c2c(-c3ccccc3)sc(-c3ccccc3)c21] | 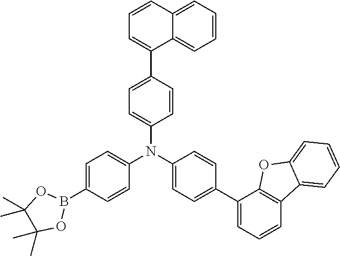 [SMILES: CC1(C)OB(c2ccc(N(c3ccc(-c4cccc5ccccc45)cc3)c3ccc(-c4cccc5c4oc4ccccc45)cc3)cc2)OC1(C)C] | 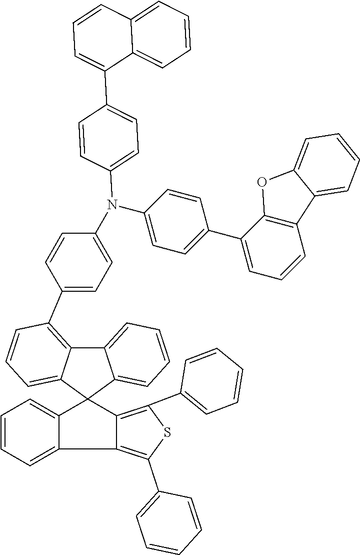 [SMILES: c1ccc(-c2sc(-c3ccccc3)c3c2-c2ccccc2C32c3ccccc3-c3c(-c4ccc(N(c5ccc(-c6cccc7ccccc67)cc5)c5ccc(-c6cccc7c6oc6ccccc67)cc5)cc4)cccc32)cc1] | 60% |
| 4o | 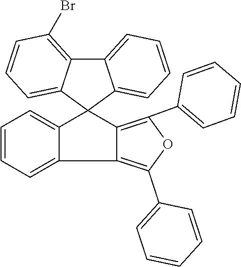 [SMILES: Brc1cccc2c1-c1ccccc1C21c2ccccc2-c2c(-c3ccccc3)oc(-c3ccccc3)c21] | 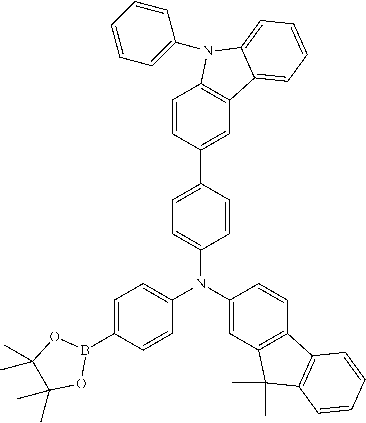 [SMILES: CC1(C)c2ccccc2-c2ccc(N(c3ccc(B4OC(C)(C)C(C)(C)O4)cc3)c3ccc(-c4ccc5c(c4)c4ccccc4n5-c4ccccc4)cc3)cc21] | 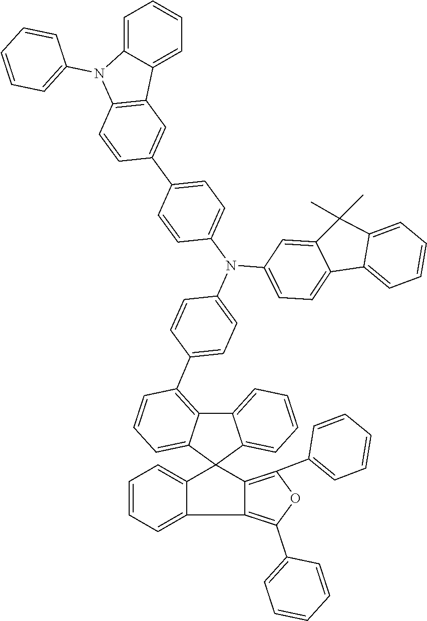 [SMILES: CC1(C)c2ccccc2-c2ccc(N(c3ccc(-c4ccc5c(c4)c4ccccc4n5-c4ccccc4)cc3)c3ccc(-c4cccc5c4-c4ccccc4C54c5ccccc5-c5c(-c6ccccc6)oc(-c6ccccc6)c54)cc3)cc21] | 50% |
| 4p | 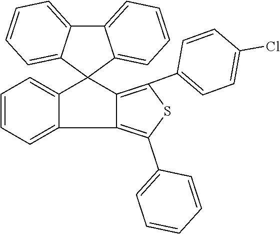 [SMILES: Clc1ccc(-c2sc(-c3ccccc3)c3c2C2(c4ccccc4-c4ccccc42)c2ccccc2-3)cc1] | 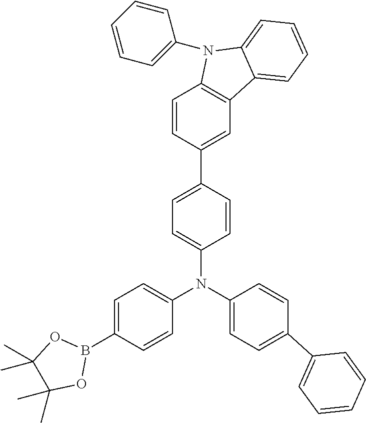 [SMILES: CC1(C)OB(c2ccc(N(c3ccc(-c4ccccc4)cc3)c3ccc(-c4ccc5c(c4)c4ccccc4n5-c4ccccc4)cc3)cc2)OC1(C)C] | 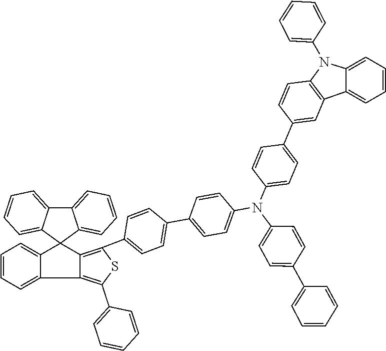 [SMILES: c1ccc(-c2ccc(N(c3ccc(-c4ccc(-c5sc(-c6ccccc6)c6c5C5(c7ccccc7-c7ccccc75)c5ccccc5-6)cc4)cc3)c3ccc(-c4ccc5c(c4)c4ccccc4n5-c4ccccc4)cc3)cc2)cc1] | 62% |
| 4q | 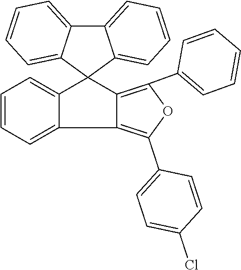 [SMILES: Clc1ccc(-c2oc(-c3ccccc3)c3c2-c2ccccc2C32c3ccccc3-c3ccccc32)cc1] | 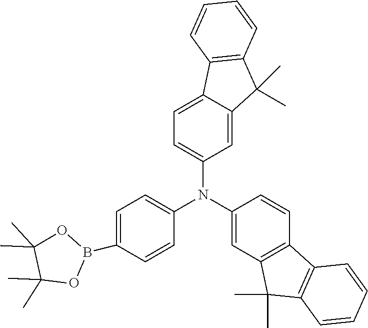 [SMILES: CC1(C)c2ccccc2-c2ccc(N(c3ccc(B4OC(C)(C)C(C)(C)O4)cc3)c3ccc4c(c3)C(C)(C)c3ccccc3-4)cc21] | 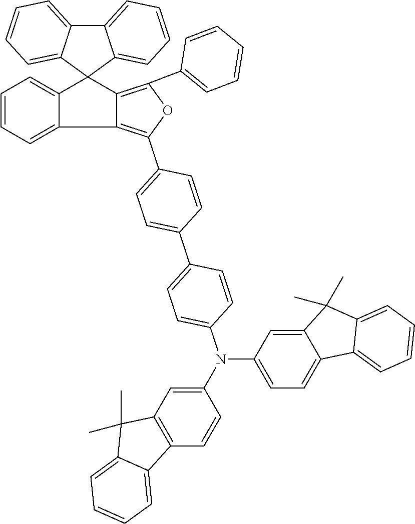 [SMILES: CC1(C)c2ccccc2-c2ccc(N(c3ccc(-c4ccc(-c5oc(-c6ccccc6)c6c5-c5ccccc5C65c6ccccc6-c6ccccc65)cc4)cc3)c3ccc4c(c3)C(C)(C)c3ccccc3-4)cc21] | 55% |
| 4r | 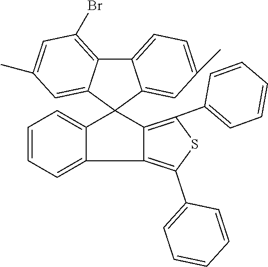 [SMILES: Cc1ccc2c(c1)C1(c3ccccc3-c3c(-c4ccccc4)sc(-c4ccccc4)c31)c1cc(C)cc(Br)c1-2] | 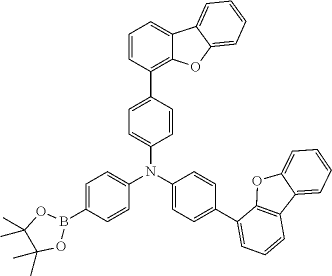 [SMILES: CC1(C)OB(c2ccc(N(c3ccc(-c4cccc5c4oc4ccccc45)cc3)c3ccc(-c4cccc5c4oc4ccccc45)cc3)cc2)OC1(C)C] | 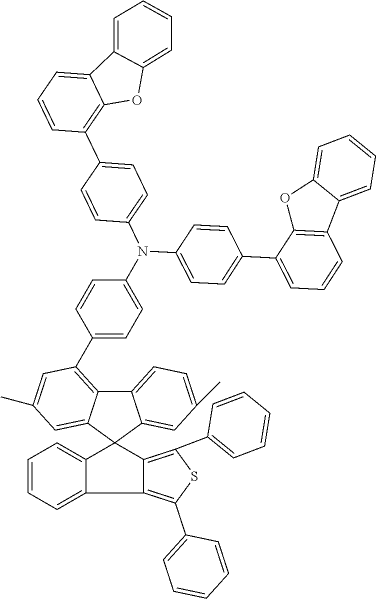 [SMILES: Cc1ccc2c(c1)C1(c3ccccc3-c3c(-c4ccccc4)sc(-c4ccccc4)c31)c1cc(C)cc(-c3ccc(N(c4ccc(-c5cccc6c5oc5ccccc56)cc4)c4ccc(-c5cccc6c5oc5ccccc56)cc4)cc3)c1-2] | 66% |
| 4s | 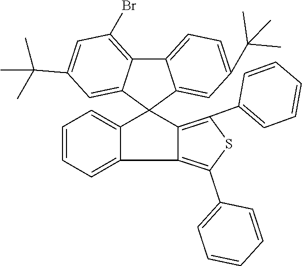 [SMILES: CC(C)(C)c1ccc2c(c1)C1(c3ccccc3-c3c(-c4ccccc4)sc(-c4ccccc4)c31)c1cc(C(C)(C)C)cc(Br)c1-2] | 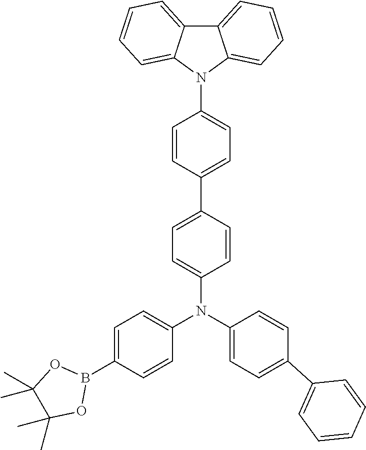 [SMILES: CC1(C)OB(c2ccc(N(c3ccc(-c4ccccc4)cc3)c3ccc(-c4ccc(-n5c6ccccc6c6ccccc65)cc4)cc3)cc2)OC1(C)C] | 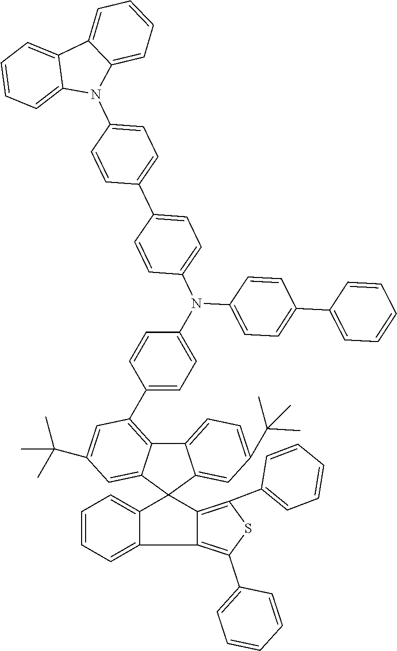 [SMILES: CC(C)(C)c1ccc2c(c1)C1(c3ccccc3-c3c(-c4ccccc4)sc(-c4ccccc4)c31)c1cc(C(C)(C)C)cc(-c3ccc(N(c4ccc(-c5ccccc5)cc4)c4ccc(-c5ccc(-n6c7ccccc7c7ccccc76)cc5)cc4)cc3)c1-2] | 52% |
| 4t | 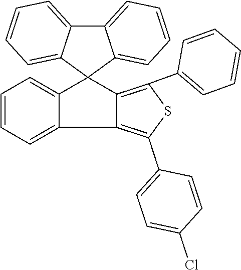 [SMILES: Clc1ccc(-c2sc(-c3ccccc3)c3c2-c2ccccc2C32c3ccccc3-c3ccccc32)cc1] | 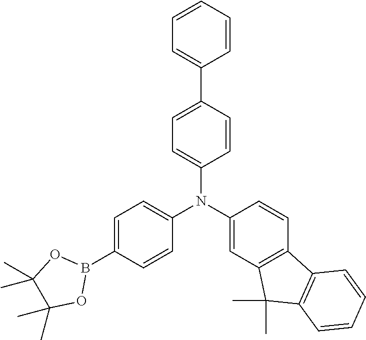 [SMILES: CC1(C)c2ccccc2-c2ccc(N(c3ccc(B4OC(C)(C)C(C)(C)O4)cc3)c3ccc(-c4ccccc4)cc3)cc21] | 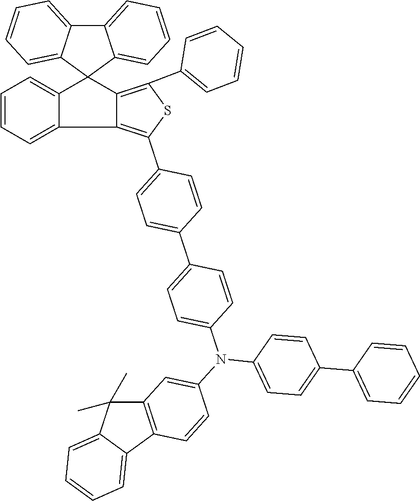 [SMILES: CC1(C)c2ccccc2-c2ccc(N(c3ccc(-c4ccccc4)cc3)c3ccc(-c4ccc(-c5sc(-c6ccccc6)c6c5-c5ccccc5C65c6ccccc6-c6ccccc65)cc4)cc3)cc21] | 48% |
| 4u | 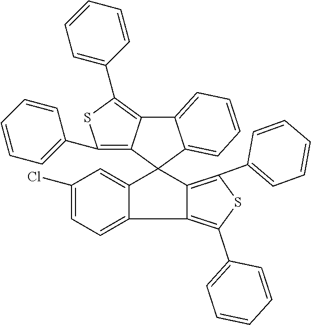 [SMILES: Clc1ccc2c(c1)C1(c3ccccc3-c3c(-c4ccccc4)sc(-c4ccccc4)c31)c1c(-c3ccccc3)sc(-c3ccccc3)c1-2] | 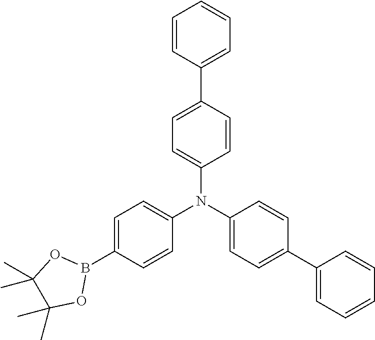 [SMILES: CC1(C)OB(c2ccc(N(c3ccc(-c4ccccc4)cc3)c3ccc(-c4ccccc4)cc3)cc2)OC1(C)C] | 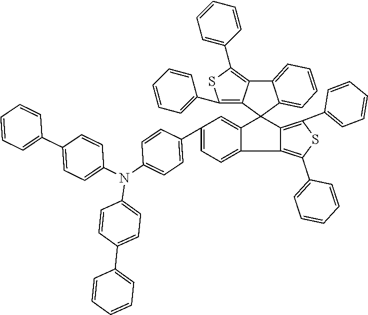 [SMILES: c1ccc(-c2ccc(N(c3ccc(-c4ccccc4)cc3)c3ccc(-c4ccc5c(c4)C4(c6ccccc6-c6c(-c7ccccc7)sc(-c7ccccc7)c64)c4c(-c6ccccc6)sc(-c6ccccc6)c4-5)cc3)cc2)cc1] | 53% |
| 4v | 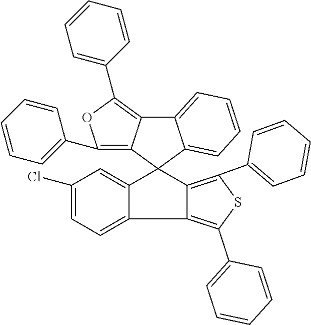 [SMILES: Clc1ccc2c(c1)C1(c3ccccc3-c3c(-c4ccccc4)oc(-c4ccccc4)c31)c1c(-c3ccccc3)sc(-c3ccccc3)c1-2] | 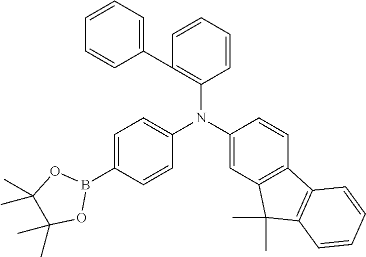 [SMILES: CC1(C)c2ccccc2-c2ccc(N(c3ccc(B4OC(C)(C)C(C)(C)O4)cc3)c3ccccc3-c3ccccc3)cc21] | 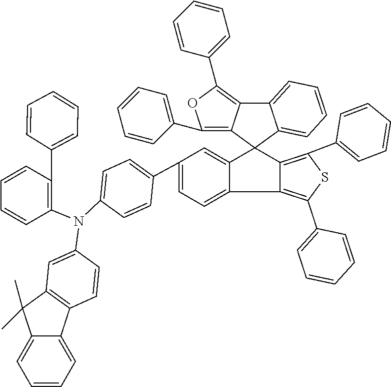 [SMILES: CC1(C)c2ccccc2-c2ccc(N(c3ccc(-c4ccc5c(c4)C4(c6ccccc6-c6c(-c7ccccc7)oc(-c7ccccc7)c64)c4c(-c6ccccc6)sc(-c6ccccc6)c4-5)cc3)c3ccccc3-c3ccccc3)cc21] | 51% |
| 4x | 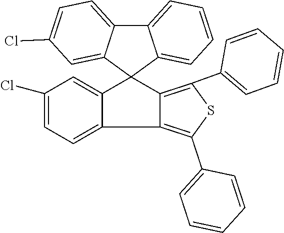 [SMILES: Clc1ccc2c(c1)C1(c3ccccc3-2)c2cc(Cl)ccc2-c2c(-c3ccccc3)sc(-c3ccccc3)c21] | 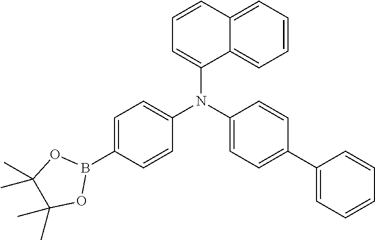 [SMILES: CC1(C)OB(c2ccc(N(c3ccc(-c4ccccc4)cc3)c3cccc4ccccc34)cc2)OC1(C)C] | 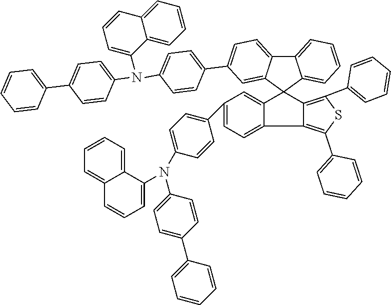 [SMILES: c1ccc(-c2ccc(N(c3ccc(-c4ccc5c(c4)C4(c6ccccc6-5)c5cc(-c6ccc(N(c7ccc(-c8ccccc8)cc7)c7cccc8ccccc78)cc6)ccc5-c5c(-c6ccccc6)sc(-c6ccccc6)c54)cc3)c3cccc4ccccc34)cc2)cc1] | 46% |
| 4y | 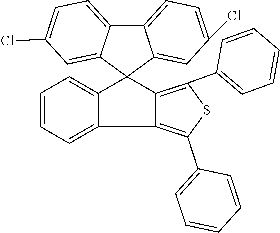 [SMILES: Clc1ccc2c(c1)C1(c3cc(Cl)ccc3-2)c2ccccc2-c2c(-c3ccccc3)sc(-c3ccccc3)c21] | 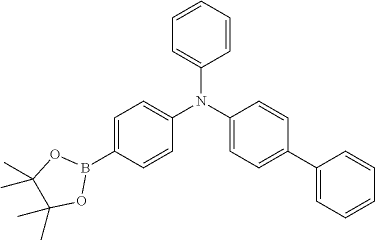 [SMILES: CC1(C)OB(c2ccc(N(c3ccccc3)c3ccc(-c4ccccc4)cc3)cc2)OC1(C)C] | 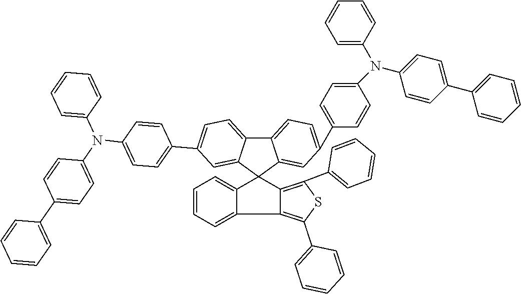 [SMILES: c1ccc(-c2ccc(N(c3ccccc3)c3ccc(-c4ccc5c(c4)C4(c6cc(-c7ccc(N(c8ccccc8)c8ccc(-c9ccccc9)cc8)cc7)ccc6-5)c5ccccc5-c5c(-c6ccccc6)sc(-c6ccccc6)c54)cc3)cc2)cc1] | 42% |
| 4z | 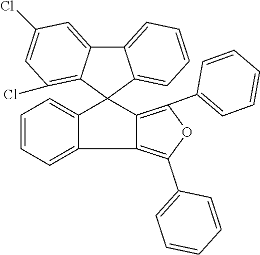 [SMILES: Clc1cc(Cl)c2c(c1)-c1ccccc1C21c2ccccc2-c2c(-c3ccccc3)oc(-c3ccccc3)c21] | 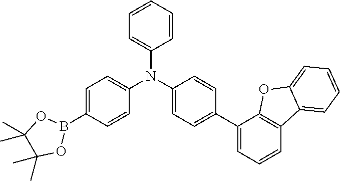 [SMILES: CC1(C)OB(c2ccc(N(c3ccccc3)c3ccc(-c4cccc5c4oc4ccccc45)cc3)cc2)OC1(C)C] | 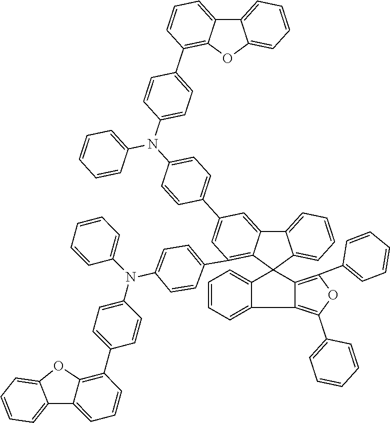 [SMILES: c1ccc(-c2oc(-c3ccccc3)c3c2-c2ccccc2C32c3ccccc3-c3cc(-c4ccc(N(c5ccccc5)c5ccc(-c6cccc7c6oc6ccccc67)cc5)cc4)cc(-c4ccc(N(c5ccccc5)c5ccc(-c6cccc7c6oc6ccccc67)cc5)cc4)c32)cc1] | 40% |

### B) Device Examples

1) General production process for the OLEDs and characterization of the OLEDs

Glass plaques which have been coated with structured ITO (indium tin oxide) in a thickness of 50 nm are the substrates to which the OLEDs are applied.

The OLEDs basically have the following layer structure: substrate/hole injection layer (HIL)/hole transport layer (HTL)/electron blocker layer (EBL)/emission layer (EML)/optional hole blocker layer (HBL)/electron transport layer (ETL)/electron injection layer (EIL) and finally a cathode. The cathode is formed by an aluminium layer of thickness 100 nm. The exact structure of the OLEDs can be found in the tables which follow. The materials used for production of the OLEDs are shown in a table below. The material H-A used here is an anthracene derivative, and the SEB-A used is a spirobifluorenediamine. The emitter TEG-A used is a derivative of Ir(PPy)₃.

All materials are applied by thermal vapour deposition in a vacuum chamber. In this case, the emission layer consists of at least one matrix material (host material) and an emitting dopant which is added to the matrix material(s) in a particular proportion by volume by co-evaporation. Details given in such a form as H:SEB (95%:5%) mean here that the material H is present in the layer in a proportion by volume of 95% and SEB in a proportion of 5%.

In an analogous manner, the electron transport layer and the hole injection layer also consist of a mixture of two materials. The structures of the materials that are used in the OLEDs are shown in Table 3.

The OLEDs are characterized in a standard manner. For this purpose, the electroluminescence spectra, the external quantum efficiency (EQE, measured in %) as a function of the luminance, calculated from current-voltage-luminance characteristics assuming Lambertian radiation characteristics, and the lifetime are determined. The parameter EQE @ 10 mA/cm² refers to the external quantum efficiency which is attained at 10 mA/cm². The parameter U @ 10 mA/cm² refers to the operating voltage at 10 mA/cm². The lifetime LT is defined as the time after which the luminance drops from the starting luminance to a certain proportion in the course of operation with constant current density. An LT80 figure means here that the lifetime reported corresponds to the time after which the luminance has dropped to 80% of its starting value. The figure LT90 has a corresponding meaning. The figure @80 or 60 or 40 mA/cm² means here that the lifetime in question is measured at 80 or 60 or mA/cm².

2) Inventive OLEDs containing a compound of the formula (I) in the EBL of green-phosphorescing OLEDs

Devices as shown in the following table are produced:

TABLE 1a
Construction of the device

| | | | | | | | |
|---|---|---|---|---|---|---|---|
| | | HTL | EBL | EML | ETL | ETL | EIL |
| | HIL | Thickness/ | Thickness/ | Thickness/ | Thickness/ | Thickness/ | Thickness/ |
| Ex. | Thickness/nm | nm | nm | nm | nm | nm | nm |

| | |
|---|---|
| 11 | HTM: p-doped (5%) |
| 12 | HTM: p-doped (5%) |
| 13 | HTM: p-doped (5%) |
| 14 | HTM: p-doped (5%) |

In the device setup shown above, the compounds of the invention give very good efficiencies and lifetimes for the OLEDs:

TABLE 2a
Data of the OLEDs

| | U @ 10 | EQE @ 10 | LT80 @ 40 |
|---|---|---|---|
| | mA/cm² (V) | mA/cm² (%) | mA/cm² (h) |
| I1 | 3.82 | 15.1 | 428 |
| I2 | 3.96 | 16.1 | 372 |
| I3 | 3.55 | 15.3 | 401 |
| I4 | 3.82 | 15.5 | 429 |

In addition, devices are produced as shown in the following table:

| | | HTL | EBL | EML | HBL | ETL | EIL |
|---|---|---|---|---|---|---|---|
| | HIL | Thickness/ | Thickness/ | Thickness/ | Thickness/ | Thickness/ | Thickness/ |
| Ex. | Thickness/nm | nm | nm | nm | nm | nm | nm |
| I4- | HTM-A: p-dopant | HTM-A | HTM-8 | TMM-A(32%) | HBM-A | ETM- | LiQ |
| 1 | (5%) 20 nm | 90 nm | 20 nm | TMM-B(60%) | 10 nm | A:LiQ(50%) | 1 nm |
| | | | | TEG-A (8%) | | 30 nm | |
| | | | | 30 nm | | | |

This gives very good efficiencies and lifetimes for the OLEDs:

| | U @ 10 | EQE @ 10 | LT90 @ 80 |
|---|---|---|---|
| | mA/cm² (V) | mA/cm² (%) | mA/cm² (h) |
| I4-1 | 3.42 | 19.9 | 125 |

3) Inventive OLEDs containing a compound of the formula (I) in the EBL of blue-fluorescing OLEDs

Devices as shown in the following table are produced:

TABLE 1b
Construction of the device

| | | HTL | EBL | EML | ETL | EIL |
|---|---|---|---|---|---|---|
| | HIL | Thickness/ | Thickness/ | Thickness/ | Thickness/ | Thickness/ |
| Ex. | Thickness/nm | nm | nm | nm | nm | nm |
| I6 | HTM: p-doped | HTM | HTM-5 | H:SEB(5%) | ETM:LiQ(50%) | LiQ |
| | (5%) | 180 nm | 10 nm | 20 nm | 30 nm | 1 nm |
| I7 | HTM: p-doped | HTM | HTM-3 | H:SEB(5%) | ETM:LiQ(50%) | LiQ |
| | (5%) | 180 nm | 10 nm | 20 nm | 30 nm | 1 nm |
| I8 | HTM: p-doped | HTM | HTM-4 | H:SEB(5%) | ETM:LiQ(50%) | LiQ |
| | (5%) | 180 nm | 10 nm | 20 nm | 30 nm | 1 nm |

In the device setup shown above, the compounds of the invention give very good efficiencies and lifetimes for the OLEDs:

TABLE 2b
Data of the OLEDs

| | U @ 10 | EQE @ 10 | LT80 @ 60 |
|---|---|---|---|
| | mA/cm² (V) | mA/cm² (%) | mA/cm² (h) |
| I6 | 3.72 | 9.80 | 335 |
| I7 | 3.89 | 5.70 | 368 |
| I8 | 3.78 | 6.25 | 330 |

In addition, devices are produced as shown in the following table:

| | | HTL | EBL | EML | HBL | ETL | EIL |
|---|---|---|---|---|---|---|---|
| | HIL | Thickness/ | Thickness/ | Thickness/ | Thickness/ | Thickness/ | Thickness/ |
| Ex. | Thickness/nm | nm | nm | nm | nm | nm | nm |
| I8-1 | HTM-A: p-dopant | HTM-A | HTM-8 | H-A:SEB- | HBM-A | ETM- | LiQ |
| | (5%) 10 nm | 180 nm | 10 nm | A(3%) | 10 nm | A:LiQ(50%) | 1 nm |
| | | | | 20 nm | | 30 nm | |

This gives very good efficiencies and lifetimes for the OLEDs:

| | U @ 10 | EQE @ 10 | LT90 @ 60 |
|---|---|---|---|
| | mA/cm² (V) | mA/cm² (%) | mA/cm² (h) |
| I8-1 | 3.6 | 7.7 | 94 |

4) Inventive OLEDs containing a compound of the formula (I) in the HIL and HTL of blue-fluorescing OLEDs

Devices as shown in the following table are produced:

TABLE 1c
Structure of the OLEDs

| | | HTL | EBL | EML | ETL | EIL |
|---|---|---|---|---|---|---|
| | HIL | Thickness/ | Thickness/ | Thickness/ | Thickness/ | Thickness/ |
| Ex. | Thickness/nm | nm | nm | nm | nm | nm |
| I9 | HTM-1: p-dopant | HTM-1 | EBM | H:SEB(5%) | ETM:LiQ(50%) | LiQ |
| | (5%) 20 nm | 180 nm | 10 nm | 20 nm | 30 nm | 1 nm |
| I10 | HTM-6: p-dopant | HTM-6 | EBM | H:SEB(5%) | ETM:LiQ(50%) | LiQ |
| | (5%) 20 nm | 180 nm | 10 nm | 20 nm | 30 nm | 1 nm |
| I11 | HTM-7: p-dopant | HTM-7 | EBM | H:SEB(5%) | ETM:LiQ(50%) | LiQ |
| | (5%) 20 nm | 180 nm | 10 nm | 20 nm | 30 nm | 1 nm |
| I12 | HTM-3: p-dopant | HTM-3 | EBM | H:SEB(5%) | ETM:LiQ(50%) | LiQ |
| | (5%) 20 nm | 180 nm | 10 nm | 20 nm | 30 nm | 1 nm |

In the device setup shown above, the compounds of the invention give very good efficiencies and lifetimes for the OLEDs:

TABLE 2c
Data of the OLEDs

| | U @ 10 | EQE @ 10 | LT80 @ 60 |
|---|---|---|---|
| | mA/cm² (V) | mA/cm² (%) | mA/cm² (h) |
| I9 | 4.27 | 9.29 | 198 |
| I10 | 9.59 | 9.88 | 171 |
| I11 | 4.37 | 8.79 | 186 |
| I12 | 4.19 | 9.58 | 195 |

In addition, devices are produced as shown in the following table:

| | | HTL | EBL | EML | HBL | ETL | EIL |
|---|---|---|---|---|---|---|---|
| | HIL | Thickness/ | Thickness/ | Thickness/ | Thickness/ | Thickness/ | Thickness/ |
| Ex. | Thickness/nm | nm | nm | nm | nm | nm | nm |
| I-12- | HTM-8: p-dopant | HTM-8 | EBM | H-A:SEB- | HBM-A | ETM- | LiQ |
| 1 | (5%) 10 nm | 180 nm | 10 nm | A(3%) | 10 nm | A:LiQ(50%) | 1 nm |
| | | | | 20 nm | | 30 nm | |

This gives very good efficiencies and lifetimes for the OLEDs:

| | U @ 10 | EQE @ 10 | LT90 @ 60 |
|---|---|---|---|
| | mA/cm² (V) | mA/cm² (%) | mA/cm² (h) |
| I12-1 | 4.07 | 8.75 | 79 |

TABLE 3
Structures of the compounds

| | |
|---|---|
| 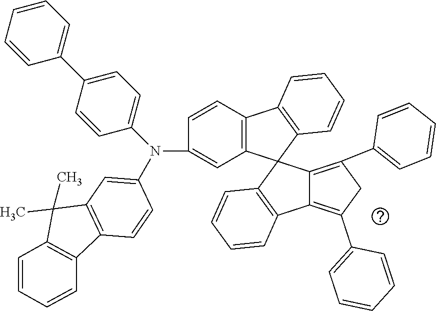 [SMILES: CC1(C)c2ccccc2-c2ccc(N(c3ccc(-c4ccccc4)cc3)c3ccc4c(c3)C3(C5=C(c6ccccc6)CC(c6ccccc6)=C5c5ccccc53)c3ccccc3-4)cc21] | HTM-1 |
| 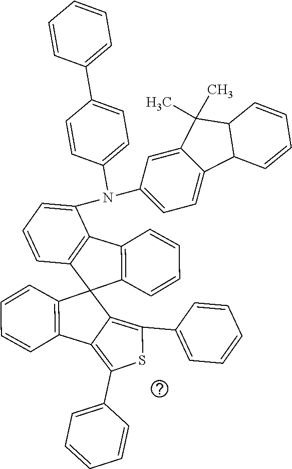 [SMILES: CC1(C)c2cc(N(c3ccc(-c4ccccc4)cc3)c3cccc4c3-c3ccccc3C43c4ccccc4-c4c(-c5ccccc5)sc(-c5ccccc5)c43)ccc2C2C=CC=CC21] | HTM-2 |
| 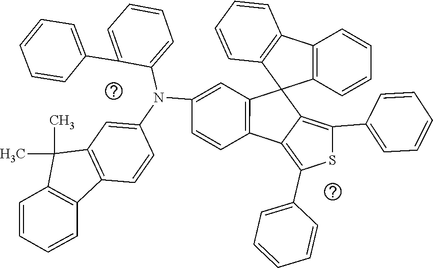 [SMILES: CC1(C)c2ccccc2-c2ccc(N(c3ccc4c(c3)C3(c5ccccc5-c5ccccc53)c3c(-c5ccccc5)sc(-c5ccccc5)c3-4)c3ccccc3-c3ccccc3)cc21] | HTM-3 |
| 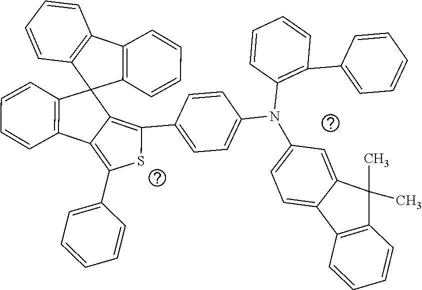 [SMILES: CC1(C)c2ccccc2-c2ccc(N(c3ccc(-c4sc(-c5ccccc5)c5c4C4(c6ccccc6-c6ccccc64)c4ccccc4-5)cc3)c3ccccc3-c3ccccc3)cc21] | HTM-4 |
| 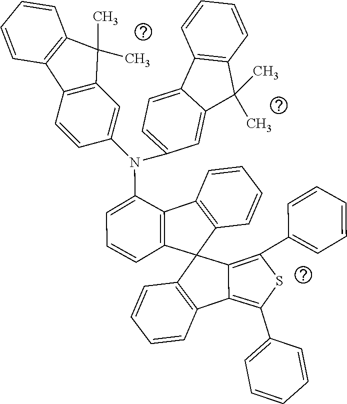 [SMILES: CC1(C)c2ccccc2-c2ccc(N(c3ccc4c(c3)C(C)(C)c3ccccc3-4)c3cccc4c3-c3ccccc3C43c4ccccc4-c4c(-c5ccccc5)sc(-c5ccccc5)c43)cc21] | HTM-5 |
| 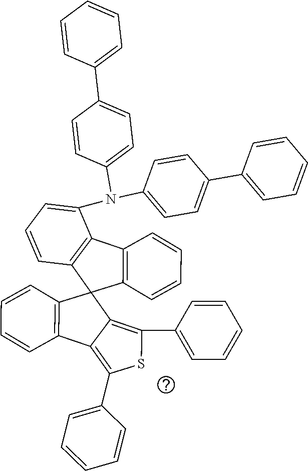 [SMILES: c1ccc(-c2ccc(N(c3ccc(-c4ccccc4)cc3)c3cccc4c3-c3ccccc3C43c4ccccc4-c4c(-c5ccccc5)sc(-c5ccccc5)c43)cc2)cc1] | HTM-6 |
| 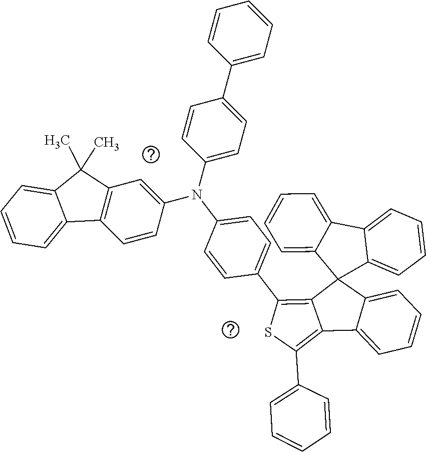 [SMILES: CC1(C)c2ccccc2-c2ccc(N(c3ccc(-c4ccccc4)cc3)c3ccc(-c4sc(-c5ccccc5)c5c4C4(c6ccccc6-c6ccccc64)c4ccccc4-5)cc3)cc21] | HTM-7 |
| 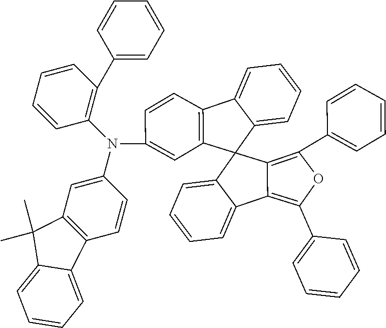 [SMILES: CC1(C)c2ccccc2-c2ccc(N(c3ccc4c(c3)C3(c5ccccc5-4)c4ccccc4-c4c(-c5ccccc5)oc(-c5ccccc5)c43)c3ccccc3-c3ccccc3)cc21] | HTM-8 |
| 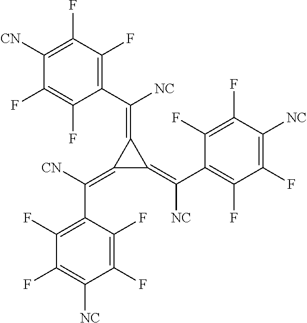 | p-dopant |
| 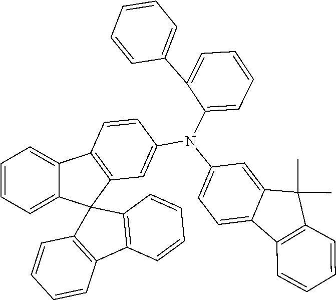 [SMILES: CC1(C)c2ccccc2-c2ccc(N(c3ccc4c(c3)C3(c5ccccc5-c5ccccc53)c3ccccc3-4)c3ccccc3-c3ccccc3)cc21] | HTM |
| 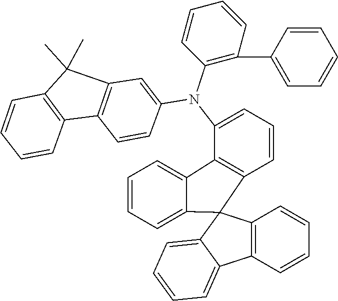 [SMILES: CC1(C)c2ccccc2-c2ccc(N(c3ccccc3-c3ccccc3)c3cccc4c3-c3ccccc3C43c4ccccc4-c4ccccc43)cc21] | EBM |
| 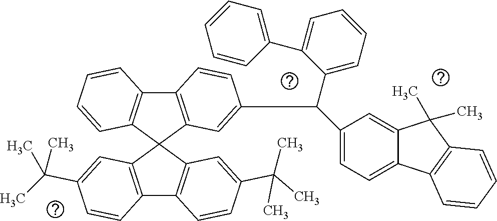 [SMILES: CC(C)(C)c1ccc2c(c1)C1(c3ccccc3-c3ccc(C(c4ccc5c(c4)C(C)(C)c4ccccc4-5)c4ccccc4-c4ccccc4)cc31)c1cc(C(C)(C)C)ccc1-2] | HTM-A |
| 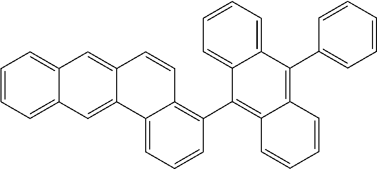 [SMILES: c1ccc(-c2c3ccccc3c(-c3cccc4c3ccc3cc5ccccc5cc34)c3ccccc23)cc1] | H |
| 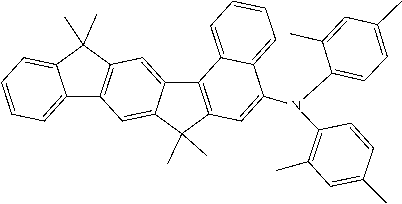 [SMILES: Cc1ccc(N(c2ccc(C)cc2C)c2cc3c(c4ccccc24)-c2cc4c(cc2C3(C)C)-c2ccccc2C4(C)C)c(C)c1] | SEB |
| 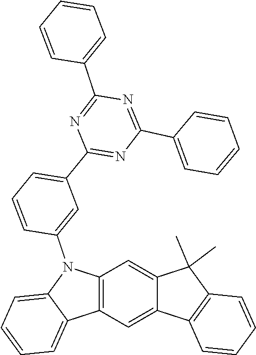 [SMILES: CC1(C)c2ccccc2-c2cc3c4ccccc4n(-c4cccc(-c5nc(-c6ccccc6)nc(-c6ccccc6)n5)c4)c3cc21] | TMM-1 |
| 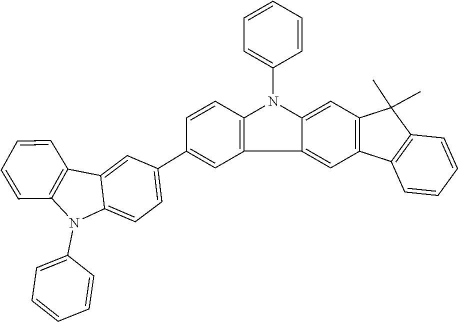 [SMILES: CC1(C)c2ccccc2-c2cc3c4cc(-c5ccc6c(c5)c5ccccc5n6-c5ccccc5)ccc4n(-c4ccccc4)c3cc21] | TMM-2 |
| 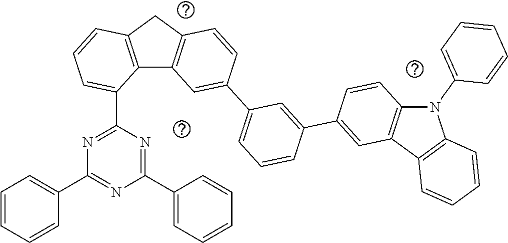 [SMILES: c1ccc(-c2nc(-c3ccccc3)nc(-c3cccc4c3-c3cc(-c5cccc(-c6ccc7c(c6)c6ccccc6n7-c6ccccc6)c5)ccc3C4)n2)cc1] | TMM-A |
| 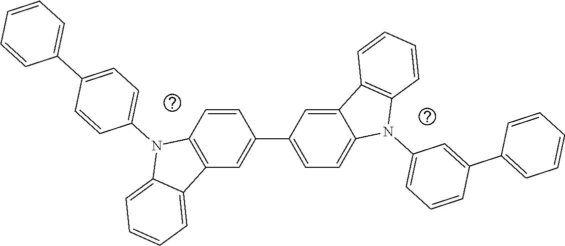 [SMILES: c1ccc(-c2ccc(-n3c4ccccc4c4cc(-c5ccc6c(c5)c5ccccc5n6-c5cccc(-c6ccccc6)c5)ccc43)cc2)cc1] | TMM-B |
| 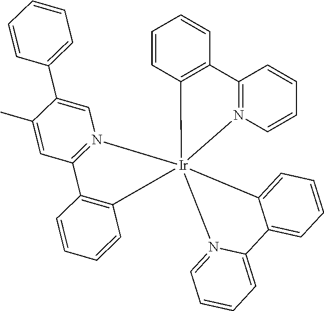 | TEG |
| 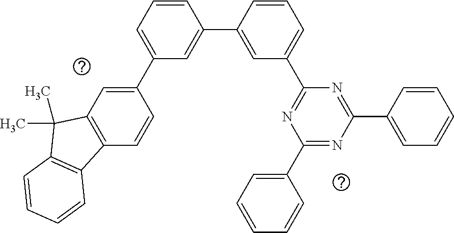 [SMILES: CC1(C)c2ccccc2-c2ccc(-c3cccc(-c4cccc(-c5nc(-c6ccccc6)nc(-c6ccccc6)n5)c4)c3)cc21] | HBM-A |
| 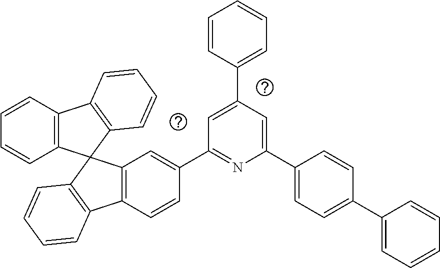 [SMILES: c1ccc(-c2ccc(-c3cc(-c4ccccc4)cc(-c4ccc5c(c4)C4(c6ccccc6-c6ccccc64)c4ccccc4-5)n3)cc2)cc1] | ETM-A |
| 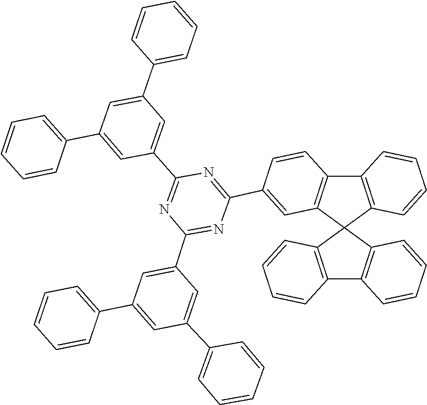 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1] | ETM |
| 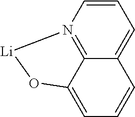 | LiQ |

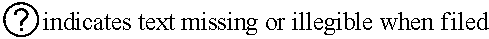

The invention claimed is:

1.-26. (canceled)

27. A compound of formula (I) 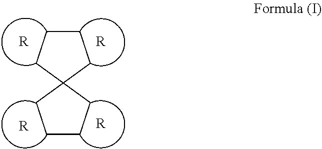
where the units R are the same or different at each instance and are selected from units of the formulae (R-1) to (R-3) 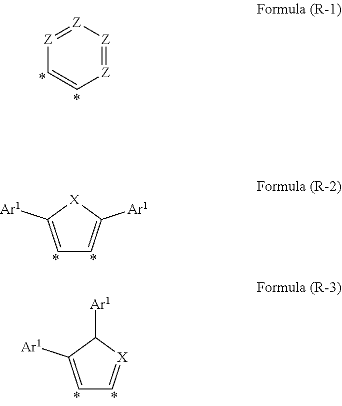
and where:
the units of the formulae (R-1) to (R-3) are each bonded to the remainder of the formula (I) via the positions identified by *;
at least one unit R in formula (I) is selected from units of the formulae (R-2) and (R-3);
X is the same or different at each instance and is selected from O, S and NAr0;
Z is the same or different at each instance and is selected from N and CR¹;
Ar⁰ is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar¹ is the same or different at each instance and is selected from H, D, F, Cl, Br, I, C(═O)R², CN, Si(R²)₃, P(═O)(R²)₂, OR², S(═O)R², S(═O)₂R², straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where the alkyl, alkoxy, alkenyl and alkynyl groups mentioned and the aromatic ring systems and heteroaromatic ring systems mentioned are each substituted by one or more R² radicals; and where one or more CH₂ groups in the alkyl, alkoxy, alkenyl and alkynyl groups mentioned may be replaced by —R²C═CR²—, —C≡C—, Si(R²)₂, C═O, C═NR², —C(═O)O—, —C(═O)NR²—, NR², P(═O)(R²), —O—, —S—, SO or SO₂;
R¹ is the same or different at each instance and is selected from H, D, F, Cl, Br, I, C(═O)R⁵, CN, Si(R⁵)₃, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R¹ radicals may be joined to one another and may form an aliphatic or heteroaliphatic ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, SO or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN; and at least one A group conforming to a formula (A) is bonded to at least one unit R, either directly to the ring of the unit R or to an Ar⁰ or Ar¹ group: 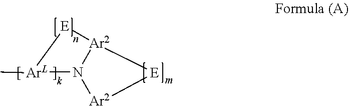
where:
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar² is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals;
E is the same or different at each instance and is a single bond or a divalent group selected from C(R⁴)₂, Si(R⁴)₂, N(R⁴), O, and S;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁴ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁴ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
k is 0 or 1, where, in the case that k=0, the Ar^{L} group is absent and the nitrogen atom of the group of the formula (A) constitutes the attachment position, and at the same time n=0; and
m is 0 or 1, where, in the case that m=0, the E group in question is absent and the Ar² groups are not bonded to one another;
n is 0 or 1, where, in the case that n=0, the E group in question is absent and the Ar^{L} and Ar² groups are not bonded to one another.

28. The compound according to claim 27, wherein exactly one unit R in formula (I) is selected from units of the formulae (R-2) and (R-3), and the remaining three units R conform to the formula (R-1).

29. The compound according to claim 27, wherein exactly one or exactly two unit(s) R in formula (I) conform to the formula (R-2), and the other units R conform to the formula (R-1).

30. The compound according to claim 27, wherein X is S or 0.

31. The compound according to o claim 27, wherein the Z groups are CR¹.

32. The compound according to o claim 27, wherein Ar¹ is phenyl substituted by R² radicals.

33. The compound according to claim 27, wherein R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals.

34. The compound according to claim 27, wherein R² is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals.

35. The compound according to claim 27, wherein R³ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals.

36. The compound according to claim 27, wherein R⁴ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals.

37. The compound according to claim 27, wherein R⁵ is the same or different at each instance and is selected from H, D, Si(R⁶)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁶ radicals.

38. The compound according to claim 27, wherein Ar^{L} is the same or different at each instance and is selected from divalent groups derived from benzene, biphenyl, terphenyl, naphthalene, fluorene, indenofluorene, indenocarbazole, spirobifluorene, dibenzofuran, dibenzothiophene, and carbazole, each of which may be substituted by R² radicals.

39. The compound according to claim 27, wherein Ar² is the same or different at each instance and is selected from monovalent groups derived from benzene, biphenyl, terphenyl, quaterphenyl, naphthalene, fluorene, especially 9,9′-dimethylfluorene and 9,9′-diphenylfluorene, 9-silafluorene, especially 9,9′-dimethyl-9-silafluorene and 9,9′-diphenyl-9-silafluorene, benzofluorene, spirobifluorene, indenofluorene, indenocarbazole, dibenzofuran, dibenzothiophene, benzocarbazole, carbazole, benzofuran, benzothiophene, indole, quinoline, pyridine, pyrimidine, pyrazine, pyridazine and triazine, where the monovalent groups are each substituted by one or more R³ radicals.

40. The compound according to claim 27, wherein n and m are 0.

41. The compound according to claim 27, wherein it conforms to one of the formulae (I-a) to (I-f) 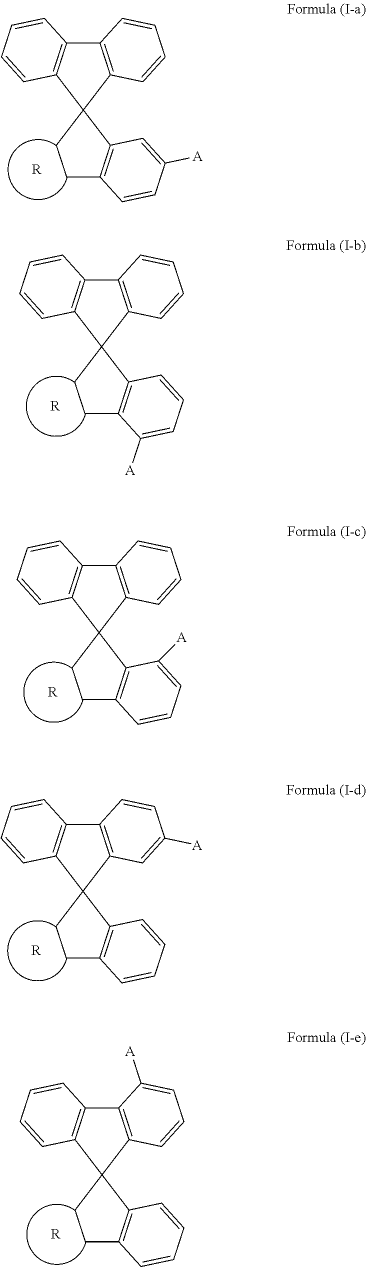 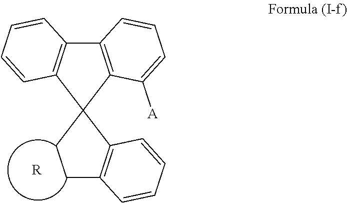
where the unit R is selected from formulae (R-2) and (R-3), as defined in claim 27, and where R¹ radicals are bonded to all unoccupied positions on the rings.

42. The compound according to o claim 27, wherein it conforms to one of the following formulae: 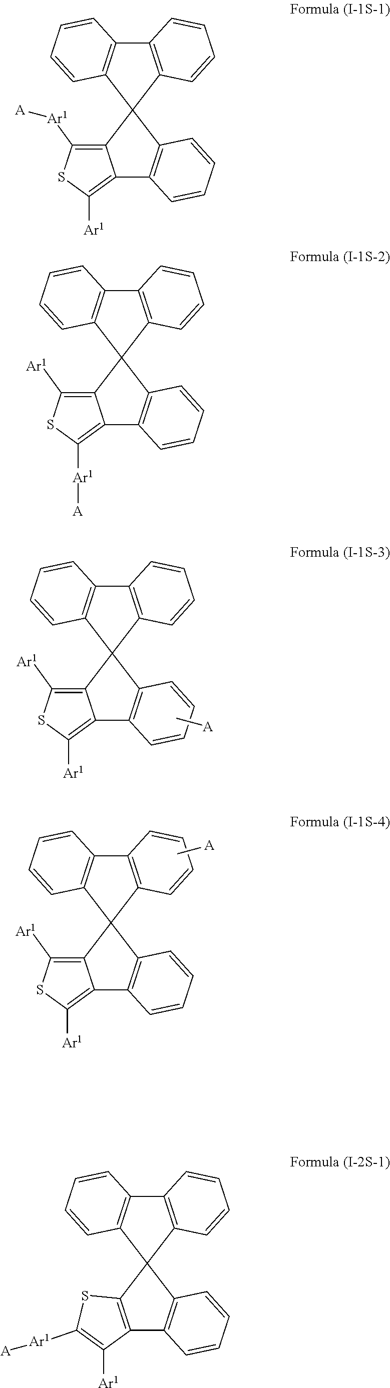 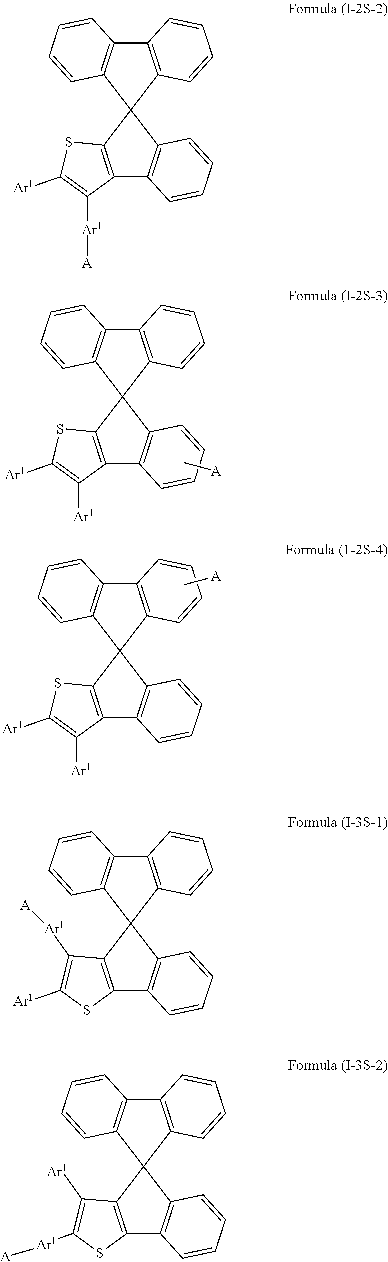 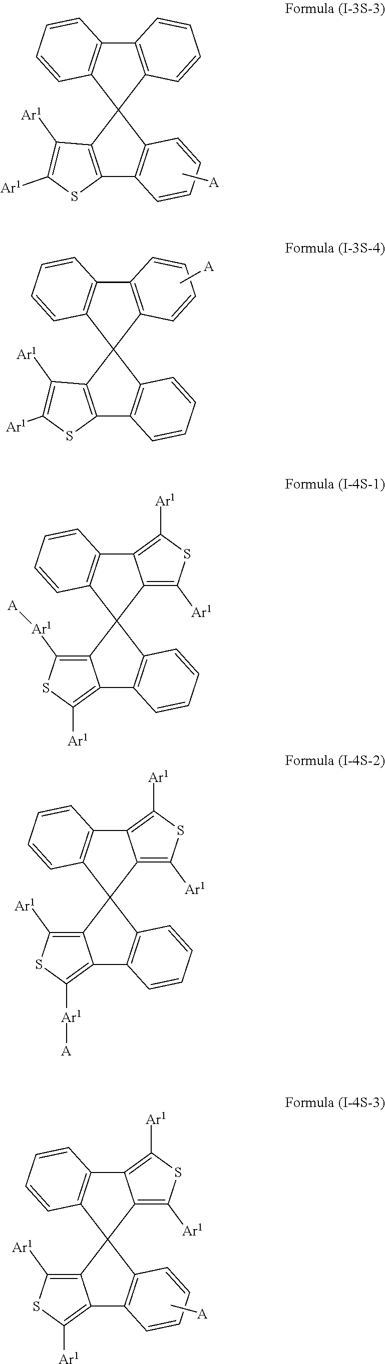 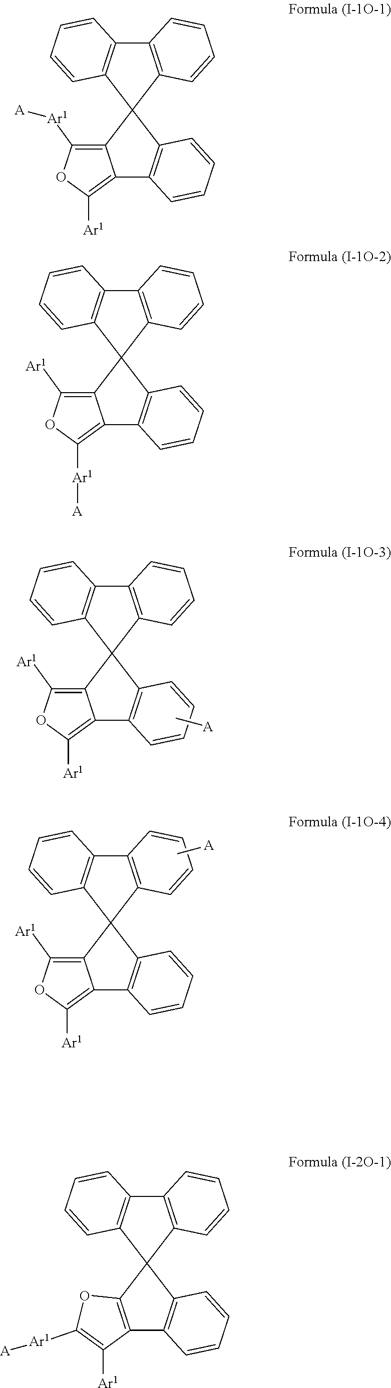 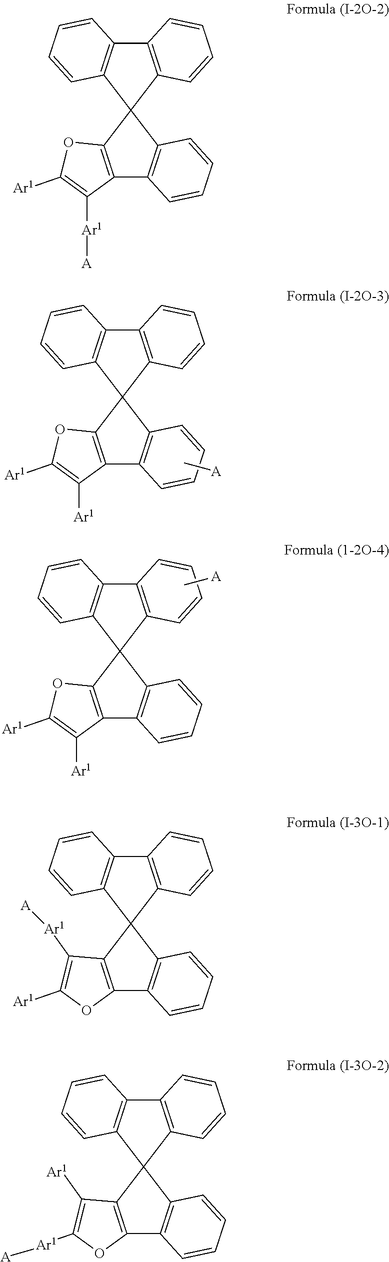 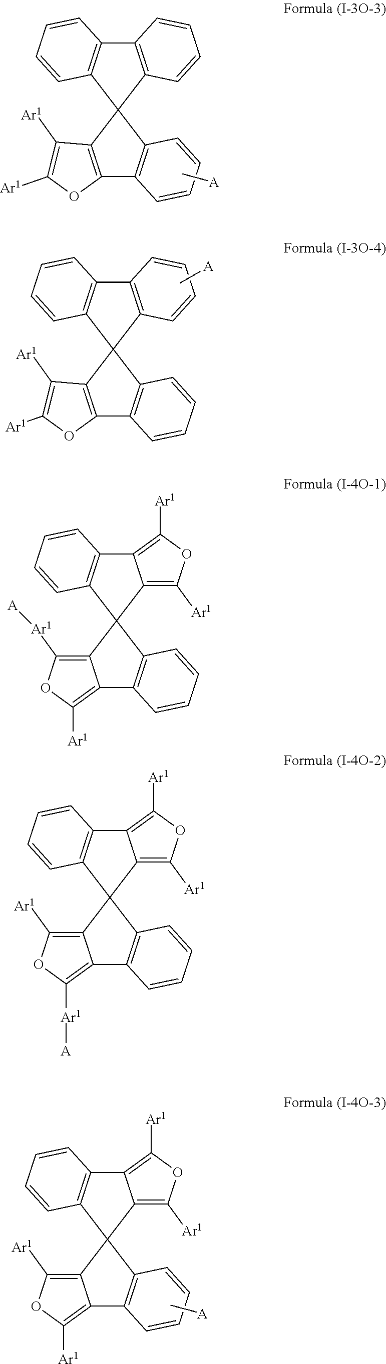 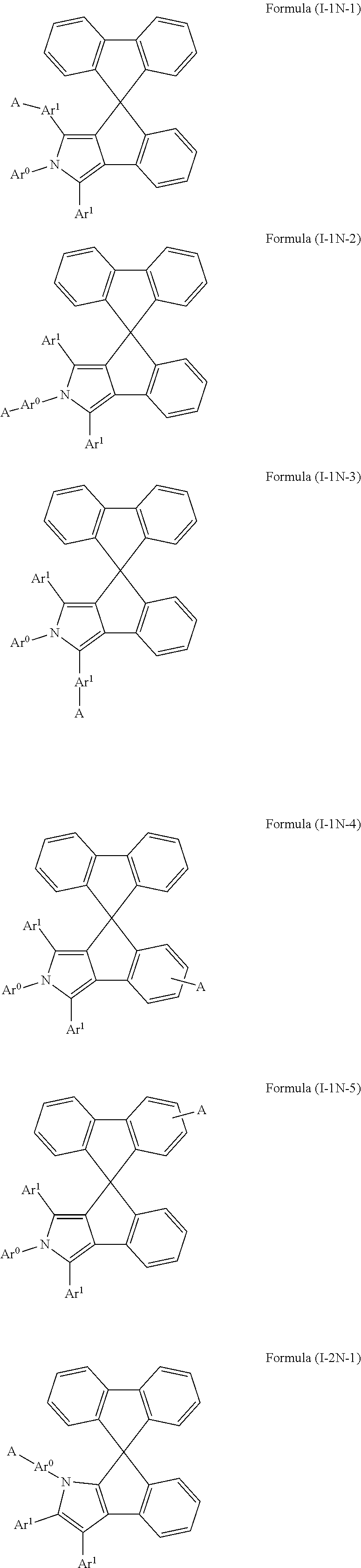 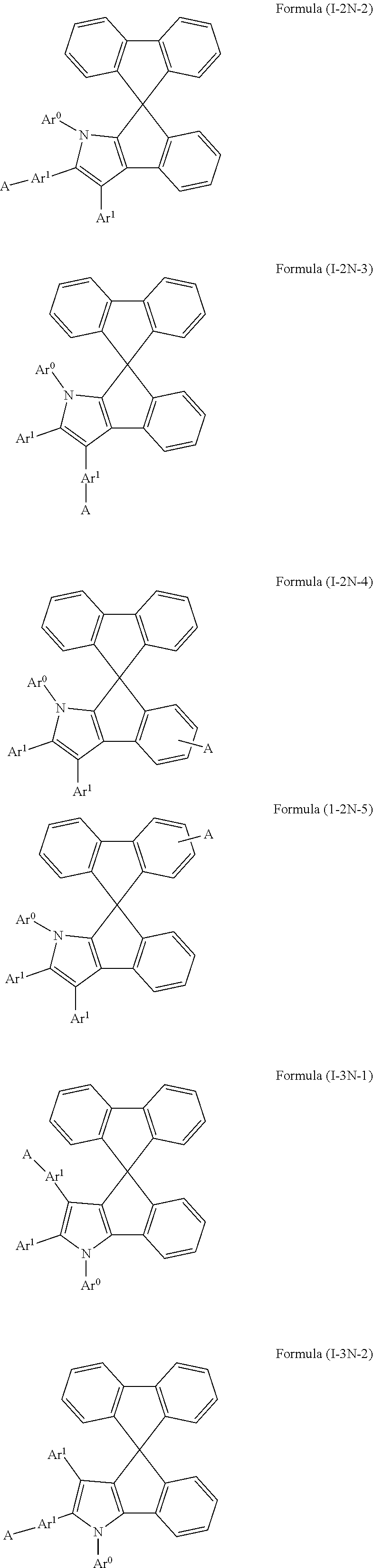 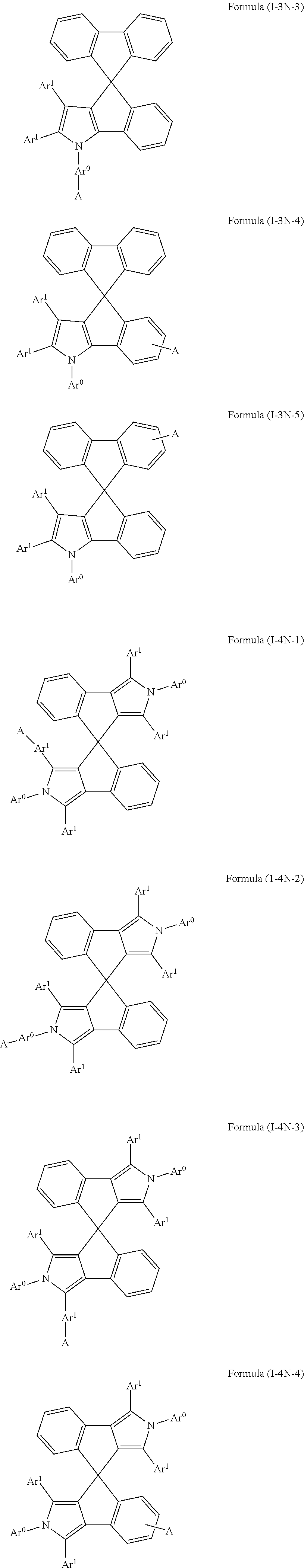
where the variables are as defined in claim 27.

43. The compound according to o claim 27, wherein it conforms to a formula selected from formulae (I-1S-1), (I-1S-2), (I-1S-3-1), (I-1S-3-2), (I-1S-3-3), (I-1S-4-1), (I-11S-4-2), (I-11S-4-3), (I-1O-1), (I-1O-2), (I-1O-3-1), (I-1O-3-2), (I-1O-3-3), (I-1O-4-1), (I-1O-4-2) and (I-1O-4-3) according to claim 42.

44. The compound according to claim 27, wherein the Z groups are CR¹1, Ar¹ is phenyl substituted by R² radicals, R¹ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, R² is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, R³ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals, R⁴ is the same or different at each instance and is selected from H, D, Si(R⁵)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to 40 aromatic ring atoms and may be deuterated, where the alkyl groups mentioned, the aromatic ring systems mentioned and the heteroaromatic ring systems mentioned are each substituted by R⁵ radicals and R⁵ is the same or different at each instance and is selected from H, D, Si(R⁶)₃, straight-chain alkyl groups which have 1 to 20 carbon atoms and may be deuterated, branched or cyclic alkyl groups which have 3 to 20 carbon atoms and may be deuterated, aromatic ring systems which have 6 to 40 aromatic ring atoms and may be deuterated, and heteroaromatic ring systems which have 5 to aromatic ring atoms and may be deuterated, where the alkyl groups mentioned, the aromatic ring systems mentioned and the heteroaromatic ring systems mentioned are each substituted by R⁶ radicals.

45. A compound of a formula (Int-4), (Int-4b), (Int-4c) or (Int-5) 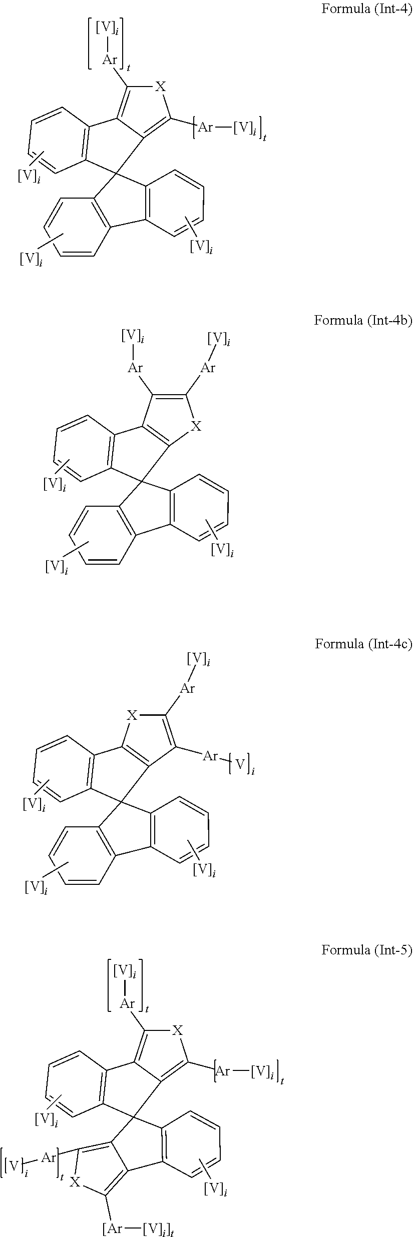
where t=0 or 1, i is 0, 1 or 2, where at least one i is 1, and where, in addition:
V is the same or different at each instance and is selected from reactive groups, preferably Cl, Br or I;
Ar is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
where the compounds are each substituted by an R¹ radical at the unoccupied positions on the benzene ring, and
X is the same or different at each instance and is selected from O, S and NAr0;
Z is the same or different at each instance and is selected from N and CR¹;
Ar⁰ is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals; R² is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R² radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂;
R⁵ is the same or different at each instance and is selected from H, D, F, Cl, Br, I, C(═O)R⁶, CN, Si(R⁶)₃, N(R⁶)₂, P(═O)(R⁶)₂, OR⁶, S(═O)R⁶, S(═O)₂R⁶, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R⁵ radicals may be joined to one another and may form a ring; where the alkyl, alkoxy, alkenyl and alkynyl groups mentioned and the aromatic ring systems and heteroaromatic ring systems mentioned are each substituted by R⁶ radicals; and where one or more CH₂ groups in the alkyl, alkoxy, alkenyl and alkynyl groups mentioned may be replaced by —R⁶C═CR⁶—, —C≡C—, Si(R⁶)₂, C═O, C═NR⁶, —C(═O)O—, —C(═O)NR⁶—, NR⁶, P(═O)(R⁶), —O—, —S—, so or SO₂;
R⁶ is the same or different at each instance and is selected from H, D, F, Cl, Br, I, CN, alkyl or alkoxy groups having 1 to 20 carbon atoms, alkenyl or alkynyl groups having 2 to 20 carbon atoms, aromatic ring systems having 6 to 40 aromatic ring atoms and heteroaromatic ring systems having 5 to 40 aromatic ring atoms; where two or more R⁶ radicals may be joined to one another and may form a ring; and where the alkyl, alkoxy, alkenyl and alkynyl groups, aromatic ring systems and heteroaromatic ring systems mentioned may be substituted by one or more radicals selected from F and CN; and at least one A group conforming to a formula (A) is bonded to at least one unit R, either directly to the ring of the unit R or to an Ar⁰ or Ar¹ group: 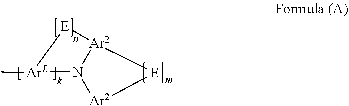
where:
Ar^{L} is the same or different at each instance and is selected from aromatic ring systems which have 6 to 40 aromatic ring atoms and are substituted by R² radicals, and heteroaromatic ring systems which have 5 to 40 aromatic ring atoms and are substituted by R² radicals;
Ar² is the same or different at each instance and is selected from aromatic ring systems which have 6 to 40 aromatic ring atoms and are substituted by R³ radicals, and heteroaromatic ring systems which have 5 to 40 aromatic ring atoms and are substituted by R³ radicals;
E is the same or different at each instance and is a single bond or a divalent group selected from C(R⁴)₂, Si(R⁴)₂, N(R⁴), O, and S;
R³ is the same or different at each instance and is selected from H, D, F, Cl, Br, I, C(═O)R⁵, CN, Si(R⁵)₃, N(R⁵)₂, P(═O)(R⁵)₂, OR⁵, S(═O)R⁵, S(═O)₂R⁵, straight-chain alkyl or alkoxy groups having 1 to 20 carbon atoms, branched or cyclic alkyl or alkoxy groups having 3 to 20 carbon atoms, alkenyl or alkynyl groups having 2 to 20 carbon atoms, aromatic ring systems having 6 to 40 aromatic ring atoms, and heteroaromatic ring systems having 5 to 40 aromatic ring atoms; where two or more R³ radicals may be joined to one another and may form a ring; where the alkyl, alkoxy, alkenyl and alkynyl groups mentioned and the aromatic ring systems and heteroaromatic ring systems mentioned are each substituted by R⁵ radicals; and where one or more CH₂ groups in the alkyl, alkoxy, alkenyl and alkynyl groups mentioned may be replaced by —R⁵C═CR⁵—, —C≡C—, Si(R⁵)₂, C═O, C═NR⁵, —C(═O)O—, —C(═O)NR⁵—, NR⁵, P(═O)(R⁵), —O—, —S—, SO or SO₂.

46. A process for preparing a compound according to claim 27, wherein
in a first step via a ring closure reaction, by action of acid, a compound (Int-1) is converted to a compound (Int-2), and further wherein, in a further step, an ortho-metallated bisaryl is added on and a further ring closure reaction is conducted, forming a compound (Int-4) or (Int-5), and further wherein, in a further step, a Suzuki coupling or a Hartwig-Buchwald coupling is conducted, giving a compound of a formula (I), or
in a first step via a ring closure reaction, by action of acid, a compound (Int-1b) is converted to a compound (Int-3b) or a compound (Int-1c) to a compound (Int-3c), and further wherein, in a further step, an ortho-metallated bisaryl is added on and a further ring closure reaction is conducted, forming a compound (Int-4b) from the compound (Int-3b) and a compound (Int-4c) from the compound (Int-3c), and further wherein, in a further step, a Suzuki coupling or a Hartwig-Buchwald coupling is conducted, giving a compound of a formula (I).

47. The process for preparing a compound according to claim 27, wherein, in a first step, an ortho-metallated heteroaryl-aryl derivative (Int-A1), (Int-A2) or (Int-A3) is reacted with a fluorenone derivative and a ring closure reaction is conducted, forming a compound selected from compounds of the formulae (Int-4), (Int-4b) and (Int-4c), and further wherein, in a further step, a Suzuki coupling or a Hartwig-Buchwald coupling is conducted, giving a compound of a formula (I).

48. An oligomer, polymer or dendrimer containing one or more compounds according to claim 27, wherein the bond(s) to the polymer, oligomer or dendrimer may be localized at any desired positions substituted by R¹, R², R³ or R⁴ in formula (I).

49. A formulation comprising at least one compound according to claim 27 and at least one solvent.

50. An electronic device comprising at least one compound according to claim 27.

51. The electronic device according to claim 50, wherein it is an organic electroluminescent device and comprises anode, cathode and at least one emitting layer, and in that the compound is present in a hole-transporting layer or in an emitting layer of the device.

52. A method comprising utilizing the compound according to claim 27 in an electronic device.
